SEQUENCE LISTING (738 sequences)

SEQ ID NO: 1 - protein, 28 aa, Artificial
  Synthetic Construct
HSDAVFTDNYTRLRKQMAVKKYLNSILN

SEQ ID NO: 2 - protein, 27 aa, Artificial
  Synthetic Construct
HSDGIFTDSYSRYRKQMAVKKYLAAVL

SEQ ID NO: 3 - protein, 37 aa, Artificial
  Synthetic Construct
HSDGIFTDSYSRYRKQMAVKKYLAAVLGKRYQRVKNK

SEQ ID NO: 4 - protein, 31 aa, Artificial
  Synthetic Construct
HXDAVFTDNYTXLXXXXAXXXYLXXXXNXXX

SEQ ID NO: 5 - protein, 31 aa, Artificial
  Synthetic Construct
XXDXXXTXNXTXXXXXXAXXXXLXXXXXXXX

SEQ ID NO: 6 - protein, 40 aa, Artificial
  Synthetic Construct
HXXAVFTXXYTXLRXXXAXXXYLXXXXXXXXXXXXXXXXX

SEQ ID NO: 7 - protein, 40 aa, Artificial
  Synthetic Construct
XXXXXXTXXXTXXXXXXAXXXXLXXXXXXXXXXXXXXXXX

SEQ ID NO: 8 - protein, 13 aa, Artificial
  Synthetic Construct
XXXXXXXXXXXXX

SEQ ID NO: 9 - protein, 11 aa, Artificial
  Synthetic Construct
XXXXXXXXXXX

SEQ ID NO: 10 - protein, 11 aa, Artificial
  Synthetic Construct
GGPSSGAPPPS

SEQ ID NO: 11 - protein, 11 aa, Artificial
  Synthetic Construct
GGPSSGAPPPS

SEQ ID NO: 12 - protein, 10 aa, Artificial
  Synthetic Construct
GGPSSGAPPS

SEQ ID NO: 13 - protein, 11 aa, Artificial
  Synthetic Construct
GGPSSGAPPPS

SEQ ID NO: 14 - protein, 9 aa, Artificial
  Synthetic Construct
SWCEPGWCR

SEQ ID NO: 15 - protein, 11 aa, Artificial
  Synthetic Construct
XXXXXXXXXXX

SEQ ID NO: 16 - protein, 11 aa, Artificial
  Synthetic Construct
GRPSSGAPPPS

SEQ ID NO: 17 - protein, 11 aa, Artificial
  Synthetic Construct
GRPSSGAPPPS

SEQ ID NO: 18 - protein, 40 aa, Artificial
  Synthetic Construct
XXXXXXTXXXTXXXXXXXXXXXXXXXXXXXXXXXXXXXXX

SEQ ID NO: 19 - protein, 13 aa, Artificial
  Synthetic Construct
XXXXXXXXXXXXX

SEQ ID NO: 20 - protein, 32 aa, Artificial
  Synthetic Construct
XXXXXXTXXXTXXXXXXXXXXXXXXXXXXXXX

SEQ ID NO: 21 - protein, 31 aa, Artificial
  Synthetic Construct
HXXXXFTXXXTXXXXXXXXXXXXXXXXXXXX

SEQ ID NO: 22 - protein, 11 aa, Artificial
  Synthetic Construct
GGPSSGAPPPC

SEQ ID NO: 23 - protein, 11 aa, Artificial
  Synthetic Construct
GGPSSGAPPPC

SEQ ID NO: 24 - protein, 39 aa, Artificial
  Synthetic Construct
HSDAVFTDNYTRLRRQVAARKYLQSIRNGGPSSGAPPPS

SEQ ID NO: 25 - protein, 39 aa, Artificial
  Synthetic Construct
HSDAVFTENYTKLRKQXAAKKYLNDLKKGGPSSGAPPPC

SEQ ID NO: 26 - protein, 40 aa, Artificial; identical to SEQ ID NO: 18
  Synthetic Construct

SEQ ID NO: 27 - protein, 40 aa, Artificial
  Synthetic Construct
HXXXXFTXXXTXXXXXXXXXXXXXXXXXXXXXXXXXXXXX

SEQ ID NO: 28 - protein, 32 aa, Artificial
  Synthetic Construct
HSXAVFTXXXTXXXXXXAXXXXLXXXXXXXXX

SEQ ID NO: 29 - protein, 13 aa, Artificial
  Synthetic Construct
XXXXXXXXXXXXX

SEQ ID NO: 30 - protein, 42 aa, Artificial
  Synthetic Construct
HSDAVFTDNYTRLRKQVAAKKYLQSIKNKRYGGPSSGAPPPS

SEQ ID NO: 31 - protein, 33 aa, Artificial
  Synthetic Construct
HSDAVFTDNYTRLRKQVAAKKYLQSIKNKRGGT

SEQ ID NO: 32 - protein, 33 aa, Artificial
  Synthetic Construct
HSDAVFTDNYTRLRKQVAAKKYLQSIKNKKGGT

SEQ ID NO: 33 - protein, 41 aa, Artificial
  Synthetic Construct
HSDAVFTDNYTRLRKQVAAKKYLQSIKNKKGGPSSGAPPPS

SEQ ID NO: 34 - protein, 42 aa, Artificial
  Synthetic Construct
HSDAVFTDNYTRLRKQVAAXKYLQSIKNKRYGGPSSGAPPPS

SEQ ID NO: 35 - protein, 42 aa, Artificial
  Synthetic Construct
HSDAVFTDNYTRLRKQVAAIKYLQSIKNKRYGGPSSGAPPPS

SEQ ID NO: 36 - protein, 42 aa, Artificial
  Synthetic Construct
HSDAVFTDNYTRLRKQVAARKYLQSIKNKRYGGPSSGAPPPS

SEQ ID NO: 37 - protein, 42 aa, Artificial
  Synthetic Construct
HSDAVFTDNYTRLRKQVAASKYLQSIKNKRYGGPSSGAPPPS

SEQ ID NO: 38 - protein, 42 aa, Artificial
  Synthetic Construct
HSDAVFTDNYTRLRKQVAAKKYLQSIXNKRYGGPSSGAPPPS

SEQ ID NO: 39 - protein, 42 aa, Artificial
  Synthetic Construct
HSDAVFTDNYTRLRKQVAAKKYLQSIRNKRYGGPSSGAPPPS

SEQ ID NO: 40 - protein, 42 aa, Artificial
  Synthetic Construct
HSDAVFTDNYTRLRKQVAAKKYLQSIKNXRYGGPSSGAPPPS

SEQ ID NO: 41 - protein, 42 aa, Artificial
  Synthetic Construct
HSDAVFTDNYTRLRKQVAAKKYLQSIKNRRYGGPSSGAPPPS

SEQ ID NO: 42 - protein, 42 aa, Artificial
  Synthetic Construct
HSDAVFTDNYTRFRKQVAAKKYLQSIKNKRYGGPSSGAPPPS

SEQ ID NO: 43 - protein, 42 aa, Artificial
  Synthetic Construct
HSDAVFTDNWTRLRKQVAAKKYLQSIKNKRYGGPSSGAPPPS

SEQ ID NO: 44 - protein, 42 aa, Artificial
  Synthetic Construct
HSDAVFTDNYTRLRKQVAAKKYLQSIKNKRHGGPSSGAPPPS

SEQ ID NO: 45 - protein, 42 aa, Artificial
  Synthetic Construct
HSDAVFTDNYTRLRKQVAAKKYLQSIKNKRQGGPSSGAPPPS

SEQ ID NO: 46 - protein, 42 aa, Artificial; identical to SEQ ID NO: 30
  Synthetic Construct

SEQ ID NO: 47 - protein, 42 aa, Artificial; identical to SEQ ID NO: 30
  Synthetic Construct

SEQ ID NO: 48 - protein, 31 aa, Artificial
  Synthetic Construct
HSDAVFTDNYTRLRKQVAAKKYLQSIKNKRF

SEQ ID NO: 49 - protein, 32 aa, Artificial
  Synthetic Construct
HSDAVFTDNYTRLRKQVAAKKYLQSIKNKRYC

SEQ ID NO: 50 - protein, 42 aa, Artificial
  Synthetic Construct
HGDAVFTDNYTRLRKQVAAKKYLQSIKNKRYGGPSSGAPPPS

SEQ ID NO: 51 - protein, 42 aa, Artificial
  Synthetic Construct
HVDAVFTDNYTRLRKQVAAKKYLQSIKNKRYGGPSSGAPPPS

SEQ ID NO: 52 - protein, 42 aa, Artificial
  Synthetic Construct
HTDAVFTDNYTRLRKQVAAKKYLQSIKNKRYGGPSSGAPPPS

SEQ ID NO: 53 - protein, 42 aa, Artificial
  Synthetic Construct
HLDAVFTDNYTRLRKQVAAKKYLQSIKNKRYGGPSSGAPPPS

SEQ ID NO: 54 - protein, 42 aa, Artificial
  Synthetic Construct
HADAVFTDNYTRLRKQVAAKKYLQSIKNKRYGGPSSGAPPPS

SEQ ID NO: 55 - protein, 42 aa, Artificial; identical to SEQ ID NO: 30
  Synthetic Construct

SEQ ID NO: 56 - protein, 42 aa, Artificial
  Synthetic Construct
HPDAVFTDNYTRLRKQVAAKKYLQSIKNKRYGGPSSGAPPPS

SEQ ID NO: 57 - protein, 42 aa, Artificial
  Synthetic Construct
HSDIVFTDNYTRLRKQVAAKKYLQSIKNKRYGGPSSGAPPPS

SEQ ID NO: 58 - protein, 42 aa, Artificial
  Synthetic Construct
HSDYVFTDNYTRLRKQVAAKKYLQSIKNKRYGGPSSGAPPPS

SEQ ID NO: 59 - protein, 42 aa, Artificial
  Synthetic Construct
HSDFVFTDNYTRLRKQVAAKKYLQSIKNKRYGGPSSGAPPPS

SEQ ID NO: 60 - protein, 42 aa, Artificial
  Synthetic Construct
HSDVVFTDNYTRLRKQVAAKKYLQSIKNKRYGGPSSGAPPPS

SEQ ID NO: 61 - protein, 42 aa, Artificial
  Synthetic Construct
HSDTVFTDNYTRLRKQVAAKKYLQSIKNKRYGGPSSGAPPPS

SEQ ID NO: 62 - protein, 42 aa, Artificial
  Synthetic Construct
HSDLVFTDNYTRLRKQVAAKKYLQSIKNKRYGGPSSGAPPPS

SEQ ID NO: 63 - protein, 42 aa, Artificial
  Synthetic Construct
HSDWVFTDNYTRLRKQVAAKKYLQSIKNKRYGGPSSGAPPPS

SEQ ID NO: 64 - protein, 42 aa, Artificial
  Synthetic Construct
HSDAFFTDNYTRLRKQVAAKKYLQSIKNKRYGGPSSGAPPPS

SEQ ID NO: 65 - protein, 42 aa, Artificial
  Synthetic Construct
HSDAIFTDNYTRLRKQVAAKKYLQSIKNKRYGGPSSGAPPPS

SEQ ID NO: 66 - protein, 42 aa, Artificial
  Synthetic Construct
HSDALFTDNYTRLRKQVAAKKYLQSIKNKRYGGPSSGAPPPS

SEQ ID NO: 67 - protein, 42 aa, Artificial
  Synthetic Construct
HSDATFTDNYTRLRKQVAAKKYLQSIKNKRYGGPSSGAPPPS

SEQ ID NO: 68 - protein, 42 aa, Artificial
  Synthetic Construct
HSDAVITDNYTRLRKQVAAKKYLQSIKNKRYGGPSSGAPPPS

SEQ ID NO: 69 - protein, 42 aa, Artificial
  Synthetic Construct
HSDAVLTDNYTRLRKQVAAKKYLQSIKNKRYGGPSSGAPPPS

SEQ ID NO: 70 - protein, 42 aa, Artificial
  Synthetic Construct
HSDAVTTDNYTRLRKQVAAKKYLQSIKNKRYGGPSSGAPPPS

SEQ ID NO: 71 - protein, 42 aa, Artificial
  Synthetic Construct
HSDAVVTDNYTRLRKQVAAKKYLQSIKNKRYGGPSSGAPPPS

SEQ ID NO: 72 - protein, 42 aa, Artificial
  Synthetic Construct
HSDAVWTDNYTRLRKQVAAKKYLQSIKNKRYGGPSSGAPPPS

SEQ ID NO: 73 - protein, 42 aa, Artificial
  Synthetic Construct
HSDAVYTDNYTRLRKQVAAKKYLQSIKNKRYGGPSSGAPPPS

SEQ ID NO: 74 - protein, 42 aa, Artificial
  Synthetic Construct
HSDAWFTDNYTRLRKQVAAKKYLQSIKNKRYGGPSSGAPPPS

SEQ ID NO: 75 - protein, 42 aa, Artificial
  Synthetic Construct
HSDAYFTDNYTRLRKQVAAKKYLQSIKNKRYGGPSSGAPPPS

SEQ ID NO: 76 - protein, 42 aa, Artificial
  Synthetic Construct
HSDAVFTDNYTRLRRQVAARRYLQSIRNRRYGGPSSGAPPPS

SEQ ID NO: 77 - protein, 42 aa, Artificial
  Synthetic Construct
HSDAVFTDNYTRLRRQVAAKKYLQSIKNKRYGGPSSGAPPPS

SEQ ID NO: 78 - protein, 42 aa, Artificial; identical to SEQ ID NO: 36
  Synthetic Construct

SEQ ID NO: 79 - protein, 42 aa, Artificial
  Synthetic Construct
HSDAVFTDNYTRLRKQVAAKKYLQSIQNKRYGGPSSGAPPPS

SEQ ID NO: 80 - protein, 42 aa, Artificial
  Synthetic Construct
HSDAVFTDNYTRLRKQVAAKKYLQSIKNNRYGGPSSGAPPPS

SEQ ID NO: 81 - protein, 42 aa, Artificial
  Synthetic Construct
HSDAVFTDNYTRLRKQVAAKKYLQSIKNKIYGGPSSGAPPPS

SEQ ID NO: 82 - protein, 39 aa, Artificial
  Synthetic Construct
HSDAVFTDNYTRLRKQVAAKKYLQSIKRGGPSSGAPPPS

SEQ ID NO: 83 - protein, 39 aa, Artificial
  Synthetic Construct
HSDAVFTDNYTRLRKQVAAKKYLQSIKNGGPSSGAPPPS

SEQ ID NO: 84 - protein, 42 aa, Artificial; identical to SEQ ID NO: 30
  Synthetic Construct

SEQ ID NO: 85 - protein, 39 aa, Artificial
  Synthetic Construct
HSDAVFTDNYTRLRKQVAAKKYLQSIKKGGPSSGAPPPS

SEQ ID NO: 86 - protein, 42 aa, Artificial
  Synthetic Construct
HSDAVFTDNYTREKEKVAAKKYLQSIKNKRYGGPSSGAPPPS

SEQ ID NO: 87 - protein, 42 aa, Artificial
  Synthetic Construct
HSDAVFTDNYTRAAAKVAAKKYLQSIKNKRYGGPSSGAPPPS

SEQ ID NO: 88 - protein, 42 aa, Artificial
  Synthetic Construct
HSDAVFTDNYTRLLAKVAAKKYLQSIKNKRYGGPSSGAPPPS

SEQ ID NO: 89 - protein, 39 aa, Artificial
  Synthetic Construct
HSDAVFTDNYTRLRKQVAAKKYLQSIKNGRPSSGAPPPS

SEQ ID NO: 90 - protein, 42 aa, Artificial
  Synthetic Construct
HSDAVFTDNYTRLLLKVAAKKYLQSIKNKRYGGPSSGAPPPS

SEQ ID NO: 91 - protein, 42 aa, Artificial
  Synthetic Construct
HSDAVFTDNYTRAKAKVAAKKYLQSIKNKRYGGPSSGAPPPS

SEQ ID NO: 92 - protein, 42 aa, Artificial; identical to SEQ ID NO: 76
  Synthetic Construct

SEQ ID NO: 93 - protein, 42 aa, Artificial; identical to SEQ ID NO: 51
  Synthetic Construct

SEQ ID NO: 94 - protein, 43 aa, Artificial
  Synthetic Construct
MHSDAVFTDQYTRLRKQVAAKKYLQSIKQKRYGGPSSGAPPPS

SEQ ID NO: 95 - protein, 42 aa, Artificial; identical to SEQ ID NO: 54
  Synthetic Construct

SEQ ID NO: 96 - protein, 42 aa, Artificial
  Synthetic Construct
HSDGVFTDNYTRLRKQVAAKKYLQSIKNKRYGGPSSGAPPPS

SEQ ID NO: 97 - protein, 42 aa, Artificial
  Synthetic Construct
HSDAVFTDNYTKLKKQVAAKKYLQSIKNKKYGGPSSGAPPPS

SEQ ID NO: 98 - protein, 43 aa, Artificial
  Synthetic Construct
MHSDAVFTDNYTRLRKQVAAKKYLQSIKNKRYGGPSSGAPPPS

SEQ ID NO: 99 - protein, 43 aa, Artificial
  Synthetic Construct
IHSDAVFTDNYTRLRKQVAAKKYLQSIKNKRYGGPSSGAPPPS

SEQ ID NO: 100 - protein, 42 aa, Artificial
  Synthetic Construct
HSDAVFTDNYTRLRKQVAAKKYLQSFKNKRYGGPSSGAPPPS

SEQ ID NO: 101 - protein, 42 aa, Artificial
  Synthetic Construct
HSDAVFTDNYTRLRKQVAAKKYLQSLKNKRYGGPSSGAPPPS

SEQ ID NO: 102 - protein, 42 aa, Artificial
  Synthetic Construct
HSDAVFTDNYTRLRKQVAAKKYLQSTKNKRYGGPSSGAPPPS

SEQ ID NO: 103 - protein, 42 aa, Artificial
  Synthetic Construct
HSDAVFTDNYTRLRKQVAAKKYLQSVKNKRYGGPSSGAPPPS

SEQ ID NO: 104 - protein, 42 aa, Artificial
  Synthetic Construct
HSDAVFTDNYTRLRKQVAAKKYLQSWKNKRYGGPSSGAPPPS

SEQ ID NO: 105 - protein, 42 aa, Artificial
  Synthetic Construct
HSDAVFTDNYTRLRKQVAAKKYLQSYKNKRYGGPSSGAPPPS

SEQ ID NO: 106 - protein, 42 aa, Artificial
  Synthetic Construct
HSDAVFTDNYTRLRKQVAAKKYLQFIKNKRYGGPSSGAPPPS

SEQ ID NO: 107 - protein, 42 aa, Artificial
  Synthetic Construct
HSDAVFTDNYTRLRKQVAAKKYLQIIKNKRYGGPSSGAPPPS

SEQ ID NO: 108 - protein, 42 aa, Artificial
  Synthetic Construct
HSDAVFTDNYTRLRKQVAAKKYLQLIKNKRYGGPSSGAPPPS

SEQ ID NO: 109 - protein, 42 aa, Artificial
  Synthetic Construct
HSDAVFTDNYTRLRKQVAAKKYLQTIKNKRYGGPSSGAPPPS

SEQ ID NO: 110 - protein, 42 aa, Artificial
  Synthetic Construct
HSDAVFTDNYTRLRKQVAAKKYLQVIKNKRYGGPSSGAPPPS

SEQ ID NO: 111 - protein, 42 aa, Artificial
  Synthetic Construct
HSDAVFTDNYTRLRKQVAAKKYLQWIKNKRYGGPSSGAPPPS

SEQ ID NO: 112 - protein, 42 aa, Artificial
  Synthetic Construct
HSDAVFTDNYTRLLAKLALQKYLQSIKNKRYGGPSSGAPPPS

SEQ ID NO: 113 - protein, 42 aa, Artificial
  Synthetic Construct
HSDAVFTDNYTRLRKQVAAKKYLQSIKNKRYGGPSSGAPPPC

SEQ ID NO: 114 - protein, 41 aa, Artificial; identical to SEQ ID NO: 33
  Synthetic Construct

SEQ ID NO: 115 - protein, 41 aa, Artificial
  Synthetic Construct
HSDAVFTDNYTRLRKQVAAKKYLQSIKNSRGGPSSGAPPPS

SEQ ID NO: 116 - protein, 41 aa, Artificial
  Synthetic Construct
HSDAVFTDNYTRLRKQVAAKKYLQSIKNKRGGPSSGAPPPS

SEQ ID NO: 117 - protein, 42 aa, Artificial
  Synthetic Construct
HSDAVFTDNYTRLRKQVAAKKYLQQIKNKRYGGPSSGAPPPS

SEQ ID NO: 118 - protein, 42 aa, Artificial
  Synthetic Construct
HSDAVFTDNYTRLRKQVAAKKYLQNIKNKRYGGPSSGAPPPS

SEQ ID NO: 119 - protein, 39 aa, Artificial
  Synthetic Construct
HSDAVFTDNYTRLRKQVAAKKYLQSIKRGRPSSGAPPPS

SEQ ID NO: 120 - protein, 42 aa, Artificial
  Synthetic Construct
HSDAVFTDNYTRLRKQVAAKKYLQYIKNKRYGGPSSGAPPPS

SEQ ID NO: 121 - protein, 42 aa, Artificial
  Synthetic Construct
HSDAVFTDNYTRLRKQVAAKKWLQSIKNKRYGGPSSGAPPPS

SEQ ID NO: 122 - protein, 42 aa, Artificial
  Synthetic Construct
HSDAVFTDNYTRLRKQVAAKKFLQSIKNKRYGGPSSGAPPPS

SEQ ID NO: 123 - protein, 42 aa, Artificial
  Synthetic Construct
HSDAVFTDNYTRLRKQVAAKKTLQSIKNKRYGGPSSGAPPPS

SEQ ID NO: 124 - protein, 42 aa, Artificial
  Synthetic Construct
HSDAVFTDNYTRLRKQVAAKKLLQSIKNKRYGGPSSGAPPPS

SEQ ID NO: 125 - protein, 42 aa, Artificial
  Synthetic Construct
HSDAVFTDNYTRLRKQVAAKKILQSIKNKRYGGPSSGAPPPS

SEQ ID NO: 126 - protein, 42 aa, Artificial
  Synthetic Construct
HSDAVFTDNYTRLRKQVAAKKVLQSIKNKRYGGPSSGAPPPS

SEQ ID NO: 127 - protein, 42 aa, Artificial; identical to SEQ ID NO: 88
  Synthetic Construct

SEQ ID NO: 128 - protein, 42 aa, Artificial
  Synthetic Construct
HSDAVFTDNYTRLRAQVAAQKYLQSIKNKRYGGPSSGAPPPS

SEQ ID NO: 129 - protein, 42 aa, Artificial
  Synthetic Construct
HSDAVFTDNYTRLRAQVAAKKYLQSIKNKRYGGPSSGAPPPS

SEQ ID NO: 130 - protein, 43 aa, Artificial
  Synthetic Construct
MHSDAVFTDNYTRLLAKLALQKYLQSIKNKRYGGPSSGAPPPS

SEQ ID NO: 131 - protein, 42 aa, Artificial
  Synthetic Construct
HSDAVFTDNYTRLKAQVAAKKYLQSIKNKRYGGPSSGAPPPS

SEQ ID NO: 132 - protein, 42 aa, Artificial
  Synthetic Construct
HSDAVFTDNYTRLRAQLAAQKYLQSIKNKRYGGPSSGAPPPS

SEQ ID NO: 133 - protein, 39 aa, Artificial
  Synthetic Construct
HSDAVFTDNYTRLRKQMAAQKYLNQLKKGGPSSGAPPPS

SEQ ID NO: 134 - protein, 39 aa, Artificial
  Synthetic Construct
HSDAVFTDNYTRLRKQVAAQKYLNQLKKGGPSSGAPPPS

SEQ ID NO: 135 - protein, 42 aa, Artificial
  Synthetic Construct
HSDAVFTDNYTRLRKQVAAVKYLQSIKNKRYGGPSSGAPPPS

SEQ ID NO: 136 - protein, 42 aa, Artificial
  Synthetic Construct
HSDAVFTDNYTRLRKQVAAYKYLQSIKNKRYGGPSSGAPPPS

SEQ ID NO: 137 - protein, 42 aa, Artificial
  Synthetic Construct
HSDAVFTDNYTRLRKQVAAFKYLQSIKNKRYGGPSSGAPPPS

SEQ ID NO: 138 - protein, 42 aa, Artificial; identical to SEQ ID NO: 35
  Synthetic Construct

SEQ ID NO: 139 - protein, 42 aa, Artificial
  Synthetic Construct
HSDAVFTDNYTRLRKQVAAQKYLQSIKNKRYGGPSSGAPPPS

SEQ ID NO: 140 - protein, 42 aa, Artificial
  Synthetic Construct
HSDAVFTDNYTRLRKQVAALKYLQSIKNKRYGGPSSGAPPPS

SEQ ID NO: 141 - protein, 42 aa, Artificial
  Synthetic Construct
HSDAVFTDNYTRLRKQVAATKYLQSIKNKRYGGPSSGAPPPS

SEQ ID NO: 142 - protein, 42 aa, Artificial
  Synthetic Construct
HSDAVFTDNYTRLRKQVAAWKYLQSIKNKRYGGPSSGAPPPS

SEQ ID NO: 143 - protein, 39 aa, Artificial; identical to SEQ ID NO: 83
  Synthetic Construct

SEQ ID NO: 144 - protein, 42 aa, Artificial
  Synthetic Construct
HSDAVFTDNYTRLRKQVALKKYLQSIKNKRYGGPSSGAPPPS

SEQ ID NO: 145 - protein, 42 aa, Artificial
  Synthetic Construct
HSDAVFTANYTRLRKQVAAKKYLQSIKNKRYGGPSSGAPPPS

SEQ ID NO: 146 - protein, 42 aa, Artificial
  Synthetic Construct
HSDAVFTENYTRLRKQVAAKKYLQSIKNKRYGGPSSGAPPPS

SEQ ID NO: 147 - protein, 42 aa, Artificial
  Synthetic Construct
HSDAVFTKNYTRLRKQVAAKKYLQSIKNKRYGGPSSGAPPPS

SEQ ID NO: 148 - protein, 42 aa, Artificial
  Synthetic Construct
HSDAVFTLNYTRLRKQVAAKKYLQSIKNKRYGGPSSGAPPPS

SEQ ID NO: 149 - protein, 42 aa, Artificial
  Synthetic Construct
HSDAVFTRNYTRLRKQVAAKKYLQSIKNKRYGGPSSGAPPPS

SEQ ID NO: 150 - protein, 42 aa, Artificial
  Synthetic Construct
HSDAVFTYNYTRLRKQVAAKKYLQSIKNKRYGGPSSGAPPPS

SEQ ID NO: 151 - protein, 42 aa, Artificial
  Synthetic Construct
HSDAVFTDNYTRLLAKLALQKYLQSIKNKRYGGPSSGAPPPC

SEQ ID NO: 152 - protein, 42 aa, Artificial
  Synthetic Construct
HSDAVFTEEYTRLQKQVAAKQYLQSIKNKRYGGPSSGAPPPS

SEQ ID NO: 153 - protein, 42 aa, Artificial
  Synthetic Construct
HXDAVFTDNYTRLRKQVAAKKYLQSIKNKRYGGPSSGAPPPS

SEQ ID NO: 154 - protein, 42 aa, Artificial
  Synthetic Construct
HSDAVFTDNYTRLXKQVAAKKYLQSIKNKRYGGPSSGAPPPS

SEQ ID NO: 155 - protein, 42 aa, Artificial
  Synthetic Construct
HSDAVFTDNYTRLRXQVAAKKYLQSIKNKRYGGPSSGAPPPS

SEQ ID NO: 156 - protein, 42 aa, Artificial; identical to SEQ ID NO: 34
  Synthetic Construct

SEQ ID NO: 157 - protein, 42 aa, Artificial
  Synthetic Construct
HSDAVFTDNYTRLRKQVAAKXYLQSIKNKRYGGPSSGAPPPS

SEQ ID NO: 158 - protein, 42 aa, Artificial
  Synthetic Construct
HSDAVFTDNYTRLKKQVAAKKYLQSIKNKRYGGPSSGAPPPS

SEQ ID NO: 159 - protein, 42 aa, Artificial
  Synthetic Construct
HSDAVFTDNYTRLQKQVAAKKYLQSIKNKRYGGPSSGAPPPS

SEQ ID NO: 160 - protein, 42 aa, Artificial
  Synthetic Construct
HSDAVFTDNYTRLAKQVAAKKYLQSIKNKRYGGPSSGAPPPS

SEQ ID NO: 161 - protein, 42 aa, Artificial
  Synthetic Construct
HSDAVFTDNYTRLLKQVAAKKYLQSIKNKRYGGPSSGAPPPS

SEQ ID NO: 162 - protein, 42 aa, Artificial
  Synthetic Construct
HSDAVFTDNYTRLFKQVAAKKYLQSIKNKRYGGPSSGAPPPS

SEQ ID NO: 163 - protein, 42 aa, Artificial; identical to SEQ ID NO: 77
  Synthetic Construct

SEQ ID NO: 164 - protein, 42 aa, Artificial
  Synthetic Construct
HSDAVFTDNYTRLRQQVAAKKYLQSIKNKRYGGPSSGAPPPS

SEQ ID NO: 165 - protein, 42 aa, Artificial
  Synthetic Construct
HSDAVFTDNYTRLRLQVAAKKYLQSIKNKRYGGPSSGAPPPS

SEQ ID NO: 166 - protein, 42 aa, Artificial
  Synthetic Construct
HSDAVFTDNYTRLRFQVAAKKYLQSIKNKRYGGPSSGAPPPS

SEQ ID NO: 167 - protein, 42 aa, Artificial
  Synthetic Construct
HSDAVFTDNYTRLRKQVAAAKYLQSIKNKRYGGPSSGAPPPS

SEQ ID NO: 168 - protein, 42 aa, Artificial
  Synthetic Construct
HSDAVFTDNYTRLRKQVAAKRYLQSIKNKRYGGPSSGAPPPS

SEQ ID NO: 169 - protein, 42 aa, Artificial
  Synthetic Construct
HSDAVFTDNYTRLRKQVAAKQYLQSIKNKRYGGPSSGAPPPS

SEQ ID NO: 170 - protein, 42 aa, Artificial
  Synthetic Construct
HSDAVFTDNYTRLRKQVAAKAYLQSIKNKRYGGPSSGAPPPS

SEQ ID NO: 171 - protein, 42 aa, Artificial
  Synthetic Construct
HSDAVFTDNYTRLRKQVAAKLYLQSIKNKRYGGPSSGAPPPS

SEQ ID NO: 172 - protein, 42 aa, Artificial
  Synthetic Construct
HSDAVFTDNYTRLRKQVAAKFYLQSIKNKRYGGPSSGAPPPS

SEQ ID NO: 173 - protein, 42 aa, Artificial
  Synthetic Construct
HSDAVFTDNYTRLRKQVAAKKYLQXIKNKRYGGPSSGAPPPS

SEQ ID NO: 174 - protein, 42 aa, Artificial
  Synthetic Construct
HSDAVFTDNYTRLRKQVAAKKYLQSXKNKRYGGPSSGAPPPS

SEQ ID NO: 175 - protein, 42 aa, Artificial
  Synthetic Construct
HSDAVFTDNYTRLRKQXAAKKYLQSIKNKRYGGPSSGAPPPS

SEQ ID NO: 176 - protein, 43 aa, Artificial
  Synthetic Construct
HHSDAVFTDNYTRLLAKLALQKYLQSIKNKRYGGPSSGAPPPS

SEQ ID NO: 177 - protein, 42 aa, Artificial
  Synthetic Construct
HSDAVFTDQYTRLLAKLALQKYLQSIKQKRYGGPSSGAPPPS

SEQ ID NO: 178 - protein, 42 aa, Artificial; identical to SEQ ID NO: 30
  Synthetic Construct

SEQ ID NO: 179 - protein, 42 aa, Artificial; identical to SEQ ID NO: 30
  Synthetic Construct

SEQ ID NO: 180 - protein, 42 aa, Artificial
  Synthetic Construct
HSDAVFTDNYTRLLAQLALQKYLQSIKNKRYGGPSSGAPPPS

SEQ ID NO: 181 - protein, 42 aa, Artificial
  Synthetic Construct
HSDAVFTDNYTRLLAKVALQKYLQSIKNKRYGGPSSGAPPPS

SEQ ID NO: 182 - protein, 42 aa, Artificial
  Synthetic Construct
HSDAVFTDNYTRLLAKLAAQKYLQSIKNKRYGGPSSGAPPPS

SEQ ID NO: 183 - protein, 40 aa, Artificial
  Synthetic Construct
HSDAVFTDNYTRLLAKLALQKYLQSIKNKGGPSSGAPPPC

SEQ ID NO: 184 - protein, 43 aa, Artificial
  Synthetic Construct
XHSDAVFTDNYTRLRKQVAAKKYLQSIKNKRYGGPSSGAPPPS

SEQ ID NO: 185 - protein, 42 aa, Artificial
  Synthetic Construct
HSDAVFTDNYTRLRKQVAAKKYLQSIKNKRFGGPSSGAPPPS

SEQ ID NO: 186 - protein, 42 aa, Artificial; identical to SEQ ID NO: 112
  Synthetic Construct

SEQ ID NO: 187 - protein, 42 aa, Artificial; identical to SEQ ID NO: 30
  Synthetic Construct

SEQ ID NO: 188 - protein, 42 aa, Artificial; identical to SEQ ID NO: 30
  Synthetic Construct

SEQ ID NO: 189 - protein, 42 aa, Artificial; identical to SEQ ID NO: 30
  Synthetic Construct

SEQ ID NO: 190 - protein, 42 aa, Artificial
  Synthetic Construct
HSDXVFTDNYTRLRKQVAAKKYLQSIKNKRYGGPSSGAPPPS

SEQ ID NO: 191 - protein, 42 aa, Artificial; identical to SEQ ID NO: 30
  Synthetic Construct

SEQ ID NO: 192 - protein, 42 aa, Artificial; identical to SEQ ID NO: 30
  Synthetic Construct

SEQ ID NO: 193 - protein, 42 aa, Artificial
  Synthetic Construct
HSDAXFTDNYTRLRKQVAAKKYLQSIKNKRYGGPSSGAPPPS

SEQ ID NO: 194 - protein, 39 aa, Artificial
  Synthetic Construct
HSDAVFTDNYTRLRKQVAAKRYLQSIRNGGPSSGAPPPS

SEQ ID NO: 195 - protein, 39 aa, Artificial
  Synthetic Construct
HSDAVFTDNYTRLRKQVAARRYLQSIRNGGPSSGAPPPS

SEQ ID NO: 196 - protein, 39 aa, Artificial
  Synthetic Construct
HSDAVFTDNYTRLRRQVAAKRYLQSIRNGGPSSGAPPPS

SEQ ID NO: 197 - protein, 39 aa, Artificial; identical to SEQ ID NO: 24
  Synthetic Construct

SEQ ID NO: 198 - protein, 39 aa, Artificial
  Synthetic Construct
HSDAVFTDQYTRLRRQVAARKYLQSIRQGGPSSGAPPPS

SEQ ID NO: 199 - protein, 42 aa, Artificial; identical to SEQ ID NO: 57
  Synthetic Construct

SEQ ID NO: 200 - protein, 42 aa, Artificial
  Synthetic Construct
HGEGTFTDNYTRLRKQVAAKKYLQSIKNKRYGGPSSGAPPPS

SEQ ID NO: 201 - protein, 42 aa, Artificial; identical to SEQ ID NO: 62
  Synthetic Construct

SEQ ID NO: 202 - protein, 42 aa, Artificial
  Synthetic Construct
HSEAVFTDNYTRLRKQVAAKKYLQSIKNKRYGGPSSGAPPPS

SEQ ID NO: 203 - protein, 42 aa, Artificial; identical to SEQ ID NO: 30
  Synthetic Construct

SEQ ID NO: 204 - protein, 42 aa, Artificial; identical to SEQ ID NO: 30
  Synthetic Construct

SEQ ID NO: 205 - protein, 42 aa, Artificial
  Synthetic Construct
HSDAVFTDNYTRLRKQVAAKKXLQSIKNKRYGGPSSGAPPPS

SEQ ID NO: 206 - protein, 42 aa, Artificial
  Synthetic Construct
HSDAVFTDNYTRLRKQVAAKKALQSIKNKRYGGPSSGAPPPS

SEQ ID NO: 207 - protein, 42 aa, Artificial
  Synthetic Construct
HSDAVFTDNYTRLRKQVAAKKYWQSIKNKRYGGPSSGAPPPS

SEQ ID NO: 208 - protein, 42 aa, Artificial
  Synthetic Construct
HSDAVFTDNYTRLRKQVAAKKYFQSIKNKRYGGPSSGAPPPS

SEQ ID NO: 209 - protein, 42 aa, Artificial
  Synthetic Construct
HSDAVFTDNYTRLRKQVAAKKYTQSIKNKRYGGPSSGAPPPS

SEQ ID NO: 210 - protein, 42 aa, Artificial
  Synthetic Construct
HSDAVFTDNYTRLRKQVAAKKYIQSIKNKRYGGPSSGAPPPS

SEQ ID NO: 211 - protein, 42 aa, Artificial
  Synthetic Construct
HSDAVFTDNYTRLRKQVAAKKYVQSIKNKRYGGPSSGAPPPS

SEQ ID NO: 212 - protein, 42 aa, Artificial
  Synthetic Construct
HSDAVFTDNYTRLRKQVAAKKYAQSIKNKRYGGPSSGAPPPS

SEQ ID NO: 213 - protein, 42 aa, Artificial
  Synthetic Construct
HSDAVFTDNYTRLRKQVAAKKYXQSIKNKRYGGPSSGAPPPS

SEQ ID NO: 214 - protein, 42 aa, Artificial
  Synthetic Construct
HSDAVFTDNYTRLXKQLAAXKYLQSIKNKRYGGPSSGAPPPS

SEQ ID NO: 215 - protein, 42 aa, Artificial
  Synthetic Construct
HSDAVFTDNYTRLXKQLAAKXYLQSIKNKRYGGPSSGAPPPS

SEQ ID NO: 216 - protein, 42 aa, Artificial
  Synthetic Construct
HSDAVFTDNYTRLXKQVAAXKYLQSIKNKRYGGPSSGAPPPS

SEQ ID NO: 217 - protein, 42 aa, Artificial
  Synthetic Construct
HSDAVFTDNYTRLXKQVAAQKYLQSIKNKRYGGPSSGAPPPS

SEQ ID NO: 218 - protein, 42 aa, Artificial
  Synthetic Construct
HSDAVFTDNYTRLXKQVAAKXYLQSIKNKRYGGPSSGAPPPS

SEQ ID NO: 219 - protein, 42 aa, Artificial
  Synthetic Construct
HSDAVFTDNYTRLXKQVAAKQYLQSIKNKRYGGPSSGAPPPS

SEQ ID NO: 220 - protein, 42 aa, Artificial
  Synthetic Construct
HSDAVFTDNYTRLQKQVAAXKYLQSIKNKRYGGPSSGAPPPS

SEQ ID NO: 221 - protein, 42 aa, Artificial
  Synthetic Construct
HSDAVFTDNYTRLQKQVAAKXYLQSIKNKRYGGPSSGAPPPS

SEQ ID NO: 222 - protein, 42 aa, Artificial
  Synthetic Construct
HSDAVFTDNYTRLQKQVAAQKYLQSIKNKRYGGPSSGAPPPS

SEQ ID NO: 223 - protein, 42 aa, Artificial
  Synthetic Construct
HSDAVFTDNYTRLQKQVAAKQYLQSIKNKRYGGPSSGAPPPS

SEQ ID NO: 224 - protein, 42 aa, Artificial
  Synthetic Construct
HSDAVFTDNYTRLQXQVAAKKYLQSIKNKRYGGPSSGAPPPS

SEQ ID NO: 225 - protein, 42 aa, Artificial
  Synthetic Construct
HSDAVFTDNYTRLXKQVAALKYLQSIKNKRYGGPSSGAPPPS

SEQ ID NO: 226 - protein, 42 aa, Artificial
  Synthetic Construct
HSDAVFTDNYTRLXKQVAAAKYLQSIKNKRYGGPSSGAPPPS

SEQ ID NO: 227 - protein, 42 aa, Artificial
  Synthetic Construct
HSDAVFTDNYTRLRXQVAAXKYLQSIKNKRYGGPSSGAPPPS

SEQ ID NO: 228 - protein, 42 aa, Artificial
  Synthetic Construct
HSDAVFTDNYTRLRXQVAAVKYLQSIKNKRYGGPSSGAPPPS

SEQ ID NO: 229 - protein, 42 aa, Artificial
  Synthetic Construct
HSDAVFTDNYTRLRXQVAAAKYLQSIKNKRYGGPSSGAPPPS

SEQ ID NO: 230 - protein, 42 aa, Artificial
  Synthetic Construct
HSDAVFTDNYTRLRXQVAAKXYLQSIKNKRYGGPSSGAPPPS

SEQ ID NO: 231 - protein, 42 aa, Artificial
  Synthetic Construct
HSDAVFTDNYTRLRXQVAALKYLQSIKNKRYGGPSSGAPPPS

SEQ ID NO: 232 - protein, 42 aa, Artificial
  Synthetic Construct
HSDAVFTDNYTRLXKQVAAVKYLQSIKNKRYGGPSSGAPPPS

SEQ ID NO: 233 - protein, 40 aa, Artificial
  Synthetic Construct
HSDAVFTDNYTRLRKQVAAKKYLQSIKQKGGPSSGAPPPS

SEQ ID NO: 234 - protein, 39 aa, Artificial; identical to SEQ ID NO: 89
  Synthetic Construct

SEQ ID NO: 235 - protein, 42 aa, Artificial
  Synthetic Construct
HSDAVFTDNYTRLRKQVAGKKYLQSIKNKRYGGPSSGAPPPS

SEQ ID NO: 236 - protein, 42 aa, Artificial
  Synthetic Construct
HSDAVFTDNYTRLXKQVAAXKYLQSIKNKRYGGPSSGAPPPC

SEQ ID NO: 237 - protein, 42 aa, Artificial
  Synthetic Construct
HSDAVFTDNYTRLRXQLAAKXYLQSIKNKRYGGPSSGAPPPS

SEQ ID NO: 238 - protein, 42 aa, Artificial
  Synthetic Construct
HSDAVFTDNYTRLRXQVAAKXYLQSIKNKRYGGPSSGAPPPC

SEQ ID NO: 239 - protein, 42 aa, Artificial; identical to SEQ ID NO: 66
  Synthetic Construct

SEQ ID NO: 240 - protein, 42 aa, Artificial
  Synthetic Construct
HSDAVFTDNYTRLRKQLAAKKYLQSIKNKRYGGPSSGAPPPS

SEQ ID NO: 241 - protein, 42 aa, Artificial
  Synthetic Construct
HSDAVFTDNYTRLRXQLAAXKYLQSIKNKRYGGPSSGAPPPS

SEQ ID NO: 242 - protein, 40 aa, Artificial
  Synthetic Construct
HSDAVFTDNYTRLRXQLAAXKYLQSIKNKGGPSSGAPPPS

SEQ ID NO: 243 - protein, 40 aa, Artificial
  Synthetic Construct
HSDAVFTDNYTRLXKQVAAXKYLQSIKQKGGPSSGAPPPS

SEQ ID NO: 244 - protein, 40 aa, Artificial
  Synthetic Construct
HSDAVFTDNYTRLRXQVAAXKYLQSIKQKGGPSSGAPPPS

SEQ ID NO: 245 - protein, 40 aa, Artificial
  Synthetic Construct
HSDAVFTDNYTRLRXQVAAKXYLQSIKQKGGPSSGAPPPS

SEQ ID NO: 246 - protein, 39 aa, Artificial
  Synthetic Construct
HSDAVFTDNYTRLXKQVAAXKYLQSIKQGGPSSGAPPPS

SEQ ID NO: 247 - protein, 39 aa, Artificial
  Synthetic Construct
HSDAVFTDNYTRLRXQVAAXKYLQSIKQGGPSSGAPPPS

SEQ ID NO: 248 - protein, 39 aa, Artificial
  Synthetic Construct
HSDAVFTDNYTRLRXQVAAKXYLQSIKQGGPSSGAPPPS

SEQ ID NO: 249 - protein, 42 aa, Artificial
  Synthetic Construct
HSDAVFTDNYTRLRXQVAAXKYLQSIKNKRYGGPSSGAPPPC

SEQ ID NO: 250 - protein, 42 aa, Artificial
  Synthetic Construct
HSDAVFTDNYTRLAXQVAAXKYLQSIKNKRYGGPSSGAPPPS

SEQ ID NO: 251 - protein, 42 aa, Artificial
  Synthetic Construct
HSDAVFTDNYTRLQXQVAAXKYLQSIKNKRYGGPSSGAPPPS

SEQ ID NO: 252 - protein, 42 aa, Artificial; identical to SEQ ID NO: 154
  Synthetic Construct

SEQ ID NO: 253 - protein, 42 aa, Artificial; identical to SEQ ID NO: 155
  Synthetic Construct

SEQ ID NO: 254 - protein, 42 aa, Artificial
  Synthetic Construct
HSDAVFTDNYTRLXXQVAAKKYLQSIKNKRYGGPSSGAPPPS

SEQ ID NO: 255 - protein, 42 aa, Artificial
  Synthetic Construct
HSEAVFTENYTRLRXQVAAXKYLQSIKNKRYGGPSSGAPPPS

SEQ ID NO: 256 - protein, 42 aa, Artificial
  Synthetic Construct
HSDAVFTDQYTRLRXQVAAXKYLQSIKQKRYGGPSSGAPPPS

SEQ ID NO: 257 - protein, 42 aa, Artificial
  Synthetic Construct
HSDAVFTDNYTRLXXQVAAXKYLQSIKNKRYGGPSSGAPPPS

SEQ ID NO: 258 - protein, 42 aa, Artificial
  Synthetic Construct
HSDAVFTDNYTRLLXQVAAXKYLQSIKNKRYGGPSSGAPPPS

SEQ ID NO: 259 - protein, 42 aa, Artificial
  Synthetic Construct
HSDAVFTDNYTRLKXQVAAXKYLQSIKNKRYGGPSSGAPPPS

SEQ ID NO: 260 - protein, 42 aa, Artificial; identical to SEQ ID NO: 257
  Synthetic Construct

SEQ ID NO: 261 - protein, 42 aa, Artificial; identical to SEQ ID NO: 257
  Synthetic Construct

SEQ ID NO: 262 - protein, 42 aa, Artificial
  Synthetic Construct
HSDAVFTDNYTRLRXKVAAXKYLQSIKNKRYGGPSSGAPPPS

SEQ ID NO: 263 - protein, 42 aa, Artificial
  Synthetic Construct
HSDAVFTDNYTRLRXQIAAXKYLQSIKNKRYGGPSSGAPPPS

SEQ ID NO: 264 - protein, 42 aa, Artificial
  Synthetic Construct
HSDAVFTDNYTRLRXQKAAXKYLQSIKNKRYGGPSSGAPPPS

SEQ ID NO: 265 - protein, 42 aa, Artificial
  Synthetic Construct
HSDAVFTDNYTRLRXQAAAXKYLQSIKNKRYGGPSSGAPPPS

SEQ ID NO: 266 - protein, 42 aa, Artificial
  Synthetic Construct
HSDAVFTDNYTRLRXQXAAXKYLQSIKNKRYGGPSSGAPPPS

SEQ ID NO: 267 - protein, 40 aa, Artificial
  Synthetic Construct
HSDAVFTDNYTXLRXQVAAXKYLQSIXNXGGPSSGAPPPS

SEQ ID NO: 268 - protein, 40 aa, Artificial
  Synthetic Construct
HSDAVFTDNYTXLRXQVAAXKYLQSIXNXGGPSSGAPPPC

SEQ ID NO: 269 - protein, 40 aa, Artificial; identical to SEQ ID NO: 267
  Synthetic Construct

SEQ ID NO: 270 - protein, 40 aa, Artificial; identical to SEQ ID NO: 268
  Synthetic Construct

SEQ ID NO: 271 - protein, 39 aa, Artificial
  Synthetic Construct
HSDAVFTDNYTXLRXQVAAXKYLQSIXXGGPSSGAPPPS

SEQ ID NO: 272 - protein, 39 aa, Artificial
  Synthetic Construct
HSDAVFTDNYTXLRXQVAAXKYLQSIXXGGPSSGAPPPC

SEQ ID NO: 273 - protein, 39 aa, Artificial; identical to SEQ ID NO: 271
  Synthetic Construct

SEQ ID NO: 274 - protein, 39 aa, Artificial; identical to SEQ ID NO: 272
  Synthetic Construct

SEQ ID NO: 275 - protein, 42 aa, Artificial
  Synthetic Construct
HSDAVFTDNYTXLRXQVAAXKYLQSIXNXXYGGPSSGAPPPS

SEQ ID NO: 276 - protein, 42 aa, Artificial
  Synthetic Construct
HSDAVFTDNYTXLRXQVAAXKYLQSIXNXXYGGPSSGAPPPC

SEQ ID NO: 277 - protein, 42 aa, Artificial
  Synthetic Construct
HSDAVFTDNYTRLRXQVAAXAYLQSIKNKRYGGPSSGAPPPS

SEQ ID NO: 278 - protein, 42 aa, Artificial
  Synthetic Construct
HSDAVFTDNYTRLRXQVAAXXYLQSIKNKRYGGPSSGAPPPS

SEQ ID NO: 279 - protein, 42 aa, Artificial
  Synthetic Construct
HSDAVFTDNYTXLRXQVAAXKYLQSIKNKRYGGPSSGAPPPS

SEQ ID NO: 280 - protein, 42 aa, Artificial; identical to SEQ ID NO: 279
  Synthetic Construct

SEQ ID NO: 281 - protein, 42 aa, Artificial; identical to SEQ ID NO: 279
  Synthetic Construct

SEQ ID NO: 282 - protein, 42 aa, Artificial
  Synthetic Construct
HSDAVFTDNYTRXRXQVAAXKYLQSIKNKRYGGPSSGAPPPS

SEQ ID NO: 283 - protein, 40 aa, Artificial
  Synthetic Construct
HSDAVFTDNYTRLRXQVAAXKYLQSIKXKGGPSSGAPPPS

SEQ ID NO: 284 - protein, 40 aa, Artificial
  Synthetic Construct
HSDAVFTDNYTRLRXQVAAXKYLQSIKPKGGPSSGAPPPS

SEQ ID NO: 285 - protein, 39 aa, Artificial
  Synthetic Construct
HSDAVFTDNYTRLRXQVAAXKYLQSIKKGGPSSGAPPPS

SEQ ID NO: 286 - protein, 39 aa, Artificial
  Synthetic Construct
HSDAVFTDNYTRLRXQVAAXKYLQSIXXGGPSSGAPPPS

SEQ ID NO: 287 - protein, 39 aa, Artificial; identical to SEQ ID NO: 285
  Synthetic Construct

SEQ ID NO: 288 - protein, 39 aa, Artificial
  Synthetic Construct
HSDAVFTDNYTRLRXQVAAXKYLQSIKXGGPSSGAPPPS

SEQ ID NO: 289 - protein, 39 aa, Artificial; identical to SEQ ID NO: 288
  Synthetic Construct

SEQ ID NO: 290 - protein, 40 aa, Artificial
  Synthetic Construct
HSDAVFTDNYTRLRXQVAAXKYLQSIXQXGGPSSGAPPPS

SEQ ID NO: 291 - protein, 40 aa, Artificial
  Synthetic Construct
HSDAVFTDNYTRLRXQVAAXKYLQSIKXXGGPSSGAPPPS

SEQ ID NO: 292 - protein, 42 aa, Artificial; identical to SEQ ID NO: 227
  Synthetic Construct

SEQ ID NO: 293 - protein, 40 aa, Artificial
  Synthetic Construct
HSEAVFTENYTXLRXQVAAXKYLQSIXNXGGPSSGAPPPS

SEQ ID NO: 294 - protein, 40 aa, Artificial
  Synthetic Construct
HSDAVFTDQYTXLRXQVAAXKYLQSIXQXGGPSSGAPPPS

SEQ ID NO: 295 - protein, 40 aa, Artificial
  Synthetic Construct
HSDAVFTDNYTXLRXQLAAXKYLQSIXNXGGPSSGAPPPS

SEQ ID NO: 296 - protein, 40 aa, Artificial
  Synthetic Construct
HSDAVFTDNYTXLRXQIAAXKYLQSIXNXGGPSSGAPPPS

SEQ ID NO: 297 - protein, 40 aa, Artificial
  Synthetic Construct
HSDAVFTDNYTALRXQVAAXKYLQSIXNXGGPSSGAPPPS

SEQ ID NO: 298 - protein, 40 aa, Artificial
  Synthetic Construct
HSDAVFTDNYTLLRXQVAAXKYLQSIXNXGGPSSGAPPPS

SEQ ID NO: 299 - protein, 40 aa, Artificial
  Synthetic Construct
HSDAVFTDNYTQLRXQVAAXKYLQSIXNXGGPSSGAPPPS

SEQ ID NO: 300 - protein, 40 aa, Artificial
  Synthetic Construct
HSDAVFTDNYTFLRXQVAAXKYLQSIXNXGGPSSGAPPPS

SEQ ID NO: 301 - protein, 40 aa, Artificial
  Synthetic Construct
HSDAVFTDNYTRLLAKLALQKYLQSIXNXGGPSSGAPPPS

SEQ ID NO: 302 - protein, 40 aa, Artificial
  Synthetic Construct
HSDAVFTDNYTXLLAKLALQKYLQSIXNXGGPSSGAPPPS

SEQ ID NO: 303 - protein, 40 aa, Artificial
  Synthetic Construct
HSDAVFTDNYTXLRXQVAAXKYLQCIXNXGGPSSGAPPPS

SEQ ID NO: 304 - protein, 40 aa, Artificial
  Synthetic Construct
HSDAVFTDNYTXLRXQVAACXYLQSIXNXGGPSSGAPPPS

SEQ ID NO: 305 - protein, 39 aa, Artificial
  Synthetic Construct
HSEAVFTEQYTXLRXQVAAXXYLQSIXXGGPSSGAPPPC

SEQ ID NO: 306 - protein, 40 aa, Artificial
  Synthetic Construct
HSDAVFTDNYTXLRXQVAAXKYLQSSXNXGGPSSGAPPPS

SEQ ID NO: 307 - protein, 40 aa, Artificial
  Synthetic Construct
HSDAVFTDNYTXLRXQVAAXKYLSSIXNXGGPSSGAPPPS

SEQ ID NO: 308 - protein, 40 aa, Artificial
  Synthetic Construct
HSDAVFTDNYTXLRXQVAAXKYSQSIXNXGGPSSGAPPPS

SEQ ID NO: 309 - protein, 40 aa, Artificial
  Synthetic Construct
HSDAVFTDNYTXLRXQVAAXKSLQSIXNXGGPSSGAPPPS

SEQ ID NO: 310 - protein, 40 aa, Artificial
  Synthetic Construct
HSDAVFTDNYTXLRXQVAAXSYLQSIXNXGGPSSGAPPPS

SEQ ID NO: 311 - protein, 40 aa, Artificial
  Synthetic Construct
HSDAVFTDNYTXLRXQVASXKYLQSIXNXGGPSSGAPPPS

SEQ ID NO: 312 - protein, 40 aa, Artificial
  Synthetic Construct
HSDAVFTDNYTXLRXQVSAXKYLQSIXNXGGPSSGAPPPS

SEQ ID NO: 313 - protein, 40 aa, Artificial
  Synthetic Construct
HSDAVFTDNYTXLRXQSAAXKYLQSIXNXGGPSSGAPPPS

SEQ ID NO: 314 - protein, 40 aa, Artificial
  Synthetic Construct
HSDAVFTDNYTXLRXSVAAXKYLQSIXNXGGPSSGAPPPS

SEQ ID NO: 315 - protein, 40 aa, Artificial
  Synthetic Construct
HSDAVFTDNYTXSRXQVAAXKYLQSIXNXGGPSSGAPPPS

SEQ ID NO: 316 - protein, 40 aa, Artificial
  Synthetic Construct
HSDAVFTDSYTXLRXQVAAXKYLQSIXNXGGPSSGAPPPS

SEQ ID NO: 317 - protein, 39 aa, Artificial
  Synthetic Construct
HSEAVFTEQYTXLRXQLAAXXYLQSIXXGGPSSGAPPPS

SEQ ID NO: 318 - protein, 39 aa, Artificial
  Synthetic Construct
HSDAVFTDQYTXLRXQLAAXXYLQSIXXGGPSSGAPPPS

SEQ ID NO: 319 - protein, 39 aa, Artificial; identical to SEQ ID NO: 318
  Synthetic Construct

SEQ ID NO: 320 - protein, 39 aa, Artificial
  Synthetic Construct
HSDAVFTDQYTXLRXQVAAXXYLQSIXXGGPSSGAPPPS

SEQ ID NO: 321 - protein, 39 aa, Artificial
  Synthetic Construct
HSDAVFTDNYTXLRXQVAAXXYLQSIXXGGPSSGAPPPS

SEQ ID NO: 322 - protein, 39 aa, Artificial
  Synthetic Construct
HSDAVFTEQYTXLRXQVAAXXYLQSIXXGGPSSGAPPPC

SEQ ID NO: 323 - protein, 40 aa, Artificial
  Synthetic Construct
HSDAVFTDNYTRXRXQVAAXKYLQSIKXKGGPSSGAPPPS

SEQ ID NO: 324 - protein, 40 aa, Artificial
  Synthetic Construct
HSDAVFTDQYTRXRXQVAAXKYLQSIKXKGGPSSGAPPPS

SEQ ID NO: 325 - protein, 40 aa, Artificial
  Synthetic Construct
HSDAVFTDQYTRXRXQLAAXKYLQSIKXKGGPSSGAPPPS

SEQ ID NO: 326 - protein, 40 aa, Artificial; identical to SEQ ID NO: 325
  Synthetic Construct

SEQ ID NO: 327 - protein, 40 aa, Artificial
  Synthetic Construct
HSEAVFTEQYTRXRXQLAAXKYLQSIKXKGGPSSGAPPPS

SEQ ID NO: 328 - protein, 40 aa, Artificial; identical to SEQ ID NO: 267
  Synthetic Construct

SEQ ID NO: 329 - protein, 39 aa, Artificial
  Synthetic Construct
HSDAVFTEQYTXLRXQLAAXXYLQSIXXGGPSSGAPPPC

SEQ ID NO: 330 - protein, 39 aa, Artificial
  Synthetic Construct
HSDAVFTEQYTXLRXQLAAXXYLQSIXXGGPSSGAPPPS

SEQ ID NO: 331 - protein, 39 aa, Artificial
  Synthetic Construct
HSDAVFTEQYTXLRXQVAAXXYLQSCXXGGPSSGAPPPS

SEQ ID NO: 332 - protein, 39 aa, Artificial
  Synthetic Construct
HSDAVFTEQYTXLRXQVAAXXYLCSIXXGGPSSGAPPPS

SEQ ID NO: 333 - protein, 39 aa, Artificial
  Synthetic Construct
HSDAVFTEQYTXLRXQVAAXXYLXSIXXGGPSSGAPPPS

SEQ ID NO: 334 - protein, 39 aa, Artificial
  Synthetic Construct
HSDAVFTEQYTXLRXQVACXXYLQSIXXGGPSSGAPPPS

SEQ ID NO: 335 - protein, 39 aa, Artificial
  Synthetic Construct
HSDAVFTEQYTXLRXQVAXXXYLQSIXXGGPSSGAPPPS

SEQ ID NO: 336 - protein, 39 aa, Artificial
  Synthetic Construct
HSDAVFTEQYTXLRXQCAAXXYLQSIXXGGPSSGAPPPS

SEQ ID NO: 337 - protein, 39 aa, Artificial
  Synthetic Construct
HSDAVFTEQYTXLRXCVAAXXYLQSIXXGGPSSGAPPPS

SEQ ID NO: 338 - protein, 40 aa, Artificial
  Synthetic Construct
HSDAVFTEQYTXLRXQVAAXXYLQSIXCXGGPSSGAPPPS

SEQ ID NO: 339 - protein, 39 aa, Artificial
  Synthetic Construct
HSDAVFTEQYTXLRXQVAAXXYLQCIXXGGPSSGAPPPC

SEQ ID NO: 340 - protein, 39 aa, Artificial
  Synthetic Construct
HSDAVFTECYTXLRXQVAAXXYLQSIXXGGPSSGAPPPS

SEQ ID NO: 341 - protein, 39 aa, Artificial
  Synthetic Construct
HSDAVFTEQYTXCRXQVAAXXYLQSIXXGGPSSGAPPPS

SEQ ID NO: 342 - protein, 32 aa, Artificial; identical to SEQ ID NO: 49
  Synthetic Construct

SEQ ID NO: 343 - protein, 42 aa, Artificial; identical to SEQ ID NO: 113
  Synthetic Construct

SEQ ID NO: 344 - protein, 42 aa, Artificial; identical to SEQ ID NO: 151
  Synthetic Construct

SEQ ID NO: 345 - protein, 40 aa, Artificial; identical to SEQ ID NO: 183
  Synthetic Construct

SEQ ID NO: 346 - protein, 39 aa, Artificial; identical to SEQ ID NO: 194
  Synthetic Construct

SEQ ID NO: 347 - protein, 39 aa, Artificial; identical to SEQ ID NO: 196
  Synthetic Construct

SEQ ID NO: 348 - protein, 39 aa, Artificial; identical to SEQ ID NO: 195
  Synthetic Construct

SEQ ID NO: 349 - protein, 39 aa, Artificial; identical to SEQ ID NO: 196
  Synthetic Construct

SEQ ID NO: 350 - protein, 39 aa, Artificial; identical to SEQ ID NO: 24
  Synthetic Construct

SEQ ID NO: 351 - protein, 42 aa, Artificial; identical to SEQ ID NO: 236
  Synthetic Construct

SEQ ID NO: 352 - protein, 42 aa, Artificial; identical to SEQ ID NO: 238
  Synthetic Construct

SEQ ID NO: 353 - protein, 42 aa, Artificial; identical to SEQ ID NO: 249
  Synthetic Construct

SEQ ID NO: 354 - protein, 40 aa, Artificial; identical to SEQ ID NO: 268
  Synthetic Construct

SEQ ID NO: 355 - protein, 40 aa, Artificial; identical to SEQ ID NO: 268
  Synthetic Construct

SEQ ID NO: 356 - protein, 39 aa, Artificial; identical to SEQ ID NO: 272
  Synthetic Construct

SEQ ID NO: 357 - protein, 42 aa, Artificial; identical to SEQ ID NO: 276
  Synthetic Construct

SEQ ID NO: 358 - protein, 40 aa, Artificial; identical to SEQ ID NO: 303
  Synthetic Construct

SEQ ID NO: 359 - protein, 39 aa, Artificial; identical to SEQ ID NO: 305
  Synthetic Construct

SEQ ID NO: 360 - protein, 40 aa, Artificial; identical to SEQ ID NO: 304
  Synthetic Construct

SEQ ID NO: 361 - protein, 43 aa, Artificial
  Synthetic Construct
XHSDAVFTDNYTRLRKQVAAKKYLQSIKNKRYGGPSSGAPPPC

SEQ ID NO: 362 - protein, 39 aa, Artificial; identical to SEQ ID NO: 322
  Synthetic Construct

SEQ ID NO: 363 - protein, 39 aa, Artificial; identical to SEQ ID NO: 322
  Synthetic Construct

SEQ ID NO: 364 - protein, 40 aa, Artificial; identical to SEQ ID NO: 304
  Synthetic Construct

SEQ ID NO: 365 - protein, 40 aa, Artificial; identical to SEQ ID NO: 267
  Synthetic Construct

SEQ ID NO: 366 - protein, 39 aa, Artificial; identical to SEQ ID NO: 329
  Synthetic Construct

SEQ ID NO: 367 - protein, 39 aa, Artificial; identical to SEQ ID NO: 331
  Synthetic Construct

SEQ ID NO: 368 - protein, 39 aa, Artificial; identical to SEQ ID NO: 332
  Synthetic Construct

SEQ ID NO: 369 - protein, 39 aa, Artificial; identical to SEQ ID NO: 333
  Synthetic Construct

SEQ ID NO: 370 - protein, 39 aa, Artificial; identical to SEQ ID NO: 334
  Synthetic Construct

SEQ ID NO: 371 - protein, 39 aa, Artificial; identical to SEQ ID NO: 335
  Synthetic Construct

SEQ ID NO: 372 - protein, 39 aa, Artificial; identical to SEQ ID NO: 336
  Synthetic Construct

SEQ ID NO: 373 - protein, 39 aa, Artificial; identical to SEQ ID NO: 337
  Synthetic Construct

SEQ ID NO: 374 - protein, 40 aa, Artificial; identical to SEQ ID NO: 338
  Synthetic Construct

SEQ ID NO: 375 - protein, 39 aa, Artificial; identical to SEQ ID NO: 339
  Synthetic Construct

SEQ ID NO: 376 - protein, 39 aa, Artificial; identical to SEQ ID NO: 340
  Synthetic Construct

SEQ ID NO: 377 - protein, 39 aa, Artificial; identical to SEQ ID NO: 337
  Synthetic Construct

SEQ ID NO: 378 - protein, 39 aa, Artificial; identical to SEQ ID NO: 341
  Synthetic Construct

SEQ ID NO: 379 - protein, 39 aa, Artificial; identical to SEQ ID NO: 333
  Synthetic Construct

SEQ ID NO: 380 - protein, 39 aa, Artificial; identical to SEQ ID NO: 335
  Synthetic Construct

SEQ ID NO: 381 - protein, 28 aa, Artificial
  Synthetic Construct
HSDAVFTDNYTRLRKQMAVKKYLNSIKK

SEQ ID NO: 382 - protein, 31 aa, Artificial
  Synthetic Construct
HSDAVFTDNYTRLRKQMAVKKYLNSIKKGGT

SEQ ID NO: 383 - protein, 31 aa, Artificial
  Synthetic Construct
HSDAVFTENYTKLRKQLAAKKYLNDLLNGGT

SEQ ID NO: 384 - protein, 31 aa, Artificial
  Synthetic Construct
HSDAVFTDNYTKLRKQLAAKKYLNDILNGGT

SEQ ID NO: 385 - protein, 40 aa, Artificial
  Synthetic Construct
HSDAVFTENYTKLRKQLAAKKYLNDLKKGGTSWCEPGWCR

SEQ ID NO: 386 - protein, 31 aa, Artificial
  Synthetic Construct
HSDAVFTDNYTRLRKQLAAKKYLNSIKKGGT

SEQ ID NO: 387 - protein, 31 aa, Artificial
  Synthetic Construct
HSDAVFTDNYTRLRKQLAAKKYLNDIKNGGT

SEQ ID NO: 388 - protein, 31 aa, Artificial
  Synthetic Construct
HSDAVFTDNYTRLRKQLAVKKYLNSIKKGGT

SEQ ID NO: 389 - protein, 31 aa, Artificial
  Synthetic Construct
HSDAVFTDNYTRLRKQMAAKKYLNSIKKGGT

SEQ ID NO: 390 - protein, 31 aa, Artificial
  Synthetic Construct
HSDAVFTDNYTRLRKQLAVKKYLNDIKNGGT

SEQ ID NO: 391 - protein, 31 aa, Artificial
  Synthetic Construct
HSDAVFTDNYTRLRKQLAAKKYLNSIKNGGT

SEQ ID NO: 392 - protein, 31 aa, Artificial
  Synthetic Construct
HSDAVFTDNYTRLRKQLAAKKYLNDIKKKRY

SEQ ID NO: 393 - protein, 28 aa, Artificial
  Synthetic Construct
HSDAVFTDNYTRLRKQMAVKKYLNSIKK

SEQ ID NO: 394 - protein, 28 aa, Artificial
  Synthetic Construct
HSDAVFTDNYTRLRKQMAVKKYLNSIKN

SEQ ID NO: 395 - protein, 28 aa, Artificial
  Synthetic Construct
HSDAVFTDNYTRLRKQMAVKKYLNSILK

SEQ ID NO: 396 - protein, 28 aa, Artificial
  Synthetic Construct
HSDAVFTDNYTELRKQMAVKKYLNSILN

SEQ ID NO: 397 - protein, 28 aa, Artificial
  Synthetic Construct
HSDAVFTDNYTRLRKQMAVKKYLNDILN

SEQ ID NO: 398 - protein, 28 aa, Artificial
  Synthetic Construct
HSDAVFTDNYTRLRKQMAAKKYLNSIKN

SEQ ID NO: 399 - protein, 28 aa, Artificial
  Synthetic Construct
HSDAVFTDNYTRLRKQMAAKKYLNSILK

SEQ ID NO: 400 - protein, 28 aa, Artificial
  Synthetic Construct
HSDAVFTDNYTRLRKQMAAKKYLNSIKK

SEQ ID NO: 401 - protein, 31 aa, Artificial
  Synthetic Construct
HSDAVFTDNYTRLRKQMAAKKYLNSIKKKRY

SEQ ID NO: 402 - protein, 30 aa, Artificial
  Synthetic Construct
HSDAVFTDNYTRLRKQMAAKKYLNSIKKKR

SEQ ID NO: 403 - protein, 29 aa, Artificial
  Synthetic Construct
HSDAVFTDNYTRLRKQMAAKKYLNSIKKK

SEQ ID NO: 404 - protein, 31 aa, Artificial
  Synthetic Construct
HSDAVFTDNYTRLRKQMAAKKYLNSIKNKRY

SEQ ID NO: 405 - protein, 31 aa, Artificial
  Synthetic Construct
HSDAVFTDNYTRLRKQMAVKKYLNSIKKKRY

SEQ ID NO: 406 - protein, 30 aa, Artificial
  Synthetic Construct
HSDAVFTDNYTRLRKQMAVKKYLNSIKKKR

SEQ ID NO: 407 - protein, 29 aa, Artificial
  Synthetic Construct
HSDAVFTDNYTRLRKQMAVKKYLNSIKKK

SEQ ID NO: 408 - protein, 31 aa, Artificial
  Synthetic Construct
HSDAVFTDNYTRLRKQMAVKKYLNSIKNKRY

SEQ ID NO: 409 - protein, 28 aa, Artificial
  Synthetic Construct
HSDAVFTDNYTRLRKQVAAKKYLQSIKK

SEQ ID NO: 410 - protein, 28 aa, Artificial
  Synthetic Construct
HSDAVFTDNYTRLRKQIAAKKYLQTIKK

SEQ ID NO: 411 - protein, 28 aa, Artificial
  Synthetic Construct
HSDAVFTENYTRLRKQMAVKKYLNSLKK

SEQ ID NO: 412 - protein, 31 aa, Artificial
  Synthetic Construct
HSDAVFTDNYTRLRKQLAAKKYLNDILKGGT

SEQ ID NO: 413 - protein, 31 aa, Artificial
  Synthetic Construct
HSDAVFTDNYTRLRKQLAAKKYLNDILNGGT

SEQ ID NO: 414 - protein, 31 aa, Artificial
  Synthetic Construct
HSDAVFTDNYTRLRKQLAVKKYLNDILKGGT

SEQ ID NO: 415 - protein, 28 aa, Artificial
  Synthetic Construct
HSDAVFTDNYTRLRKQVAAKKYLNSIKK

SEQ ID NO: 416 - protein, 30 aa, Artificial
  Synthetic Construct
HSDAVFTDNYTRLRKQMAAKKYLNSIKNKR

SEQ ID NO: 417 - protein, 31 aa, Artificial
  Synthetic Construct
HSDAVFTDNYTRLRKQVAAKKYLQSIKNKRY

SEQ ID NO: 418 - protein, 31 aa, Artificial
  Synthetic Construct
HSDAVFTDNYTRLRKQLAAKKYLNTIKNKRY

SEQ ID NO: 419 - protein, 31 aa, Artificial
  Synthetic Construct
HSDAVFTDNYTRLRKQVAAKKYLNSIKNKRY

SEQ ID NO: 420 - protein, 31 aa, Artificial
  Synthetic Construct
HSDAVFTDNYTRLRKQMAAKKYLQSIKNKRY

SEQ ID NO: 421 - protein, 31 aa, Artificial
  Synthetic Construct
HSDAVFTDNYTRLRKQMAAKKYLNTIKNKRY

SEQ ID NO: 422 - protein, 31 aa, Artificial
  Synthetic Construct
HSDAVFTDQYTRLRKQMAAKKYLNSIKNKRY

SEQ ID NO: 423 - protein, 31 aa, Artificial
  Synthetic Construct
HSDAVFTDQYTRLRKQLAAKKYLNTIKNKRY

SEQ ID NO: 424 - protein, 31 aa, Artificial
  Synthetic Construct
HSDAVFTDNYTRLRKQMAAHKYLNSIKNKRY

SEQ ID NO: 425 - protein, 31 aa, Artificial
  Synthetic Construct
HSDAVFTDNYTRLRKQMAAKHYLNSIKNKRY

SEQ ID NO: 426 - protein, 31 aa, Artificial
  Synthetic Construct
HSDAVFTDQYTRLRKQLAAHKYLNTIKNKRY

SEQ ID NO: 427 - protein, 31 aa, Artificial
  Synthetic Construct
HSDAVFTDQYTRLRKQLAAKHYLNTIKNKRY

SEQ ID NO: 428 - protein, 30 aa, Artificial
  Synthetic Construct
HSDAVFTDNYTRLRKQVAAKKYLQSIKKKR

SEQ ID NO: 429 - protein, 30 aa, Artificial
  Synthetic Construct
HSDAVFTDNYTRLRKQVAAKKYLNSIKKKR

SEQ ID NO: 430 - protein, 31 aa, Artificial; identical to SEQ ID NO: 419
  Synthetic Construct

SEQ ID NO: 431 - protein, 30 aa, Artificial
  Synthetic Construct
HSDAVFTDNYTRLRKQVAVKKYLQSIKKKR

SEQ ID NO: 432 - protein, 29 aa, Artificial
  Synthetic Construct
HSDAVFTDNYTRLRKQVAVKKYLQSIKKK

SEQ ID NO: 433 - protein, 31 aa, Artificial
  Synthetic Construct
HSDAVFTDNYTRLRKQVAVKKYLQSIKNKRY

SEQ ID NO: 434 - protein, 31 aa, Artificial
  Synthetic Construct
HSDAVFTDNYTRLRKQVAAKKYLQSILKKRY

SEQ ID NO: 435 - protein, 30 aa, Artificial
  Synthetic Construct
HSDAVFTDNYTRLRKQVAAKKYLQSILKKR

SEQ ID NO: 436 - protein, 29 aa, Artificial
  Synthetic Construct
HSDAVFTDNYTRLRKQVAAKKYLQSILKK

SEQ ID NO: 437 - protein, 29 aa, Artificial
  Synthetic Construct
HSDAVFTDNYTRLRKQVAAKKYLQSIKNK

SEQ ID NO: 438 - protein, 31 aa, Artificial
  Synthetic Construct
HSDAVFTDNYTRLRKQVAVKKYLQSILKKRY

SEQ ID NO: 439 - protein, 30 aa, Artificial
  Synthetic Construct
HSDAVFTDNYTRLRKQVAVKKYLQSILKKR

SEQ ID NO: 440 - protein, 29 aa, Artificial
  Synthetic Construct
HSDAVFTDNYTRLRKQVAVKKYLQSILKK

SEQ ID NO: 441 - protein, 29 aa, Artificial
  Synthetic Construct
HSDAVFTDNYTRLRKQVAVKKYLQSIKNK

SEQ ID NO: 442 - protein, 31 aa, Artificial
  Synthetic Construct
HSDAVFTDNYTRLRKQVAAKKYLQSILNKRY

SEQ ID NO: 443 - protein, 30 aa, Artificial
  Synthetic Construct
HSDAVFTDNYTRLRKQVAAKKYLQSILNKR

SEQ ID NO: 444 - protein, 29 aa, Artificial
  Synthetic Construct
HSDAVFTDNYTRLRKQVAAKKYLQSILNK

SEQ ID NO: 445 - protein, 30 aa, Artificial
  Synthetic Construct
HSDAVFTDNYTRLRKQMAEKKYLNSIKNKR

SEQ ID NO: 446 - protein, 30 aa, Artificial
  Synthetic Construct
HSDAVFTDNYTRLRKQMAFKKYLNSIKNKR

SEQ ID NO: 447 - protein, 30 aa, Artificial
  Synthetic Construct
HSDAVFTDNYTRLRKQMAGKKYLNSIKNKR

SEQ ID NO: 448 - protein, 30 aa, Artificial
  Synthetic Construct
HSDAVFTDNYTRLRKQMAHKKYLNSIKNKR

SEQ ID NO: 449 - protein, 30 aa, Artificial
  Synthetic Construct
HSDAVFTDNYTRLRKQMAIKKYLNSIKNKR

SEQ ID NO: 450 - protein, 30 aa, Artificial
  Synthetic Construct
HSDAVFTDNYTRLRKQMAKKKYLNSIKNKR

SEQ ID NO: 451 - protein, 30 aa, Artificial
  Synthetic Construct
HSDAVFTDNYTRLRKQMALKKYLNSIKNKR

SEQ ID NO: 452 - protein, 30 aa, Artificial
  Synthetic Construct
HSDAVFTDNYTRLRKQMAMKKYLNSIKNKR

SEQ ID NO: 453 - protein, 30 aa, Artificial
  Synthetic Construct
HSDAVFTDNYTRLRKQMANKKYLNSIKNKR

SEQ ID NO: 454 - protein, 30 aa, Artificial
  Synthetic Construct
HSDAVFTDNYTRLRKQMAPKKYLNSIKNKR

SEQ ID NO: 455 - protein, 30 aa, Artificial
  Synthetic Construct
HSDAVFTDNYTRLRKQMAQKKYLNSIKNKR

SEQ ID NO: 456 - protein, 30 aa, Artificial
  Synthetic Construct
HSDAVFTDNYTRLRKQMARKKYLNSIKNKR

SEQ ID NO: 457 - protein, 30 aa, Artificial
  Synthetic Construct
HSDAVFTDNYTRLRKQMASKKYLNSIKNKR

SEQ ID NO: 458 - protein, 30 aa, Artificial
  Synthetic Construct
HSDAVFTDNYTRLRKQMATKKYLNSIKNKR

SEQ ID NO: 459 - protein, 30 aa, Artificial
  Synthetic Construct
HSDAVFTDNYTRLRKQMAVKKYLNSIKNKR

SEQ ID NO: 460 - protein, 30 aa, Artificial
  Synthetic Construct
HSDAVFTDNYTRLRKQMAWKKYLNSIKNKR

SEQ ID NO: 461 - protein, 30 aa, Artificial
  Synthetic Construct
HSDAVFTDNYTRLRKQMAYKKYLNSIKNKR

SEQ ID NO: 462 - protein, 30 aa, Artificial
  Synthetic Construct
HSDAVFTDNYTRLRKQMAAKKYLNSIANKR

SEQ ID NO: 463 - protein, 30 aa, Artificial
  Synthetic Construct
HSDAVFTDNYTRLRKQMAAKKYLNSIDNKR

SEQ ID NO: 464 - protein, 30 aa, Artificial
  Synthetic Construct
HSDAVFTDNYTRLRKQMAAKKYLNSIENKR

SEQ ID NO: 465 - protein, 30 aa, Artificial
  Synthetic Construct
HSDAVFTDNYTRLRKQMAAKKYLNSIFNKR

SEQ ID NO: 466 - protein, 30 aa, Artificial
  Synthetic Construct
HSDAVFTDNYTRLRKQMAAKKYLNSIGNKR

SEQ ID NO: 467 - protein, 30 aa, Artificial
  Synthetic Construct
HSDAVFTDNYTRLRKQMAAKKYLNSIHNKR

SEQ ID NO: 468 - protein, 30 aa, Artificial
  Synthetic Construct
HSDAVFTDNYTRLRKQMAAKKYLNSIINKR

SEQ ID NO: 469 - protein, 30 aa, Artificial
  Synthetic Construct
HSDAVFTDNYTRLRKQMAAKKYLNSIMNKR

SEQ ID NO: 470 - protein, 30 aa, Artificial
  Synthetic Construct
HSDAVFTDNYTRLRKQMAAKKYLNSINNKR

SEQ ID NO: 471 - protein, 30 aa, Artificial
  Synthetic Construct
HSDAVFTDNYTRLRKQMAAKKYLNSIPNKR

SEQ ID NO: 472 - protein, 30 aa, Artificial
  Synthetic Construct
HSDAVFTDNYTRLRKQMAAKKYLNSIQNKR

SEQ ID NO: 473 - protein, 30 aa, Artificial
  Synthetic Construct
HSDAVFTDNYTRLRKQMAAKKYLNSIRNKR

SEQ ID NO: 474 - protein, 30 aa, Artificial
  Synthetic Construct
HSDAVFTDNYTRLRKQMAAKKYLNSISNKR

SEQ ID NO: 475 - protein, 30 aa, Artificial
  Synthetic Construct
HSDAVFTDNYTRLRKQMAAKKYLNSITNKR

SEQ ID NO: 476 - protein, 30 aa, Artificial
  Synthetic Construct
HSDAVFTDNYTRLRKQMAAKKYLNSIVNKR

SEQ ID NO: 477 - protein, 30 aa, Artificial
  Synthetic Construct
HSDAVFTDNYTRLRKQMAAKKYLNSIWNKR

SEQ ID NO: 478 - protein, 30 aa, Artificial
  Synthetic Construct
HSDAVFTDNYTRLRKQMAAKKYLNSIYNKR

SEQ ID NO: 479 - protein, 30 aa, Artificial
  Synthetic Construct
HSDAVFTDNYTRLRKQMAAKKYLNSIKNAR

SEQ ID NO: 480 - protein, 30 aa, Artificial
  Synthetic Construct
HSDAVFTDNYTRLRKQMAAKKYLNSIKNDR

SEQ ID NO: 481 - protein, 30 aa, Artificial
  Synthetic Construct
HSDAVFTDNYTRLRKQMAAKKYLNSIKNER

SEQ ID NO: 482 - protein, 30 aa, Artificial
  Synthetic Construct
HSDAVFTDNYTRLRKQMAAKKYLNSIKNFR

SEQ ID NO: 483 - protein, 30 aa, Artificial
  Synthetic Construct
HSDAVFTDNYTRLRKQMAAKKYLNSIKNGR

SEQ ID NO: 484 - protein, 30 aa, Artificial
  Synthetic Construct
HSDAVFTDNYTRLRKQMAAKKYLNSIKNHR

SEQ ID NO: 485 - protein, 30 aa, Artificial
  Synthetic Construct
HSDAVFTDNYTRLRKQMAAKKYLNSIKNIR

SEQ ID NO: 486 - protein, 30 aa, Artificial
  Synthetic Construct
HSDAVFTDNYTRLRKQMAAKKYLNSIKNLR

SEQ ID NO: 487 - protein, 30 aa, Artificial
  Synthetic Construct
HSDAVFTDNYTRLRKQMAAKKYLNSIKNMR

SEQ ID NO: 488 - protein, 30 aa, Artificial
  Synthetic Construct
HSDAVFTDNYTRLRKQMAAKKYLNSIKNNR

SEQ ID NO: 489 - protein, 30 aa, Artificial
  Synthetic Construct
HSDAVFTDNYTRLRKQMAAKKYLNSIKNPR

SEQ ID NO: 490 - protein, 30 aa, Artificial
  Synthetic Construct
HSDAVFTDNYTRLRKQMAAKKYLNSIKNQR

SEQ ID NO: 491 - protein, 30 aa, Artificial
  Synthetic Construct
HSDAVFTDNYTRLRKQMAAKKYLNSIKNRR

SEQ ID NO: 492 - protein, 30 aa, Artificial
  Synthetic Construct
HSDAVFTDNYTRLRKQMAAKKYLNSIKNSR

SEQ ID NO: 493 - protein, 30 aa, Artificial
  Synthetic Construct
HSDAVFTDNYTRLRKQMAAKKYLNSIKNTR

SEQ ID NO: 494 - protein, 30 aa, Artificial
  Synthetic Construct
HSDAVFTDNYTRLRKQMAAKKYLNSIKNVR

SEQ ID NO: 495 - protein, 30 aa, Artificial
  Synthetic Construct
HSDAVFTDNYTRLRKQMAAKKYLNSIKNWR

SEQ ID NO: 496 - protein, 30 aa, Artificial
  Synthetic Construct
HSDAVFTDNYTRLRKQMAAKKYLNSIKNYR

SEQ ID NO: 497 - protein, 30 aa, Artificial
  Synthetic Construct
HSDAVFTDNYTRLRKQMAAKKYLNSIKNKA

SEQ ID NO: 498 - protein, 30 aa, Artificial
  Synthetic Construct
HSDAVFTDNYTRLRKQMAAKKYLNSIKNKD

SEQ ID NO: 499 - protein, 30 aa, Artificial
  Synthetic Construct
HSDAVFTDNYTRLRKQMAAKKYLNSIKNKE

SEQ ID NO: 500 - protein, 30 aa, Artificial
  Synthetic Construct
HSDAVFTDNYTRLRKQMAAKKYLNSIKNKF

SEQ ID NO: 501 - protein, 30 aa, Artificial
  Synthetic Construct
HSDAVFTDNYTRLRKQMAAKKYLNSIKNKG

SEQ ID NO: 502 - protein, 30 aa, Artificial
  Synthetic Construct
HSDAVFTDNYTRLRKQMAAKKYLNSIKNKH

SEQ ID NO: 503 - protein, 30 aa, Artificial
  Synthetic Construct
HSDAVFTDNYTRLRKQMAAKKYLNSIKNKI

SEQ ID NO: 504 - protein, 30 aa, Artificial
  Synthetic Construct
HSDAVFTDNYTRLRKQMAAKKYLNSIKNKK

SEQ ID NO: 505 - protein, 30 aa, Artificial
  Synthetic Construct
HSDAVFTDNYTRLRKQMAAKKYLNSIKNKL

SEQ ID NO: 506 - protein, 30 aa, Artificial
  Synthetic Construct
HSDAVFTDNYTRLRKQMAAKKYLNSIKNKM

SEQ ID NO: 507 - protein, 30 aa, Artificial
  Synthetic Construct
HSDAVFTDNYTRLRKQMAAKKYLNSIKNKN

SEQ ID NO: 508 - protein, 30 aa, Artificial
  Synthetic Construct
HSDAVFTDNYTRLRKQMAAKKYLNSIKNKP

SEQ ID NO: 509 - protein, 30 aa, Artificial
  Synthetic Construct
HSDAVFTDNYTRLRKQMAAKKYLNSIKNKQ

SEQ ID NO: 510 - protein, 30 aa, Artificial
  Synthetic Construct
HSDAVFTDNYTRLRKQMAAKKYLNSIKNKS

SEQ ID NO: 511 - protein, 30 aa, Artificial
  Synthetic Construct
HSDAVFTDNYTRLRKQMAAKKYLNSIKNKT

SEQ ID NO: 512 - protein, 30 aa, Artificial
  Synthetic Construct
HSDAVFTDNYTRLRKQMAAKKYLNSIKNKV

SEQ ID NO: 513 - protein, 30 aa, Artificial
  Synthetic Construct
HSDAVFTDNYTRLRKQMAAKKYLNSIKNKW

SEQ ID NO: 514 - protein, 30 aa, Artificial
  Synthetic Construct
HSDAVFTDNYTRLRKQMAAKKYLNSIKNKY

SEQ ID NO: 515 - protein, 40 aa, Artificial
  Synthetic Construct
HSDAVFTDNYTRLRKQVAAKKYLQSIKNKRYSWCEPGWCR

SEQ ID NO: 516 - protein, 31 aa, Artificial
  Synthetic Construct
HSDAVFTDDYTRLRKEVAAKKYLESIKDKRY

SEQ ID NO: 517 - protein, 30 aa, Artificial
  Synthetic Construct
HSDAVFTDNYTRLRKQMAAKKYLNSIKNRI

SEQ ID NO: 518 - protein, 30 aa, Artificial
  Synthetic Construct
HSDAVFTDNYTRLRKQMAGKKYLNSIKNRI

SEQ ID NO: 519 - protein, 30 aa, Artificial
  Synthetic Construct
HSDAVFTDNYTRLRKQMAKKKYLNSIKNRI

SEQ ID NO: 520 - protein, 30 aa, Artificial
  Synthetic Construct
HSDAVFTDNYTRLRKQMARKKYLNSIKNRI

SEQ ID NO: 521 - protein, 30 aa, Artificial
  Synthetic Construct
HSDAVFTDNYTRLRKQMASKKYLNSIKNRI

SEQ ID NO: 522 - protein, 30 aa, Artificial
  Synthetic Construct
HSDAVFTDNYTRLRKQMAAKKYLNSIPNRI

SEQ ID NO: 523 - protein, 30 aa, Artificial
  Synthetic Construct
HSDAVFTDNYTRLRKQMAGKKYLNSIPNRI

SEQ ID NO: 524 - protein, 30 aa, Artificial
  Synthetic Construct
HSDAVFTDNYTRLRKQMAKKKYLNSIPNRI

SEQ ID NO: 525 - protein, 30 aa, Artificial
  Synthetic Construct
HSDAVFTDNYTRLRKQMARKKYLNSIPNRI

SEQ ID NO: 526 - protein, 30 aa, Artificial
  Synthetic Construct
HSDAVFTDNYTRLRKQMASKKYLNSIPNRI

SEQ ID NO: 527 - protein, 30 aa, Artificial
  Synthetic Construct
HSDAVFTDNYTRLRKQMAAKKYLNSIQNRI

SEQ ID NO: 528 - protein, 30 aa, Artificial
  Synthetic Construct
HSDAVFTDNYTRLRKQMAGKKYLNSIQNRI

SEQ ID NO: 529 - protein, 30 aa, Artificial
  Synthetic Construct
HSDAVFTDNYTRLRKQMAKKKYLNSIQNRI

SEQ ID NO: 530 - protein, 30 aa, Artificial
  Synthetic Construct
HSDAVFTDNYTRLRKQMARKKYLNSIQNRI

SEQ ID NO: 531 - protein, 30 aa, Artificial
  Synthetic Construct
HSDAVFTDNYTRLRKQMASKKYLNSIQNRI

SEQ ID NO: 532 - protein, 30 aa, Artificial
  Synthetic Construct
HSDAVFTDNYTRLRKQMAAKKYLNSIRNRI

SEQ ID NO: 533 - protein, 30 aa, Artificial
  Synthetic Construct
HSDAVFTDNYTRLRKQMAGKKYLNSIRNRI

SEQ ID NO: 534 - protein, 30 aa, Artificial
  Synthetic Construct
HSDAVFTDNYTRLRKQMAKKKYLNSIRNRI

SEQ ID NO: 535 - protein, 30 aa, Artificial
  Synthetic Construct
HSDAVFTDNYTRLRKQMARKKYLNSIRNRI

SEQ ID NO: 536 - protein, 30 aa, Artificial
  Synthetic Construct
HSDAVFTDNYTRLRKQMASKKYLNSIRNRI

SEQ ID NO: 537 - protein, 31 aa, Artificial
  Synthetic Construct
HSDAVFTENYTKLRKQLAAKKYLNDLKKGGT

SEQ ID NO: 538 - protein, 31 aa, Artificial; identical to SEQ ID NO: 537
  Synthetic Construct

SEQ ID NO: 539 - protein, 31 aa, Artificial; identical to SEQ ID NO: 537
  Synthetic Construct

SEQ ID NO: 540 - protein, 28 aa, Artificial
  Synthetic Construct
HSDAVFTENYTKLRKQLAAKKYLNDLKK

SEQ ID NO: 541 - protein, 31 aa, Artificial
  Synthetic Construct
HSDAVFTDNYTRLRKQLAAKKYLNDIKKGGT

SEQ ID NO: 542 - protein, 28 aa, Artificial
  Synthetic Construct
HSDAVFTDNYTRLRKQLAAKKYLNDIKK

SEQ ID NO: 543 - protein, 31 aa, Artificial
  Synthetic Construct
HSDAVFTDNYTRLRKQMAVKKYLNDLKKGGT

SEQ ID NO: 544 - protein, 31 aa, Artificial
  Synthetic Construct
HSDAVFTDNYTRLRKQMAAKKYLNDIKKGGT

SEQ ID NO: 545 - protein, 31 aa, Artificial
  Synthetic Construct
HSDAVFTDNYTRLRKQLAVKKYLNDIKKGGT

SEQ ID NO: 546 - protein, 30 aa, Artificial
  Synthetic Construct
HSDAVFTDNYTRLRKQLAAKKYLNDIKKGG

SEQ ID NO: 547 - protein, 29 aa, Artificial
  Synthetic Construct
HSDAVFTDNYTRLRKQLAAKKYLNDIKKG

SEQ ID NO: 548 - protein, 28 aa, Artificial
  Synthetic Construct
HSDAVFTDNYTRLRKQLAAKKYLNDIKK

SEQ ID NO: 549 - protein, 29 aa, Artificial
  Synthetic Construct
HSDAVFTDNYTRLRKQLAAKKYLNDIKKQ

SEQ ID NO: 550 - protein, 30 aa, Artificial
  Synthetic Construct
HSDAVFTDNYTRLRKQLAAKKYLNDIKKNQ

SEQ ID NO: 551 - protein, 28 aa, Artificial
  Synthetic Construct
HSDAVFTDNYTRLREQMAVKKYLNSILN

SEQ ID NO: 552 - protein, 28 aa, Artificial
  Synthetic Construct
HSDAVFTDNYTRLRKQLAVKKYLNSILN

SEQ ID NO: 553 - protein, 28 aa, Artificial
  Synthetic Construct
HSDAVFTDNYTRLRKQMAAKKYLNSILN

SEQ ID NO: 554 - protein, 31 aa, Artificial; identical to SEQ ID NO: 537
  Synthetic Construct

SEQ ID NO: 555 - protein, 30 aa, Artificial
  Synthetic Construct
HSDAVFTDNYTRLRKQMACKKYLNSIKNKR

SEQ ID NO: 556 - protein, 30 aa, Artificial
  Synthetic Construct
HSDAVFTDNYTRLRKQMADKKYLNSIKNKR

SEQ ID NO: 557 - protein, 30 aa, Artificial
  Synthetic Construct
HSDAVFTDNYTRLRKQMAAKKYLNSICNKR

SEQ ID NO: 558 - protein, 30 aa, Artificial
  Synthetic Construct
HSDAVFTDNYTRLRKQMAAKKYLNSIKNCR

SEQ ID NO: 559 - protein, 31 aa, Artificial
  Synthetic Construct
HSDAVFTDQYTRLRKQVAAKKYLQSIKQKRY

SEQ ID NO: 560 - protein, 31 aa, Artificial
  Synthetic Construct
HTDAVFTDQYTRLRKQVAAKKYLQSIKQKRY

SEQ ID NO: 561 - protein, 31 aa, Artificial
  Synthetic Construct
HSDAVFTDQYTRLRKQMAAKKYLQSIKQKRY

SEQ ID NO: 562 - protein, 29 aa, Artificial
  Synthetic Construct
HSDAVFTDQYTRLRKQVAAKKYLQSIKQK

SEQ ID NO: 563 - protein, 31 aa, Artificial
  Synthetic Construct
HTEAVFTDQYTRLRKQVAAKKYLQSIKQKRY

SEQ ID NO: 564 - protein, 31 aa, Artificial
  Synthetic Construct
HSDAVFTDQYTRLRKQLAVKKYLQDIKQGGT

SEQ ID NO: 565 - protein, 30 aa, Artificial
  Synthetic Construct
HSDAVFTDQYTRLRKQMAAKKYLQSIKQKR

SEQ ID NO: 566 - protein, 31 aa, Artificial
  Synthetic Construct
HSDAVFTDQYTRLRKQLAAKKYLQTIKQKRY

SEQ ID NO: 567 - protein, 31 aa, Artificial
  Synthetic Construct
HSDAVFTDQYTRLRKQMAAKKYLQTIKQKRY

SEQ ID NO: 568 - protein, 31 aa, Artificial
  Synthetic Construct
HSDAVFTDQYTRLRKQMAAHKYLQSIKQKRY

SEQ ID NO: 569 - protein, 31 aa, Artificial
  Synthetic Construct
HSDAVFTDQYTRLRKQMAAKHYLQSIKQKRY

SEQ ID NO: 570 - protein, 30 aa, Artificial
  Synthetic Construct
HSDAVFTDQYTRLRKQMAGKKYLQSIKQKR

SEQ ID NO: 571 - protein, 30 aa, Artificial
  Synthetic Construct
HSDAVFTDQYTRLRKQMAKKKYLQSIKQKR

SEQ ID NO: 572 - protein, 30 aa, Artificial
  Synthetic Construct
HSDAVFTDQYTRLRKQMARKKYLQSIKQKR

SEQ ID NO: 573 - protein, 30 aa, Artificial
  Synthetic Construct
HSDAVFTDQYTRLRKQMASKKYLQSIKQKR

SEQ ID NO: 574 - protein, 30 aa, Artificial
  Synthetic Construct
HSDAVFTDQYTRLRKQMAAKKYLQSIPQKR

SEQ ID NO: 575 - protein, 30 aa, Artificial
  Synthetic Construct
HSDAVFTDQYTRLRKQMAAKKYLQSIQQKR

SEQ ID NO: 576 - protein, 30 aa, Artificial
  Synthetic Construct
HSDAVFTDQYTRLRKQMAAKKYLQSIRQKR

SEQ ID NO: 577 - protein, 30 aa, Artificial
  Synthetic Construct
HSDAVFTDQYTRLRKQMAAKKYLQSIKQRR

SEQ ID NO: 578 - protein, 30 aa, Artificial
  Synthetic Construct
HSDAVFTDQYTRLRKQMAAKKYLQSIKQKA

SEQ ID NO: 579 - protein, 30 aa, Artificial
  Synthetic Construct
HSDAVFTDQYTRLRKQMAAKKYLQSIKQKF

SEQ ID NO: 580 - protein, 30 aa, Artificial
  Synthetic Construct
HSDAVFTDQYTRLRKQMAAKKYLQSIKQKH

SEQ ID NO: 581 - protein, 30 aa, Artificial
  Synthetic Construct
HSDAVFTDQYTRLRKQMAAKKYLQSIKQKI

SEQ ID NO: 582 - protein, 30 aa, Artificial
  Synthetic Construct
HSDAVFTDQYTRLRKQMAAKKYLQSIKQKK

SEQ ID NO: 583 - protein, 30 aa, Artificial
  Synthetic Construct
HSDAVFTDQYTRLRKQMAAKKYLQSIKQKL

SEQ ID NO: 584 - protein, 30 aa, Artificial
  Synthetic Construct
HSDAVFTDQYTRLRKQMAAKKYLQSIKQKM

SEQ ID NO: 585 - protein, 30 aa, Artificial
  Synthetic Construct
HSDAVFTDQYTRLRKQMAAKKYLQSIKQKP

SEQ ID NO: 586 - protein, 30 aa, Artificial
  Synthetic Construct
HSDAVFTDQYTRLRKQMAAKKYLQSIKQKQ

SEQ ID NO: 587 - protein, 30 aa, Artificial
  Synthetic Construct
HSDAVFTDQYTRLRKQMAAKKYLQSIKQKS

SEQ ID NO: 588 - protein, 30 aa, Artificial
  Synthetic Construct
HSDAVFTDQYTRLRKQMAAKKYLQSIKQKT

SEQ ID NO: 589 - protein, 30 aa, Artificial
  Synthetic Construct
HSDAVFTDQYTRLRKQMAAKKYLQSIKQKV

SEQ ID NO: 590 - protein, 30 aa, Artificial
  Synthetic Construct
HSDAVFTDQYTRLRKQMAAKKYLQSIKQKW

SEQ ID NO: 591 - protein, 30 aa, Artificial
  Synthetic Construct
HSDAVFTDQYTRLRKQMAAKKYLQSIKQKY

SEQ ID NO: 592 - protein, 30 aa, Artificial
  Synthetic Construct
HSDAVFTDQYTRLRKQMAGKKYLQSIKQRI

SEQ ID NO: 593 - protein, 30 aa, Artificial
  Synthetic Construct
HSDAVFTDQYTRLRKQMAKKKYLQSIKQRI

SEQ ID NO: 594 - protein, 30 aa, Artificial
  Synthetic Construct
HSDAVFTDQYTRLRKQMASKKYLQSIKQRI

SEQ ID NO: 595 - protein, 30 aa, Artificial
  Synthetic Construct
HSDAVFTDQYTRLRKQMAAKKYLQSIPQRI

SEQ ID NO: 596 - protein, 30 aa, Artificial
  Synthetic Construct
HSDAVFTDQYTRLRKQMASKKYLQSIRQRI

SEQ ID NO: 597 - protein, 31 aa, Artificial
  Synthetic Construct
HSDAVFTDNYTRLRKQVAAKKYLQSIKQKRY

SEQ ID NO: 598 - protein, 31 aa, Artificial
  Synthetic Construct
HTDAVFTDNYTRLRKQVAAKKYLQSIKQKRY

SEQ ID NO: 599 - protein, 31 aa, Artificial
  Synthetic Construct
HSDAVFTDNYTRLRKQMAAKKYLQSIKQKRY

SEQ ID NO: 600 - protein, 29 aa, Artificial
  Synthetic Construct
HSDAVFTDNYTRLRKQVAAKKYLQSIKQK

SEQ ID NO: 601 - protein, 31 aa, Artificial
  Synthetic Construct
HTEAVFTDNYTRLRKQVAAKKYLQSIKQKRY

SEQ ID NO: 602 - protein, 31 aa, Artificial
  Synthetic Construct
HSDAVFTDNYTRLRKQLAVKKYLQDIKQGGT

SEQ ID NO: 603 - protein, 30 aa, Artificial
  Synthetic Construct
HSDAVFTDNYTRLRKQMAAKKYLQSIKQKR

SEQ ID NO: 604 - protein, 31 aa, Artificial
  Synthetic Construct
HSDAVFTDNYTRLRKQLAAKKYLQTIKQKRY

SEQ ID NO: 605 - protein, 31 aa, Artificial
  Synthetic Construct
HSDAVFTDNYTRLRKQMAAKKYLQTIKQKRY

SEQ ID NO: 606 - protein, 31 aa, Artificial
  Synthetic Construct
HSDAVFTDNYTRLRKQMAAHKYLQSIKQKRY

SEQ ID NO: 607 - protein, 31 aa, Artificial
  Synthetic Construct
HSDAVFTDNYTRLRKQMAAKHYLQSIKQKRY

SEQ ID NO: 608 - protein, 30 aa, Artificial
  Synthetic Construct
HSDAVFTDNYTRLRKQMAGKKYLQSIKQKR

SEQ ID NO: 609 - protein, 30 aa, Artificial
  Synthetic Construct
HSDAVFTDNYTRLRKQMAKKKYLQSIKQKR

SEQ ID NO: 610 - protein, 30 aa, Artificial
  Synthetic Construct
HSDAVFTDNYTRLRKQMARKKYLQSIKQKR

SEQ ID NO: 611 - protein, 30 aa, Artificial
  Synthetic Construct
HSDAVFTDNYTRLRKQMASKKYLQSIKQKR

SEQ ID NO: 612 - protein, 30 aa, Artificial
  Synthetic Construct
HSDAVFTDNYTRLRKQMAAKKYLQSIPQKR

SEQ ID NO: 613 - protein, 30 aa, Artificial
  Synthetic Construct
HSDAVFTDNYTRLRKQMAAKKYLQSIQQKR

SEQ ID NO: 614 - protein, 30 aa, Artificial
  Synthetic Construct
HSDAVFTDNYTRLRKQMAAKKYLQSIRQKR

SEQ ID NO: 615 - protein, 30 aa, Artificial
  Synthetic Construct
HSDAVFTDNYTRLRKQMAAKKYLQSIKQRR

SEQ ID NO: 616 - protein, 30 aa, Artificial
  Synthetic Construct
HSDAVFTDNYTRLRKQMAAKKYLQSIKQKA

SEQ ID NO: 617 - protein, 30 aa, Artificial
  Synthetic Construct
HSDAVFTDNYTRLRKQMAAKKYLQSIKQKF

SEQ ID NO: 618 - protein, 30 aa, Artificial
  Synthetic Construct
HSDAVFTDNYTRLRKQMAAKKYLQSIKQKH

SEQ ID NO: 619 - protein, 30 aa, Artificial
  Synthetic Construct
HSDAVFTDNYTRLRKQMAAKKYLQSIKQKI

SEQ ID NO: 620 - protein, 30 aa, Artificial
  Synthetic Construct
HSDAVFTDNYTRLRKQMAAKKYLQSIKQKK

SEQ ID NO: 621 - protein, 30 aa, Artificial
  Synthetic Construct
HSDAVFTDNYTRLRKQMAAKKYLQSIKQKL

SEQ ID NO: 622 - protein, 30 aa, Artificial
  Synthetic Construct
HSDAVFTDNYTRLRKQMAAKKYLQSIKQKM

SEQ ID NO: 623 - protein, 30 aa, Artificial
  Synthetic Construct
HSDAVFTDNYTRLRKQMAAKKYLQSIKQKP

SEQ ID NO: 624 - protein, 30 aa, Artificial
  Synthetic Construct
HSDAVFTDNYTRLRKQMAAKKYLQSIKQKQ

SEQ ID NO: 625 - protein, 30 aa, Artificial
  Synthetic Construct
HSDAVFTDNYTRLRKQMAAKKYLQSIKQKS

SEQ ID NO: 626 - protein, 30 aa, Artificial
  Synthetic Construct
HSDAVFTDNYTRLRKQMAAKKYLQSIKQKT

SEQ ID NO: 627 - protein, 30 aa, Artificial
  Synthetic Construct
HSDAVFTDNYTRLRKQMAAKKYLQSIKQKV

SEQ ID NO: 628 - protein, 30 aa, Artificial
  Synthetic Construct
HSDAVFTDNYTRLRKQMAAKKYLQSIKQKW

SEQ ID NO: 629 - protein, 30 aa, Artificial
  Synthetic Construct
HSDAVFTDNYTRLRKQMAAKKYLQSIKQKY

SEQ ID NO: 630 - protein, 30 aa, Artificial
  Synthetic Construct
HSDAVFTDNYTRLRKQMAGKKYLQSIKQRI

SEQ ID NO: 631 - protein, 30 aa, Artificial
  Synthetic Construct
HSDAVFTDNYTRLRKQMAKKKYLQSIKQRI

SEQ ID NO: 632 - protein, 30 aa, Artificial
  Synthetic Construct
HSDAVFTDNYTRLRKQMASKKYLQSIKQRI

SEQ ID NO: 633 - protein, 30 aa, Artificial
  Synthetic Construct
HSDAVFTDNYTRLRKQMAAKKYLQSIPQRI

SEQ ID NO: 634 - protein, 30 aa, Artificial
  Synthetic Construct
HSDAVFTDNYTRLRKQMASKKYLQSIRQRI

SEQ ID NO: 635 - protein, 31 aa, Artificial
  Synthetic Construct
HSDAVFTDQYTRLRKQVAAKKYLQSIKNKRY

SEQ ID NO: 636 - protein, 31 aa, Artificial
  Synthetic Construct
HTDAVFTDQYTRLRKQVAAKKYLQSIKNKRY

SEQ ID NO: 637 - protein, 31 aa, Artificial
  Synthetic Construct
HSDAVFTDQYTRLRKQMAAKKYLQSIKNKRY

SEQ ID NO: 638 - protein, 29 aa, Artificial
  Synthetic Construct
HSDAVFTDQYTRLRKQVAAKKYLQSIKNK

SEQ ID NO: 639 - protein, 31 aa, Artificial
  Synthetic Construct
HTEAVFTDQYTRLRKQVAAKKYLQSIKNKRY

SEQ ID NO: 640 - protein, 31 aa, Artificial
  Synthetic Construct
HSDAVFTDQYTRLRKQLAVKKYLQDIKNGGT

SEQ ID NO: 641 - protein, 30 aa, Artificial
  Synthetic Construct
HSDAVFTDQYTRLRKQMAAKKYLQSIKNKR

SEQ ID NO: 642 - protein, 31 aa, Artificial
  Synthetic Construct
HSDAVFTDQYTRLRKQLAAKKYLQTIKNKRY

SEQ ID NO: 643 - protein, 31 aa, Artificial
  Synthetic Construct
HSDAVFTDQYTRLRKQMAAKKYLQTIKNKRY

SEQ ID NO: 644 - protein, 31 aa, Artificial
  Synthetic Construct
HSDAVFTDQYTRLRKQMAAHKYLQSIKNKRY

SEQ ID NO: 645 - protein, 31 aa, Artificial
  Synthetic Construct
HSDAVFTDQYTRLRKQMAAKHYLQSIKNKRY

SEQ ID NO: 646 - protein, 30 aa, Artificial
  Synthetic Construct
HSDAVFTDQYTRLRKQMAGKKYLQSIKNKR

SEQ ID NO: 647 - protein, 30 aa, Artificial
  Synthetic Construct
HSDAVFTDQYTRLRKQMAKKKYLQSIKNKR

SEQ ID NO: 648 - protein, 30 aa, Artificial
  Synthetic Construct
HSDAVFTDQYTRLRKQMARKKYLQSIKNKR

SEQ ID NO: 649 - protein, 30 aa, Artificial
  Synthetic Construct
HSDAVFTDQYTRLRKQMASKKYLQSIKNKR

SEQ ID NO: 650 - protein, 30 aa, Artificial
  Synthetic Construct
HSDAVFTDQYTRLRKQMAAKKYLQSIPNKR

SEQ ID NO: 651 - protein, 30 aa, Artificial
  Synthetic Construct
HSDAVFTDQYTRLRKQMAAKKYLQSIQNKR

SEQ ID NO: 652 - protein, 30 aa, Artificial
  Synthetic Construct
HSDAVFTDQYTRLRKQMAAKKYLQSIRNKR

SEQ ID NO: 653 - protein, 30 aa, Artificial
  Synthetic Construct
HSDAVFTDQYTRLRKQMAAKKYLQSIKNRR

SEQ ID NO: 654 - protein, 30 aa, Artificial
  Synthetic Construct
HSDAVFTDQYTRLRKQMAAKKYLQSIKNKA

SEQ ID NO: 655 - protein, 30 aa, Artificial
  Synthetic Construct
HSDAVFTDQYTRLRKQMAAKKYLQSIKNKF

SEQ ID NO: 656 - protein, 30 aa, Artificial
  Synthetic Construct
HSDAVFTDQYTRLRKQMAAKKYLQSIKNKH

SEQ ID NO: 657 - protein, 30 aa, Artificial
  Synthetic Construct
HSDAVFTDQYTRLRKQMAAKKYLQSIKNKI

SEQ ID NO: 658 - protein, 30 aa, Artificial
  Synthetic Construct
HSDAVFTDQYTRLRKQMAAKKYLQSIKNKK

SEQ ID NO: 659 - protein, 30 aa, Artificial
  Synthetic Construct
HSDAVFTDQYTRLRKQMAAKKYLQSIKNKL

SEQ ID NO: 660 - protein, 30 aa, Artificial
  Synthetic Construct
HSDAVFTDQYTRLRKQMAAKKYLQSIKNKM

SEQ ID NO: 661 - protein, 30 aa, Artificial
  Synthetic Construct
HSDAVFTDQYTRLRKQMAAKKYLQSIKNKP

SEQ ID NO: 662 - protein, 30 aa, Artificial
  Synthetic Construct
HSDAVFTDQYTRLRKQMAAKKYLQSIKNKQ

SEQ ID NO: 663 - protein, 30 aa, Artificial
  Synthetic Construct
HSDAVFTDQYTRLRKQMAAKKYLQSIKNKS

SEQ ID NO: 664 - protein, 30 aa, Artificial
  Synthetic Construct
HSDAVFTDQYTRLRKQMAAKKYLQSIKNKT

SEQ ID NO: 665 - protein, 30 aa, Artificial
  Synthetic Construct
HSDAVFTDQYTRLRKQMAAKKYLQSIKNKV

SEQ ID NO: 666 - protein, 30 aa, Artificial
  Synthetic Construct
HSDAVFTDQYTRLRKQMAAKKYLQSIKNKW

SEQ ID NO: 667 - protein, 30 aa, Artificial
  Synthetic Construct
HSDAVFTDQYTRLRKQMAAKKYLQSIKNKY

SEQ ID NO: 668 - protein, 30 aa, Artificial
  Synthetic Construct
HSDAVFTDQYTRLRKQMAGKKYLQSIKNRI

SEQ ID NO: 669 - protein, 30 aa, Artificial
  Synthetic Construct
HSDAVFTDQYTRLRKQMAKKKYLQSIKNRI

SEQ ID NO: 670 - protein, 30 aa, Artificial
  Synthetic Construct
HSDAVFTDQYTRLRKQMASKKYLQSIKNRI

SEQ ID NO: 671 - protein, 30 aa, Artificial
  Synthetic Construct
HSDAVFTDQYTRLRKQMAAKKYLQSIPNRI

SEQ ID NO: 672 - protein, 30 aa, Artificial
  Synthetic Construct
HSDAVFTDQYTRLRKQMASKKYLQSIRNRI

SEQ ID NO: 673 - protein, 32 aa, Artificial
  Synthetic Construct
HSDAVFTDQYTRLRKQVAAKKYLQSIKQKRYC

SEQ ID NO: 674 - protein, 32 aa, Artificial
  Synthetic Construct
HTDAVFTDQYTRLRKQVAAKKYLQSIKQKRYC

SEQ ID NO: 675 - protein, 32 aa, Artificial
  Synthetic Construct
HSDAVFTDQYTRLRKQMAAKKYLQSIKQKRYC

SEQ ID NO: 676 - protein, 30 aa, Artificial
  Synthetic Construct
HSDAVFTDQYTRLRKQVAAKKYLQSIKQKC

SEQ ID NO: 677 - protein, 32 aa, Artificial
  Synthetic Construct
HTEAVFTDQYTRLRKQVAAKKYLQSIKQKRYC

SEQ ID NO: 678 - protein, 32 aa, Artificial
  Synthetic Construct
HSDAVFTDQYTRLRKQLAVKKYLQDIKQGGTC

SEQ ID NO: 679 - protein, 31 aa, Artificial
  Synthetic Construct
HSDAVFTDQYTRLRKQMAAKKYLQSIKQKRC

SEQ ID NO: 680 - protein, 32 aa, Artificial
  Synthetic Construct
HSDAVFTDQYTRLRKQLAAKKYLQTIKQKRYC

SEQ ID NO: 681 - protein, 32 aa, Artificial
  Synthetic Construct
HSDAVFTDQYTRLRKQMAAKKYLQTIKQKRYC

SEQ ID NO: 682 - protein, 32 aa, Artificial
  Synthetic Construct
HSDAVFTDQYTRLRKQMAAHKYLQSIKQKRYC

SEQ ID NO: 683 - protein, 32 aa, Artificial
  Synthetic Construct
HSDAVFTDQYTRLRKQMAAKHYLQSIKQKRYC

SEQ ID NO: 684 - protein, 31 aa, Artificial
  Synthetic Construct
HSDAVFTDQYTRLRKQMAGKKYLQSIKQKRC

SEQ ID NO: 685 - protein, 31 aa, Artificial
  Synthetic Construct
HSDAVFTDQYTRLRKQMAKKKYLQSIKQKRC

SEQ ID NO: 686 - protein, 31 aa, Artificial
  Synthetic Construct
HSDAVFTDQYTRLRKQMARKKYLQSIKQKRC

SEQ ID NO: 687 - protein, 31 aa, Artificial
  Synthetic Construct
HSDAVFTDQYTRLRKQMASKKYLQSIKQKRC

SEQ ID NO: 688 - protein, 31 aa, Artificial
  Synthetic Construct
HSDAVFTDQYTRLRKQMAAKKYLQSIPQKRC

SEQ ID NO: 689 - protein, 31 aa, Artificial
  Synthetic Construct
HSDAVFTDQYTRLRKQMAAKKYLQSIQQKRC

SEQ ID NO: 690 - protein, 31 aa, Artificial
  Synthetic Construct
HSDAVFTDQYTRLRKQMAAKKYLQSIRQKRC

SEQ ID NO: 691 - protein, 31 aa, Artificial
  Synthetic Construct
HSDAVFTDQYTRLRKQMAAKKYLQSIKQRRC

SEQ ID NO: 692 - protein, 31 aa, Artificial
  Synthetic Construct
HSDAVFTDQYTRLRKQMAAKKYLQSIKQKAC

SEQ ID NO: 693 - protein, 31 aa, Artificial
  Synthetic Construct
HSDAVFTDQYTRLRKQMAAKKYLQSIKQKFC

SEQ ID NO: 694 - protein, 31 aa, Artificial
  Synthetic Construct
HSDAVFTDQYTRLRKQMAAKKYLQSIKQKHC

SEQ ID NO: 695 - protein, 31 aa, Artificial
  Synthetic Construct
HSDAVFTDQYTRLRKQMAAKKYLQSIKQKIC

SEQ ID NO: 696 - protein, 31 aa, Artificial
  Synthetic Construct
HSDAVFTDQYTRLRKQMAAKKYLQSIKQKKC

SEQ ID NO: 697 - protein, 31 aa, Artificial
  Synthetic Construct
HSDAVFTDQYTRLRKQMAAKKYLQSIKQKLC

SEQ ID NO: 698 - protein, 31 aa, Artificial
  Synthetic Construct
HSDAVFTDQYTRLRKQMAAKKYLQSIKQKMC

SEQ ID NO: 699 - protein, 31 aa, Artificial
  Synthetic Construct
HSDAVFTDQYTRLRKQMAAKKYLQSIKQKPC

SEQ ID NO: 700 - protein, 31 aa, Artificial
  Synthetic Construct
HSDAVFTDQYTRLRKQMAAKKYLQSIKQKQC

SEQ ID NO: 701 - protein, 31 aa, Artificial
  Synthetic Construct
HSDAVFTDQYTRLRKQMAAKKYLQSIKQKSC

SEQ ID NO: 702 - protein, 31 aa, Artificial
  Synthetic Construct
HSDAVFTDQYTRLRKQMAAKKYLQSIKQKTC

SEQ ID NO: 703 - protein, 31 aa, Artificial
  Synthetic Construct
HSDAVFTDQYTRLRKQMAAKKYLQSIKQKVC

SEQ ID NO: 704 - protein, 31 aa, Artificial
  Synthetic Construct
HSDAVFTDQYTRLRKQMAAKKYLQSIKQKWC

SEQ ID NO: 705 - protein, 31 aa, Artificial
  Synthetic Construct
HSDAVFTDQYTRLRKQMAAKKYLQSIKQKYC

SEQ ID NO: 706 - protein, 31 aa, Artificial
  Synthetic Construct
HSDAVFTDQYTRLRKQMAGKKYLQSIKQRIC

SEQ ID NO: 707 - protein, 31 aa, Artificial
  Synthetic Construct
HSDAVFTDQYTRLRKQMAKKKYLQSIKQRIC

SEQ ID NO: 708 - protein, 31 aa, Artificial
  Synthetic Construct
HSDAVFTDQYTRLRKQMASKKYLQSIKQRIC

SEQ ID NO: 709 - protein, 31 aa, Artificial
  Synthetic Construct
HSDAVFTDQYTRLRKQMAAKKYLQSIPQRIC

SEQ ID NO: 710 - protein, 31 aa, Artificial
  Synthetic Construct
HSDAVFTDQYTRLRKQMASKKYLQSIRQRIC

SEQ ID NO: 711 - protein, 42 aa, Artificial; identical to SEQ ID NO: 151
  Synthetic Construct

SEQ ID NO: 712 - protein, 42 aa, Artificial; identical to SEQ ID NO: 113
  Synthetic Construct

SEQ ID NO: 713 - protein, 42 aa, Artificial
  Synthetic Construct
HSDAVFTDNYTRLLAKLALQRYLQSIRNKRYGGPSSGAPPPS

SEQ ID NO: 714 - protein, 42 aa, Artificial
  Synthetic Construct
HSDAVFTDNYTRLLAKLALQKYLQSIRNKRYGGPSSGAPPPS

SEQ ID NO: 715 - protein, 42 aa, Artificial; identical to SEQ ID NO: 30
  Synthetic Construct

SEQ ID NO: 716 - protein, 42 aa, Artificial
  Synthetic Construct
HSDAVFTDNYTRLRRQVAAKRYLQSIKNKRYGGPSSGAPPPS

SEQ ID NO: 717 - protein, 42 aa, Artificial
  Synthetic Construct
HSDAVFTDNYTRLRRQVAARKYLQSIRNKRYGGPSSGAPPPS

SEQ ID NO: 718 - protein, 42 aa, Artificial
  Synthetic Construct
HSDAVFTDNYTRLRKQVAAKRYLQSIRNKRYGGPSSGAPPPS

SEQ ID NO: 719 - protein, 39 aa, Artificial; identical to SEQ ID NO: 194
  Synthetic Construct

SEQ ID NO: 720 - protein, 39 aa, Artificial; identical to SEQ ID NO: 195
  Synthetic Construct

SEQ ID NO: 721 - protein, 39 aa, Artificial; identical to SEQ ID NO: 196
  Synthetic Construct

SEQ ID NO: 722 - protein, 39 aa, Artificial
  Synthetic Construct
HSDAVFTEQYTXLRXQVAAXXYLQSXXXGGPSSGAPPPS

SEQ ID NO: 723 - protein, 39 aa, Artificial
  Synthetic Constructs
HSDAVFTEQYTXLRXQVAAXXYLQXIXXGGPSSGAPPPS

SEQ ID NO: 724 - protein, 39 aa, Artificial
  Synthetic Constructs
HSDAVFTEQYTXLRXQXAAXXYLQSIXXGGPSSGAPPPS

SEQ ID NO: 725 - protein, 39 aa, Artificial
  Synthetic Constructs
HSDAVFTEQYTXLRXXVAAXXYLQSIXXGGPSSGAPPPS

SEQ ID NO: 726 - protein, 40 aa, Artificial
  Synthetic Constructs
HSDAVFTEQYTXLRXQVAAXXYLQSIXXXGGPSSGAPPPS

SEQ ID NO: 727 - protein, 39 aa, Artificial
  Synthetic Constructs
HSDAVFTEQYTXXRXQVAAXXYLQSIXXGGPSSGAPPPS

SEQ ID NO: 728 - protein, 39 aa, Artificial; identical to SEQ ID NO: 722
  Synthetic Constructs

SEQ ID NO: 729 - protein, 39 aa, Artificial; identical to SEQ ID NO: 723
  Synthetic Constructs

SEQ ID NO: 730 - protein, 39 aa, Artificial; identical to SEQ ID NO: 724
  Synthetic Constructs

SEQ ID NO: 731 - protein, 39 aa, Artificial; identical to SEQ ID NO: 725
  Synthetic Constructs

SEQ ID NO: 732 - protein, 40 aa, Artificial; identical to SEQ ID NO: 726
  Synthetic Constructs

SEQ ID NO: 733 - protein, 39 aa, Artificial; identical to SEQ ID NO: 727
  Synthetic Constructs

SEQ ID NO: 734 - protein, 39 aa, Artificial; identical to SEQ ID NO: 722
  Synthetic Constructs

SEQ ID NO: 735 - protein, 39 aa, Artificial; identical to SEQ ID NO: 723
  Synthetic Constructs

SEQ ID NO: 736 - protein, 39 aa, Artificial; identical to SEQ ID NO: 724
  Synthetic Constructs

SEQ ID NO: 737 - protein, 39 aa, Artificial; identical to SEQ ID NO: 725
  Synthetic Constructs

SEQ ID NO: 738 - protein, 40 aa, Artificial; identical to SEQ ID NO: 726
  Synthetic Constructs

(19) United States
(12) Patent Application Publication
(10) Pub. No.: US 2008/0146500 A1
(43) Pub. Date: Jun. 19, 2008

(54) Selective Vpac2 Receptor Peptide Agonists

(76) Inventors: Bengt Krister Bokvist, Hamburg (DE); John Philip Mayer, Indianapolis, IN (US); Lianshan Zhang, Carmel, IN (US); Jorge Alsina-Fernandez, Indianapolis, IN (US); Andrew Mark Vick, Fishers, IN (US)

Correspondence Address: ELI LILLY & COMPANY, PATENT DIVISION, P.O. BOX 6288, INDIANAPOLIS, IN, 46206-6288, US

(21) Appl. No.: 11/573,830

(22) PCT Filed: Aug. 11, 2005
(86) PCT No.: PCT/US05/28520

§ 371 Date: Feb. 16, 2007

Related U.S. Application Data
(60) Provisional application No. 60/602,461, filed on Aug. 18, 2004.
(60) Provisional application No. 60/602,350, filed on Aug. 18, 2004.

(51) Int. Cl. A61K 38/18; C07K 14/48

(52) U.S. Cl. 514/12; 530/324

(57) ABSTRACT

The present invention encompasses peptides that selectively activate the VPAC2 receptor and are useful in the treatment of diabetes.

The present invention relates to selective VPAC2 receptor peptide agonists.

In particular, the present invention relates to selective VPAC2 receptor peptide agonists which are covalently attached to one or more molecules of polyethylene glycol or a derivative thereof.

Type 2 diabetes, or non-insulin dependent diabetes mellitus (NIDDM), is the most common form of diabetes, affecting 90% of people with diabetes. With NIDDM, patients have impaired β-cell function resulting in insufficient insulin production and/or decreased insulin sensitivity. If NIDDM is not controlled, excess glucose accumulates in the blood, resulting in hyperglycemia. Over time, more serious complications may arise including renal dysfunction, cardiovascular problems, visual loss, lower limb ulceration, neuropathy, and ischemia. Treatments for NIDDM include improving diet, exercise, and weight control as well as using a variety of oral medications. Individuals with NIDDM can initially control their blood glucose levels by taking such oral medications. These medications, however, do not slow the progressive loss of β-cell function that occurs in type 2 diabetes patients and, thus, are not sufficient to control blood glucose levels in the later stages of the disease. Also, treatment with currently available medications exposes NIDDM patients to potential side effects such as hypoglycemia, gastrointestinal problems, fluid retention, oedebia, and/or weight gain.

Compounds, such as peptides that are selective for a particular G-protein coupled receptor known as the VPAC2 receptor, were initially identified by modifying vasoactive intestinal peptide (VIP) and/or pituitary adenylate cyclase-activating polypeptide (PACAP). (See, for example, Xia et al., J Pharmacol Exp Ther., 281:629-633 (1997); Tsutsumi et al., Diabetes, 51:1453-1460 (2002), WO 01/23420, WO 2004/006839.)

PACAP belongs to the secretin/glucagon/vasoactive intestinal peptide (VIP) family of peptides and works through three G-protein-coupled receptors that exert their action through the cAMP-mediated and other Ca²⁺-mediated signal transduction pathways. These receptors are known as the PACAP-preferring type 1 (PAC1) receptor (Isobe, et al., Regul. Pept., 110:213-217 (2003); Ogi, et al., Biochem. Biophys. Res. Commun., 196:1511-1521 (1993)) and the two VIP-shared type 2 receptors (VPAC1 and VPAC2) (Sherwood et al., Endocr. Rev., 21:619-670 (2000); Hammar et al., Pharmacol Rev, 50:265-270 (1998); Couvineau, et al., J. Biol. Chem., 278:24759-24766 (2003); Sreedharan, et al., Biochem. Biophys. Res. Commun., 193:546-553 (1993); Lutz, et al., FEBS Lett., 458: 197-203 (1999); Adamou, et al., Biochem. Biophys. Res. Commun., 209: 385-392 (1995)).

PACAP has comparable activities towards all three receptors, whilst VIP selectively activates the two VPAC receptors (Tsutsumi et al., Diabetes, 51:1453-1460 (2002)). Both VIP (Eriksson et al., Peptides, 10: 481-484 (1989)) and PACAP (Filipsson et al., JCEM, 82:3093-3098 (1997)) have been shown to not only stimulate insulin secretion in man when given intravenously but also increase glucagon secretion and hepatic glucose output. As a consequence, PACAP or VIP stimulation generally does not result in a net improvement of glycemia. Activation of multiple receptors by PACAP or VIP also has broad physiological effects on nervous, endocrine, cardiovascular, reproductive, muscular, and immune systems (Gozes et al., Curr. Med. Chem., 6:1019-1034 (1999)). Furthermore, it appears that VIP-induced watery diarrhoea in rats is mediated by only one of the VPAC receptors, VPAC1 (Ito et al., Peptides, 22:1139-1151 (2001); Tsutsumi et al., Diabetes, 51:1453-1460 (2002)). In addition, the VPAC1 and PAC1 receptors are expressed on α-cells and hepatocytes and, thus, are most likely involved in the effects on hepatic glucose output.

Exendin-4 is found in the salivary excretions from the Gila Monster, Heloderma Suspectum, (Eng et al., J Biol. Chem., 267 (11): 7402-7405 (1992)). It is a 39 amino acid peptide, which has glucose dependent insulin secretagogue activity. Particular PEGylated exendin and exendin agonist peptides are described in WO 2000/66629.

Recent studies have shown that peptides selective for the VPAC2 receptor are able to stimulate insulin secretion from the pancreas without gastrointestinal (GI) side effects and without enhancing glucagon release and hepatic glucose output (Tsutsumi et al., Diabetes, 51:1453-1460 (2002)).

Many of the VPAC2 receptor peptide agonists reported to date have, however, less than desirable potency, selectivity, and stability profiles, which could impede their clinical viability. In addition, many of these peptides are not suitable for commercial candidates as a result of stability issues associated with the polypeptides in formulation, as well as issues with the short half-life of these polypeptides in vivo. It has, furthermore, been identified that some VPAC2 receptor peptide agonists are inactivated by dipeptidyl-peptidase (DPP-IV). A short serum half-life could hinder the use of these agonists as therapeutic agents. There is, therefore, a need for new therapies, which overcome the problems associated with current medications for NIDDM.

The present invention seeks to provide improved compounds that are selective for the VPAC2 receptor and which induce insulin secretion from the pancreas only in the presence of high blood glucose levels. The compounds of the present invention are peptides, which are believed to also improve beta cell function. These peptides can have the physiological effect of inducing insulin secretion without GI side effects or a corresponding increase in hepatic glucose output and also generally have enhanced selectivity, potency, and/or in vivo stability of the peptide compared to known VPAC2 receptor peptide agonists.

The present invention also seeks to provide selective VPAC2 receptor peptide agonists, which have reduced clearance and improved in vivo stability. It is desirable that the agonists of the present invention be administered a minimum number of times during a prolonged period of time.

According to a first aspect of the present invention, there is provided a PEGylated VPAC2 receptor peptide agonist comprising a sequence of the formula:

Formula 10

(SEQ ID NO:18)

Xaa₁-Xaa₂-Xaa₃-Xaa₄-Xaa₅-Xaa₆-Thr-Xaa₈-Xaa₉-Xaa₁₀-
Thr-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Xaa₂₂-Xaa₂₃-Xaa₂₄-Xaa₂₅-Xaa₂₆-
Xaa₂₇-Xaa₂₈-Xaa₂₉-Xaa₃₀-Xaa₃₁-Xaa₃₂-Xaa₃₃-Xaa₃₄-
Xaa₃₅-Xaa₃₆-Xaa₃₇-Xaa₃₈-Xaa₃₉-Xaa₄₀

wherein:
Xaa₁ is: H is, dH, or is absent;
Xaa₂ is: dA, Ser, Val, Gly, Thr, Leu, dS, Pro, or Aib;
Xaa₃ is: Asp or Glu;
Xaa₄ is: Ala, Re, Tyr, Phe, Val, Thr, Leu, Trp, Gly, dA, Aib, or NMeA;
Xaa₅ is: Val, Leu, Phe, Ile, Thr, Trp, Tyr, dV, Aib, or NMeV;
Xaa₆ is: Phe, Re, Leu, Thr, Val, Trp, or Tyr;
Xaa₈ is: Asp, Glu, Ala, Lys, Leu, Arg, or Tyr;
Xaa₉ is: Asn, Gln, Asp, Glu, Ser, Cys, Lys, or K(CO(CH₂)₂SH);
Xaa₁₀ is: Tyr, Trp, Tyr(OMe), Ser, Cys, or Lys;
Xaa₁₂ is: Arg, Lys, Glu, hR, Orn, Lys (isopropyl), Aib, Cit, Ala, Leu, Gln, Phe, Ser, or Cys;
Xaa₁₃ is: Leu, Phe, Glu, Ala, Aib, Ser, Cys, Lys, or K(CO(CH₂)₂SH);
Xaa₁₄ is: Arg, Leu, Lys, Ala, hR, Orn, Lys (isopropyl), Phe, Gln, Aib, Cit, Ser, or Cys;
Xaa₁₅ is: Lys, Ala, Arg, Glu, Leu, hR, Orn, Lys (isopropyl), Phe, Gln, Aib, K(Ac), Cit, Ser, Cys, K(W), or K(CO(CH₂)₂SH);
Xaa₁₆ is: Gln, Lys, Glu, Ala, hR, Orn, Lys (isopropyl), Cit, Ser, Cys, K(CO(CH₂)₂SH), or K(W);
Xaa₁₇ is: Val, Ala, Leu, Ile, Met, Nle, Lys, Aib, Ser, Cys, K(CO(CH₂)₂SH), or K(W);
Xaa₁₈ is: Ala, Ser, Cys, Lys, K(CO(CH₂)₂SH), or K(W);
Xaa₁₉ is: Val, Ala, Glu, Phe, Gly, H is, Ile, Lys, Leu, Met, Asn, Pro, Gln, Arg, Ser, Thr, Trp, Tyr, Cys, Asp, K(CO(CH₂)₂SH), or K(W);
Xaa₂₀ is: Lys, Gln, hR, Arg, Ser, His, Orn, Lys (isopropyl), Ala, Aib, Trp, Thr, Leu, Ile, Phe, Tyr, Val, K(Ac), Cit, Cys, K(CO(CH₂)₂SH), or K(W);
Xaa₂₁ is: Lys, His, Arg, Ala, Phe, Aib, Leu, Gln, Orn, hR, K(Ac), Cit, Ser, Cys, Val, Tyr, Ile, Thr, Trp, K(W), or K(CO(CH₂)₂SH);
Xaa₂₂ is: Tyr, Trp, Phe, Thr, Leu, Ile, Val, Tyr(OMe), Ala, Aib, Ser, Cys, Lys, K(W), or K(CO(CH₂)₂SH);
Xaa₂₃ is: Leu, Phe, Ile, Ala, Trp, Thr, Val, Aib, Ser, Cys, Lys, K(W), or K(CO(CH₂)₂SH);
Xaa₂₄ is: Gln, Glu, Asn, Ser, Cys, Lys, K(CO(CH₂)₂SH, or K(W),
Xaa₂₅ is: Ser, Asp, Phe, Ile, Leu, Thr, Val, Trp, Gln, Asn, Tyr, Aib, Glu, Cys, Lys, K(CO(CH₂)₂SH), or K(W);
Xaa₂₆ is: Ile, Leu, Thr, Val, Trp, Tyr, Phe, Aib, Ser, Cys, Lys, K(CO(CH₂)₂SH), or K(W);
Xaa₂₇ is: Lys, hR, Arg, Gln, Ala, Asp, Glu, Phe, Gly, His, Ile, Met, Asn, Pro, Ser, Thr, Val, Trp, Tyr, Lys (isopropyl), Cys, Leu, Orn, dK, K(W), or K(CO(CH₂)₂SH);
Xaa₂₈ is: Asn, Asp, Gln, Lys, Arg, Aib, Orn, hR, Cit, Pro, dK, Ser, Cys, K(CO(CH₂)₂SH), or K(W);
Xaa₂₉ is: Lys, Ser, Arg, Asn, hR, Ala, Asp, Glu, Phe, Gly, His, Ile, Leu, Met, Pro, Gln, Thr, Val, Trp, Tyr, Cys, Orn, Cit, Aib, K(W), K(CO(CH₂)₂SH), or is absent;
Xaa₃₀ is: Arg, Lys, Ile, Ala, Asp, Glu, Phe, Gly, His, Leu, Met, Asn, Pro, Gln, Ser, Thr, Val, Trp, Tyr, Cys, hR, Cit, Aib, Orn, K(W), K(CO(CH₂)₂SH), or is absent;
Xaa₃₁ is: Tyr, His, Phe, Thr, Cys, Ser, Lys, Gln, K(W), K(CO(CH₂)₂SH), or is absent;
Xaa₃₂ is: Ser, Cys, Lys, or is absent;
Xaa₃₃ is: Trp, or is absent;
Xaa₃₄ is: Cys or is absent;
Xaa₃₅ is: Glu or is absent;
Xaa₃₆ is: Pro or is absent;
Xaa₃₇ is: Gly or is absent;
Xaa₃₈ is: Trp or is absent;
Xaa₃₉ is: Cys or is absent; and
Xaa₄₀ is: Arg or is absent

provided that if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈, or Xaa₃₉ is absent, the next amino acid present downstream is the next amino acid in the peptide agonist sequence,

and a C-terminal extension wherein the N-terminus of the C-terminal extension is linked to the C-terminus of the peptide of Formula 10 and wherein the C-terminal extension comprises an amino acid sequence of the formula:

Formula 17

(SEQ ID NO:29)

Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃

wherein:
Xaa₁ is: Gly, Cys, Lys, K(W), K(CO(CH₂)₂SH) or absent:
Xaa₂ is: Gly, Arg, Cys, Lys, K(W), K(CO(CH₂)₂SH), or absent;
Xaa₃ is: Pro, Thr, Ser, Ala, Cys, Lys, K(W), K(CO(CH₂)₂SH), or absent;
Xaa₄ is: Ser, Pro, H is, Cys, Lys, K(W), K(CO(CH₂)₂SH), or absent;
Xaa₅ is: Ser, Arg, Thr, Trp, Lys, Cys, K(W), K(CO(CH₂)₂SH), or absent;
Xaa₆ is: Gly, Ser, Cys, Lys, K(W), K(CO(CH₂)₂SH), or absent;
Xaa₇ is: Ala, Asp, Arg, Glu, Lys, Gly, Cys, K(W), K(CO(CH₂)₂SH), or absent;
Xaa₈ is: Pro, Ser, Ala, Cys, Lys, K(W), K(CO(CH₂)₂SH), or absent;
Xaa₉ is: Pro, Ser, Ala, Cys, Lys, K(W), K(CO(CH₂)₂SH), or absent;
Xaa₁₀ is: Pro, Ser, Ala, Arg, Lys, His, Cys, K(W), K(CO(CH₂)₂SH), or absent;
Xaa₁₁ is: Ser, Cys, His, Pro, Lys, Arg, K(W), K(CO(CH₂)₂SH), or absent;
Xaa₁₂ is: His, Ser, Arg, Lys, Cys, K(W), K(CO(CH₂)₂SH), or absent; and
Xaa₁₃ is: His, Ser, Arg, Lys, Cys, K(W), K(CO(CH₂)₂SH), or absent;

provided that at least five of Xaa₁ to Xaa₁₃ of the C-terminal extension are present and provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ is absent, the next amino acid present downstream is the next amino acid in the C-terminal extension and wherein the C-terminal amino acid may be amidated,

and wherein;

at least one of the Cys residues in the peptide agonist is covalently attached to a PEG molecule, or

at least one of the Lys residues in the peptide agonist is covalently attached to a PEG molecule, or

at least one of the K(W) in the peptide agonist is covalently attached to a PEG molecule, or

at least one of the K(CO(CH₂)₂SH) in the peptide agonist is covalently attached to a PEG molecule, or the carboxy-terminal amino acid of the peptide agonist is covalently attached to a PEG molecule, or a combination thereof.

Preferably, at least six of Xaa₁ to Xaa₁₃ of the C-terminal extension are present. More preferably at least seven, eight, nine, ten, eleven, twelve or all of Xaa₁ to Xaa₁₃ of the C-terminal extension are present.

It is preferable that the C-terminal extension has no more than three of any one of the following; Cys, Lys, K(W) or K(CO(CH₂)₂SH). It is more preferable that the C-terminal extension has no more than two of any of these residues. It is even more preferable that the C-terminal extension has no more than one of any of these residues. If there is only one Cys residue in the C-terminal extension, it is preferred that the Cys residue is at the C-terminus.

Preferably, the VPAC2 receptor peptide agonist comprises a sequence of the formula:

Formula 12

(SEQ ID NO:20)

Xaa₁-Xaa₂-Xaa₃-Xaa₄-Xaa₅-Xaa₆-Thr-Xaa₈-Xaa₉-Xaa₁₀-
Thr-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Xaa₂₂-Xaa₂₃-Xaa₂₄-Xaa₂₅-Xaa₂₆-
Xaa₂₇-Xaa₂s-Xaa₂₉-Xaa₃₀-Xaa₃₁-Xaa₃₂

wherein:
Xaa₁ is: His, dH, or is absent;
Xaa₂ is: dA, Ser, Val, Gly, Thr, Leu, dS, Pro, or Aib;
Xaa₃ is: Asp or Glu;
Xaa₄ is: Ala, Ile, Tyr, Phe, Val, Thr, Leu, Trp, Gly, dA, Aib, or NMeA;
Xaa₅ is: Val, Leu, Phe, Ile, Thr, Trp, Tyr, dV, Aib, or NMeV;
Xaa₆ is: Phe, Ile, Leu, Thr, Val, Trp, or Tyr;
Xaa₈ is: Asp, Glu, Ala, Lys, Leu, Arg, or Tyr;
Xaa₉ is: Asn, Gln, Glu, Ser, Cys, or Lys;
Xaa₁₀ is: Tyr, Trp, Tyr(OMe), Ser, Cys, or Lys;
Xaa₁₂ is: Arg, Lys, hR, Orn, Aib, Cit, Ala, Leu, Gln, Phe, Ser, or Cys;
Xaa₁₃ is: Leu, Phe, Glu, Ala, Aib, Ser, Cys, Lys, or K(CO(CH₂)₂SH);
Xaa₁₄ is: Arg, Leu, Lys, Ala, hR, Orn, Phe, Gln, Aib, Cit, Ser, or Cys;
Xaa₁₅ is: Lys, Ala, Arg, Glu, Leu, hR, Orn, Phe, Gln, Aib, K(Ac), Cit, Ser, Cys, or K(W);
Xaa₁₆ is: Gln, Lys, Ala, hR, Orn, Cit, Ser, Cys, or K(CO(CH₂)₂SH);
Xaa₁₇ is: Val, Ala, Leu, Ile, Met, Nle, Lys, Aib, Ser, Cys, or K(CO(CH₂)₂SH);
Xaa₁₈ is: Ala, Ser, Cys, or Lys;
Xaa₁₉ is: Ala, Gly, Leu, Ser, Cys, Lys, or K(CO(CH₂)₂SH);
Xaa₂₀ is: Lys, Gln, hR, Arg, Ser, Orn, Ala, Aib, Trp, Thr, Leu, Ile, Phe, Tyr, Val, K(Ac), Cit, or Cys;
Xaa₂₁ is: Lys, Arg, Ala, Phe, Aib, Leu, Gln, Orn, hR, K(Ac), Cit, Ser, or Cys;
Xaa₂₂ is: Tyr, Trp, Phe, Thr, Leu Ile, Val, Tyr(OMe), Ala, Aib, Ser, Cys, or Lys:
Xaa₂₃ is: Leu, Phe, Ile, Ala, Trp, Thr, Val, Aib, Ser, Cys, or Lys;
Xaa₂₄ is: Gln, Asn, Ser, Cys, Lys, or K(CO(CH₂)₂SH);
Xaa₂₅ is: Ser, Asp, Phe, Ile, Leu, Thr, Val, Trp, Gln, Asn, Tyr, Aib, Glu, Cys, Lys, or K(CO(CH₂)₂SH);
Xaa₂₆ is: Ile, Leu, Thr, Val, Trp, Tyr, Phe, Aib, Ser, Cys, Lys, or K(CO(CH₂)₂SH);
Xaa₂₇ is: Lys, hR, Arg, Gln, Orn, dK, Ser, or Cys;
Xaa₂₈ is: Asn, Gln, Lys, Arg, Aib, Orn, hR, Cit, Pro, dK, Ser, Cys, or K(CO(CH₂)₂SH);
Xaa₂₉ is: Lys, Ser, Arg, Asn, hR, Orn, Cit, Aib, Cys, or is absent;
Xaa₃₀ is: Arg, Lys, Ile, hR, Cit, Aib, Orn, Ser, Cys, or is absent;
Xaa₃₁ is: Tyr, His, Phe, Lys, Ser, Cys, Gln, or is absent; and
Xaa₃₂ is: Cys, Ser, Lys, or is absent;

provided that if Xaa₂₉, Xaa₃₀, or Xaa₃₁ is absent, the next amino acid present downstream is the next amino acid in the peptide agonist sequence,

and a C-terminal extension wherein the N-terminus of the C-terminal extension is linked to the C-terminus of the peptide of Formula 12 and wherein the C-terminal extension comprises an amino acid sequence of the formula:

Formula 11

(SEQ ID NO:19)

Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃

wherein:
Xaa₁ is: Gly, Cys, Lys, or absent;
Xaa₂ is: Gly, Arg, Cys, Lys, or absent;
Xaa₃ is: Pro, Thr, Ser, Ala, Cys, Lys, or absent;
Xaa₄ is: Ser, Pro, His, Cys, Lys, or absent;
Xaa₅ is: Ser, Arg, Tbr, Trp, Lys, Cys, or absent;
Xaa₆ is: Gly, Ser, Cys, Lys, or absent;
Xaa₇ is: Ala, Asp, Arg, Glu, Lys, Gly, Cys, or absent;
Xaa₈ is: Pro, Ser, Ala, Cys, Lys, or absent;
Xaa₉ is: Pro, Ser, Ala, Cys, Lys, or absent;
Xaa₁₀ is: Pro, Ser, Ala, Arg, Lys, Mis, Cys, or absent;
Xaa₁₁ is: Ser, Cys, His, Pro, Lys, Arg, or absent;
Xaa₁₂ is: His, Ser, Arg, Lys, Cys, or absent; and
Xaa₁₃ is: Bis, Ser, Arg, Lys, Cys, or absent;

provided that at least five of Xaa₁ to Xaa₁₃ of the C-terminal extension are present and provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ is absent, the next amino acid present downstream is the next amino acid in the C-terminal extension and wherein the C-terminal amino acid may be amidated,

and wherein:

at least one of the Cys residues in the peptide agonist is covalently attached to a PEG molecule, or

at least one of the Lys residues in the peptide agonist is covalently attached to a PEG molecule, or

at least one of the K(CO(CH₂)₂SH) in the peptide agonist is covalently attached to a PEG molecule, or

the K(W) in the peptide agonist is covalently attached to a PEG molecule, or

the carboxy-terminal amino acid of the peptide agonist is covalently attached to a PEG molecule, or a combination thereof.

Preferably, at least six of Xaa₁ to Xaa₁₃ of the C-terminal extension are present. More preferably at least seven, eight, nine, ten, eleven, twelve or all of Xaa₁ to Xaa₁₃ of the C-terminal extension are present.

The VPAC2 receptor peptide agonist preferably comprises a sequence of the formula:

Formula 13

(SEQ ID NO:21)

His-Xaa₂-Xaa₃-Xaa₄-Xaa₅-Phe-Thr-Xaa₈-Xaa₉-Xaa₁₀-
Thr-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Xaa₂₂-Xaa₂₃-Xaa₂₄-Xaa₂₅-Xaa₂₆-
Xaa₂₇-Xaa₂₈-Xaa₂₉-Xaa₃₀-Xaa₃₁

wherein:
Xaa₂ is: dA, Ser, Val, dS, or Aib;
Xaa₃ is: Asp or Glu;
Xaa₄ is: Ala, dA, or Aib;
Xaa₅ is: Val, Leu, dV, or Aib;
Xaa₈ is: Asp, Glu, or Ala;
Xaa₉ is: Asn, Gln, Glu, Ser, Cys, or Lys;
Xaa₁₀ is: Tyr, or Tyr(OMe);
Xaa₁₂ is: Ala, Arg, Lys, hR, Orn, Ser, or Cys;
Xaa₁₃ is: Leu, Ser, Cys, Lys, or K(CO(CH₂)₂SH);
Xaa₁₄ is: Arg, Leu, Lys, Ala, hR, Orn, Phe, Gln, Aib, Cit, Ser, or Cys;
Xaa₁₅ is: Lys, Ala, Arg, Leu, Orn, Phe, Gln, Aib, K(Ac), Ser, Cys, or K(W);
Xaa₁₆ is: Gln, Lys, Ser, Cys, or K(CO(CH₂)₂SH);
Xaa₁₇ is: Val, Ala, Leu, Ile, Met, Nle, Lys, Ser, Cys, or K(CO(CH₂)₂SH);
Xaa₁₈ is: Ala, Ser, Cys, or Lys;
Xaa₁₉ is: Ala, Leu, Ser, Cys, Lys, or K(CO(CH₂)₂SH);
Xaa₂₀ is: Lys, Gln, hR, Arg, Ser, Ala, Aib, Trp, Thr, Leu, Ile, Phe, Tyr, Val, K(Ac), or Cys;
Xaa₂₁ is: Lys, Arg, Ala, Phe, Aib, Leu, Gln, K(Ac), Orn, Ser, or Cys;
Xaa₂₂ is: Tyr, Trp, Phe, Leu, Ile, Val, Ser, Cys, Lys, or Tyr(OMe);
Xaa₂₃ is: Leu, Ser, Cys, or Lys;
Xaa₂₄ is: Gln, Asn, Ser, Cys, Lys, or K(CO(CH₂)₂SH);
Xaa₂₅ is: Ser, Phe, Ile, Leu, Thr, Val, Trp, Gln, Asn, Tyr, Aib, Cys, Lys, or K(CO(CH₂)₂SH);
Xaa₂₆ is: Ile, Leu, Thr, Val, Trp, Tyr, Phe, Aib, Ser, Cys, Lys, or K(CO(CH₂)₂SH);
Xaa₂₇ is: Lys, hR, Arg, dK, Orn, Ser, or Cys;
Xaa₂₈ is: Asn, Gln, Lys, hR, Aib, Orn, dK, Pro, Ser, Cys, or K(CO(CH₂)₂SH);
Xaa₂₉ is: Lys, Ser, Arg, hR, Orn, Cys, or is absent;
Xaa₃₀ is: Arg, Lys, hR, Ser, Cys, or is absent;
Xaa₃₁ is: Tyr, Phe, Lys, Ser, Cys, or is absent; and
Xaa₃₂ is: Cys, Ser, Lys, or is absent;

provided that if Xaa₂₉, Xaa₃₀, or Xaa₃₁ is absent, the next amino acid present downstream is the next amino acid in the peptide agonist sequence,

and a C-terminal extension wherein the N-terminus of the C-terminal extension is linked to the C-terminus of the peptide of Formula 13 and wherein the C-terminal extension comprises an amino acid sequence of the formula:

Formula 11

(SEQ ID NO:19)

Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃

wherein:
Xaa₁ is: Gly, Cys, Lys, or absent;
Xaa₂ is: Gly, Arg, Cys, Lys, or absent;
Xaa₃ is: Pro, Thr, Ser, Ala, Cys, Lys, or absent;
Xaa₄ is: Ser, Pro, His, Cys, Lys, or absent;
Xaa₅ is: Ser, Arg, Thr, Trp, Lys, Cys, or absent;
Xaa₆ is: Gly, Ser, Cys, Lys, or absent;
Xaa₇ is: Ala, Asp, Arg, Glu, Lys, Gly, Cys, or absent;
Xaa₈ is: Pro, Ser, Ala, Cys, Lys, or absent;
Xaa₉ is: Pro, Ser, Ala, Cys, Lys, or absent;
Xaa₁₀ is: Pro, Ser, Ala, Arg, Lys, His, Cys, or absent;
Xaa₁₁ is: Ser, Cys, His, Pro, Lys, Arg, or absent;
Xaa₁₂ is: His, Ser, Arg, Lys, Cys, or absent; and
Xaa₁₃ is: His, Ser, Arg, Lys, Cys, or absent;

provided that at least five of Xaa₁ to Xaa₁₃ of the C-terminal extension are present and provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ is absent, the next amino acid present downstream is the next amino acid in the C-terminal extension and wherein the C-terminal amino acid may be amidated,

and wherein:

at least one of the Cys residues in the peptide agonist is covalently attached to a PEG molecule, or

at least one of the Lys residues in the peptide agonist is covalently attached to a PEG molecule, or

at least one of the K(CO(CH₂)₂SH) in the peptide agonist is covalently attached to a PEG molecule, or

the K(W) in the peptide agonist is covalently attached to a PEG molecule, or

the carboxy-terminal amino acid of the peptide agonist is covalently attached to a PEG molecule, or a combination thereof.

Preferably, the VPAC2 receptor peptide agonist of the present invention comprises a sequence of the Formula 10 (SEQ ID NO: 18), Formula 12 (SEQ ID NO: 20) or Formula 13(SEQ ID NO: 21) wherein Xaa₁ is Asp or Glu, Xaa₈ is Asp or Glu, Xaa₁₂ is Arg, hR, Lys, or Orn, Xaa₁₄ is Arg, Gln, Aib, hR, Orn, Cit, Lys, Ala, or Leu, Xaa₁₅ is Lys, Aib, Orn, or Arg, Xaa₁₆ is Gln or Lys, Xaa₁₇ is Val, Leu, Ala, Ile, Lys, or Nle, Xaa₂₀ is Lys, Val, Leu, Aib, Ala, Gln, or Arg, Xaa₂₁ is Lys, Aib, Orn, Ala, Gln, or Arg, Xaa₂₇ is Lys, Orn, hR, or Arg, Xaa₂₈ is Asn, Gln, Lys, hR, Aib, Orn, or Pro and Xaa₂₉ is Lys, Orn, hR, or is absent.

More preferably, the VPAC2 receptor peptide agonist of the present invention comprises a sequence of the Formula 10 (SEQ ID NO: 18), Formula 12 (SEQ ID NO: 20) or Formula 13 (SEQ ID NO: 21) wherein Xaa₁₂ is Arg, hR, or Orn, Xaa₁₄ is Arg, Aib, Gln, Ala, Leu, Lys, or Orn, Xaa₁₅ is Lys or Aib, Xaa₁₇ is Val or Leu, Xaa₂₀ is Lys or Aib, Xaa₂₁ is Lys, Aib, or Gln and Xaa₂₈ is Asn or Gln.

Preferably, the VPAC2 receptor peptide agonist of the present invention comprises a sequence of the Formula 10 (SEQ ID NO: 18), Formula 12 (SEQ ID NO: 20) or Formula 13 (SEQ ID NO: 21) wherein Xaa₃₀ and/or Xaa₃₁ are absent. Alternatively, Xaa₂₉, Xaa₃₀ and Xaa₃₁ are all absent.

Preferably, the VPAC2 receptor peptide agonist of the present invention comprises a sequence of the Formula 10 (SEQ ID NO:18), Formula 12 (SEQ ID NO: 20) or Formula 13 (SEQ ID NO: 21) wherein either Xaa₁₄ or Xaa₁₅ is Aib.

Also preferably, the VPAC2 receptor peptide agonist of the present invention comprises a sequence of the Formula 10 (SEQ ID NO: 18), Formula 12 (SEQ ID NO: 20) or Formula 13 (SEQ ID NO: 21) wherein either Xaa₂₀ or Xaa₂₁ is Aib.

More preferably, either Xaa₁₄ or Xaa₁₅ is Aib and either Xaa₂₀ or Xaa₂₁ is Aib. It is especially preferred that Xaa₁₅ is Aib and Xaa₂₀ is Aib.

Preferably, the VPAC2 receptor peptide agonist of the present invention comprises a sequence of the Formula 10 (SEQ ID NO: 18), Formula 12 (SEQ ID NO: 20) or Formula 13 (SEQ ID NO: 21) wherein Xaa₂₈ is Gln and Xaa₂₉ is Lys or is absent.

More preferably, Xaa₂₈ is Gln and Xaa₂₉ is Lys or is absent, and either Xaa₁₄ or Xaa₁₅ is Aib and either Xaa₂₀ or Xaa₂₁ is Aib.

Preferably, the VPAC2 receptor peptide agonist of the present invention comprises a sequence of the Formula 10 (SEQ ID NO: 18), Formula 12 (SEQ ID NO: 20) or Formula 13 (SEQ ID NO: 21) wherein Xaa₁₂ is hR or Orn, Xaa₂₇ is hR or Orn and Xaa₂₉ is hR or Orn. Alternatively, any one of Xaa₁₂, Xaa₂₇ and Xaa₂₉ may be a PEGylated Lys, Cys, K(CO(CH₂)₂SH or K(W), whilst all the other two positions have the preferred amino acid substitutions as described.

More preferably, Xaa₁₂ is hR or Orn, Xaa₂₇ is hR or Orn and Xaa₂₉ is hR or Orn, and either Xaa₁₄ or Xaa₁₅ is Aib and either Xaa₂₀ or Xaa₂₁ is Aib.

Preferably, the VPAC2 receptor peptide agonist of the present invention comprises a sequence of the Formula 10 (SEQ ID NO: 18), Formula 12 (SEQ ID NO: 20) or Formula 13 (SEQ ID NO: 21) wherein Xaa₁₅ is Aib, Xaa₂₀ is Aib, and Xaa₁₂, Xaa₂₁, Xaa₂₇ and Xaa₂₈ are all Orn. More preferably, Xaa₁₅ is Aib, Xaa₂₀ is Aib, Xaa₁₂, Xaa₂₁, Xaa₂₇ and Xaa₂₈ are all Orn, Xaa₈ is Glu, Xaa₉ is Gln and Xaa₁₀ is Tyr(OMe). Alternatively, any one or more of Xaa₈, Xaa₉, Xaa₁₀, Xaa₁₂, Xaa₁₅, Xaa₂₀, Xaa₂₁, Xaa₂₇ and Xaa₂₈ may be a PEGylated Lys, Cys, K(CO(CH₂)₂SH) or K(W), whilst all the other positions have the preferred amino acid substitutions as described.

The PEGylated VPAC2 receptor peptide agonist of the invention more preferably comprises a sequence of the formula:

Formula 16

(SEQ ID NO:28)

His-Ser-Xaa₃-Ala-Val-Phe-Thr-Xaa₈-Xaa₉-Xaa₁₀-Thr-
Xaa₁₂-Xaa₁₃-Xaa₁₄-Xaa₁₅-Xaa₁₆-Xaa₁₇-Ala-Xaa₁₉-
Xaa₂₀-Xaa₂₁-Xaa₂₂-Leu-Xaa₂₄-Xaa₂₅-Xaa₂₆-Xaa₂₇-
Xaa₂₈-Xaa₂₉-Xaa₃₀-Xaa₃₁-Xaa₃₂

Xaa₃ is: Asp, or Glu;
Xaa₈ is: Asp, or Glu;
Xaa₉ is: Asn, Gln, or Cys;
Xaa₁₀ is: Tyr, or Tyr(OMe);
Xaa₁₂ is: Arg, Orn, or hR;
Xaa₁₃ is: Leu, Cys, or K(CO(CH₂)₂SH);
Xaa₁₄ is: Arg, Leu, or Aib;
Xaa₁₅ is: Lys, Ala, Arg, Aib, or K(W);
Xaa₁₆ is: Gln, Lys, or K(CO(CH₂)₂SH);
Xaa₁₇ is: Val, Leu, Cys, or K(CO(CH₂)₂SH);
Xaa₁₉ is: Ala, Leu, Cys, or K(CO(CH₂)₂SH);
Xaa₂₀ is: Lys, Gln, Arg, Aib, or Cys;
Xaa₂₁ is: Lys, Arg, Aib, or Orn;
Xaa₂₂ is: Tyr, or Tyr(OMe);
Xaa₂₄ is: Gln, Cys, or K(CO(CH₂)₂SH);
Xaa₂₅ is: Ser, Cys, or K(CO(CH₂)₂SH);
Xaa₂₆ is: Ile, Cys, or K(CO(CH₂)₂SH);
Xaa₂₇ is: Lys, Arg, Orn, or hR;
Xaa₂₈ is: Asn, hR, Orn, Cys, or K(CO(CH₂)₂SH);
Xaa₂₉ is: Orn, Lys, hR, or is absent;
Xaa₃₀ is: Arg, hR, or is absent; and
Xaa₃₁ is: Tyr, or is absent; and
Xaa₃₂ is: Cys, or is absent

provided that if Xaa₂₉, Xaa₃₀, or Xaa₃₁ is absent, the next amino acid present downstream is the next amino acid in the peptide agonist sequence,

and a C-terminal extension wherein the N-terminus of the C-terminal extension is linked to the C-terminus of the peptide of Formula 16 and wherein the C-terminal extension comprises an amino acid sequence of the formula:

Formula 11

(SEQ ID NO:19)

Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃

wherein:
Xaa₁ is: Gly, Cys, Lys, or absent;
Xaa₂ is: Gly, Arg, Cys, Lys, or absent;
Xaa₃ is: Pro, Thr, Ser, Ala, Cys, Lys, or absent;
Xaa₄ is: Ser, Pro, His, Cys, Lys, or absent;
Xaa₅ is: Ser, Arg, Thr, Trp, Lys, Cys, or absent;
Xaa₆ is: Gly, Ser, Cys, Lys, or absent;
Xaa₇ is: Ala, Asp, Arg, Glu, Lys, Gly, Cys, or absent;
Xaa₈ is: Pro, Ser, Ala, Cys, Lys, or absent;
Xaa₉ is: Pro, Ser, Ala, Cys, Lys, or absent;
Xaa₁₀ is: Pro, Ser, Ala, Arg, Lys, His, Cys, or absent;
Xaa₁₁ is: Ser, Cys, His, Pro, Lys, Arg, or absent;
Xaa₁₂ is: His, Ser, Arg, Lys, Cys, or absent; and
Xaa₁₃ is: His, Ser, Arg, Lys, Cys, or absent;

provided that at least five of Xaa₁ to Xaa₁₃ of the C-terminal extension are present and provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ is absent, the next amino acid present downstream is the next amino acid in the C-terminal extension and wherein the C-terminal amino acid may be amidated,

and wherein:

at least one of the Cys residues in the peptide agonist is covalently attached to a PEG molecule, or

at least one of the Lys residues in the peptide agonist is covalently attached to a PEG molecule, or

at least one of the K(CO(CH₂)₂SH) in the peptide agonist is covalently attached to a PEG molecule, or

the K(W) in the peptide agonist is covalently attached to a PEG molecule, or

the carboxy-terminal amino acid of the peptide agonist is covalently attached to a PEG molecule, or a combination thereof.

Preferably, the C-terminal extension of the VPAC2 receptor peptide agonist comprises an amino acid sequence of the formula:

Formula 7

(SEQ ID NO:15)

Xaa₁-Xaa₂-Xaa₃-Xaa₄-Xaa₅-Xaa₆-Xaa₇-Xaa₈-Xaa₉-
Xaa₁₀-Xaa₁₁

wherein:
Xaa₁ is: Gly, Cys, or absent;
Xaa₂ is: Gly, Arg, or absent;
Xaa₃ is: Pro, Thr, or absent;
Xaa₄ is: Ser, or absent;
Xaa₅ is: Ser, or absent;
Xaa₆ is: Gly, or absent;
Xaa₇ is: Ala, or absent;
Xaa₈ is: Pro, or absent;
Xaa₉ is: Pro, or absent;
Xaa₁₀ is: Pro, or absent; and
Xaa₁₁ is: Ser, Cys, or absent;

provided that at least five of Xaa₁ to Xaa₁₁ of the C-terminal extension are present and provided that if Xaa₁, Xaa₂, Xaa₃, Xaa₄, Xaa₅, Xaa₆, Xaa₇, Xaa₈, Xaa₉, or Xaa₁₀absent, the next amino acid present downstream is the next amino acid in the C-terminal extension and wherein the C-terminal amino acid may be amidated.

Preferably, at least six of Xaa₁ to Xaa₁₁ of the C-terminal extension are present. More preferably at least seven, eight, nine, ten, or all of Xaa₁ to Xaa₁₁ of the C-terminal extension are present.

More preferably, the C-terminal extension of the VPAC2 receptor peptide agonist is selected from:

| | |
|---|---|
| SEQ ID NO:10 | GGPSSGAPPPS |
| SEQ ID NO:11 | GGPSSGAPPPS-NH₂ |
| SEQ ID NO:22 | GGPSSGAPPPC |
| SEQ ID NO:23 | GGPSSGAPPPC-NH₂ |
| SEQ ID NO:16 | GRPSSGAPPPS |
| SEQ ID NO:17 | GRPSSGAPPPS-NH₂ |

It is especially preferred that the C-terminal extension is GGPSSGAPPPS (SEQ ID NO: 10) or GGPSSGAPPPS-NH₂ (SEQ ID NO: 11).

The PEG molecule(s) may be covalently attached to any Lys, Cys, K(W), or K(CO(CH₂)₂SH) residues at any position in the peptide agonist. In particular, the PEG molecule(s) may be covalently attached to any Lys, Cys, K(W), or K(CO(CH₂)₂SH) residue at positions 9, 13, 15, 16, 17, 18, 19, 20, 21, 24, 25, 26 and/or 28 of Formula 10, 12, 13, or 16. Alternatively, the PEG molecule(s) may be covalently attached to a residue in the C-terminal extension.

Preferably, there is at least one PEG molecule covalently attached to Xaa₂₅ or any subsequent residue in Formula 10, 12, 13, or 16.

Preferably, there is at least one PEG molecule covalently attached to a residue in the C-terminal extension of the VPAC2 receptor peptide agonist.

Any Lys residue in the VPAC2 receptor peptide agonist may be substituted for a K(W) or a K(CO(CH₂)₂SH), which may be PEGylated. In addition, any Cys residue in the peptide agonist may be substituted for a modified cysteine residue, for example, hC. The modified Cys residue may be covalently attached to a PEG molecule.

It is preferred that two of the Cys residues are each covalently attached to a PEG molecule or two of the Lys residues are each covalently attached to a PEG molecule. Alternatively, one of the Cys residues may be covalently attached to a PEG molecule or one of the Lys residues may be covalently attached to a PEG molecule.

It is preferred that there is a K(CO(CH₂)₂SH) is the VPAC2 receptor peptide agonist and that this is PEGylated.

Where there is more than one PEG molecule, there may be a combination of Lys, Cys, K(CO(CH₂)₂SH), K(W) and carboxy-terminal amino acid PEGylation. For example, if there are two PEG molecules, one may be attached to a Lys residue and one may be attached to a Cys residue.

Preferably, the PEG molecule is branched. Alternatively, the PEG molecule may be linear.

Preferably, the PEG molecule is between 1,000 daltons and 100,000 daltons in molecular weight. More preferably the PEG molecule is selected from 10,000, 20,000, 30,000, 40,000, 50,000 and 60,000 daltons. Even more preferably, it is selected from 20,000, 40,000, or 60,000. Where there are two PEG molecules covalently attached to the peptide agonist of the present invention, each is 1,000 to 40,000 daltons and preferably, they have molecular weights of 20,000 and 20,000 daltons, 10,000 and 30,000 daltons, 30,000 and 30,000 daltons, or 20,000 and 40,000 daltons.

Preferably, the VPAC2 receptor peptide agonist sequence further comprises a histidine residue at the N-terminal extension region of the peptide sequence before Xaa₁.

Preferably, the VPAC2 receptor peptide agonist of the present invention further comprises a N-terminal modification at the N-terminus of the peptide agonist wherein the N-terminal modification is selected from:
(a) addition of D-histidine, isoleucine, methionine, or norleucine;
(b) addition of a peptide comprising the sequence Ser-Trp-Cys-Glu-Pro-Gly-Trp-Cys-Arg (SEQ ID NO: 14) wherein the Arg is linked to the N-terminus of the peptide agonist;
(c) addition of C₁-C₁₆ alkyl optionally substituted with one or more substituents independently selected from aryl, C₁-C₆ alkoxy, —NH₂, —OH, halogen and —CF₃;
(d) addition of —C(O)R¹ wherein R¹ is a C₁-C₁₆ alkyl optionally substituted with one or more substituents independently selected from aryl, C₁-C₆ alkoxy, —NH₂—OH, halogen, —SH and —CF₃; an aryl or aryl C₁-C₄ alkyl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; —NR²R³ wherein R² and R³ are independently hydrogen, C₁-C₆ alkyl, aryl or aryl C₁-C₄ alkyl; —OR⁴ wherein R⁴ is C₁-C₁₆ alkyl optionally substituted with one or more substituents independently selected from aryl, C₁-C₆ alkoxy, —NH₂, —OH, halogen and —CF₃, aryl or aryl C₁-C₄ alkyl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; or 5-pyrrolidin-2-one;
(e) addition of —SO₂R⁵ wherein R⁵ is aryl, aryl C₁-C₄ alkyl or C₁-C₁₆ alkyl;
(f) formation of a succinimide group optionally substituted with C₁-C₆ alkyl or —SR⁶, wherein R⁶ is hydrogen or C₁-C₆ alkyl;
(g) addition of methionine sulfoxide;
(h) addition of biotinyl-6-aminohexanoic acid (b-aminocaproic acid); and
(i) addition of —C(═NH)—NH₂.

Preferably, the N-terminal modification is the addition of a group selected from: acetyl, propionyl, butyryl, pentanoyl, hexanoyl, methionine, methionine sulfoxide, 3-phenylpropionyl, phenylacetyl, benzoyl, norleucine, D-histidine, isoleucine, -3-mercaptopropionyl, biotinyl-6-aminohexanoic acid, and —C(═NH)—NH₂, and more preferably is the addition of acetyl or hexanoyl. It is especially preferred that the N-terminal modification is the addition of hexanoyl.

It will be appreciated by the person skilled in the art that PEGylated VPAC2 receptor peptide agonists comprising various combinations of peptide sequence according to Formula 10, 12, 13 or 16, C-terminal extensions and N-terminal modifications as described herein, may be made based on the above disclosure.

The following VPAC2 receptor peptide agonists may be PEGylated:

| | | |
|---|---|---|
| | SEQ | |
| Agonist | ID | |
| # | NO | Sequence |

| | | |
|---|---|---|
| P6 | 30 | HSDAVFTDNYTRLRKQVAAKKYLQSIKNKRYGGPSSGAP |
| | | PPS |
| P7 | 31 | HSDAVFTDNYTRLRKQVAAKKYLQSIKNKRGGT |
| P8 | 32 | HSDAVFTDNYTRLRKQVAAKKYLQSIKNKKGGT |
| P9 | 33 | HSDAVFTDNYTRLRKQVAAKKYLQSIKNKKGGPSSGAPP |
| | | PS |
| P18 | 34 | HSDAVFTDNYTRLRKQVAAhRKYLQSIKNKRYGGPSSGA |
| | | PPPS |
| P19 | 35 | HSDAVFTDNYTRLRKQVAAIKYLQSIKNKRYGGPSSGAP |
| | | PPS |
| P20 | 36 | HSDAVFTDNYTRLRKQVAARKYLQSIKNKRYGGPSSGAP |
| | | PPS |
| P21 | 37 | HSDAVFTDNYTRLRKQVAASKYLQSIKNKRYGGPSSGAP |
| | | PPS |
| P22 | 38 | HSDAVFTDNYTRLRKQVAAKKYLQSIhRNKRYGGPSSGA |
| | | PPPS |
| P23 | 39 | HSDAVFTDNYTRLRKQVAAKKYLQSIRNKRYGGPSSGAP |
| | | PPS |
| P24 | 40 | HSDAVFTDNYTRLRKQVAAKKYLQSIKNhRRYGGPSSGA |
| | | PPPS |
| P25 | 41 | HSDAVFTDNYTRLRKQVAAKKYLQSIKNRRYGGPSSGAP |
| | | PPS |
| P26 | 42 | HSDAVFTDNYTRFRKQVAAKKYLQSIKNKRYGGPSSGAP |
| | | PPS |
| P27 | 43 | HSDAVFTDNWTRLRKQVAAKKYLQSIKNKRYGGPSSGAP |
| | | PPS |
| P28 | 44 | HSDAVFTDNYTRLRKQVAAKKYLQSIKNKRHGGPSSGAP |
| | | PPS |
| P29 | 45 | HSDAVFTDNYTRLRKQVAAKKYLQSIKNKRQGGPSSGAP |
| | | PPS |
| P31 | 46 | C6-HSDAVFTDNYTRLRKQVAAKKYLQSIKNKRYGGPSS |
| | | GAPPPS |
| P33 | 47 | Ac-HSDAVFTDNYTRLRKQVAAKKYLQSIKNKRYGGPSS |
| | | GAPPPS |
| P34 | 48 | HSDAVFTDNYTRLRKQVAAKKYLQSIKNKRR |
| P37 | 49 | HSDAVFTDNYTRLRKQVAAKKYLQSIKNKRYC |
| P43 | 50 | HGDAVFTDNYTRLRKQVAAKKYLQSIKNKRYGGPSSGAP |
| | | PPS |
| P44 | 51 | HVDAVFTDNYTRLRKQVAAKKYLQSIKNKRYGGPSSGAP |
| | | PPS |
| P45 | 52 | HTDAVFTDNYTRLRKQVAAKKYLQSIKNKRYGGPSSGAP |
| | | PPS |
| P46 | 53 | HLDAVFTDNYTRLRKQVAAKKYLQSIKNKRYGGPSSGAP |
| | | PPS |
| P47 | 54 | HdADAVFTDNYTRLRKQVAAKKYLQSIKNKRYGGPSSGA |
| | | PPPS |
| P48 | 55 | HdSDAVFTDNYTRLRKQVAAKKYLQSIKNKRYGGPNSGA |
| | | PPPS |
| P49 | 56 | HPDAVFTDNYTRLRKQVAAKKYLQSIKNKRYGGPSSGAP |
| | | PPS |
| P50 | 57 | HSDIVFTDNYTRLRKQVAAKKYLQSIKNKRYGGPSSGAP |
| | | PPS |
| P51 | 58 | HSDYVFTDNYTRLRKQVAAKKYLQSIKNKRYGGPSSGAP |
| | | PPS |
| P52 | 59 | HSDFVFTDNYTRLRKQVAAKKYLQSIKNKRYGGPSSGAP |
| | | PPS |
| P53 | 60 | HSDVVFTDNYTRLRKQYAAKKYLQSIKNKRYGGPSSGAP |
| | | PPS |
| P54 | 61 | HSDTVFTDNYTRLRKQVAAKKYLQSIKNKRYGGPSSGAP |
| | | PPS |
| P55 | 62 | HSDLVFTDNYTRLRKQVAAKKYLQSIKNKRYGGPSSGAP |
| | | PPS |
| P56 | 63 | HSDWVFTDNYTRLRKQVAAKKYLQSIKNKRYGGPSSGAP |
| | | PPS |
| P58 | 64 | HSDAFFTDNYTRLRKQVAAKKYLQSIKNKRYGGPSSGAP |
| | | PPS |
| P60 | 65 | HSDALFTDNYTRLRKQVAAKKYLQSIKNKRYGGPSSGAP |
| | | PPS |
| P61 | 66 | HSDALFTDNYTRLRKQVAAKKYLQSIKNKRYGGPSSGAP |
| | | PPS |
| P62 | 67 | HSDATFTDNYTRLRKQVAAKKYLQSIKNKRYGGPSSGAP |
| | | PPS |
| P63 | 68 | HSDAVITDNYTRLRKQVAAKKYLQSIKNKRYGGPSSGAP |
| | | PPS |
| P64 | 69 | HSDAVLTDNYTRLRKQVAAKKYLQSIKNKRYGGPSSGAP |
| | | PPS |
| P65 | 70 | HSDAVTTDNYTRLRKQVAAKKYLQSIKNKRYGGPSSGAP |
| | | PPS |
| P66 | 71 | HSDAVVTDNYTRLRKQVAAKKYLQSIKNKRYGGPSSGAP |
| | | PPS |
| P67 | 72 | HSDAVWTDNYTRLRKQVAAKKYLQSIKNKRYGGPSSGAP |
| | | PPS |
| P68 | 73 | HSDAVYTDNYTRLRKQVAAKKYLQSIKNKRYGGPSSGAP |
| | | PPS |
| P69 | 74 | HSDAWFTDNYTRLRKQVAAKKYLQSIKNKRYGGPSSGAP |
| | | PPS |
| P70 | 75 | HSDAYFTDNYTRLRKQVAAKKYLQSIKNKRYGGPSSGAP |
| | | PPS |
| P71 | 76 | HSDAVFTDNYTRLRRQVAARRYLQSIRNRRYGGPSSGAP |
| | | PPS |
| P72 | 77 | HSDAVFTDNYTRLRRQVAAKKYLQSIKNKRYGGPSSGAP |
| | | PPS |
| P73 | 78 | HSDAVFTDNYTRLRKQVAARKYLQSIKNKRYGGPSSGAP |
| | | PPS |
| P74 | 79 | HSDAVFTDNYTRLRKQVAAKKYLQSIQNKRYGGPSSGAP |
| | | PPS |
| P75 | 80 | HSDAVFTDNYTRLRKQVAAKKYLQSIKNNRYGGPSSGAP |
| | | PPS |
| P76 | 81 | HSDAVFTDNYTRLRKQVAAKKYLQSIKNKIYGGPSSGAP |
| | | PPS |
| P82 | 82 | HSDAVFTDNYTRLRKQVAAKIKYLQSIKRGGPSSGAPPP |
| | | S |
| P83 | 83 | HSDAVFTDNYTRLRKQVAAKIKYLQSIKNGGPSSGAPPP |
| | | S |
| P84 | 84 | dHSDAVFTDNYTRLRKQVAAKKYLQSIKNKRYGGPSSGA |
| | | PPPS |
| P85 | 85 | HSDAVFTDNYTRLRKQVAAKKYLQSIKKGGPSSGAPPPS |
| P87 | 86 | HSDAVFTDNYTREKEKVAAKKYLQSIKNKRYGGPSSGAP |
| | | PPS |
| P88 | 87 | HSDAVFTDNYTRAAAKVAAKKYLQSIKNKRYGGPSSGAP |
| | | PPS |
| P89 | 88 | HSDAVFTDNYTRLLAKVAAKKYLQSIKNKRYGGPSSGAP |
| | | PPS |
| P92 | 89 | HSDAVFTDNYTRLRKQVAAKKYLQSIKNGRPSSGAPPPS |
| P93 | 90 | HSDAVFTDNYTRLLLKVAAKKYLQSIKNKRYGGPSSGAP |
| | | PPS |
| P94 | 91 | HSDAVFTDNYTRAKAKVAAKKYLQSIKNKRYGGPSSGAP |
| | | PPS |
| P98 | 92 | C6-HSDAVFTDNYTRLRRQVAARRYLQSTRNRRYGGPSS |
| | | GAPPPS |
| P99 | 93 | C6-HVDAVFTDNYTRLRKQVAAKKYLQSIKNKRYGGPSS |
| | | GAPPPS |
| P100 | 94 | M-HSDAVFTDQYTRLRKQVAAKKYLQSIKQKRYGGPSSG |
| | | APPPS |
| P101 | 95 | C6-HdADAVFTDNYTRLRKQVAAKKYLQSIKNKRYGGPS |
| | | SGAPPPS |
| P102 | 96 | HSDGVFTDNYTRLRKQVAAKKYLQSIKNKRYGGPSSGAP |
| | | PPS |
| P103 | 97 | C6-HSDAVFTDNYTKLKKQVAAKKYLQSIKNKKYGGPSS |
| | | GAPPPS |
| P104 | 98 | M-HSDAVFTDNYTRLRKQVAAKKYLQSIKNKRYGGPSSG |
| | | APPPS |
| P105 | 99 | I-HSDAVFTDNYTRLRKQVAAKKYLQSIKNKRYGGPSSG |
| | | APPPS |
| P106 | 100 | C6-HDAVGTDNYTRLRKQVAAKKYLQSFKNKRYGGPSSG |
| | | APPPS |
| P107 | 101 | C6-HSDAVFTDNYTRLRKQVAAKKYLQSLKNKRYGGPSS |
| | | GAPPPS |
| P108 | 102 | C6-HSDAVFTDNYTRLRKQVAAKKYLQSTKNKRYGGPSS |
| | | GAPPPS |
| P109 | 103 | C6-HSDAVFTDNYTRLRKQVAAKKYLQSVKNKRYGGPSS |
| | | GAPPPS |
| P110 | 104 | C6-HSDAVFTDNYTRLRKQVAAKKYLQSWKNKRYGGPSS |
| | | SAPPPS |
| P11 | 105 | C6-HSDAVFTDNYTRLRKQVAAKKYLQSYKNKRYGGPSS |
| | | GAPPPS |
| P112 | 106 | C6-HSDAVFTDNYTRLRKQVAAKKYLQFIKNKRYGGPSS |
| | | GAPPPS |
| P113 | 107 | C6-HSDAVFTDNYTRLRKQVAAKKYLQIIKNKRYGGPSS |
| | | GAPPPS |
| P114 | 108 | C6-HSDAVFTDNYTRLRKQVAAKKYLQLIIKNKRYGGPS |
| | | SGAPPPS |
| P115 | 109 | C6-HSDAVFTDNYTRLRKQVAAKKYLQTIKNKRYGGPSS |
| | | GAPPPS |
| P116 | 110 | C6-HSDAVFTDNYTRLRKQVAAKKYLQVIKNKRYGGPSS |
| | | GAPPPS |
| P117 | 111 | C6-HSDAVFTDNYTRLRKQVAAKKYLQWIKNKRYGGPSS |
| | | GAPPPS |
| P119 | 112 | C6-HSDAVFTDNYTRLLAKLALQKYLQSIKNKRYGGPSS |
| | | GAPPPS |
| P120 | 113 | C6-HSDAVFTDNYTRLRKQVAAKKYLQSIKNKRYGGPSS |
| | | GAPPPC |
| P121 | 114 | C6-HSDAVFTDNYTRLRKQVAAKKYLQSIKNKKGGPSSG |
| | | APPPS |
| P122 | 115 | C6-HSDAVFTDNYTRLRKQVAAKKYLQSIKNSRGGPSSG |
| | | APPPS |
| P123 | 116 | C6-HSDAVFTDNYTRLRKQVAAKKYLQSIKNKRGGPSSG |
| | | APPPS |
| P124 | 117 | C6-HSDAVFTDNYTRLRKQVAAKKYLQQIKNKRYGGPSS |
| | | GAPPPS |
| P125 | 118 | C6-HSDAVFTDNYTRLRKQVAAKKYLQNIKNKRYGGPSS |
| | | GAPPPS |
| P126 | 119 | HSDAVFTDNYTRLRKQVAAKKYLQSIKRGRPSSGAPPPS |
| P127 | 120 | C6-HSDAVFTDNYTRLRKQVAAKKYLQYIKNKRYGGPSS |
| | | GAPPPS |
| P129 | 121 | C6-HSDAVFTDNYTRLRKQVAAKKWLQSIKNKRYGGPSS |
| | | GAPPPS |
| P130 | 122 | C6-HSDAVFTDNYTRLRKQVAAKKFLQSIKNKRYGGPSS |
| | | GAPPS |
| P131 | 123 | C6-HSDAVFTDNYTRLRKQVAAKKTLQSIKNKRYGGPSS |
| | | GAPPPS |
| P132 | 124 | C6-HSDAVFTDNYTRLRKQVAAKKLLQSIKNKRYGGPSS |
| | | GAPPPS |
| P133 | 125 | C6-HSDAVFTDNYTRLRKQVAAKKILQSIKNKRYGGPSS |
| | | GAPPPS |
| P134 | 126 | C6-HSDAVFTDNYTRLRKQVAAKKVLQSIKNKRYGGPSS |
| | | GAPPPS |
| P135 | 127 | C6-HSDAVFTDNYTRLLAKVAAKKYLQSIKNKRYGGPSS |
| | | GAPPPS |
| P138 | 128 | C6-HSDAVFTDNYTRLRAQVAAQKYLQSIKNKRYGGPSS |
| | | GAPPPS |
| P139 | 129 | C6-HSDAVFTDNYTRLRAQVAAKKYLQSIKNKRYGGPSS |
| | | GAPPPS |
| P140 | 130 | M-HISDAVFTDNYTRLLAKLALQKYLQSIKNKRYGGPSS |
| | | GAPPPS |
| P141 | 131 | C6-HSDAVFTDNYTRLKAQVAAKKYLQSIKNKRYGGPSS |
| | | GAPPPS |
| P142 | 132 | C6-HSDAVFTDNYTRLRAQLAAQKYLQSIKNKRYGGPSS |
| | | GAPPPS |
| P143 | 133 | C6-HSDAVFTDNYTRLRKQMAAQKYLNQLKKGGPSSGAP |
| | | PPS |
| P144 | 134 | C6-HSDAVFTDNYTRLRKQVAAQKYLNQLKKGGPSSGAP |
| | | PPS |
| P146 | 135 | C6-HSDAVFTDNYTRLRKQVAAVKYLQSIKNKRYGGPSS |
| | | GAPPPS |
| P147 | 136 | C6-HSDAVFTDNYTRLRKQVAAYKYLQSIKNKRYGGPSS |
| | | GAPPPS |
| P148 | 137 | C6-HSDAVFTDNYTRLRKQVAAFKYLQSIKNKRYGGPSS |
| | | GAPPPS |
| P149 | 138 | C6-HSDAVFTDNYTRLRKQVAAIKYLQSTKNKRYGGPSS |
| | | GAPPPS |
| P150 | 139 | C6-HSDAVFTDNYTRLRKQVAAQKYLQSIKNKRYGGPSS |
| | | GAPPPS |
| P151 | 140 | C6-HSDAVFTDNYTRLRKQVAALKYLQSIKNKRYGGPSS |
| | | GAPPPS |
| P152 | 141 | C6-HSDAVFTDNYTRLRKQVAATKYLQSIKNKRYGGPSS |
| | | GAPPPS |
| P153 | 142 | C6-HSDAVFTDNYTRLRKQVAAWKYLQSIKNKRYGGPSS |
| | | GAPPPS |
| P154 | 143 | C6-HSDAVFTDNYTRLRKQVAARKYLQSIKNGGPSSGAP |
| | | PPS |
| P155 | 144 | C6-HSDAVFTDNYTRLRKQVALKKYLQSIKNKRYGGPSS |
| | | GAPPPS |
| P158 | 145 | C6-HSDAVFTANYTRLRKQVAAKKYLQSIKNKRYGGPSS |
| | | GAPPPS |
| P159 | 146 | C6-HSDAVFTENYTRLRKQVAAKKYLQSIKNKRYGGPSS |
| | | GAPPPS |
| P160 | 147 | C6-HSDAVFTKNYTRLRKQVAAKKYLQSIKNKRYGGPSS |
| | | GAPPPS |
| P161 | 148 | C6-HSDAVFTLNYTRLRKQVAAKKYLQSIKNKRYGGPSS |
| | | GAPPPS |
| P162 | 149 | C6-HSDAVFTRNYTRLRKQVAAKKYLQSIKNKRYGGPSS |
| | | GAPPPS |
| P163 | 150 | C6-HSDAVFTYNYTRLRKQVAAKKYLQSIKNKRYGGPSS |
| | | GAPPPS |
| P164 | 151 | C6-HSDAVFTDNYTRLLAKLALQKYLQSIKNKRYGGPSS |
| | | GAPPPC |
| P165 | 152 | C6-HSDAVFTEEYTRLQKQVAAKQYLQSIKNKRYGGPSS |
| | | GAPPPS |
| P166 | 153 | C6-HAibDAVFTDNYTRLRKQVAAKKYLQSIKNKRYGGP |
| | | SSGAPPPS |
| P167 | 154 | C6-HSDAVFTDNYTRLAibKQVAAKKYLQSIKNKRYGGP |
| | | SSGAPPPS |
| P168 | 155 | C6-HSDAVFTDNYTRLRAibQVAAKKYLQSIKNKRYGGP |
| | | SSGAPPPS |
| P169 | 156 | C6-HSDAVFTDNYTRLRKQVAAAibKYLQSIKNKRYGGP |
| | | SSGAPPPS |
| P170 | 157 | C6-HSDAVFTDNYTRLRKQVAAKAibYLQSIKNKRYGGP |
| | | SSGAPPPS |
| P171 | 158 | C6-HSDAVFTDNYTRLKKQVAAKKYLQSIKNKRYGGPSS |
| | | GAPPPS |
| P172 | 159 | C6-HSDAVFTDNYTRLQKQVAAKKYLQSIKNKRYGGPSS |
| | | GAPPPS |
| P173 | 160 | C6-HSDAVFTDNYTRLAKQVAAKKYLQSIKNKRYGGPSS |
| | | GAPPPS |
| P174 | 161 | C6-HSDAVFTDNYTRLLKQVAAKKYLQSIKNKRYGGPSS |
| | | GAPPPS |
| P175 | 162 | C6-HSDAVFTDNYTRLKQVAAKKYLQSIKNKRYGGPSSG |
| | | APPPS |
| P176 | 163 | C6-HSDAVFTDNYTRLRRQVAAKKYLQSIKNKRYGGPSS |
| | | GAPPPS |
| P177 | 164 | C6-HSDAVFTDNYTRLRQQVAAKKYLQSIKNKRYGGPSS |
| | | GAPPPS |
| P179 | 165 | C6-HSDAVFTDNYTRLRLQVAAKKYLQSIKNKRYGGPSS |
| | | GAPPPS |
| P180 | 166 | C6-HSDAVFTDNYTRLRFQVAAKKYLQSIKNKRYGGPSS |
| | | GAPPPS |
| P181 | 167 | C6-HSDAVFTDNYTRLRKQVAAAKYLQSIKNKRYGGPSS |
| | | GAPPPS |
| P182 | 168 | C6-HSDAVFTDNYTRLRKQVAAKRYLQSIKNKRYGGPSS |
| | | GAPPPS |
| P183 | 169 | C6-HSDAVFTDNYTRLRKQVAAKQYLQSIKNKRYGGPSS |
| | | GAPPPS |
| P184 | 170 | C6-HSDAVFTDNYTRLRKQVAAKAYLQSIKNKRYGGPSS |
| | | GAPPPS |
| P185 | 171 | C6-HSDAVFTDNYTRLRKQVAAKLYLQSIKNKRYGGPSS |
| | | GAPPPS |
| P186 | 172 | C6-HSDAVFTDNYTRLRKQVAAKFYLQSIKNKRYGGPSS |
| | | GAPPPS |
| P187 | 173 | C6-HSDAVFTDNYTRLRKQVAAKKYLQAibIKNKRYGGP |
| | | SSGAPPPS |
| P188 | 174 | C6-HSDAVFTDNYTRLRKQVAAKKYLQSAibKNKRYGGP |
| | | SSGAPPPS |
| P189 | 175 | C6-HSDAVFTDNYTRLRKQAibAAKKYLQSIKNKRYGGP |
| | | SSGAPPPS |
| P191 | 176 | C6-HHSDAVFTDNYTRLLAKLALQKYLQSIKNKRYGGPS |
| | | SGAPPPS |
| P192 | 177 | C6-HSTDAVFTDQYTRLLAKLALQKYLQSIKQKRYGGPS |
| | | GAPPPS |
| P193 | 178 | C6-HSDAVFTDNYTRLRK(Ac)QVAAK(Ac)KYLQSIKN |
| | | KRYGGPSSGAPPPS |
| P194 | 179 | C6-HSDAVFTDNYTRLRK(Ac)QVAAKK(Ac)YLQSIKN |
| | | KRYGGPSSGAPPPS |
| P195 | 180 | C6-HSDAVFTDNYTRLLAQLALQKYLQSIKNKRYGGPSS |
| | | GAPPPS |
| P196 | 181 | C6-HSDAVFTDNYTRLLAKVALQKYLQSIKNKRYGGPSS |
| | | GAPPPS |
| P197 | 182 | C6-HSDAVFTDNYTRLLAKLAAQKYLQSIKNKRYGGPSS |
| | | GAPPPS |
| P198 | 183 | C6-HSDAVFTDNYTRLLAKLALQKYLQSIKNKGGPSSG |
| | | APPPC |
| P199 | 184 | Met(O)-HSDAVFTDNYTRLRKQVAAKKYLQSIKNKRY |
| | | GGPSSGAPPPS |
| P203 | 185 | C6-HSDAVFTDNYTRLRKQVAAKKYLQSIKNKRFGGPS |
| | | SGAPPPS |
| P205 | 186 | HS(CH₂)₂CO-HSDAVFTDNYTRLLAKLALQKYLQSIK |
| | | NKRYGGPSSGAPPPS |
| P206 | 187 | HS(CH₂)₂CO-HSDAVFTDNYTRLRKQVAAKKYLQSIK |
| | | NKRYGGPSSGAPPPS |
| P207 | 188 | C6-HSDdAVFTDNYTRLRKQVAAKKYLQSIKNKRYGGPS |
| | | SGAPPPS |
| P208 | 189 | C6-HSDNMeAVFTDNYTRLRKQVAAKKYLQSIKNKRYGG |
| | | PSSGAPPPS |
| P209 | 190 | C6-HSDAibVFTDNYTRLRKQVAAKKYLQSIKNKRYGGP |
| | | SSGAPPPS |
| P210 | 191 | C6-HSDAdVFTDNYTRLRKQVAAKKYLQSIKNKRYGGPS |
| | | SGAPPPS |
| P211 | 192 | C6-HSDANMeVFTDNYTRLRKQVAAKKYLQSIKNKRYGG |
| | | PSSGAPPPS |
| P212 | 193 | C6-HSDAAibFTDNYTRLRKQVAAKKYLQSIKNKRYGGP |
| | | SSGAPPPS |
| P213 | 194 | C6-HSDAVFTDNYTRLRKQVAAKRYLQSIRNGGPSSGAP |
| | | PPS |
| P214 | 195 | C6-HSDAVFTDNYTRLRKQVAARRYLQSIRNGGPSSGAP |
| | | PPS |
| P215 | 196 | C6-HSDAVFTDNYTRLRRQVAAKRYLQSIRNGGPSSGAP |
| | | PPS |
| P216 | 197 | C6-HSDAVFTDNYTRLRRQVAARKYLQSTRNGGPSSGAP |
| | | PPS |
| P220 | 198 | C6-HSDAVFTDQYTRLRRQVAARKYLQSIRQGGPSSGAP |
| | | PPS |
| P221 | 199 | C6-HSDIVFTDNYTRLRKQVAAKKYLQSIKNKRYGGPSS |
| | | GAPPPS |
| P222 | 200 | C6-HGEGTFTDNYTRLRKQVAAKKYLQSIKNKRYGGPSS |
| | | GAPPPS |
| P223 | 201 | C6-HSDLVFTDNYTRLRKQVAAKKYLQSIKNKRYGGPSS |
| | | GAPPPS |
| P224 | 202 | C6-HSEAVFTDNYTRLRKQVAAKKYLQSIKNKRYGGPSS |
| | | GAPPPS |
| P226 | 203 | C6-HSDAVFTDNY(OMe)TRLRKQVAAKKYLQSIKNKRY |
| | | GGPSSGAPPPS |
| P227 | 204 | C6-HSDAVFTDNYTRLRKQVAAKKY(OMe)LQSIKNKRY |
| | | GGPSSGAPPPS |
| P228 | 205 | C6-HSDAVFTDNYTRLRKQVAAKKAibLQSIKNKRYGGP |
| | | SSGAPPPS |
| P229 | 206 | C6-HSDAVFTDNYTRLRKQVAAKKALQSIKNKRYGGPSS |
| | | GAPPPS |
| P230 | 207 | C6-HSDAVFTDNYTRLRKQVAAKKYWQSIKNKRYGGPSS |
| | | GAPPPS |
| P231 | 208 | C6-HSDAVFTDNYTRLRKQVAAKKYFQSIKNKRYGGPSS |
| | | GAPPPS |
| P232 | 209 | C6-HSDAVFTDNYTRLRKQVAAKKYTQSIKNKRYGGPSS |
| | | GAPPPS |
| P233 | 210 | C6-HSDAVFTDNYTRLRKQVAAKKYIQSIKNKRYGGPSS |
| | | GAPPPS |
| P234 | 211 | C6-HSDAVGTDNYTRLRKQVAAKKYVQSIKNKRYGGPSS |
| | | GAPPPS |
| P235 | 212 | C6-HSDAVFTDNYTRLRKQVAAKKYAQSIKNKRYGGPSS |
| | | GAPPPS |
| P236 | 213 | C6-HSDAVFTDNYTRLRKQVAAKKYAibQSIKNKRYGGP |
| | | SSGAPPPS |
| P240 | 214 | C6-HSDAVFTDNYTRLAibKQLAAAibKYLQSIKNKRYG |
| | | GPSSGAPPPS |
| P241 | 215 | C6-HSDAVFTDNYTRLAibKQLAAKAibYLQSIKNKRYG |
| | | GPSSGAPPPS |
| P242 | 216 | C6-HSDAVFTDNYTRLAibKQVAAAibKYLQSIKNKRYG |
| | | GPSSGAPPPS |
| P243 | 217 | C6-HSDAVFTDNYTRLAibKQVAAQKYLQSIKNKRYGGP |
| | | SSGAPPPS |
| P244 | 218 | C6-HSDAVFTDNYTRLAibKQVAAKAibYLQSIKNKYGG |
| | | PSSGAPPPS |
| P249 | 219 | C6-HSDAVFTDNYTRLAibKQVAAKQYLQSIKRYGGPSS |
| | | GAPPPS |
| P250 | 220 | C6-HSDAVFTDNYTRLQKQVAAAibKYLQSIKNKRYGGP |
| | | SSGAPPPS |
| P251 | 221 | C6-HSDAVFTDNYTRLQKQVAAKAibYLQSIKNKRYGGP |
| | | SSGAPPPS |
| P252 | 222 | C6-HSDAVFTDNYTRLQKQVAAQKYLQSIKNKRYGGPSS |
| | | GAPPPS |
| P253 | 223 | C6-HSDAVFTDNYTRLQKQVAAKQYLQSIKNKRYGGPSS |
| | | GAPPPS |
| P258 | 224 | C6-HSDAVFTDNYTRLQAibQVAAKKYLQIKNKRYGGPS |
| | | SGAPPPS |
| P259 | 225 | C6-HSDAVFTDNYTRLAibKQVAALKYLQSIKNKRYGGP |
| | | SSGAPPPS |
| P260 | 226 | C6-HSDAVFTDNYTRLAibKQVAAAKYLQSIKNKRYGGP |
| | | SSGAPPPS |
| P261 | 227 | C6-HSDAVFTDNYTRLRAibQVAAAibKYLQSIKNKRYG |
| | | GPSSGAPPPS |
| P262 | 228 | C6-HSDAVFTDNYTRLRAibQVAAVKYLQSIKNKRYGGP |
| | | SSGAPPPS |
| P263 | 229 | C6-HSDAVFTDNYTRLRAibQVAAAKYLQSIKNKRYGGP |
| | | SSGAPPPS |
| P264 | 230 | C6-HSDAVFTDNYTRLRAibQVAAKAibYLQSIKNKRYG |
| | | GPSSGAPPPS |
| P265 | 231 | C6-HSDAVFTDNYTRLRAibQVAALKYLQSIKNKRYGGP |
| | | SSGAPPPS |
| P269 | 232 | C6-HSDAVFTDNYTRLAibKQVAAVKYLQSIKNKRYGGP |
| | | SSGAPPPS |
| P270 | 233 | C6-HSDAVFTDNYTRLRKQVAAKKYLQSIKQKGGPSSGA |
| | | PPPS |
| P271 | 234 | C6-HSDAVFTDNYTRLRKQVAAKKYLQSIKNGRPSSGAP |
| | | PPS |
| P275 | 235 | C6-HSDAVFTDNYTRLRKQVAGKKYLQSIKNKRYGGPSS |
| | | GAPPPS |
| P282 | 236 | C6-HSDAVGTDNYTRLAibKQYAAAibKYLQSIKNKRYG |
| | | GPSSGAPPPC-NH₂ |
| P284 | 237 | C6-HSDAVFTDNYTRLRAibQLAAKAibYLQSIKNKRYG |
| | | GPSSGAPPPS |
| P285 | 238 | C6-HSDAVFTDNYTRLRAibQVAAKAibYLQSIKNKRYG |
| | | GPSSGAPPPC-NH₂ |
| P289 | 239 | C6-HSDALFTDNYTRLRKQVAAKKYLQSIKNKRYGGPSS |
| | | GAPPPS |
| P290 | 240 | C6-HSDAVFTDNYTRLRKQLAAKKYLQSIKNKRYGGPSS |
| | | GAPPPS |
| P291 | 241 | C6-HSDAVFTDNYTRLRAibQLAAAibKYLQSIKNKRYG |
| | | GPSSGAPPPS |
| P292 | 242 | C6-HSDAVFTDNYTRLRAibQLAAAibKYLQSIKNKGGP |
| | | SSGAPPPS |
| P293 | 243 | C6-HSDAVFTDNYTRLAibKQVAAAibKYLQSIKQKGGP |
| | | SSGAPPPS |
| P294 | 244 | C6-HSDAVFTDNYTRLRAibQVAAAibKYLQSIKQKGGP |
| | | SSGAPPPS |
| P295 | 245 | C6-HSDAVFTDNYTRLRAibQVAAKAibYLQSIKQKGGP |
| | | SSGAPPPS |
| P296 | 246 | C6-HSDAVFTDNYTRLAibKQVAAAibKYLQSIKQGGPS |
| | | SGAPPPS |
| P297 | 247 | C6-HSDAVFTDNYTRLRAibQVAAAibKYLQSIKQGGPS |
| | | SGAPPPS |
| P298 | 248 | C6-HSDAVFTDNYTRLRAibQVAAKAibYLQSIKQGGPS |
| | | SGAPPPS |
| P299 | 249 | C6-HSDAVFTDNYTRLRAibQVAAAibKYLQSIKNKRYG |
| | | GPSSGAPPPC-NH₂ |
| P301 | 250 | C6-HSDAVFTDNYTRLAAibQVAAAibKYLQSIKNKRYG |
| | | GPSSGAPPPS |
| P302 | 251 | C6-HSDAVFTDNYTRLQAibQVAAAibKYLQSIKNKRYG |
| | | GPSSGAPPPS |
| P305 | 252 | C6-HSDAVFTDNYTRLhRKQVAAKKYLQSIKNKRYGGPS |
| | | SGAPPPS |
| P307 | 253 | C6-HSDAVFTDNYTRLROrnQVAAKKYLQSIKNKRYGGP |
| | | SSGAPPPS |
| P308 | 254 | C6-HSDAVFTDNYTRLhROrnQVAAKKYLQSIKNKRYGG |
| | | PSSGAPPPS |
| P314 | 255 | C6-HSFAVFTENYTRLRAibQVAAAibKYLQSIKNKRYG |
| | | GPSSGAPPPS |
| P315 | 256 | C6-HSDAVFTDQYTRLRAibQVAAAibKYLQSIKQKRYG |
| | | GPSSGAPPPS |
| P316 | 257 | C6-HSDAVFTDNYTRLhRAibQVAAAibKYLQSIKNKRY |
| | | GGPSSGAPPPS |
| P317 | 258 | C6-HSDAVFTDNYTRLLAibQVAAAibKYLQSIKNKRYG |
| | | GPSSGAPPPS |
| P318 | 259 | C6-HSDAVFTDNYTRLKAibQVAAAibKYLQSIKNKRYG |
| | | GPSSGAPPPS |
| P319 | 260 | C6-HSDAVFTDNYTRLOrnAibQVAAAibKYLQSIKNKR |
| | | YGGPSSGAPPPS |
| P320 | 261 | C6-HSDAVFTDNYTRLCitAibQVAAAibKYLQSIKNKR |
| | | YGGPSSGAPPPS |
| P321 | 262 | C6-HSDAVFTDNYTRLRAibKVAAAibKYLQSIKNKRYG |
| | | GPSSGAPPPS |
| P322 | 263 | C6-HSDAVFTDNYTRLRAibQIAAAibKYLQSIKNKRYG |
| | | GPSSGAPPS |
| P323 | 264 | C6-HSDAVFTDNYTRLRAibQKAAAibKYLQSIKNKRYG |
| | | GPSSGAPPPS |
| P324 | 265 | C6-HSDAVFTDNYTRLRAibQAAAAibKYLQSIKNKRYG |
| | | GPSSGAPPPS |
| P325 | 266 | C6-HSDAVFTDNYTRLRAibQNleAAAibKYLQSIKNKR |
| | | YGGPSSGAPPPS |
| P326 | 267 | C6-HSDAVFTDNYTOrnLRAibQVAAAibKYLQSIOrnN |
| | | OrnGGPSSGAPPPS |
| P327 | 268 | C6-HSDAVFTDNYTOrnLRAibQVAAAibKYLQSIOrnN |
| | | OrnGGPSSGAPPPC-NH₂ |
| P329 | 269 | C6-HSDAVFTDNYThRLRAibQVAAAibKYLQSIhRNhR |
| | | GGPSSGAPPPS |
| P330 | 270 | C6-HSDAVFTDNYThRLRAibQVAAAibKYLQSIhRNhR |
| | | GGPSSGAPPPC-NH₂ |
| P332 | 271 | HSDAVFTDNYThRLRAibQVAAAibKYLQSIhRhRGGPS |
| | | SGAPPPS |
| P333 | 272 | C6-HSDAVFTDNYThRLRAibQVAAAibKYLQSIhRhRG |
| | | GPSSGAPPPC-NH₂ |
| P335 | 273 | C6-HSDAVFTDNYTOrnLRAibQVAAAibKYLQSIhRhR |
| | | GGPSSGAPPPS |
| P336 | 274 | C6-HSDAVFTDNYTOrnLRAibQVAAAibKYLQSIhRhR |
| | | GGPSSGAPPPC-NH |
| P338 | 275 | C6-HSDAVFTDNYThRLRAibQVAAAibKYLQSIhRNhR |
| | | hRYGGPSSGAPPPS |
| P339 | 276 | C6-HSDAVFTDNYThRLRAibQVAAA1bKYLQSThRNhR |
| | | hRYGGPSSGAPPPC-NH₂ |
| P341 | 277 | C6-HSDAVFTDNYTRLRAibQVAAAibAYLQSIKNKRYG |
| | | GPSSGAPPPS |
| P342 | 278 | C6-HSDAVFTDNYTRLRAibQVAAAibOrnYLQSIKNKR |
| | | YGGPSSGAPPPS |
| P343 | 279 | C6-HSDAVFTDNYThRLRAibQVAAAibKYLQSIKNKRY |
| | | GGPSSGAPPPS |
| P344 | 280 | C6-HSDAVFTDNYTOrnLRAibQVAAAibKYLQSIKNKR |
| | | YGGPSSGAPPPS |
| P345 | 281 | C6-HSDAVFTDNYTAibLRAibQVAAAibKYLQSIKNKR |
| | | YGGPSSGAPPPS |
| P346 | 282 | C6-HSDAVFTDNYTRAibRAibQVAAAibKYLQSIKNKR |
| | | YGGPSSGAPPPS |
| P349 | 283 | C6-HSDAVFTDNYTRLRAibQVAAAibKYLQSIKAibKG |
| | | GPSSGAPPPS |
| P350 | 284 | C6-HSDAVFTDNYTRLRAibQVAAAibKYLQSIKPKGGP |
| | | SSGAPPPS |
| P351 | 285 | C6-HSDAVFTDNYTRLRAibQVAAAibKYLQSIKKGGPS |
| | | SGAPPPS |
| P352 | 286 | C6-HSDAVFTDNYTRLAibQVAAAibKYLQSIOrnOrnG |
| | | GPSSGAPPPS |
| P353 | 287 | C6-HSDAVFTDNYTRLRAibQVAAAibKYLQSIdKdKGG |
| | | PSSGAPPPS |
| P354 | 288 | C6-HSDAVFTDNYTRLRAibQVAAAibKYLQSIKhRGGP |
| | | SSGAPPPS |
| P355 | 289 | C6-HSDAVFTDNYTRLRAibQVAAAibKYLQSIKAibGG |
| | | PSSGAPPPS |
| P356 | 290 | C6-HSDAVFTDNYTRLRAibQVAAAibKYLQSIOrnQ |
| | | OrnGGPSSGAPPPS |
| P357 | 291 | C6-HSDAVFTDNYTRLRAibQVAAAibKYLQSIKAib |
| | | OrnGGPSSGAPPPS |
| P358 | 292 | C6-HSDAVFTDNY(OMe)TRLRAibQVAAAibKYLQSIK |
| | | NKRYGGPSSGAPPPS |
| P362 | 293 | C6-HSEAVFTENYTOrnLRAibQVAAAibKYLQSIOrnN |
| | | OrnGGPSSGAPPPS |
| P363 | 294 | C6-HSDAVFTDQYTOrnLRAibQVAAAibKYLQSIOrnQ |
| | | OrnGGPSSGAPPPS |
| P364 | 295 | C6-HSDAVFTDNYTOrnLRAibQLAAAibKYLQSIOrnN |
| | | OrnGGPSSGAPPPS |
| P365 | 296 | C6-HSDAVFTDNYTOrnLRAibQIAAAibKYLQSIOrnN |
| | | OrnGGPSSGAPPPS |
| P366 | 297 | C6-HSDAVFTDNYTALRAibQVAAAibKYLQSIOrnN |
| | | OrnGGPSSGAPPPS |
| P367 | 298 | C6-HSDAVFTDNYTLLRAibQVAAAibKYLQSIOrnN |
| | | OrnGGPSSGAPPPS |
| P368 | 299 | C6-HSDAVFTDNYTQLRAibQVAAAibKYLQSIOrnN |
| | | OrnGGPSSGAPPPS |
| P369 | 300 | C6-HSDAVFTDNYTFLRAibQVAAAibKYLQSIOrnN |
| | | OrnGGPSSGAPPPS |
| P370 | 301 | C6-HSDAVFTDNYTRLLAKLALQKYLQSIOrnNOrnGGP |
| | | SSGAPPPS |
| P371 | 302 | C6-HSDAVFTDNYTOrnLLAKLALQKYLQSIOrnNOrnG |
| | | GPSSGAPPPS |
| P372 | 303 | C6-HSDAVFTDNYTOrnLRAibQVAAAibKYLQCIOrnN |
| | | OrnGGPSSGAPPPS |
| P377 | 304 | C6-HSDAVFTDNYTOrnLRAibQVAACOrnYLQSIOrNN |
| | | OrNGGPSSGAPPPS-NH₂ |
| P379 | 305 | C6-HSEAVFTEQYTOrnLRAibQVAAAibOrnYLQSI |
| | | OrnOrnGGPSSGAPPPC-NH₂ |
| P382 | 306 | C6-HSDAVFTDNYTOrnLRAibQVAAAibKYLQSSOrnN |
| | | OrnGGPSSGAPPPS |
| P383 | 307 | C6-HSDAVFTDNYTOrnLRAibQVAAAibKYLSSIOrnN |
| | | OrnGGPSSGAPPPS |
| P384 | 308 | C6-HSDAVFTDNYTOrnLRAibQVAAAibKYSQSIOrnN |
| | | OrnGGPSSGAPPPS |
| P385 | 309 | C6-HSDAVFTDNYTOrnLRAibQVAAAibKSLQSIOrnN |
| | | OrnGGPSSGAPPPS |
| P386 | 310 | C6-HSDAVFTDNYTOrnLRAibQVAAAibSYLQSIOrnN |
| | | OrnGGPSSGAPPPS |
| P387 | 311 | C6-HSDAVFTDNYTOrnLRAibQVASAibKYLQSIOrnN |
| | | OrnGGPSSGAPPPS |
| P388 | 312 | C6-HSDAVFTDNYTOrnLRAibQVSAAibKYLQSIOrnN |
| | | OrnGGPSSGAPPPS |
| P389 | 313 | C6-HSDAVFTDNYTOrnLRAibQSAAAibKYLQSIOrnN |
| | | OrnGGPSSGAPPPS |
| P390 | 314 | C6-HSDAVFTDNYTOrnLRAibSVAAAibKYLQSIOrnN |
| | | OrrnGGPSSGAPPPS |
| P391 | 315 | C6-HSDAVFTDNYTOrnSRAibQVAAAibKYLQSIOrnN |
| | | OrnGGPSSGAPPPS |
| P392 | 316 | C6-HSDAVFTDSYTOrnLRAibQVAAAibKYLQSIOrnN |
| | | OrnGGPSSGAPPPS |
| P393 | 317 | C6-HSEAVFTEQY(OMe)TOrnLRAibQLAAAibOrnYL |
| | | QSIOrnOrnGGPSSGAPPS |
| P394 | 318 | C6-HSDAVFTDQY(OMe)TOrnLRAibQLAAAibOrnYL |
| | | QSIOrnOrnGGPSSGAPPPS |
| P395 | 319 | C6-HSDAVFTDQYTOrnLRAibQLAAAibOrnYLQSI |
| | | OrnOrnGGPSSGAPPPS |
| P396 | 320 | C6-HSDAVFTDQYTOrnLRAibQVAAAibOrnYLQSI |
| | | OrnOrnGGPSSGAPPPS |
| P397 | 321 | C6-HSDAVFTDNYTOrnLRAibQVAAAibOrnYLQSI |
| | | OrnOrnGGPSSGAPPPS |
| P398 | 322 | C6-HSDAVFTEQY(OMe)TOrnLRAibQVAAAibOrnYL |
| | | QSIOrnOrnGGPSSGAPPPC-NH₂ |
| P405 | 323 | C6-HSDAVFTDNYTRAibRAibQVAAAibKYLQSIKAib |
| | | KGGPSSGAPPPS |
| P406 | 324 | C6-HSDAVFTDQYTRAibRAibQVAAAibKYLQSIKAib |
| | | KGGPSSGAPPPS |
| P407 | 325 | C6-HSDAVFTDQYTRAibRAibQLAAAibKYLQSIKAib |
| | | KGGPSSGAPPPS |
| P408 | 326 | C6-HSDAVFTDQY(OMe)TRAibRAibQLAAAibKYLQS |
| | | IKAibKGGPSSGAPPPS |
| P409 | 327 | C6-HSEAVFTEQY(OMe)TRAibRAibQLAAAibKYLQS |
| | | IKAibKGGPSSGAPPPS |
| P412 | 328 | C6-HSDAVFTDNYTOrnLRK(W)QVAAAibKYLQSIOrn |
| | | NOrnGGPSSGAPPPS |
| P414 | 329 | C6-HSDAVFTEQY(OMe)TOrnLRAibQLAAAibOrnY |
| | | (OMe)LQSIOrnOrnGGPSSGAPPPC-NH₂ |
| P418 | 330 | C6-HSDAVFTEQY(OMe)TOrnLRAibQLAAAibOrnY |
| | | (OMe)LQSIOrnOrnGGPSSGAPPPS |
| P419 | 331 | C6-HSDAVTFEQY(OMe)TOrnLRAibVAAAibYLQKC |
| | | OrnOrnGGPSSGAPPPS-NH₂ |
| P425 | 332 | C6-HSDAVFTEQY(OMe)TOrnLRAibQVAAAibOrnYL |
| | | CSIOrnOrnGGPSSGAPPPS-NH₂ |
| P427 | 333 | C6-HSDAVFTEQY(OMe)TOrnLRAibQVAAAibOrnYL |
| | | K(CO(CH₂)₂SH)SIOrnOrnGGPSSGAPPPS-NH₂ |
| P429 | 334 | C6-HSDAVFTEQY(OMe)TOrnLRAibQVACAibOrnYL |
| | | QSIOrnOrnGGPSSGAPPPS-NH₂ |
| P431 | 335 | C6-HSDAVFTEQY(OMe)TOrnLRAibQVAK(CO |
| | | (CH₂)₂SH)AibOrnYLQSIOrnOrnGGPSSGAPPPS- |
| | | NH₂ |
| P433 | 336 | C6-HSDAVFTEQY(OMe)TOrnLRAibQCAAAibOrnYL |
| | | QSIOrnOrnGGPSSGAPPPS-NH₂ |
| P437 | 337 | C6-HSDAVFTEQY(OMe)TOrnLRAibCVAAAibOrnYL |
| | | QSIOrnOrnGGPSSGAPPPS-NH₂ |
| P442 | 338 | C6-HSDAVFTEQY(OMe)TOrnLRAibQVAAAibOrnYL |
| | | QSIOrnCOrnGGPSSGAPPPS-NH₂ |
| P446 | 339 | C6-HSDAVFTEQY(OMe)TOrnLRAibQVAAAibOrnYL |
| | | QCIOrnOrnGGPSSGAPPPC-NH₂ |
| P448 | 340 | C6-HSDAVFTECY(OMe)TOrnLRAibQVAAAibOrnYL |
| | | QSIOrnOrnGGPSSGAPPPS-NH₂ |
| P455 | 341 | C6-HSDAVFTEQY(OMe)TOrnCRAibQVAAAibOrnYL |
| | | QSIOrnOrnGGPSSGAPPPS-NH₂ |
| P421 | 722 | C6-HSDAVFTEQY(OMe)TOrnLRAibQVAAAibOrnYL |
| | | QSK(CO(CH₂)₂SH)OrnOrnGGPSSGAPPPS-NH₂ |
| P423 | 723 | C6-HSDAVFTEQY(OMe)TOrnLRAibQVAAAibOrnYL |
| | | QK(CO(CH₂)₂SH)IOrnOrnGGPSSGAPPPS-NH₂ |
| P435 | 724 | C6-HSDAVFTEQY(OMe)TOrnLRAibQK(CO(CH₂)₂S |
| | | H)AAAibOrnYLQSIOrnOrnGGPSSGAPPPS-NH2 |
| P439 | 725 | C6-HSDAVFTEQY(OMe)TOrnLRAibK(CO(CH₂)₂S |
| | | H)VAAAibOrnYLQSIOrnOrnGGPSSGAPPPS-NH₂ |
| P444 | 726 | C6-HSDAVFTEY(OMe)TOrnLRAibQVAAAibOrnYLQ |
| | | GIOrnK(CO(CH₂)₂SH)OrnGGPSSGAPPPS-NH₂ |
| P457 | 727 | C6-HSDAVFTEQY(OMe)TOrnK(CO(CH₂)₂SH)RAib |
| | | QVAAAibOrnYLQSIOrnOrnGGPSSGAPPPS-NH₂ |

Preferably, the following VPAC2 receptor peptide agonists may be PEGylated:

| | | |
|---|---|---|
| | SEQ | |
| Agonist | ID | |
| # | NO: | Sequence |

| | | |
|---|---|---|
| P18 | 34 | HSDAVFTDNYTRLRKQVAAhRKYLQSIKNKRYGGPSSGA |
| | | PPPS |
| P20 | 36 | HSDAVFTDNYTRLRKQVAARKYLQSIKNKRYGGPSSGAP |
| | | PPS |
| P21 | 37 | HSDAVFTDNYTRLRKQVAASKYLQSIKNKRYGGPSSGAP |
| | | PPS |
| P22 | 38 | HSDAVFTDNYTRLRKQVAAKKYLQSIhRNKRYGGPSSGA |
| | | PPPS |
| P23 | 39 | HSDAVFTDNYTRLRKQVAAKKYLQSIRNKRYGGPSSGAP |
| | | PPS |
| P31 | 46 | C6-HSDAVFTDNYTRLRKQVAAKKYLQSIKNKRYGGPSS |
| | | GAPPPS |
| P33 | 47 | Ac-HSDAVFTDNYTRLRKQVAAKKYLQSIKNKRYGGPSS |
| | | GAPPPS |
| P34 | 48 | HSDAVFTDNYTRLRKQVAAKKYLQSIKNKRF |
| P44 | 51 | HVDAVFTDNYTRLRKQVAAKKYLQSIKNKRYGGPSSGAP |
| | | PPS |
| P47 | 54 | HdADAVFTDNYTRLRKQVAAKKYLQSIKNKRYGGPSSGA |
| | | PPPS |
| P48 | 55 | HdSDAVFTDNYTRLRKQVAAKKYLQSIKNKRYGGPSSGA |
| | | PPPS |
| P61 | 66 | HSDALFTDNYTRLRKQVAAKKYLQSIKNKRYGGPSSGAP |
| | | PPS |
| P72 | 77 | HSDAVFTDNYTRLRRQVAAKKYLQSIKNKRYGGPSSGAP |
| | | PPS |
| P89 | 88 | HSDAVFTDNYTRLLAKVAAKKYLQSIKNKRYGGPSSGAP |
| | | PPS |
| P98 | 92 | C6-HSDAVFTDNYTRLRRQVAARRYLQSIRNRRYGGPSS |
| | | GAPPPS |
| P99 | 93 | C6-HVDAVFTDNYTRLRKQVAAKKYLQSIKNRYGPSSGA |
| | | PPPS |
| P100 | 94 | M-HSDAVFTDQYTRLRKQVAAKKYLQSIKQKRYGGPSSG |
| | | APPPS |
| P101 | 95 | C6-HdADAVFTDNYTRLRKQVAAKKYLQSIKNKRYGGPS |
| | | SGAPPPS |
| P103 | 97 | C6-HSDAVFTDNYTKLKKQVAAKKYLQSIKNKKYGGPSS |
| | | GAPPPS |
| P104 | 98 | M-HSDAVFTDNYTRLRKQVAAKKYLQSIKNIKRYGGPSS |
| | | GAPPPS |
| P106 | 100 | C6-HSDAVFTDNYTRLRKQVAAKKYLQSFKNKRYGGPSS |
| | | GAPPPS |
| P107 | 101 | C6-HSDAVFTDNYTRLRKQVAAKKYLQSLKNKRYGGPSS |
| | | GAPPPS |
| P108 | 102 | C6-HSDAVFTDNYTRLRKQVAAKKYLQSTKNKRYGGPSS |
| | | GAPPPS |
| P109 | 103 | C6-HSDAVFTDNYTRLRKQVAAKKYLQSVKNKRYGGPSS |
| | | GAPPPS |
| P110 | 104 | C6-HSDAVFTDNYTRLRKQVAAKKYLQSWKNKRYGGPSS |
| | | GAPPPS |
| P111 | 105 | C6-HSDAVFTDNYTRLRKQVAAKKYLQSYKNKRYGGPSS |
| | | GAPPPS |
| P112 | 106 | C6-HSDAVFTDNYTRLRKQVAAKKYLQFIKNKRYGGPSS |
| | | GAPPPS |
| P113 | 107 | C6-HSDAVFTDNYTRLRKQVAAKKYLQIIKNKRYGGPSS |
| | | GAPPPS |
| P114 | 108 | C6-HSDAVFTDNYTRLRKQVAAKKYLQLIKNKRYGGPSS |
| | | GAPPPS |
| P115 | 109 | C6-HSDAVFTDNYTRLRKQVAAKKYLQTIKNKRYGGPSS |
| | | GAPPPS |
| P116 | 110 | C6-HSDAVFTDNYTRLRKQVAAKKYLQVIKNKRYGGPSS |
| | | GAPPPS |
| P117 | 111 | C6-HSDAVFTDNYTRLRKQVAAKKYLQWIKNKRYGGPSS |
| | | GAPPPS |
| P119 | 112 | C6-HSDAVFTDNYTRLLAKLALQKYLQSIKNKRYGGPSS |
| | | GAPPPS |
| P120 | 113 | C6-HSDAVFTDNYTRLRKQVAAKKYLQSIKNKRYGGPSS |
| | | GAPPPC |
| P121 | 114 | C6-HSDAVFTDNYTRLRKQVAAKKYLQSIKNKKGGPSSG |
| | | APPPS |
| P122 | 115 | C6-HSDAVFTDNYTRLRKQVAAKKYLQSIKNSRGGPSSG |
| | | APPPS |
| P123 | 116 | C6-HSDAVFTDNYTRLRKQVAAKKYLQSIKNKRGGPSSG |
| | | APPPS |
| P124 | 117 | C6-HSDAVFTDNYTRLRKQVAAKKYLQQIKNKRYGGPSS |
| | | GAPPPS |
| P125 | 118 | C6-HSDAVFTDNYTRLRKQVAAKKYLQNIKNKRYGGPSS |
| | | GAPPPS |
| P127 | 120 | C6-HSDAVFTDNYTRLRKQVAAKKYLQYIKNKRYGGPSS |
| | | GAPPPS |
| P129 | 121 | C6-HSDAVFTDNYTRLRKQVAAKKWLQSIKNKRYGGPSS |
| | | GAPPPS |
| P130 | 122 | C6-HSDAVFTDNYTRLRKQVAAKKFLQSIKNKRYGGPSS |
| | | GAPPPS |
| P132 | 124 | C6-HSDAVFTDNYTRLRKQVAAKKLLQSIKNKRYGGPSS |
| | | GAPPPS |
| P133 | 125 | C6-HSDAVFTDNYTRLRKQVAAKKILQSIKNKRYGGPSS |
| | | GAPPPS |
| P134 | 126 | C6-HSDAVFTDNYTRLRKQVAAKKVLQSIKNKRYGGPSS |
| | | GAPPPS |
| P135 | 127 | C6-HSDAVFTDNYTRLLAKVAAKKYLQSIKNKRYGGPSS |
| | | GAPPPS |
| P138 | 128 | C6-HSDAVFTDNYTRLRAQVAAQKYLQSIKNKRYGGPSS |
| | | GAPPPS |
| P139 | 129 | C6-HSDAVFTDNYTRLRAQVAAKKYLQSIKNKRYGGPSS |
| | | GAPPPS |
| P142 | 132 | C6-HSDAVFTDNYTRLRAQLAAQKYLQSIKNKRYGGPSS |
| | | GAPPPS |
| P143 | 133 | C6-HSDAVFTDNYTRLRKQMAAQKYLNQLKKGGPSSGAP |
| | | PPS |
| P144 | 134 | C6-HSDAVFTDNYTRLRKQVAAQKYLNQLKKGGPSSGAP |
| | | PPS |
| P146 | 135 | C6-HSDAVFTDNYTRLRKQVAAVKYLQSIKNKRYGGPSS |
| | | GAPPPS |
| P147 | 136 | C6-HSDAVFTDNYTRLRKQVAAYKYLQSIKNKRYGGPSS |
| | | GAPPPS |
| P148 | 137 | C6-HSDAVFTDNYTRLRKQVAAFKYLQSIKNKRYGGPSS |
| | | GAPPPS |
| P149 | 138 | C6-HSDAVFTDNYTRLRKQVAAIKYLQSIKNKRYGGPSS |
| | | GAPPPS |
| P150 | 139 | C6-HSDAVFTDNYTRLRKQVAAQKYLQSIKNKRYGGPSS |
| | | GAPPPS |
| P151 | 140 | C6-HSDAVFTDNYTRLRKQVAALKYLQSIKNKRYGGPSS |
| | | GAPPPS |
| P152 | 141 | C6-HSDAVFTDNYTRLRKQVAATKYLQSIKNKRYGGPSS |
| | | GAPPPS |
| P153 | 142 | C6-HSDAVFTDNYTRLRKQVAAWKYLQSIKNKRYGGPSS |
| | | GAPPPS |
| P154 | 143 | C6-HSDAVFTDNYTRLRKQVAAKKYLQSIKNGGPSSGAP |
| | | PPS |
| P155 | 144 | C6-HSDAVFTDNYTRLRKQVALKKYLQSIKNKRYGGPSS |
| | | GAPPPS |
| P158 | 145 | C6-HSDAVFTANYTRLRKQVAAKKYLQSIKNKRYGGPSS |
| | | GAPPPS |
| P159 | 146 | C6-HSDAVFTENYTRLRKQVAAKKYLQSIKNKRYGGPSS |
| | | GAPPPS |
| P164 | 151 | C6-HSDAVFTDNYTRLLAKLALQKYLQSIKNKRYGGPSS |
| | | GAPPPC |
| P165 | 152 | C6-HSDAVFTEEYTRLQKQVAAKQYLQSIKNKRYGGPSS |
| | | GAPPPS |
| P166 | 153 | C6-HAibDAVFTDNYTRLRKQVAAKKYLQSIKNKRYGGP |
| | | SSGAPPPS |
| P167 | 154 | C6-HSDAVFTDNYTRLAibKQVAAKKYLQSIKNKRYGGP |
| | | SSGAPPPS |
| P168 | 155 | C6-HSDAVFTDNYTRLRAibQVAAKKYLQSIKNKRYGGP |
| | | SSGAPPPS |
| P169 | 156 | C6-HSDAVFTDNYTRLRKQVAAAibKYLQSIKNKRYGGP |
| | | SSGAPPPS |
| P170 | 157 | C6-HSDAVFTDNYTRLRKQVAAKAibYLQSIKNKRYGGP |
| | | SSGAPPPS |
| P171 | 158 | C6-HSDAVFTDNYTRLKKQVAAKKYLQSIKNKRYGGPSS |
| | | GAPPPS |
| P172 | 159 | C6-HSDAVFTDNYTRLQKQVAAKKYLQSIKNKRYGGPSS |
| | | GAPPPS |
| P173 | 160 | C6-HSDAVFTDNYTRLAKQVAAKKYLQSIKNKRYGGPSS |
| | | GAPPPS |
| P175 | 162 | C6-HSDAVFTDNYTRLFKQVAAKKYLQSIKNKRYGGPSS |
| | | GAPPPS |
| P176 | 163 | C6-HSDAVFTDNYTRLRRQVAAKKYLQSIKNKRYGGPSS |
| | | GAPPPS |
| P177 | 164 | C6-HSDAVFTDNYTRLRQQVAAKKYLQSIKNKRYGGPSS |
| | | GAPPPS |
| P179 | 165 | C6-HSDAVFTDNYTRLRLQVAAKKYLQSIKNKRYGGPSS |
| | | GAPPPS |
| P180 | 166 | C6-HSDAVFTDNYTRLRFQVAAKKYLQSIKNKRYGGPSS |
| | | GAPPPS |
| P181 | 167 | C6-HSDAVFTDNYTRLRKQVAAAKYLQSIKNKRYGGPSS |
| | | GAPPPS |
| P182 | 168 | C6-HSDAVFTDNYTRLRKQVAAKRYLQSIKNKRYGGPSS |
| | | GAPPPS |
| P183 | 169 | C6-HSDAVFTDNYTRLRKQVAAKQYLQSIKNKRYGGPSS |
| | | GAPPPS |
| P184 | 170 | C6-HSDAVFTDNYTRLRKQVAAKAYLQSIKNKRYGGPSS |
| | | GAPPPS |
| P185 | 171 | C6-HSDAVFTDNYTRLRKQVAAKLYLQSIKNKRYGGPSS |
| | | GAPPPS |
| P186 | 172 | C6-HSDAVFTDNYTRLRKQVAAKFYLQSIKNKRYGGPSS |
| | | GAPPPS |
| P187 | 173 | C6-HSDAVFTDNYTRLRKQVAAKKYLQAibIKNKRYGGP |
| | | SSGAPPPS |
| P188 | 174 | C6-HSDAVFTDNYTRLRKQVAAKKYLQSAibKNKRYGGP |
| | | SSGAPPPS |
| P192 | 177 | C6-HSDAVFTDQYTRLLAKLALQKYLQSIKQKRYGGPSS |
| | | GAPPPS |
| P193 | 178 | C6-HSDAVFTDNYTRLRK(Ac)QYAAK(Ac)KYLQSIKN |
| | | KRYGGPSSGAPPPS |
| P194 | 179 | C6-HSDAVFTDNYTRLRK(Ac)QVAAKK(Ac)YLQSIKN |
| | | KRYGGPSSGAPPPS |
| P195 | 180 | C6-HSDAVFTDNYTRLLAQLALQKYLQSIKNKRYGGPSS |
| | | GAPPPS |
| P196 | 181 | C6-HSDAVFTDNYTRLLAKVALQKYLQSIKNKRYGGPSS |
| | | GAPPPS |
| P197 | 182 | C6-HSDAVFTDNYTRLLAKLAAQKYLQSIKNKRYGGPSS |
| | | GAPPPS |
| P207 | 188 | C6-HSDdAVFTDNYTRLRKQVAAKKYLQSIKNKRYGGPS |
| | | SGAPPPS |
| P209 | 190 | C6-HSDAibVFTDNYTRLRKQVAAKKYLQSIKNKRYGGP |
| | | SSGAPPPS |
| P210 | 191 | C6-HSDAdVFTDNYTRLRKQVAAKKYLQSIKNKRYGGPS |
| | | SGAPPPS |
| P212 | 193 | C6-HSDAAibFTDNYTRLRKQVAAKKYLQSIKNKRYGGP |
| | | SSGAPPPS |
| P213 | 194 | C6-HSDAVFTDNYTRLRKQVAAKRYLQSIRNGGPSSGAP |
| | | PPS |
| P214 | 195 | C6-HSDAVFTDNYTRLRKQVAARRYLQSIRNGGPSSGAP |
| | | PPS |
| P215 | 196 | C6-HSDAVFTDNYTRLRRQVAAKRYLQSIRNGGPSSGAP |
| | | PPS |
| P216 | 197 | C6-HSDAVFTDNYTRLRRQVAARKYLQSIRNGGPSSGAP |
| | | PPS |
| P240 | 214 | C6-HSDAVFTDNYTRLAibKQLAAAibKYLQSIKNKRYG |
| | | GPSSGAPPPS |
| P241 | 215 | C6-HSDAVFTDNYTRLAibKQLAAKAibYLQSIKNKRYG |
| | | GPSSGAPPPS |
| P242 | 216 | C6-HSDAVFTDNYTRLAibKQVAAAibKYLQSIKNKRYG |
| | | GPSSGAPPPS |
| P243 | 217 | C6-HSDAVFTDNYTRLAibKQVAAQKYLQSIKNKRYGGP |
| | | SSGAPPPS |
| P244 | 218 | C6-HSDAVFTDNYTRLAibKQVAAKAibYLQSIKNKRYG |
| | | GPSSGAPPPS |
| P249 | 219 | C6-HSDAVFTDNYTRLAibKQVAAKQYLQSIKNKRYGGP |
| | | SSGAPPPS |
| P250 | 220 | C6-HSDAVFTDNYTRLQKQVAAAibKYLQSIKNKRYGGP |
| | | SSGAPPPS |
| P251 | 221 | C6-HSDAVFTDNYTRLQKQVAAKAibYLQSIKNKRYGGP |
| | | SSGAPPPS |
| P258 | 224 | C6-HSDAVFTDNYTRLQAibQVAAKKYLQSIKNKRYGGP |
| | | SSGAPPPS |
| P259 | 225 | C6-HSDAVFTDNYTRLAibKQVAALKYLQSIKNKRYGGP |
| | | SSGAPPPS |
| P260 | 226 | C6-HSDAVFTDNYTRLAibKQVAAAKYLQSIKNKRYGGP |
| | | SSGAPPPS |
| P261 | 227 | C6-HSDAVFTDNYTRLRAibQVAAAibKYLQSIKNKRYG |
| | | GPSSGAPPPS |
| P262 | 228 | C6-HSDAVFTDNYTRLRAibQVAAVKYLQSIKNKRYGGP |
| | | SSGAPPPS |
| P263 | 229 | C6-HSDAVFTDNYTRLRAibQVAAAKYLQSIKNKRYGGP |
| | | SSGAPPPS |
| P264 | 230 | C6-HSDAVFTDNYTRLRAibQVAAKAibYLQSIKNKRYG |
| | | GPSSGAPPPS |
| P265 | 231 | C6-HSDAVFTDNYTRLRAibQVAALKYLQSIKNKRYGGP |
| | | SSGAPPPS |
| P269 | 232 | C6-HSDAVFTDNYTRLAibKQVAAVKYLQSIKNKRYGGP |
| | | SSGAPPPS |
| P284 | 237 | C6-HSDAVFTDNYTRLRAibQLAAKAibYLQSIKNKRYG |
| | | GPSSGAPPPS |
| P291 | 241 | C6-HSDAVFTDNYTRLRAibQLAAAibKYLQSIKNKRYG |
| | | GPSSGAPPPS |
| P292 | 242 | C6-HSDAVFTDNYTRLRAibQLAAAibKYLQSIKNKGGP |
| | | SSGAPPPS |
| P293 | 243 | C6-HSDAVFTDNYTRLAibKQVAAAibKYLQSIKQKGGP |
| | | SSGAPPPS |
| P294 | 244 | C6-HSDAVFTDNYTRLRAibQVAAAibKYLQSILKQKGG |
| | | PSSGAPPPS |
| P295 | 245 | C6-HSDAVFTDNYTRLRAibQVAAKAibYLQSIKQKGGP |
| | | SSGAPPPS |
| P296 | 246 | C6-HSDAVFTDNYTRLAibKQVAAAibKYLQSIKQGGPS |
| | | SGAPPPS |
| P297 | 247 | C6-HSDAVFTDNYTRLRAibQVAAAibKYLQSIKQGGPS |
| | | SGAPPPS |
| P298 | 248 | C6-HSDAVFTDNYTRLRAibQVAAKAibYLQSIKQGGPS |
| | | SGAPPPS |
| P301 | 250 | C6-HSDAVFTDNYTRLAAibQVAAAibKYLQSIKNKRYG |
| | | GPSSGAPPPS |
| P302 | 251 | C6-HSDAVFTDNYTRLQAibQVAAAibKYLQSIKNKRYG |
| | | GPSSGAPPPS |
| P305 | 252 | C6-HSDAVFTDNYTRLhRKQVAAKKYLQSIKNKRYGGPS |
| | | SGAPPPS |
| P307 | 253 | C6-HSDAVFTDNYTRLROrnQVAAKKYLQSIKNKRYGGP |
| | | SSGAPPPS |
| P314 | 255 | C6-HSEAVFTENYTRLRAibQVAAAibKYLQSIKNKRYG |
| | | GPSSGAPPPS |
| P317 | 258 | C6-HSDAVFTDNYTRLLAibQVAAAibKYLQSIKNKRYG |
| | | GPSSGAPPPS |
| P318 | 259 | C6-HSDAVFTDNYTRLKAibQVAAAibKYLQSIKNKRYG |
| | | GPSSGAPPPS |
| P319 | 260 | C6-HSDAVFTDNYTRLOrnAibQVAAAibKYLQSIKNKR |
| | | YGGPSSGAPPPS |
| P321 | 262 | C6-HSDAVFTDNYTRLRAibKVAAAibKYLQSIKNKRYG |
| | | GPSSGAPPPS |
| P322 | 263 | C6-HSDAVFTDNYTRLRAibQIAAAibKYLQSIKNKRYG |
| | | GPSSGAPPPS |
| P323 | 264 | C6-HSDAVFTDNYTRLRAibQKAAAibKYLQSIKNKRYG |
| | | GPSSGAPPPS |
| P324 | 265 | C6-HSDAVFTDNYTRLRAibQAAAAibKYLQSIKNKRYG |
| | | GPSSGAPPPS |
| P325 | 266 | C6-HSDAVFTDNYTRLRAibQNleAAAibKYLQSIKNKR |
| | | YGGPSSGAPPPS |
| P326 | 267 | C6-HSDAVFTDNYTOrnLRAibQVAAAibKYLQSIOrnN |
| | | OrnGGPSSGAPPPS |
| P329 | 269 | C6-HSDAVFTDNYThRLRAibQVAAAibKYLQSIhRNhR |
| | | GGPSSGAPPPS |
| P343 | 279 | C6-HSDAVFTDNYThRLRAibQVAAAibKYLQSIKNKRY |
| | | GGPSSGAPPPS |
| P344 | 280 | C6-HSDAVFTDNYTOrnLRAibQVAAAibKYLQSIKNKR |
| | | YGGPSSGAPPPS |
| P346 | 282 | C6-HSDAVFTDNYTRAibRAibQVAAAibKYLQSIKNKR |
| | | YGGPSSGAPPPS |
| P349 | 283 | C6-HSDAVFTDNYTRLRAibQVAAAibKYLQSIKAibKG |
| | | GPSSGAPPPS |
| P350 | 284 | C6-HSDAVFTDNYTRLRAibQVAAAibKYLQSIKPKGGP |
| | | SSGAPPPS |
| P351 | 285 | C6-HSDAVFTDNYTRLRAibQVAAAibKYLQSIKKGGPS |
| | | SGAPPPS |
| P353 | 287 | C6-HSDAVFTDNYTRLRAibQVAAAibKYLQSIdKdKGG |
| | | PSSGAPPPS |
| P354 | 288 | C6-HSDAVFTDNYTRLRAibQVAAAibKYLQSIKhRGGP |
| | | SSGAPPPS |
| P355 | 289 | C6-HSDAVFTDNYTRLRAibQVAAAibKYLQSIKAibGG |
| | | PSSGAPPPS |
| P357 | 291 | C6-HSDAVFTDNYTRLRAibQVAAAibKYLQSIKAibOr |
| | | nGGPSSGAPPPS |
| P358 | 292 | C6-HSDAVFTDNY(OMe)TRLRAibQVAAAibKYLQSIK |
| | | NKRYGGPSSGAPPPS |
| P362 | 293 | C6-HSEAVFTENYTOrnLRAibQVAAAibKYLQSIOrnN |
| | | OrnGGPSSGAPPPS |
| P363 | 294 | C6-HSDAVFTDQYTOrnLRAibQVAAAibKYLQSIOrnQ |
| | | OrnGGPSSGAPPPS |
| P364 | 295 | C6-HSDAVFTDNYTOrnLRAibQLAAAibKYLQSIOrnO |
| | | rnGGPSSGAPPPS |
| P365 | 296 | C6-HSDAVFTDNYTOrnLRAibQIAAAibKYLQSIOrnN |
| | | OrnGGPSSGAPPPS |
| P372 | 303 | C6-HSDAVFTDNYTOrnLRAibQVAAAibKYLQCIOrnN |
| | | OrnGGPSSGAPPPS |
| P379 | 305 | C6-HSEAVFTEQYTOrnLRAibQVAAAibOrnYLQSIOr |
| | | nOrnGGPSSGAPPPC-NH₂ |
| P393 | 317 | C6-HSEAVFTEQY(OMe)TOrnLRAibQLAAAibOrnYL |
| | | QSIOrnOrnGGPSSGAPPPS |
| P394 | 318 | C6-HSDAVFTDQY(OMe)TOrnLRAibQLAAAibOrnYL |
| | | QSIOrnOrnGGPSSGAPPPS |
| P395 | 319 | C6-HSDAVFTDQYTOrnLRAibQLAAAibOrnYLQSIOr |
| | | nOrnGGPSSGAPPPS |
| P396 | 320 | C6-HSDAVFTDQYTOrnLRAibQVAAAibOrnYLQSIOr |
| | | nOrnGGPSSGAPPPS |
| P397 | 321 | C6-HSDAVFTDNYTOrnLRAibQVAAAibOrnYLQSIOr |
| | | nOrnGGPSSGAPPPS |
| P398 | 322 | C6-HSDAVFTEQY(OMe)TOrnLRAibQVAAAibOrnYL |
| | | QSIOrnOrnGGPSSGAPPPC-NH₂ |

According to a second aspect of the present invention, the preferred PEGylated VPAC2 receptor peptide agonists comprise an amino acid sequence selected from:

| | SEQ | |
|---|---|---|
| Agonist | ID | |
| # | NO: | Sequence |
| P41 | 342 | HSDAVFTDNYTRLRKQVAAKKYLQSIKNKRYC |
| | | (PEG40K) |
| P137 | 343 | C6-HSDAVFTDNYTRLRKQVAAKKYLQSIKNKRYGGPSS |
| | | GAPPPC(PEG40K) |
| P190 | 344 | C6-HSDAVFTDNYRLLAKLALQKYLQSIKNKRYGGPSSG |
| | | APPPC(PEG40K) |
| P202 | 345 | C6-HSDAVFTDNYTRLLAKLALQKYLQSIKNKGGPSSGA |
| | | PPPC(PEG40K) |
| P217 | 346 | C6-HSDAVFTDNYTRLRK(PEG20K)QVAAK(PEG20K) |
| | | RYLQSIRNGGPSSGAPPPS |
| P219 | 347 | C6-HSDAVFTDNYTRLRRQVAAK(PEG20K)RYLQSIRN |
| | | GGPSSGAPPPS |
| P245 | 348 | C6-HSDAVFTDNYTRLRK(PEG40K)QVAARRYLQSIRN |
| | | GGPSSGAPPPS |
| P246 | 349 | C6-HSDAVFTDNYTRLRRQVAAK(PEG40K)RYLQSIRN |
| | | GGPSSGAPPPS |
| P247 | 350 | C6-HSDAVFTDNYTRLRRQVAARK(PEG40K)YLQSIRN |
| | | GGPSSGAPPPS |
| P283 | 351 | C6-HSDAVFTDNYTRLAibKQVAAAibKYLQSIKNKRYG |
| | | GPSSGAPPPC(PEG40K)-NH₂ |
| P286 | 352 | C6-HSDAVFTDNYTRLRAibQVAAKAibYLQSIKNKRYG |
| | | GPSSGAPPPC(PEG40K)-NH₂ |
| P300 | 353 | C6-HSDAVFTDNYTRLRAibQVAAAibKYLQSIKNKRYG |
| | | GPSSGAPPPC(PEG40K)-NH₂ |
| P328 | 354 | C6-HSDAVFTDNYTOrnLRAibQVAAAibKYLQSIOrnN |
| | | OrnGGPSSGAPPPC(PEG40K)-NH₂ |
| P331 | 355 | C6-HSDAVFTDNYThRLRAibQVAAAibKYLQSIhRNhR |
| | | GGPSSGAPPPC(PEG40K)-NH₂ |
| P337 | 356 | C6-HSDAVFTDNYTOrnLRAibQVAAAibKYLQSIhRhR |
| | | GGPSSGAPPPC(PEG40K)-NH₂ |
| P340 | 357 | C6-HSDAVFTDNYThRLRAibQVAAAibKYLQSIhRNhR |
| | | hRYGGPSSGAPPPC(PEG40K)-NH₂ |
| P373 | 358 | C6-HSDAVFTDNYTOrnLRAibQVAAAibKYLQC |
| | | (PEG40K)IOrnNOrnGGPSSGAPPPS |
| P380 | 359 | C6-HSEAVFTEQYTOrnLRAibQVAAAibOrnYLQSIOr |
| | | nOrnGGPSSGAPPPC(PEG40K)-NH₂ |
| P378 | 360 | C6-HSDAVFTDNYTOrnLRAibQVAAC(PEG40K)OrnY |
| | | LQSIOrnNOrnGGPSSGAPPPS-NH₂ |
| P272 | 361 | Biotin-Acp-HSDAVFTDNYTRLRKQVAAKKYLQSIKN |
| | | KRYGGPSSGAPPPC(PEG40K) |
| P399 | 362 | C6-HSDAVFTEQY(OMe)TOrnLRAibQVAAAibOrnYL |
| | | QSIOrnOrnGGPSSGAPPPC(PEG40K)-NH₂ |
| P404 | 363 | C6-HSDAVFTEQY(OMe)TOrnLRAibQVAAAibOrnYL |
| | | QSIOrnOrnGGPSSGAPPPC(PEG60K)-NH₂ |
| P411 | 364 | C6-HSDAVFTDNYTOrnLRAibQVAAC(PEG20K)OrnY |
| | | LQSIOrnNOrnGGPSSGAPPPS-NH₂ |
| P413 | 365 | C6-HSDAVFTDNYTOrnLRK(WPEG40K)QVAAAibKYL |
| | | QSIOrnNOrnGGPSSGAPPPS |
| P415 | 366 | C6-HSDAVFTEQY(OMe)TOrnLRAibQLAAAibOrnY |
| | | (OMe)LQSIOrnOrnGGPSSGAPPPC(PEG40K)-NH₂ |
| P420 | 367 | C6-HSDAVFTEQY(OMe)TOrnLRAibQVAAAibOrnYL |
| | | QSC(PEG40K)OrnOrnGGPSSGAPPPS-NH₂ |
| P426 | 368 | C6-HSDAVFTEQY(OMe)TOrnLRAibQVAAAibOrnYL |
| | | C(PEG40K)SIOrnOrnGGPSSGAPPPS-NH₂ |
| P428 | 369 | C6-HSDAVFTEQY(OMe)TOrnLRAibQVAAAibOrnYL |
| | | K(CO(CH₂)₂SPEG40K)SIOrnOrnGGPSSGAPPPS- |
| | | NH₂ |
| P430 | 370 | C6-HSDAVFTEQY(OMe)TOrnLRAibQVAc(PEG40K) |
| | | AibOrnYLQSIOrnOrnGGPSSGAPPPS-NH₂ |
| P432 | 371 | C6-HSDAVFTEQY(OMe)TOrnLRAibQVAK(CO |
| | | (CH₂)₂SPEG40K)AibOrnYLQSIOrnOrnGGPSSGAP |
| | | PPS-NH₂ |
| P434 | 372 | C6-HSDAVFTEQY(OMe)TOrnLRAibQC(PEG40K)AA |
| | | AibOrnYLQSIOrnOrnGGPSSGAPPPS-NH₂ |
| P438 | 373 | C6-HSDAVFTEQY(OMe)TOrnLRAibC(PEG40K)VAA |
| | | AibOrnYLQSIOrnOrnGGPSSGAPPPS-NH₂ |
| P443 | 374 | C6-HSDAVFTEQY(OMe)TOrnLRAibQVAAAibOrnYL |
| | | QSIOrnC(PEG40K)OrnGGPSSGAPPPS-NH₂ |
| P447 | 375 | C6-HSDAVFTEQY(OMe)TOrnLRAibQVAAAibOrnYL |
| | | QC(PEG20K)OrnOrnGGPSSGAPPPC(PEG20K)-NH₂ |
| P449 | 376 | C6-HSDAVFTEC(PEG40K)Y(OMe)TOrnLRAibQVAA |
| | | AibOrnYLQSIOrnOrnGGPSSGAPPPS-NH₂ |
| P452 | 377 | C6-HSDAVFTEQY(OMe)TOrnLRAibC(PEG20K)VAA |
| | | AibOrnYLQSIOrnOrnGGPSSGAPPPS-NH₂ |
| P456 | 378 | C6-HSDAVFTEQY(OMe)TOrnC(PEG40K)RAibQVAA |
| | | AibOrnYLQSIOrnOrnGGPSSGAPPPS-NH₂ |
| P461 | 379 | C6-HSDAVFTEQY(OMe)TOrnLRAibQVAAAibOrnYL |
| | | K(CO(CH₂)₂SPEG20K)SIOrnOrnGGPSSGAPPPS- |
| | | NH₂ |
| P462 | 380 | C6-HSDAVFTEQY(OMe)TOrnLRAibQVAK(CO |
| | | (CH₂)₂SPEG20K)AibOrnYLQSIOrnOrnGGPSSGAP |
| | | PPS-NH₂ |
| P422 | 728 | C6-HSDAVFTEQY(OMe)TOrnLRAibQVAAAibOrnYL |
| | | QSK(CO(CH₂)₂SPEG40K)OrnOrnGGPSSGAPPPS- |
| | | NH₂ |
| P424 | 729 | C6-HSDAVFTEQY(OMe)TOrnLRAibQVAAAibOrnYL |
| | | QK(CO(CH₂)₂SPEG40K)IOrnOrnGGPSSGAPPPS- |
| | | NH₂ |
| P436 | 730 | C6-HSDAVFTEQY(OMe)TOrnLRAibQK(CO(CH₂)₂ |
| | | SPEG40K)AAAibOrnYLQSIOrnOrnGGPSSGAPPPS- |
| | | NH₂ |
| P440 | 731 | C6-HSDAVFTEQY(OMe)TOrnLRAibK(CO(CH₂)₂ |
| | | SPEG40K)VAAAibOrnYLQSIOrnOrnGGPSSGAPPP |
| | | S-NH₂ |
| P445 | 732 | C6-HSDAVFTEQY(OMe)TOrnLRAibQVAAAibOrnYL |
| | | QSIOrnK(CO(CH₂)₂SPEG40K)OrnGGPSSGAPPPS- |
| | | NH₂ |
| P458 | 733 | C6-HSDAVFTEQY(OMe)TOrnK(CO(CH₂)₂ |
| | | SPEG40K)RAibQVAAAibOrnYLQSIOrnOrnGGPSSG |
| | | APPPS-NH₂ |
| P464 | 734 | C6-HSDAVFTEQY(OMe)TOrnLRAibQVAAAibOrnYL |
| | | QSK(CO(CH₂)₂SPEG20K)OrnOrnGGPSSGAPPPS- |
| | | NH₂ |
| P465 | 735 | C6-HSDAVFTEQY(OMe)TOrnLRAibQVAAAibOrnYL |
| | | QK(CO(CH₂)₂SPEG20K)IOrnOrnGGPSSGAPPPS- |
| | | NH₂ |
| P466 | 736 | C6-HSDAVFTEQY(OMe)TOrnLRAibQK(CO(CH₂)₂ |
| | | SPEG20K)AAAibOrnYLQSIOrnOrnGGPSSGAPPPS- |
| | | NH₂ |
| P467 | 737 | C6-HSDAVFTEQY(OMe)TOrnLRAibK(CO(CH₂)₂ |
| | | SPEG20K)VAAAibOrnYLQSIOrnOrnGGPSSGAPPP |
| | | S-NH₂ |
| P468 | 738 | C6-HSDAVFTEQY(OMe)TOrnLRAibQVAAAibOrnYL |
| | | QSIOrnK(CO(CH₂)₂SPEG20K)OrnGGPSSGAPPPS- |
| | | NH₂ |

More preferred PEGylated VPAC2 receptor peptide agonists according to the second aspect of the present invention comprise an amino acid sequence selected from:

| | SEQ | |
|---|---|---|
| Agonist | ID | |
| # | NO: | Sequence |
| P137 | 343 | C6-HSDAVFTDNYTRLRKQVAAKKYLQSIKNKRYGGPSS |
| | | GAPPPC(PEG40K) |
| P190 | 344 | C6-HSDAVFTDNYTRLLAKLALQKYLQSIKNKRYGGPSS |
| | | GAPPPC(PEG40K) |
| P217 | 346 | C6-HSDAVFTDNYTRLRK(PEG20K)QVAAK(PEG20K) |
| | | RYLQSIRNGGPSSGAPPPS |
| P219 | 347 | C6-HSDAVFTDNYTRLRRQVAAK(PEG20K)RYLQSIRN |
| | | GGPSSGAPPPS |
| P245 | 348 | C6-HSDAVFTDNYTRLRK(PEG40K)QVAARRYLQSIRN |
| | | GGPSSGAPPPS |
| P246 | 349 | C6-HSDAVFTDNYTRLRRQVAAK(PEG40K)RYLQSIRN |
| | | GGPSSGAPPPS |
| P247 | 350 | C6-HSDAVFTDNYTRLRRQVAARK(PEG40K)YLQSIRN |
| | | GGPSSGAPPPS |
| P283 | 351 | C6-HSDAVFTDNYTRLAibKQVAAAibKYLQSIKNKRYG |
| | | GPSSGAPPPC(PEG40K)-NH₂ |
| P286 | 352 | C6-HSDAVFTDNYTRLRAibQVAAKAibYLQSIKNKRYG |
| | | GPSSGAPPPC(PEG40K)-NH₂ |
| P300 | 353 | C6-HSDAVFTDNYTRLRAibQVAAAibKYLQSIKNKRYG |
| | | GPSSGAPPPC(PEG40K)-NH₂ |
| P328 | 354 | C6-HSDAVFTDNYTOrnLRAibQVAAAIbKYLQSIOrnN |
| | | OrnGGPSSGAPPPC(PEG40K)-NH₂ |
| P331 | 355 | C6-HSDAVFTDNYThRLRAibQVAAAibKYLQSIhRNhR |
| | | GGPSSGAPPPC(PEG40K)-NH₂ |
| P337 | 356 | C6-HSDAVFTDNYTOrnLRAibQVAAAibKYLQSIhRhR |
| | | GGPSSGAPPPC(PEG40K)-NH₂ |
| P340 | 357 | C6-HSDAVFTDNYThRLRAibQVAAAibKYLQSIhRNhR |
| | | hRYGGPSSGAPPPC(PEG40K)-NH₂ |
| P373 | 358 | C6-HSDAVFTDNYTOrnLRAibQVAAAibKYLQC |
| | | (PEG40K)IOrnNOrnGGPSSGAPPPS |
| P380 | 359 | C6-HSEAVETEQYTOrnLRAibQVAAAibOrnYLQSIOr |
| | | nOrnGGPSSGAPPPC(PEG40K)-NH₂ |
| P378 | 360 | C6-HSDAVFTDNYTOrnLRAibQVAAC(PEG40K)OrnY |
| | | LQSIOrnNOrnGGPSSGAPPPS-NH₂ |
| P272 | 361 | Biotin-Acp-HSDAVFTDNYTRLRKQVAAKKYLQSIKN |
| | | KRYGGPSSGAPPPC(PEG40K) |
| P399 | 362 | C6-HSDAVFTEQY(OMe)TOrnLRAibQVAAAibOrnYL |
| | | QSIOrnOrnGGPSSGAPPPC(PEG40K)-NH₂ |
| P404 | 363 | C6-HSDAVFTEQY(OMe)TOrnLRAibQVAAAibOrnYL |
| | | QSIOrnOrnGGPSSGAPPPC(PEG60K)-NH₂ |
| P432 | 371 | C6-HSDAVFTEQY(GMe)TOrnLRAibQVAK(CO |
| | | (CH₂)₂SPEG40K)AibOrnYLQSIOrnOrnGGPSSGAP |
| | | PPS-NH₂ |

Even more preferred PEGylated VPAC2 receptor peptide agonists according to the second aspect of the present invention comprise an amino acid sequence selected from:

| | | |
|---|---|---|
| | SEQ | |
| Agonist | ID | |
| # | NO: | Sequence |

| | | |
|---|---|---|
| P190 | 344 | C6-HSDAVFTDNYTRLLAKLALQKYLQSIKNKRYGGPSS |
| | | GAPPPC(PEG40K) |
| P286 | 352 | C6-HSDAVFTDNYTRLRAibQVAAKAibYLQSIKNKRYG |
| | | GPSSGAPPPC(PEG40K)-NH₂ |
| P340 | 357 | C6-HSDAVFTDNYThRLRAibQVAAAibKYLQSIhRNhR |
| | | hRYGGPSSGAPPPC(PEG40K)-NH₂ |
| P373 | 358 | C6-HSDAVFTDNYTOrnLRAibQVAAAibKYLQC(PEG |
| | | 40K)IOrnNOrnGGPSSGAPPPS |
| P404 | 363 | C6-HDAVFTEQY(OMe)TOrnLRAibQVAAAibOrnYLQ |
| | | SIOrnOrnGGPSSGAPPPC(PEG60K)-NH₂ |
| P432 | 371 | C6-HSDAVFTEQY(OMe)TOrnLRAibQVAK(CO |
| | | (CH₂)₂SPEG40K)AibOrnYLQSIOrnOrnGGPSSGAP |
| | | PPS-NH₂ |

According to a third aspect of the present invention, there is provided a PEGylated VPAC2 receptor peptide agonist comprising a sequence of the formula:

Formula 14

(SEQ ID NO: 26)

Xaa₁-Xaa₂-Xaa₃-Xaa₄-Xaa₅-Xaa₆-Thr-Xaa₈-Xaa₉-Xaa₁₀-
Thr-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Xaa₂₂-Xaa₂₃-Xaa₂₄-Xaa₂₅-Xaa₂₆-
Xaa₂₇-Xaa₂₈-Xaa₂₉-Xaa₃₀-Xaa₃₁-Xaa₃₂-Xaa₃₃-Xaa₃₄-
Xaa₃₅-Xaa₃₆-Xaa₃₇-Xaa₃₈-Xaa₃₉-Xaa₄₀

wherein:
Xaa₁ is: any naturally occurring amino acid, dH, or is absent;
Xaa₂ is: any naturally occurring amino acid, dA, dS, or Aib;
Xaa₃ is: Asp or Glu;
Xaa₄ is: any naturally occurring amino acid, dA, Aib, or NMeA;
Xaa₅ is: any naturally occurring amino acid, dV, or Aib;
Xaa₆ is: any naturally occurring amino acid;
Xaa₈ is: Asp, Glu, Ala, Lys, Leu, Arg, or Tyr;
Xaa₉ is: Asn, Gln, Asp, Glu, Ser, or Cys;
Xaa₁₀ is: any naturally occurring aromatic amino acid, or Tyr (OMe);
Xaa₁₂ is: hR, Orn, Lys (isopropyl), Aib, Cit, or any naturally occurring amino acid except Pro;
Xaa₁₃ is: Aib, K(CO(CH₂)₂SH), or any naturally occurring amino acid except Pro;
Xaa₁₄ is: hR, Orn, Lys (isopropyl), Aib, Cit, or any naturally occurring amino acid except Pro;
Xaa₁₅ is: hR, Orn, Lys (isopropyl), Aib, K (Ac), Cit, K(W), or any naturally occurring amino acid except Pro;
Xaa₁₆ is: hR, Orn, Lys (isopropyl), Cit, K(CO(CH₂)₂SH), or any naturally occurring amino acid except Pro;
Xaa₁₇ is: Nle, Aib, K(CO(CH₂)₂SH), or any naturally occurring amino acid except Pro;
Xaa₁₈ is: any naturally occurring amino acid;
Xaa₁₉ is: K(CO(CH₂)₂SH), or any naturally occurring amino acid except Pro;
Xaa₂₀ is: hR, Orn, Lys (isopropyl), Aib, K(Ac), Cit, or any naturally occurring amino acid except Pro;
Xaa₂₁ is: hR, Orn, Aib, K(Ac), Cit, or any naturally occurring amino acid except Pro;
Xaa₂₂ is: Aib, Tyr (OMe), or any naturally occurring amino acid except Pro;
Xaa₂₃ is: Aib or any naturally occurring amino acid except Pro;
Xaa₂₄ is: K(CO(CH₂)₂SH), or any naturally occurring amino acid except Pro;
Xaa₂₅ is: Aib, K(CO(CH₂)₂SH), or any naturally occurring amino acid except Pro;
Xaa₂₆ is: K(CO(CH₂)₂SH), or any naturally occurring amino acid except Pro;
Xaa₂₇ is: hR, Lys (isopropyl), Orn, dK, or any naturally occurring amino acid except Pro;
Xaa₂₈ is: any naturally occurring amino acid, Aib, hR, Cit, Orn, dK, or K(CO(CH₂)₂SH);
Xaa₂₉ is: any naturally occurring amino acid, hR, Orn, Cit, Aib, or is absent;
Xaa₃₀ is: any naturally occurring amino acid, hR, Orn, Cit, Aib, or is absent; and
Xaa₃₁ to Xaa₄₀ are any naturally occurring amino acid or are absent;

provided that if Xaa₂₉, Xaa₃₀, Xaa₃₁, Xaa₃₂, Xaa₃₃, Xaa₃₄, Xaa₃₅, Xaa₃₆, Xaa₃₇,
Xaa₃₈ or Xaa₃₉ is absent, the next amino acid present downstream is the next amino acid in the peptide agonist sequence and that the peptide agonist comprises at least one amino acid substitution selected from:
Xaa₂ is: dA, Val, Gly, Leu, dS, or Aib;
Xaa₄ is: Ile, Tyr, Phe, Val, Thr, Leu, Trp, dA, Aib, or NMeA;
Xaa₅ is: Leu, Phe, Thr, Trp, Tyr, dV, or Aib;
Xaa₈ is: Leu, Arg, or Tyr;
Xaa₉ is: Glu, Ser, or Cys;
Xaa₁₀ is: Trp;
Xaa₁₂ is: Ala, hR, Aib, Lys (isopropyl), Cit, Gln, or Phe;
Xaa₁₃ is: Phe, Glu, Ala, Aib, Ser, Cys, or K(CO(CH₂)₂SH);
Xaa₁₄ is: Leu, Lys, Ala, hR, Orn, Lys (isopropyl), Phe, Gln, Aib, or Cit;
Xaa₁₅ is: Ala, Arg, Leu, hR, Orn, Lys (isopropyl), Phe, Gln, Aib, K(Ac), Cit, or K(W);
Xaa₁₆ is: Lys, Lys (isopropyl), hR, Orn, Cit, Ser, Cys, or K(CO(CH₂)₂SH);
Xaa₁₇ is: Lys, Aib, Ser, Cys, or K(CO(CH₂)₂SH);
Xaa₁₈ is: Ser, or Cys;
Xaa₁₉ is: K(CO(CH₂)₂SH);
Xaa₂₀ is: Gln, hR, Arg, Ser, Orn, Lys(isopropyl), Ala, Aib, Trp, Thr, Leu, Ile, Phe, Tyr, Val, K(Ac), Cit, or Cys;
Xaa₂₁ is: Arg, Ala, Phe, Aib, Leu, Gln, Orn, hR, K(Ac), Cit, Ser, or Cys;
Xaa₂₂ is: Trp, Thr, Leu, Ile, Val, Tyr(OMe), Ala, Aib, Ser, or Cys;
Xaa₂₃ is: Phe, Ile, Ala, Trp, Thr, Val, Aib, Ser, or Cys;
Xaa₂₄ is: Ser, Cys, or K(CO(CH₂)₂SH);
Xaa₂₅ is: Phe, Ile, Leu, Val, Trp, Gln, Asn, Tyr, Aib, Glu, Cys, or K(CO(CH₂)₂SH);
Xaa₂₆ is: Thr, Trp, Tyr, Phe, Ser, Cys, or K(CO(CH₂)₂SH);
Xaa₂₇ is: hR, Orn, or dK;
Xaa₂₈ is: Pro, Arg, Aib, Orn, hR, Cit, dK, Cys, or K(CO(CH₂)₂SH);
Xaa₂₉ is: hR, Cys, Orn, Cit, or Aib;
Xaa₃₀ is: hR, Cit, Aib, or Orn; and
Xaa₃₁ is: Mis, or Phe,
   and wherein:
   at least one of the Cys residues in the peptide agonist is covalently attached to a PEG molecule, or
   at least one of the Lys residues in the peptide agonist is covalently attached to a PEG molecule, or
   at least one of the K(CO(CH₂)₂SH) in the peptide agonist is covalently attached to a PEG molecule, or
   the K(W) in the peptide agonist is covalently attached to a PEG molecule, or
   the carboxy-terminal amino acid of the peptide agonist is covalently attached to a PEG molecule, or
   any combination thereof.

Preferably, the PEGylated VPAC2 receptor peptide agonist according to the third aspect of the present invention comprises a sequence of the formula:

Formula 15

(SEQ ID NO: 27)

Thr-Xaa₂-Xaa₃-Xaa₄-Xaa₅-Phe-Thr-Xaa₈-Xaa₉-Xaa₁₀-
Thr-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Xaa₂₂-Xaa₂₃-Xaa₂₄-Xaa₂₅-Xaa₂₆-
Xaa₂₇-Xaa₂₈-Xaa₂₉-Xaa₃₀-Xaa₃₁-Xaa₃₂-Xaa₃₃-Xaa₃₄-
Xaa₃₅-Xaa₃₆-Xaa₃₇-Xaa₃₈-Xaa₃₉-Xaa₄₀

wherein:
Xaa₂ is: dA, Ser, Val, Gly, Thr, Leu, dS, Pro, or Aib;
Xaa₃ is: Asp or Glu;
Xaa₄ is: Ala, Ile, Tyr, Phe, Val, Thr, Leu, Trp, Gly, dA, Aib, or NMeA;
Xaa₅ is: Val, Leu, Phe, Ile, Thr, Trp, Tyr, dV, or Aib;
Xaa₈ is: Asp, Glu, Ala, Lys, Leu, Arg, or Tyr;
Xaa₉ is: Asn, Gln, Asp, Glu, Ser, or Cys;
Xaa₁₀ is: Tyr, Trp, or Tyr(OMe);
Xaa₁₂ is: Arg, Lys, Glu, hR, Orn, Lys (isopropyl), Aib, Cit, Ala, Leu, Gln, or Phe;
Xaa₁₃ is: Leu, Phe, Glu, Ala, Aib, Ser, Cys, or K(CO(CH₂)₂SH);
Xaa₁₄ is: Arg, Leu, Lys, Ala, hR, Orn, Lys (isopropyl), Phe, Gln, Aib, or Cit;
Xaa₁₅ is: Lys, Ala, Arg, Glu, Leu, hR, Orn, Lys (isopropyl), Phe, Gln, Aib, K(Ac), Cit, or K(W);
Xaa₁₆ is: Gln, Lys, Glu, Ala, hR, Orn, Lys (isopropyl), Cit, Ser, Cys, or K(CO(CH₂)₂SH);
Xaa₁₇ is: Val, Ala, Leu, Ile, Met, Nle, Lys, Aib, Ser, Cys, or K(CO(CH₂)₂SH);
Xaa₁₈ is: Ala, Ser, or Cys;
Xaa₁₉ is: Val, Ala, Glu, Phe, Gly, His, Ile, Lys, Leu, Met, Asn, Gln, Arg, Ser, Thr, Trp, Tyr, Cys, Asp, or K(CO(CH₂)₂SH);
Xaa₂₀ is: Lys, Gln, hR, Arg, Ser, His, Orn, Lys (isopropyl), Ala, Aib, Trp, Thr, Leu, Ile, Phe, Tyr, Val, K(Ac), Cit, or Cys;
Xaa₂₁ is: Lys, His, Arg, Ala, Phe, Aib, Leu, Gln, Orn, hR, K(Ac), Cit, Ser, or Cys;
Xaa₂₂ is: Tyr, Trp, Phe, Thr, Leu, Ile, Val, Tyr(OMe), Ala, Aib, Ser, or Cys;
Xaa₂₃ is: Leu, Phe, Ile, Ala, Trp, Thr, Val, Aib, Ser, or Cys;
Xaa₂₄ is: Gln, Glu, Asn, Ser, Cys, or K(CO(CH₂)₂SH);
Xaa₂₅ is: Ser, Asp, Phe, Ile, Leu, Thr, Val, Trp, Gln, Asn, Tyr, Aib, Glu, Cys, or K(CO(CH₂)₂SH);
Xaa₂₆ is: Ile, Leu, Thr, Val, Trp, Tyr, Phe, Ser, Cys, or K(CO(CH₂)₂SH);
Xaa₂₇ is: Lys, hR, Arg, Gln, Ala, Asp, Glu, Phe, Gly, His, Ile, Met, Asn, Ser, Thr, Val, Trp, Tyr, Lys (isopropyl), Cys, Len, Orn, or dK;
Xaa₂₈ is: Asn, Asp, Gln, Lys, Arg, Aib, Orn, hR, Cit, Pro, dK, Cys, or K(CO(CH₂)₂SH);
Xaa₂₉ is: Lys, Ser, Arg, Asn, hR, Ala, Asp, Glu, Phe, Gly, His, Ile, Leu, Met, Pro, Gln, Thr, Val, Trp, Tyr, Cys, Orn, Cit, Aib or is absent;
Xaa₃₀ is: Arg, Lys, Ile, Ala, Asp, Glu, Phe, Gly, His, Leu, Met, Asn, Pro, Gln, Ser, Thr, Val, Trp, Tyr, Cys, hR, Cit, Aib, Orn, or is absent;
Xaa₃₁ is: Tyr, His, Phe, Thr, Cys, or is absent;
Xaa₃₂ is: Ser, Cys, or is absent;
Xaa₃₃ is: Trp or is absent;
Xaa₃₄ is: Cys or is absent;
Xaa₃₅ is: Glu or is absent;
Xaa₃₆ is: Pro or is absent;
Xaa₃₇ is: Gly or is absent;
Xaa₃₈ is: Trp or is absent;
Xaa₃₉ is: Cys or is absent; and
Xaa₄₀ is: Arg or is absent

provided that if Xaa₂₉, Xaa₃₀, Xaa₃₁, Xaa₃₂, Xaa₃₃, Xaa₃₄, Xaa₃₅, Xaa₃₆, Xaa₃₇,
Xaa₃₈, or Xaa₃₉ is absent, the next amino acid present downstream is the next amino acid in the peptide agonist sequence,

and that the peptide agonist comprises at least one amino acid substitution selected from:
Xaa₂ is: dA, Val, Gly, Leu, dS, or Aib;
Xaa₄ is: Ile, Tyr, Phe, Val, Thr, Leu, Trp, dA, Aib, or NMeA;
Xaa₅ is: Leu, Phe, Thr, Trp, Tyr, dV, or Aib;
Xaa₈ is: Leu, Arg, or Tyr;
Xaa₉ is: Glu, Ser, or Cys;
Xaa₁₀ is: Trp;
Xaa₁₂ is: Ala, hR, Aib, Lys (isopropyl), Cit, Gln, or Phe;
Xaa₁₃ is: Phe, Glu, Ala, Aib, Ser, Cys, or K(CO(CH₂)₂SH);
Xaa₁₄ is: Leu, Lys, Ala, hR, Orn, Lys (isopropyl), Phe, Gln, Aib, or Cit;
Xaa₁₅ is: Ala, Arg, Leu, hR, Orn, Lys (isopropyl), Phe, Gln, Aib, K(Ac), Cit, or K(W);
Xaa₁₆ is: Lys, Lys (isopropyl), hR, Orn, Cit, Ser, Cys, or K(CO(CH₂)₂SH);
Xaa₁₇ is: Lys, Aib, Ser, Cys, or K(CO(CH₂)₂SH);
Xaa₁₈ is: Ser, or Cys;
Xaa₁₉ is: K(CO(CH₂)₂SH);
Xaa₂₀ is: Gln, hR, Arg, Ser, Orn, Lys(isopropyl), Ala, Aib, Trp, Thr, Leu, Ile, Phe, Tyr, Val, K(Ac), Cit, or Cys;
Xaa₂₁ is: Arg, Ala, Phe, Aib, Leu, Gln, Orn, hR, K (Ac), Cit, Ser, or Cys;
Xaa₂₂ is: Trp, Thr, Leu, Ile, Val, Tyr (OMe), Ala, Aib, Ser, or Cys;
Xaa₂₃ is: Phe, Ile, Ala, Trp, Thr, Val, Aib, Ser, or Cys;
Xaa₂₄ is: Ser, Cys, or K(CO(CH₂)₂SH);
Xaa₂₅ is: Phe, Ile, Leu, Val, Trp, Gln, Asn, Tyr, Aib, Glu, Cys, or K(CO(CH₂)₂SH);
Xaa₂₆ is: Thr, Trp, Tyr, Phe, Ser, Cys, or K(CO(CH₂)₂SH);
Xaa₂₇ is: hR, Orn, or dK;
Xaa₂₈ is: Pro, Arg, Aib, Orn, hR, Cit, dK, Cys, or K(CO(CH₂)₂SH);
Xaa₂₉ is: hR, Cys, Orn, Cit, or Aib;
Xaa₃₀ is: hR, Cit, Aib, or Orn; and
Xaa₃₁ is: His, or Phe,
   and wherein:
   at least one of the Cys residues in the peptide agonist is covalently attached to a PEG molecule, or at least one of the Lys residues in the peptide agonist is covalently attached to a PEG molecule, or
   at least one of the K(CO(CH₂)₂SH) in the peptide agonist is covalently attached to a PEG molecule, or
   the K(W) in the peptide agonist is covalently attached to a PEG molecule, or
   the carboxy-terminal amino acid of the peptide agonist is covalently attached to the PEG molecule, or any combination thereof.

According to a fourth aspect of the present invention, there is provided a PEGylated VPAC2 receptor peptide agonist of the present invention for use as a medicament.

According to a further aspect of the present invention, there is provided the use of a PEGylated VPAC2 receptor peptide agonist of the present invention for the manufacture of a medicament for the treatment non-insulin-dependent diabetes.

According to yet a further aspect of the present invention, there is provided the use of a PEGylated VPAC2 receptor peptide agonist of the present invention for the manufacture of a medicament for the treatment of insulin-dependent diabetes.

Alternative embodiments of the present invention are described below.

A first alternative embodiment of the present invention is a PEGylated VPAC2 receptor peptide agonist comprising a sequence of the formula:

Formula 4

(SEQ ID NO: 7)

Xaa₁-Xaa₂-Xaa₃-Xaa₄-Xaa₅-Xaa₆-Thr-Xaa₈-Xaa₉-Xaa₁₀-
Thr-Xaa₁₂-Xaa₁₃-Xaa₁₄-Xaa₁₅-Xaa₁₆-Xaa₁₇-Ala-Xaa₁₉-
Xaa₂₀-Xaa₂₁-Xaa₂₂-Leu-Xaa₂₄-Xaa₂₅-Xaa₂₆-Xaa₂₇-
Xaa₂₈-Xaa₂₉-Xaa₃₀-Xaa₃₁-Xaa₃₂-Xaa₃₃-Xaa₃₄-Xaa₃₅-
Xaa₃₆-Xaa₃₇-Xaa₃₈-Xaa₃₉-Xaa₄₀

wherein:
Xaa₁ is: His or is absent;
Xaa₂ is: dA, Ser, Val, Gly, Thr, Leu, dS, or Pro;
Xaa₃ is: Asp, or Glu;
Xaa₄ is: Ala, Ile, Tyr, Phe, Val, Thr, Leu, Trp, or Gly;
Xaa₅ is: Val, Leu, Phe, Ile, Thr, Trp, or Tyr;
Xaa₆ is: Phe, Ile, Leu, Thr, Val, Trp, or Tyr;
Xaa₈ is: Asp or Glu;
Xaa₉ is: Asn, Gln, or Asp;
Xaa₁₀ is: Tyr or Trp;
Xaa₁₂ is: Arg, Lys, Glu, hR, Orn, or Lys (isopropyl);
Xaa₁₃ is: Leu, Phe, Glu, or Ala;
Xaa₁₄ is: Arg, Leu, Lys, Ala, hR, Orn, or Lys (isopropyl);
Xaa₁₅ is: Lys, Ala, Arg, Glu, Leu, hR, Orn, or Lys (isopropyl);
Xaa₁₆ is: Gln, Lys, Glu, Ala, hR, Orn, or Lys (isopropyl);
Xaa₁₇ is: Val, Ala, Leu, Ile, or Met;
Xaa₁₈ is: Val, Ala, Glu, Phe, Gly, Mis, Ile, Lys, Leu, Met, Asn, Pro, Gln, Arg, Ser, Thr, Trp, Tyr, Cys, or Asp;
Xaa₂₀ is: Lys, Gln, hR, Arg, Ser, His, Orn, or Lys (isopropyl);
Xaa₂₁ is: Lys, His, or Arg;
Xaa₂₂ is: Tyr, Trp, Phe, Thr, Leu, Ile, or Val;
Xaa₂₄ is: Gln, Glu, or Asn;
Xaa₂₅ is: Ser, Asp, Phe, Ile, Leu, Thr, Val, Trp, Gln, Asn, or Tyr;
Xaa₂₆ is: Ile, Leu, Thr, Val, Trp, Tyr, or Phe;
Xaa₂₇ is: Lys, hR, Arg, Gln, Ala, Asp, Glu, Phe, Gly, His, Ile, Met, Asn, Pro, Ser, Thr, Val, Trp, Tyr, Lys (isopropyl), Cys, or Leu;
Xaa₂₈ is: Asn, Asp, Gln, Lys, or Arg;
Xaa₂₉ is: Lys, Ser, Arg, Asn, hR, Gly, Ala, Asp, Glu, Phe, His, Ile, Leu, Met, Pro, Gln, Thr, Val, Trp, Tyr, Cys, or is absent;
Xaa₃₀ is: Arg, Lys, Ile, Gly, Ala, Asp, Glu, Phe, His, Leu, Met, Asn, Pro, Gln, Ser, Thr, Val, Trp, Tyr, Cys, or is absent;
Xaa₃₁ is: Tyr, His, Phe, Thr, Cys, or is absent;
Xaa₃₂ is: Ser, Cys, or is absent;
Xaa₃₃ is: Trp or is absent;
Xaa₃₄ is: Cys or is absent;
Xaa₃₅ is: Glu or is absent;
Xaa₃₆ is: Pro or is absent;
Xaa₃₇ is: Gly or is absent;
Xaa₃₈ is: Trp or is absent;
Xaa₃₉ is: Cys or is absent; and
Xaa₄₀ is: Arg or is absent

provided that if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈, or Xaa₃₉ is absent, the next amino acid present downstream is the next amino acid in the sequence;

and a C-terminal extension wherein the N-terminus of the C-terminal extension is linked to the C-terminus of the sequence and wherein the C-terminal extension comprises an amino acid sequence selected from the group consisting of:

Formula 7

(SEQ ID NO: 15)

| | |
|---|---|
| a) | Xaa₁-Xaa₂-Xaa₃-Xaa₄-Xaa₅-Xaa₆-Xaa₇-Xaa₈-Xaa₉- |
| | Xaa₁₀-Xaa₁₁ |

wherein:
Xaa₁ is: Gly, Cys, or absent;
Xaa₂ is: Gly, Arg, or absent;
Xaa₃ is: Pro, Thr, or absent;
Xaa₄ is: Ser or absent;
Xaa₅ is: Ser or absent;
Xaa₆ is: Gly or absent;
Xaa₇ is: Ala or absent;
Xaa₈ is: Pro, or absent;
Xaa₉ is: Pro, or absent;
Xaa₁₀ is: Pro or absent; and
Xaa₁₁ is: Ser, Cys, or absent;

provided that if Xaa₁, Xaa₂, Xaa₃, Xaa₄, Xaa₅, Xaa₆, Xaa₇, Xaa₈, Xaa₉, or Xaa₁₀ is absent, the next amino acid present downstream is the next amino acid in the C-terminal extension and wherein the C-terminal amino acid may be amidated;

Formula 5

(SEQ ID NO: 8)

| | |
|---|---|
| b) | Xaa₁-Xaa₂-Xaa₃-Xaa₄-Xaa₅-Xaa₆-Xaa₇-Xaa₈-Xaa₉- |
| | Xaa₁₀-Xaa₁₁-Xaa₁₂-Xaa₁₃ |

wherein:
Xaa₁ is: Gly or absent;
Xaa₂ is: Gly or absent;
Xaa₃ is: Pro, Ser, Ala, or absent;
Xaa₄ is: Ser, Pro, His, or absent;
Xaa₅ is: Ser, Arg, Thr, Trp, Lys, or absent;
Xaa₆ is: Gly, Ser, or absent;
Xaa₇ is: Ala, Asp, Arg, Glu, Lys, Gly, or absent;
Xaa₈ is: Pro, Ser, Ala, or absent;
Xaa₉ is: Pro, Ser, Ala, or absent;
Xaa₁₀ is: Pro, Ser, Ala, Arg, Lys, His, or absent;
Xaa₁₁ is: Ser, His, Pro, Lys, Arg, or absent;
Xaa₁₂ is: His, Ser, Arg, Lys, or absent; and
Xaa₁₃ is: His, Ser, Arg, Lys, or absent;

provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ is absent, the next amino acid present downstream is the next amino acid in the C-terminal extension and wherein the C-terminal amino acid may be amidated; and

Formula 6

(SEQ ID NO: 9)

| | |
|---|---|
| c) | Xaa₁-Xaa₂-Xaa₃-Xaa₄-Xaa₅-Xaa₆-Xaa₇-Xaa₈-Xaa₉- |
| | Xaa₁₀-Xaa₁₁ |

wherein:
Xaa₁ is: Gly or absent;
Xaa₂ is: Gly or absent;
Xaa₃ is: Pro, Ser, Ala, or absent;
Xaa₄ is: Ser or absent;
Xaa₅ is: Ser or absent;
Xaa₆ is: Gly or absent;
Xaa₇ is: Ala or absent;
Xaa₈ is: Pro, Ser, Ala, or absent;
Xaa₉ is: Pro, Ser, Ala, or absent;
Xaa₁₀ is: Pro, Ser, Ala, or absent; and
Xaa₁₁ is: Ser or absent;

provided that if Xaa₁, Xaa₂, Xaa₃, Xaa₄, Xaa₅, Xaa₆, Xaa₇, Xaa₈, Xaa₉, or Xaa₁₀ is absent, the next amino acid present downstream is the next amino acid in the sequence and wherein the C-terminal amino acid may be amidated,

and wherein:

at least one of the Cys residues is covalently attached to a PEG molecule, or

at least one of the Lys residues is covalently attached to a PEG molecule, or

the carboxy-terminal amino acid is covalently attached to a PEG molecule, or any combination thereof.

Preferably, an alternative selective VPAC2 receptor peptide agonist of the present invention has the amino acid sequence of Formula 4 (SEQ ID NO: 7), modified so that from one, two, three, four, five, six, seven, eight, nine, or ten amino acids differ from the amino acid in the corresponding position of SEQ ID NO: 1.

Another alternative embodiment of the present invention is a PEGylated VPAC2 receptor peptide agonist comprising a sequence of the formula:

Formula 2

(SEQ ID NO: 5)

Xaa₁-Xaa₂-Asp-Xaa₄-Xaa₅-Xaa₆-Thr-Xaa₈-Asn-Xaa₁₀-
Thr-Xaa₁₂-Xaa₁₃-Xaa₁₄-Xaa₁₅-Xaa₁₆-Xaa₁₇-Ala-Xaa₁₉-
Xaa₂₀-Xaa₂₁-Xaa₂₂-Leu-Xaa₂₄-Xaa₂₅-Xaa₂₆-Xaa₂₇-
Xaa₂₈-Xaa₂₉-Xaa₃₀-Xaa₃₁

wherein:
Xaa₁ is: His or is absent;
Xaa₂ is: dA, Ser, Val, Gly, Thr, Leu, dS, or Pro;
Xaa₄ is: Ala, Ile, Tyr, Phe, Val, Thr, Leu, Trp, or Gly;
Xaa₅ is: Val, Leu, Phe, Ile, Thr, Trp, or Tyr;
Xaa₆ is: Phe, Ile, Leu, Thr, Val, Trp, or Tyr;
Xaa₈ is: Asp;
Xaa₁₀ is: Tyr or Trp;
Xaa₁₂ is: Arg or Lys;
Xaa₁₃ is: Leu, Phe, Glu, or Ala;
Xaa₁₄ is: Arg, Leu, Lys or Ala;
Xaa₁₅ is: Lys, Ala, Arg, Glu, or Leu;
Xaa₁₆ is: Gln, Lys, or Ala;
Xaa₁₇ is: Val, Ala, Leu, or Met;
Xaa₁₉ is: Ala or Leu;
Xaa₂₀ is: Lys, Gln, hR, Arg, or Ser;
Xaa₂₁ is: Lys or Arg;
Xaa₂₂ is: Tyr, Trp, Phe, Thr, Leu, Ile, or Val;
Xaa₂₄ is: Gln or Asn;
Xaa₂₅ is: Ser, Phe, Ile, Leu, Thr, Val, Trp, Gln, Asn, or Tyr;
Xaa₂₆ is: Ile, Leu, Thr, Val, Trp, Tyr, or Phe;
Xaa₂₇ is: Lys, hR, Arg, Gln, or Leu;
Xaa₂₈ is: Asn, Lys, or Arg;
Xaa₂₉ is: Lys, Ser, Arg, Asn, hR, or is absent;
Xaa₃₀ is: Arg, Lys, Ile, or is absent; and
Xaa₃₁ is: Tyr, His, Phe, or is absent,

provided that if Xaa₂₉ is absent then Xaa₃₀ and Xaa₃₁ are also absent and if Xaa₃₀ is absent then Xaa₃₁ is absent;

and a C-terminal extension wherein the N-terminus of the C-terminal extension is linked to the C-terminus of the sequence and wherein the C-terminal extension comprises an amino acid sequence of the Formula 7 (SEQ ID NO: 15);

provided that if Xaa₁, Xaa₂, Xaa₃, Xaa₄, Xaa₅, Xaa₆, Xaa₇, Xaa₈, Xaa₉, or Xaa₁₀ of Formula 7 is absent, the next amino acid present downstream is the next amino acid in the C-terminal extension and wherein the C-terminal amino acid may be amidated,

and wherein:

at least one of the Cys residues is covalently attached to a PEG molecule, or

at least one of the Lys residues is covalently attached to a PEG molecule, or

the carboxy-terminal amino acid is covalently attached to a PEG molecule, or any combination thereof.

Preferably, an alternative selective VPAC2 receptor peptide agonist of the present invention has the amino acid sequence of Formula 2 (SEQ ID NO: 5), modified so that from one, two, three, four, five, six, seven, eight, nine, or ten amino acids differ from the amino acid in the corresponding position of SEQ ID NO: 1.

Yet another alternative embodiment of the present invention is a PEGylated VPAC2 receptor peptide agonist comprising a sequence of the formula:

Formula 3

(SEQ ID NO: 6)

His-Xaa₂-Xaa₃-Ala-Val-Phe-Thr-Xaa₈-Xaa₉-Tyr-Thr-
Xaa₁₂-Leu-Arg-Xaa₁₅-Xaa₁₆-Xaa₁₇-Ala-Xaa₁₉-Xaa₂₀-
Xaa₂₁-Tyr-Leu-Xaa₂₄-Xaa₂₅-Xaa₂₆-Xaa₂₇-Xaa₂₈-Xaa₂₉-
Xaa₃₀-Xaa₃₁-Xaa₃₂-Xaa₃₃-Xaa₃₄-Xaa₃₅-Xaa₃₆-Xaa₃₇-
Xaa₃₈-Xaa₃₉-Xaa₄₀

wherein:
Xaa₂ is: Ser or Thr;
Xaa₃ is: Asp or Glu;
Xaa₈ is: Asp or Glu;
Xaa₉ is: Asn, Gln, or Asp;
Xaa₁₂ is: Arg, Lys, or Glu;
Xaa₁₅ is: Lys or Glu;
Xaa₁₆ is: Gln or Glu;
Xaa₁₇ is: Met, Leu, Ile, or Val;
Xaa₁₉ is: Val, Ala, Glu, Phe, Gly, His, Ile, Lys, Leu, Met, Asn, Pro, Gln, Arg, Ser, Thr, Trp, Tyr, Cys, or Asp;
Xaa₂₀ is: Lys or His;
Xaa₂₁ is: Lys or His;
Xaa₂₄ is: Asn, Gln, or Glu;
Xaa₂₅ is: Ser, Asp, or Thr;
Xaa₂₆ is: Ile or Leu;
Xaa₂₇ is: Leu, Lys, Ala, Asp, Glu, Phe, Gly, His, Ile, Met, Asn, Pro, Gln, Arg, Ser, Thr, Val, Trp, Tyr, or Cys;
Xaa₂₈ is: Asn, Asp, Gln, or Lys;
Xaa₂₉ is: Gly, Lys, Ala, Asp, Glu, Phe, His, Ile, Leu, Met, Asn, Pro, Gln, Arg, Ser, Thr, Val, Trp, Tyr, Cys, or is absent;
Xaa₃₀ is: Gly, Arg, Ala, Asp, Glu, Phe, His, Ile, Lys, Leu, Met, Asn, Pro, Gln, Ser, Thr, Val, Trp, Tyr, Cys, or is absent;
Xaa₃₁ is: Thr, Tyr, Cys, or is absent;
Xaa₃₂ is: Ser, Cys, or is absent;
Xaa₃₃ is: Trp or is absent;
Xaa₃₄ is: Cys or is absent;
Xaa₃₅ is: Glu or is absent;
Xaa₃₆ is: Pro or is absent;
Xaa₃₇ is: Gly or is absent;
Xaa₃₈ is: Trp or is absent;
Xaa₃₉ is: Cys or is absent;
Xaa₄₀ is: Arg or is absent;

provided that if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈, or Xaa₃₉ is absent, the next amino acid present downstream is the next amino acid in the sequence;

and a C-terminal extension wherein the N-terminus of the C-terminal extension is linked to the C-terminus of the sequence and wherein the C-terminal extension comprises an amino acid sequence selected from the group consisting of:

### a) Formula 7 (SEQ ID NO: 15);

provided that if Xaa₁, Xaa₂, Xaa₃, Xaa₄, Xaa₅, Xaa₆, Xaa₇, Xaa₈, Xaa₉ or Xaa₁₀ of Formula 7 is absent, the next amino acid present downstream is the next amino acid in the C-terminal extension and wherein the C-terminal amino acid may be amidated;

### b) Formula 5 (SEQ ID NO: 8);

provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ of Formula 5 is absent, the next amino acid present downstream is the next amino acid in the C-terminal extension and wherein the C-terminal amino acid may be amidated; and

### c) Formula 6 (SEQ ID NO: 9);

provided that if Xaa₁, Xaa₂, Xaa₃, Xaa₄, Xaa₅, Xaa₆, Xaa₇, Xaa₈, Xaa₉, or Xaa₁₀ of Formula 6 is absent, the next amino acid present downstream is the next amino acid in the sequence and wherein the C-terminal amino acid may be amidated,

and wherein:

at least one of the Cys residues is covalently attached to a PEG molecule, or

at least one of the Lys residues is covalently attached to a PEG molecule, or

the carboxy-terminal amino acid is covalently attached to a PEG molecule, or any combination thereof.

For example, if Xaa₂₉ of the peptide sequence is Gly and Xaa₃₀ is absent, the next amino acid bonded to Gly at position 29 is an amino acid listed for position 31 or, if position 31 is also absent, an amino acid listed for position 32 is bonded to Gly at position 29, and so forth. Additionally, for example, if Xaa₂₉ is Gly and Xaa₃₀ through Xaa₄₀ are absent, Gly may be the C-terminal amino acid and may be amidated.

Preferably, an alternative selective VPAC2 receptor peptide agonist of the present invention has the amino acid sequence of Formula 3 (SEQ ID NO: 6), modified so that from one, two, three, four, five, six, seven, eight, nine, or ten amino acids differ from the amino acid in the corresponding position of SEQ ID NO: 1.

Another alternative embodiment of the present invention is a PEGylated VPAC2 receptor peptide agonist comprising a sequence of the formula:

Formula 1

(SEQ ID NO: 4)

His-Xaa₂-Asp-Ala-Val-Phe-Thr-Asp-Asn-Tyr-Thr-
Xaa₁₂-Leu-Xaa₁₄-Xaa₁₅-Xaa₁₆-Xaa₁₇-Ala-Xaa₁₉-Xaa₂₀-
Xaa₂₁-Tyr-Leu-Xaa₂₄-Xaa₂₅-Xaa₂₆-Xaa₂₇-Asn-Xaa₂₉-
Xaa₃₀-Xaa₃₁

wherein:
Xaa₂ is: Ser, Val, dA, or dS;
Xaa₁₂ is: Arg, Lys, hR, Orn, or Lys (isopropyl);
Xaa₁₄ is: Arg, Leu, Lys, hR, Orn, or Lys (isopropyl);
Xaa₁₅ is: Lys, Ala, Arg, hR, Orn, or Lys(isopropyl);
Xaa₁₆ is: Gln, Lys, Ala, hR, Orn, or Lys (isopropyl);
Xaa₁₇ is: Met, Val, Ala, or Leu;
Xaa₁₉ is: Val, Ala or Leu;
Xaa₂₀ is: Lys, Gln, Arg, hR, Orn, or Lys (isopropyl);
Xaa₂₁ is: Lys or Arg;
Xaa₂₄ is: Asn or Gln;
Xaa₂₅ is: Ser, Phe, Ile, Leu, Thr, Val, Trp, Gln, Asn, or Tyr;
Xaa₂₆ is: Ile, Leu, Thr, Val, Trp, Tyr, or Phe;
Xaa₂₇ is: Leu, hR, Arg, Lys, or Lys (isopropyl);
Xaa₂₉ is: Lys, Ser, Arg, hR, or absent;
Xaa₃₀ is: Arg, Lys, or absent; and
Xaa₃₁ is: Tyr, Phe, or absent,

provided that at least one Xaa selected from the group consisting of: Xaa₂, Xaa₁₄, Xaa₁₅, Xaa₁₆, Xaa₁₇, Xaa₂₀, Xaa₂₅, Xaa₂₆, Xaa₂₇, and Xaa₃₁ is an amino acid that differs from the amino acid at the corresponding position in SEQ ID NO: 1,

provided that if Xaa₂₉ is absent then Xaa₃₀ and Xaa₃₁ are also absent, and if Xaa₃₀ is absent then Xaa₃₁ is also absent;

and a C-terminal extension wherein the N-terminus of the C-terminal extension is linked to the C-terminus of the sequence and wherein the C-terminal extension comprises an amino acid sequence selected from the group consisting of:

### a) Formula 5 (SEQ ID NO: 8);

provided that if Xaa₁, Xaa₂, Xaa₃, Xaa₄, Xaa₅, Xaa₆, Xaa₇, Xaa₈, Xaa₉, Xaa₁₀, Xaa₁₁, Xaa₁₂, or Xaa₁₃ of Formula 5 is absent, the next amino acid present downstream is the next amino acid in the C-terminal extension and wherein the C-terminal amino acid may be amidated; and

### b) Formula 6 (SEQ ID NO: 9);

provided that if Xaa₁, Xaa₂, Xaa₃, Xaa₄, Xaa₅, Xaa₆, Xaa₇, Xaa₈, Xaa₉, or Xaa₁₀ of Formula 6 is absent, the next amino acid present downstream is the next amino acid in the sequence and wherein the C-terminal amino acid may be amidated,

and wherein:

at least one of the Cys residues is covalently attached to a PEG molecule, or

at least one of the Lys residues is covalently attached to a PEG molecule, or

the carboxy-terminal amino acid is covalently attached to a PEG molecule, or any combination thereof.

Preferably, an alternative selective VPAC2 receptor peptide agonist of the present invention has the amino acid sequence of Formula 1 (SEQ ID NO: 4), modified so that from one, two, three, four, five, six, seven, eight, nine, or ten amino acids differ from the amino acid in the corresponding position of SEQ ID NO: 1.

A further alternative embodiment of the present invention is a VPAC2 receptor peptide agonist comprising a sequence of the Formula 1 (SEQ ID NO: 4), wherein the sequence has at least one amino acid substitution selected from the group consisting of:

Xaa₂ is: Val or dA;

Xaa₁₄ is: Leu;

Xaa₁₅ is: Ala;

Xaa₁₆ is: Lys;

Xaa₁₇ is: Ala;

Xaa₂₀ is: Gln;

Xaa₂₅ is: Phe, Ile, Leu, Val, Trp, or Tyr;

Xaa₂₆ is: Thr, Trp, or Tyr;

Xaa₂₇ is: hR; and

Xaa₃₁ is: Phe,

and provided that if Xaa₂₉ is absent then Xaa₃₀ and Xaa₃₁ are also absent and if Xaa₃₀ is absent then Xaa₃₁ is absent.

The peptide of Formula 1 (SEQ ID NO: 4) can further comprise a C-terminal extension wherein the N-terminus of the C-terminal extension is linked to the C-terminus of the peptide of Formula 1 (SEQ ID NO: 4) and wherein the C-terminal extension comprises an amino acid sequence selected from the group consisting of:

### a) Formula 5 (SEQ ID NO: 8);

provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ of Formula 5 is absent, the next amino acid present downstream is the next amino acid in the C-terminal extension and wherein the C-terminal amino acid may be amidated; and

### b) Formula 6 (SEQ ID NO: 9);

provided that if Xaa₁, Xaa₂, Xaa₃, Xaa₄, Xaa₅, Xaa₆, Xaa₇, Xaa₈, Xaa₉, or Xaa₁₀ of Formula 6 is absent, the next amino acid present downstream is the next amino acid in the sequence and wherein the C-terminal amino acid may be amidated.

Additional alternative embodiments of the present invention include a VPAC2 receptor peptide agonist further comprising a N-terminal modification linked to the N-terminus of the peptide sequence wherein the N-terminal modification involves acylation, alkylation, acetylation, a carbobenzoyl group, a succinimide group, a sulfonamide group, a carbamate group, or a urea group. N-terminal modification includes, but is not limited to eighteen carbons (C-18), ten carbons (C-10), and six carbons (C-6). N-terminal modification also includes HS(CH₂)₂CO.

Other alternative embodiments of the present invention include a VPAC2 receptor peptide agonist further comprising a N-terminal modification linked to the N-terminus of the peptide sequence wherein the N-terminal modification is selected from the group consisting of D-histidine and isoleucine.

Alternative embodiments of the present invention also include a VPAC2 receptor peptide agonist further comprising a N-terminal modification linked to the N-terminus of the peptide sequence wherein the N-terminal modification is selected from the group consisting of acetyl, propionyl, butyryl, pentanoyl, hexanoyl, Met, 3-phenylpropionyl, phenylacetyl, benzoyl, and norleucine.

The VPAC2 receptor peptide agonists of the present invention, therefore, have the advantage that they have enhanced selectivity, potency and/or stability over known VPAC2 receptor peptide agonists. In particular, the addition of the extension sequence of exendin-4 as the c-capping sequence surprisingly increased the VPAC2 receptor selectivity as well as increasing proteolytic stability. The covalent attachment of one or more molecules of PEG to particular residues of a VPAC2 receptor peptide agonist results in a biologically active, PEGylated VPAC2 receptor peptide agonist with an extended half-life and reduced clearance when compared to that of non-PEGylated VPAC2 receptor peptide agonists.

A “selective VPAC2 receptor peptide agonist” of the present invention is a peptide that selectively activates the VPAC2 receptor to induce insulin secretion. Preferably, the sequence for a selective VPAC2 receptor peptide agonist of the present invention has from about twenty-eight to about thirty-five naturally occurring and/or non-naturally occurring amino acids and may or may not additionally comprise a C-terminal extension. More preferably, the selective VPAC2 receptor peptide agonist has from twenty-eight to thirty-one naturally occurring and/or non-naturally occurring amino acids and may or may not additionally comprise a C-terminal extension.

A “selective PEGylated VPAC2 receptor peptide agonist” is a selective VPAC2 receptor peptide agonist covalently attached to one or more molecules of polyethylene glycol (PEG), or a derivative thereof, wherein each PEG is attached to a cysteine or lysine amino acid, to a K(W) or K(CO(CH₂)₂SH), or to the carboxy terminus of a peptide.

Selective PEGylated VPAC2 receptor peptide agonists may have a C-terminal extension. The “C-terminal extension” of the present invention comprises a sequence having from one to thirteen naturally occurring or non-naturally occurring amino acids linked to the C-terminus of the sequence of Formula 10, 12, 13 or 16 at the N-terminus of the C-terminal extension via a peptide bond. Any one of the Cys, Lys, K(W), or K(CO(CH₂)₂SH) residues in the C-terminal extension can be covalently attached to a PEG molecule, or the carboxy-terminal amino acid of the C-terminal extension can be covalently attached to a PEG molecule.

As used herein, the term “linked to” with reference to the term C-terminal extension, includes the addition or attachment of amino acids or chemical groups directly to the C-terminus of the peptide of the Formula 10, 12, 13, or 16.

Optionally, the selective PEGylated VPAC2 receptor peptide agonist may also have an N-terminus modification. The term “N-terminal modification” as used herein includes the addition or attachment of amino acids or chemical groups directly to the N-terminal of a peptide and the formation of chemical groups, which incorporate the nitrogen at the N-terminus of a peptide.

The N-terminal modification may comprise the addition of one or more naturally occurring or non-naturally occurring amino acids to the VPAC2 receptor peptide agonist sequence, preferably there are not more than ten amino acids, with one amino acid being more preferred. Naturally occurring amino acids which may be added to the N-terminus include methionine and isoleucine. A modified amino acid added to the N-terminus may be D-histidine. Alternatively, the following amino acids may be added to the N-terminus: (SEQ ID NO: 14) Ser-Trp-Cys-Glu-Pro-Gly-Trp-Cys-Arg, wherein the Arg is linked to the N-terminus of the peptide agonist. Preferably, any amino acids added to the N-terminus are linked to the N-terminus by a peptide bond.

The term “linked to” as used herein, with reference to the term N-terminal modification, includes the addition or attachment of amino acids or chemical groups directly to the N-terminus of the PEGylated VPAC2 receptor agonist. The addition of the above N-terminal modifications may be achieved under normal coupling conditions for peptide bond formation.

The N-terminus of the peptide agonist may also be modified by the addition of an alkyl group (R), preferably a C₁-C₁₆ alkyl group, to form (R)NH—.

Alternatively, the N-terminus of the peptide agonist may be modified by the addition of a group of the formula —C(O)R¹ to form an amide of the formula R¹C(O)NH—. The addition of a group of the formula —C(O)R¹ may be achieved by reaction with an organic acid of the formula R¹COOH. Modification of the N-terminus of an amino acid sequence using acylation is demonstrated in the art (e.g. Gozes et al., J. Pharmacol Exp Ther, 273:161-167 (1995)). Addition of a group of the formula —C(O)R¹ may result in the formation of a urea group (see WO 01/23240, WO 2004/006839) or a carbamate group at the N-terminus. Also, the N-terminus may be modified by the addition of pyroglutamic acid or 6-aminohexanoic acid.

The N-terminus of the peptide agonist may be modified by the addition of a group of the formula —SO₂R⁵, to form a sulfonamide group at the N-terminus.

The N-terminus of the peptide agonist may also be modified by reacting with succinic anhydride to form a succinimide group at the N-terminus. The succinimide group incorporates the nitrogen at the N-terminus of the peptide.

The N-terminus may alternatively be modified by the addition of methionine sulfoxide, biotinyl-6-aminohexanoic acid, or —C(═NH)—NH₂. The addition of —C(═ONNH)—NH₂ is a guanidation modification, where the terminal NH₂ of the N-terminal amino acid becomes —NH—C(═NH)—NH₂.

Most of the sequences of the present invention, including the N-terminal modifications and the C-terminal extensions contain the standard single letter or three letter codes for the twenty naturally occurring amino acids. The other codes used are defined as follows:
Ac=Acetyl
C6=hexanoyl
d=the D isoform (non-naturally occurring) of the respective amino acid, e.g., dA=D-alanine, dS=D-serine, dK=D-lysine
hR=homoarginine
_=position not occupied
Aib=amino isobutyric acid
CH₂=methylene
Met(O)=methionine sulfoxide
OMe=methoxy
Nle=Nor-leucine
NMe=N-methyl attached to the alpha amino group of an amino acid, e.g., NMeA=N-methyl alanine, NMeV=N-methyl valine
Orn=ornithine
Cit=citrulline
K (Ac)=ε-acetyl lysine
M=methionine
I=isoleucine
PEG=polyethylene glycol
Biotin-Acp=Biotinyl-6-aminohexanoic acid (6-aminocaproic acid)
K(W)=ε-(L-tryptophyl)-lysine
K(CO(CH₂)₂SH)=ε-(3′-mercaptopropionyl)-lysine
[symbol] =a lactam bridge

The term “VPAC2” is used to refer to and in conjunction with the particular receptor (Lutz, et al., FEBS Lett., 458: 197-203 (1999); Adamou, et al., Biochem. Biophys. Res. Commun., 209: 385-392 (1995)) that the agonists of the present invention activate. This term also is used to refer to and in conjunction with the agonists of the present invention.

VIP naturally occurs as a single sequence having 28 amino acids. However, PACAP exists as either a 38 amino acid peptide (PACAP-38) or as a 27 amino acid peptide (PACAP-27) with an amidated carboxyl (Miyata, et al., Biochein Biophys Res Commun, 170:643-648 (1990)). The sequences for VIP, PACAP-27, and PACAP-38 are as follows

| | Seq.ID | |
|---|---|---|
| Peptide | # | Sequence |
| VIP | SEQ ID | HSDAYFTDNYTRLRKQMAVKKYLNSILN |
| | NO: 1 | |
| PACAP-27 | SEQ ID | HSDGIFTDSYSRYRKQMAVKKYLAAVL-NH2 |
| | NO: 2 | |
| PACAP-38 | SEQ ID | HSDGIFTDSYSRYRKQMAVKKYLAAVLGKRYQRVKN |
| | NO: 3 | K-NH₂ |

The term “naturally occurring amino acid” as used herein means the twenty amino acids coded for by the human genetic code (i.e. the twenty standard amino acids). These twenty amino acids are: Alanine, Arginine, Asparagine, Aspartic Acid, Cysteine, Glutamine, Glutamic Acid, Glycine, Histidine, Isoleucine, Leucine, Lysine, Methionine, Phenylalanine, Proline, Serine, Threonine, Tryptophan, Tyrosine and Valine.

Examples of “non-naturally occurring amino acids” include both synthetic amino acids and those modified by the body. These include D-amino acids, arginine-like amino acids (e.g., homoarginine), and other amino acids having an extra methylene in the side chain (“homo” amino acids), and modified amino acids (e.g norleucine, lysine (isopropyl)—wherein the side chain amine of lysine is modified by an isopropyl group). Also included are amino acids such as ornithine and amino isobutyric acid.

“Selective” as used herein refers to a VPAC2 receptor peptide agonist with increased selectivity for the VPAC2 receptor compared to other known receptors. The degree of selectivity is determined by a ratio of VPAC2 receptor binding affinity to VPAC1 receptor binding affinity and by a ratio of VPAC2 receptor binding affinity to PAC1 receptor binding affinity. Preferably, the agonists of the present invention have a selectivity ratio where the affinity for the VPAC2 receptor is at least 50 times greater than for the VPAC1 and/or for PAC1 receptors. More preferably, the affinity is at least 100 times greater for VPAC2 than for VPAC1 and/or for PAC1. Even more preferably, the affinity is at least 200 times greater for VPAC2 than for VPAC1 and/or for PAC1. Still more preferably, the affinity is at least 500 times greater for VPAC2 than for VPAC1 and/or for PAC1. Yet more preferably, the affinity is at least 1000 times greater for VPAC2 than for VPAC1 and/or for PAC1. Binding affinity is determined as described below in Example 4.

“Percent (%) sequence identity” as used herein is used to denote sequences which when aligned have similar (identical or conservatively replaced) amino acids in like positions or regions, where identical or conservatively replaced amino acids are those which do not alter the activity or function of the protein as compared to the starting protein. For example, two amino acid sequences with at least 85% identity to each other have at least 85% similar (identical or conservatively replaced residues) in a like position when aligned optimally allowing for up to 3 gaps, with the proviso that in respect of the gaps a total of not more than 15 amino acid residues is affected. Percent sequence identity may be calculated by determining the number of residues that differ between a peptide encompassed by the present invention and a reference peptide such as P83 (SEQ ID NO: 83), taking that number and dividing it by the number of amino acids in the reference peptide (e.g. 39 amino acids for P83), multiplying the result by 100, and subtracting that resulting number from 100. For example, a sequence having 39 amino acids with four amino acids that are different from P83 would have a percent (%) sequence identity of 90% (e.g. 100˜((4/39)×100)). For a sequence that is longer than 39 amino acids, the number of residues that differ from the P83 sequence will include the additional amino acids over 39 for purposes of the aforementioned calculation. For example, a sequence having 41 amino acids, with four amino acids different from the 39 amino acids in the P83 sequence and with two additional amino acids at the carboxy terminus which are not present in the P83 sequence, would have a total of six amino acids that differ from P83. Thus, this sequence would have a percent (%) sequence identity of 84% (e.g. 100−((6/39)×100)). The degree of sequence identity may be determined using methods well known in the art (see, for example, Wilbur, W. J. and Lipman, D. J., Proc. Natl. Acad. Sci. USA 80:726-730 (1983) and Myers E. and Miller W., Comput. Appl. Biosci. 4:11-17 (1988)). One program which may be used in determining the degree of similarity is the MegAlign Lipman-Pearson one pair method (using default parameters) which can be obtained from DNAstar Inc, 1128, Selfpark Street, Madison, Wis., 53715, USA as part of the Lasergene system. Another program, which may be used, is Clustal W. This is a multiple sequence alignment package developed by Thompson et al (Nucleic Acids Research, 22(22):4673-4680 (1994)) for DNA or protein sequences. This tool is useful for performing cross-species comparisons of related sequences and viewing sequence conservation. Clustal W is a general purpose multiple sequence alignment program for DNA or proteins. It produces biologically meaningful multiple sequence alignments of divergent sequences. It calculates the best match for the selected sequences, and lines them up so that the identities, similarities and differences can be seen. Evolutionary relationships can be seen via viewing Cladograms or Phylograms.

The sequence for a selective PEGylated VPAC2 receptor peptide agonist of the present invention is selective for the VPAC2 receptor and preferably has a sequence identity in the range of 60% to 70%, 60% to 65%, 65% to 70%, 70% to 80%, 70% to 75%, 75% to 80%, 80% to 90%, 80% to 85%, 85% to 90%, 90% to 97%, 90% to 95%, or 95% to 97%, with P83 (SEQ ID NO: 83). Preferably, the sequence has a sequence identity of greater than 71% with P83 (SEQ ID NO: 83). More preferably, the sequence has greater than 74% sequence identity with P83 (SEQ ID NO: 83). Even more preferably, the sequence has greater than 76% sequence identity with P83 (SEQ ID NO: 83). Yet more preferably, the sequence has greater than 79% sequence identity or 84% sequence identity with P83 (SEQ ID NO: 83).

The term “C₁-C₁₆ alkyl” as used herein means a monovalent saturated straight, branched or cyclic chain hydrocarbon radical having from 1 to 16 carbon atoms. Thus the term “C₁-C₁₆ alkyl” includes, for example, methyl, ethyl, n-propyl, isopropyl, n-butyl, isobutyl, sec-butyl, tert-butyl, n-heptyl, n-octyl, cyclopropyl, cyclobutyl, cyclopentyl and cyclohexyl. The C₁-C₁₆ alkyl group may be optionally substituted with one or more substituents.

The term “C₁-C₆ alkyl” as used herein means a monovalent saturated straight, branched or cyclic chain hydrocarbon radical having from 1 to 6 carbon atoms. Thus the term “C₁-C₆ alkyl” includes, for example, methyl, ethyl, n-propyl, isopropyl, n-butyl, isobutyl, sec-butyl, tert-butyl, cyclopropyl, cyclobutyl, cyclopentyl and cyclohexyl. The C₁-C₆ alkyl group may be optionally substituted with one or more substituents.

The term “C₂-C₆ alkenyl” as used herein means a monovalent straight, branched or cyclic chain hydrocarbon radical having at least one double bond and having from 2 to 6 carbon atoms. Thus the term “C₂-C₆ alkenyl” includes vinyl, prop-2-enyl, but-3-enyl, pent-4-enyl and isopropenyl. The C₂-C₆ alkenyl group may be optionally substituted with one or more substituents.

The term “C₂-C₆ alkynyl” as used herein means a monovalent straight or branched chain hydrocarbon radical having at least one triple bond and having from 2 to 6 carbon atoms. Thus the term “C₂-C₆ alkynyl” includes prop-2-ynyl, but-3-ynyl and pent-4-ynyl. The C₂-C₆ alkynyl may be optionally substituted with one or more substituents.

The term “halo” or “halogen” means fluorine, chlorine, bromine or iodine.

The term “aryl” when used alone or as part of a group is a 5 to 10 membered aromatic or heteroaromatic group including a phenyl group, a 5 or 6-membered monocyclic heteroaromatic group, each member of which may be optionally substituted with 1, 2, 3, 4 or 5 substituents (depending upon the number of available substitution positions), a naphthyl group or an 8-, 9- or 10-membered bicyclic heteroaromatic group, each member of which may be optionally substituted with 1, 2, 3, 4, 5 or 6 substituents (depending on the number of available substitution positions). Within this definition of aryl, suitable substitutions include C₁-C₆ alkyl, C₂-C₆ alkenyl, C₂-C₆ alkynyl, amino, hydroxy, halogen, —SH and CF₃.

The term “aryl C₁-C₄ alkyl” as used herein means a C₁-C₄ alkyl group substituted with an aryl. Thus the term “aryl C₁-C₄ alkyl” includes benzyl, 1-phenylethyl (α-methylbenzyl), 2-phenylethyl, 1-naphthalenemethyl or 2-naphthalenemethyl.

The term “naphthyl” includes 1-naphthyl, and 2-naphthyl. 1-naphthyl is preferred.

The term “benzyl” as used herein means a monovalent unsubstituted phenyl radical linked to the point of substitution by a —CH₂— group.

The term “5- or 6-membered monocyclic heteroaromatic group” as used herein means a monocyclic aromatic group with a total of 5 or 6 atoms in the ring wherein from 1 to 4 of those atoms are each independently selected from N, O and S. Preferred groups have 1 or 2 atoms in the ring which are each independently selected from N, O and S. Examples of 5-membered monocyclic heteroaromatic groups include pyrrolyl (also called azolyl), furanyl, thienyl, pyrazolyl (also called 1H-pyrazolyl and 1,2-diazolyl), imidazolyl, oxazolyl (also called 1,3-oxazolyl), isoxazolyl (also called 1,2-oxazolyl), thiazolyl (also called 1,3-thiazolyl), isothiazolyl (also called 1,2-thiazolyl), triazolyl, oxadiazolyl, thiadiazolyl, tetrazolyl, oxatriazolyl and thiatriazolyl. Examples of 6-membered monocyclic heteroaromatic groups include pyridinyl, pyrimidyl, pyrazinyl, pyridinyl and triazinyl.

The term “8-, 9- or 10-membered bicyclic heteroaromatic group” as used herein means a fused bicyclic aromatic group with a total of 8, 9 or 10 atoms in the ring system wherein from 1 to 4 of those atoms are each independently selected from N, O and S. Preferred groups have from 1 to 3 atoms in the ring system which are each independently selected from N, O and S. Suitable 8-membered bicyclic heteroaromatic groups include imidazo[2,1-b][1,3]thiazolyl, thieno[3,2-b]thienyl, thieno[2,3-d][1,3]thiazolyl and thieno[2,3-d]imidazolyl. Suitable 9-membered bicyclic heteroaromatic groups include indolyl, isoindolyl, benzofuranyl (also called benzo[b]furanyl), isobenzofuranyl (also called benzo[c]furanyl), benzothienyl (also called benzo[b]thienyl), isobenzothienyl (also called benzo[c]thienyl), indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl and imidazo[1,2-a]pyridine. Suitable 10-membered bicyclic heteroaromatic groups include quinolinyl, isoquinolinyl, cinnolinyl, quinazolinyl, quinoxalinyl, 1,5-naphthyridyl, 1,6-naphthyridyl, 1,7-naphthyridyl and 1,8-naphthyridyl.

The term “C₁-C₆ alkoxy” as used herein means a monovalent unsubstituted saturated straight-chain or branched-chain hydrocarbon radical having from 1 to 6 carbon atoms linked to the point of substitution by a divalent O radical. Thus the term “C₁-C₆ alkoxy” includes, for example, methoxy, ethoxy, n-propoxy, isopropoxy, n-butoxy, isobutoxy, sec-butoxy and tert-butoxy. The C₁-C₆ alkoxy group may be optionally substituted with one or more substituents.

The term “PEG” as used herein means a polyethylene glycol molecule. In its typical form, PEG is a linear polymer with terminal hydroxyl groups and has the formula HO—CH₂CH₂—(CH₂CH₂O)n—CH₂CH₂—OH, where it is from about 8 to about 4000. The terminal hydrogen may be substituted with a protective group such as an alkyl or alkanol group. Preferably, PEG has at least one hydroxy group, more preferably it is a terminal hydroxy group. It is this hydroxy group which is preferably activated to react with the peptide. There are many forms of PEG useful for the present invention. Numerous derivatives of PEG exist in the art and are suitable for use in the invention. (See, e.g., U.S. Pat. Nos. 5,445,090; 5,900,461; 5,932,462; 6,436,386; 6,448,369; 6,437,025; 6,448,369; 6,495,659; 6,515,100 and 6,514,497 and Zalipsky, S. Bioconjugate Chem. 6:150-165, 1995). The PEG molecule covalently attached to VPAC2 receptor peptide agonists in the present invention is not intended to be limited to a particular type. The molecular weight of the PEG molecule is preferably from 500-100,000 daltons and more preferably 10,000, 20,000, 30,000, 40,000, 50,000, or 60,000 daltons and most preferably 20,000 or 40,000 daltons. PEG may be linear or branched and PEGylated VPAC2 receptor peptide agonists of the invention may have one, two or three PEG molecules attached to the peptide. It is more preferable that there be one or two PEG molecules per PEGylated VPAC2 receptor peptide agonist, however, when there is more than one PEG molecule per peptide molecule, it is preferred that there be no more than three. It is further contemplated that both ends of the PEG molecule may be homo- or hetero-functionalized for crosslinking two or more VPAC2 receptor peptide agonists together. Where there are two PEG molecules present, the PEG molecules will preferably be 20,000 dalton PEG molecules. However, PEG molecules having a different molecular weight may be used, for example, one 10,000 dalton PEG molecule and one 30,000 PEG molecule.

In the present invention, a PEG molecule may be covalently attached to a Cys or Lys residue or to the C-terminal residue. The PEG molecule may also be covalently attached to a Trp residue which is coupled to the side chain of a Lys residue (K(W)). Alternatively, a K(CO(CH₂)₂SH) group may be PEGylated to form K(CO(CH₂)₂S-PEG). Any Lys residue in the peptide agonist may be substituted for a K(W) or K(CO(CH₂)₂SH), which may then be PEGylated. In addition, any Cys residue in the peptide agonist may be substituted for a modified cysteine residue, for example, hC. The modified Cys residue may be covalently attached to a PEG molecule.

The term “PEGylation” as used herein means the covalent attachment of one or more PEG molecules as described above to the VPAC2 receptor peptide agonists of the present invention.

“Insulinotropic activity” refers to the ability to stimulate insulin secretion in response to elevated glucose levels, thereby causing glucose uptake by cells and decreased plasma glucose levels. Insulinotropic activity can be assessed by methods known in the art, including using experiments that measure VPAC2 receptor binding activity or receptor activation (e.g. insulin secretion by insulinoma cell lines or islets, intravenous glucose tolerance test (IVGTT)). Intraperitoneal glucose tolerance test (IPGTT), and oral glucose tolerance test (OGTT)). Insulinotropic activity is routinely measured in humans by measuring insulin levels or C-peptide levels. Selective VPAC2 receptor peptide agonists of the present invention have insulinotropic activity.

“In vitro potency” as used herein is the measure of the ability of a peptide to activate the VPAC2 receptor in a cell-based assay. In vitro potency is expressed as the “EC₅₀” which is the effective concentration of compound that results in a 50% of maximum increase in activity in a single dose-response experiment. For the purposes of the present invention, in vitro potency is determined using the Alpha Screen assay. See Example 3 for further details of this assay.

The term “plasma half-life” refers to the time in which half of the relevant molecules circulate in the plasma prior to being cleared. An alternatively used term is “elimination half-life.” The term “extended” or “longer” used in the context of plasma half-life or elimination half-life indicates there is a statistically significant increase in the half-life of a PEGylated VPAC2 receptor peptide agonist relative to that of the reference molecule (e.g., the non-PEGylated form of the peptide or the native peptide) as determined under comparable conditions. Preferably a PEGylated VPAC2 receptor peptide agonist of the present invention has an elimination half-life of at least one hour, more preferably at least 3, 5, 7, 10, 15, 20 or 24 hours and most preferably at least 48 hours. The half-life reported herein is the elimination half-life; it is that which corresponds to the terminal log-linear rate of elimination. The person skilled in the art appreciates that half-life is a derived parameter that changes as a function of both clearance and volume of distribution.

Clearance is the measure of the body's ability to eliminate a drug. As clearance decreases due, for example, to modifications to a drug, half-life would be expected to increase. However, this reciprocal relationship is exact only when there is no change in the volume of distribution. A useful approximate relationship between the terminal log-linear half-life (t_{1/2}), clearance (C), and volume of distribution (V) is given by the equation: t_{1/2}≈0.693 (V/C). Clearance does not indicate how much drug is being removed but, rather, the volume of biological fluid such as blood or plasma that would have to be completely freed of drug to account for the elimination. Clearance is expressed as a volume per unit of time. The PEGylated VPAC2 receptor peptide agonists of the present invention preferably have a clearance value of 200 ml/h/kg or less, more preferably 180, 150, 120, 100, 80, 60 ml/h/kg or less and most preferably 50, 40 or 20 ml/h/kg or less.

According to a preferred embodiment of the present invention, there is provided a PEGylated VPAC2 receptor peptide agonist comprising an amino acid sequence of Formula 10 (SEQ ID NO: 18), Formula 12 (SEQ ID NO: 20) or Formula 13 (SEQ ID NO: 21) wherein Xaa₃ is Asp or Glu, Xaa₈ is Asp or Glu, Xaa₁₂ is Arg, hR, Lys, or Orn, Xaa₁₄ is Arg, Gln, Aib, hR, Orn, Cit, Lys, Ala, or Leu, Xaa₁₅ is Lys, Aib, Orn, or Arg, Xaa₁₆ is Gln or Lys, Xaa₁₇ is Val, Leu, Ala, Ile, Lys, or Nle, Xaa₂₀ is Lys, Val, Leu, Aib, Ala, Gln, or Arg, Xaa₂₁ is Lys, Aib, Orn, Ala, Gln, or Arg, Xaa₂₇ is Lys, Orn, hR, or Arg, Xaa₂₈ is Asn, Gln, Lys, hR, Aib, Pro, or Orn and Xaa₂₉ is Lys, Orn, hR, or absent, and a C-terminal extension comprising an amino acid sequence of Formula 7 (SEQ ID NO: 15).

It is more preferred that the C-terminal extension in this embodiment is selected from: GGPSSGAPPPS (SEQ ID NO: 10), GGPSSGAPPPS-NH₂ (SEQ ID NO: 11), GGPSSGAPPPC (SEQ ID NO: 22), GGPSSGAPPPC-NH₂ (SEQ ID NO: 23), GRPSSGAPPPS (SEQ ID NO: 16) and GRPSSGAPPPS-NH₂ (SEQ ID NO: 17).

According to another embodiment of the present invention, the PEGylated VPAC2 receptor peptide agonist comprises a sequence of the Formula 10 (SEQ ID NO: 18), Formula 12 (SEQ ID NO: 20) or Formula 13 (SEQ ID NO: 21) wherein Xaa₁₄ is Leu, Xaa₁₅ is Ala, Xaa₁₆ is Lys, Xaa₁₇ is Leu, and Xaa₂₀ is Gln.

According to another preferred embodiment of the present invention, there is provided a PEGylated VPAC2 receptor peptide agonist comprising an amino acid sequence of Formula 10 (SEQ ID NO: 18), Formula 12 (SEQ ID NO: 20) or Formula 13 (SEQ ID NO: 21) wherein Xaa₃₀ and Xaa₃₁ are absent, and a C-terminal extension comprising an amino acid sequence of Formula 7 (SEQ ID NO: 15).

Alternatively, in yet another preferred embodiment of the present invention, the PEGylated VPAC2 receptor peptide agonist comprises an amino acid sequence of Formula 10 (SEQ ID NO: 18), Formula 12 (SEQ ID NO: 20) or Formula 13 (SEQ ID NO: 21) wherein Xaa₂₉, Xaa₃₀ and Xaa₃₁ are absent, and a C-terminal extension comprising an amino acid sequence of Formula 7 (SEQ ID NO: 15).

It is more preferred that the C-terminal extension in the above embodiments is selected from: GGPSSGAPPPS (SEQ ID: 10), GGPSSGAPPPS-NH₂ (SEQ ID NO: 11), GGPSSGAPPPC (SEQ ID NO: 22), GGPSSGAPPPC-NH₂ (SEQ ID NO: 23), GRPSSGAPPPS (SEQ ID NO: 16) and GRPSSGAPPPS—NH₂ (SEQ ID NO: 17).

According to another preferred embodiment of the present invention, there is provided a PEGylated VPAC2 receptor peptide agonist comprising an amino acid sequence of Formula 10 (SEQ ID NO: 18), Formula 12 (SEQ ID NO: 20) or Formula 13 (SEQ ID NO: 21) wherein either Xaa₁₄ or Xaa₁₅ is Aib and either Xaa₂₀ or Xaa₂₁ is Aib, more preferably Xaa₁₅ is Aib and Xaa₂₀ is Aib, and a C-terminal extension comprising an amino acid sequence of Formula 7 (SEQ ID NO: 15).

According to yet another preferred embodiment of the present invention, there is provided a PEGylated VPAC2 receptor peptide agonist comprising an amino acid sequence of Formula 10 (SEQ ID NO: 18), Formula 12 (SEQ ID NO: 20) or Formula 13 (SEQ ID NO: 21) wherein either Xaa₁₄ or Xaa₁₅ is Aib and either Xaa₂₀ or Xaa₂₁ is Aib and Xaa₂₈ is Gln and Xaa₂₉ is Lys or absent, and a C-terminal extension comprising an amino acid sequence of Formula 7 (SEQ ID NO: 15).

In a further preferred embodiment of the present invention, there is provided a PEGylated VPAC2 receptor peptide agonist comprising an amino acid sequence of Formula 10 (SEQ ID NO: 18), Formula 12 (SEQ ID NO: 20) or Formula 13 (SEQ ID NO: 21) wherein either Xaa₁₄ or Xaa₁₅ is Aib and either Xaa₂₀ or Xaa₂₁ is Aib and Xaa₁₂ of the peptide sequence is hR or Orn, Xaa₂₇ is hR or Orn and Xaa₂₉ is hR or Orn and a C-terminal extension comprising an amino acid sequence of Formula 7 (SEQ ID NO: 15).

According to yet another preferred embodiment of the present invention, there is provided a PEGylated VPAC2 receptor peptide agonist comprising an amino acid sequence of Formula 10 (SEQ ID NO: 18), Formula 12 (SEQ ID NO: 20) or Formula 13 (SEQ ID NO: 21) wherein Xaa₁₅ is Aib, Xaa₂₀ is Aib, and Xaa₁₂, Xaa₂₁, Xaa₂₇, and Xaa₂₈ are all Orn, and a C-terminal extension comprising an amino acid sequence of Formula 7 (SEQ ID NO: 15). In this embodiment, it is especially preferred that Xaa₈ is Glu, Xaa₉ is Gln, and Xaa₁₀ is Tyr(OMe).

In another preferred embodiment of the present invention, there is provided a PEGylated VPAC2 receptor peptide agonist comprising an amino acid sequence of Formula 16 (SEQ ID NO: 28) and a C-terminal extension comprising an amino acid sequence of Formula 7 (SEQ ID NO: 15).

In the above preferred embodiments of the present invention, it is especially preferred that the VPAC2 receptor peptide agonist further comprises a N-terminal modification, wherein the N-terminal modification is the addition of a group selected from: acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid and —C(═NH)—NH₂, and more preferably is the addition of acetyl or hexanoyl.

In a preferred embodiment, there is provided a PEGylated VPAC2 receptor peptide agonist comprising an amino acid sequence of Formula 10 (SEQ ID NO: 18), Formula 12 (SEQ ID NO: 20) or Formula 13 (SEQ ID NO: 21) wherein either Xaa₁₄ or Xaa₁₅ is Aib and either Xaa₂₀ or Xaa₂₁ is Aib, more preferably Xaa₁₅ is Aib and Xaa₂₀ is Aib, and a C-terminal extension selected from: GGPSSGAPPPS (SEQ ID NO: 10), GGPSSGAPPPS-NH₂ (SEQ ID NO: 11), GGPSSGAPPPC (SEQ ID NO: 22), and GGPSSGAPPPC-NH₂ (SEQ ID NO: 23), GRPSSGAPPPS (SEQ ID NO: 16) and GRPSSGAPPPS-NH₂ (SEQ ID NO: 17) and wherein the PEGylated VPAC2 receptor peptide agonist further comprises a N-terminal modification which modification is the addition of hexanoyl or acetyl.

In another preferred embodiment, there is provided a PEGylated VPAC2 receptor peptide agonist comprising an amino acid sequence of Formula 10 (SEQ ID NO: 18), Formula 12 (SEQ ID NO: 20) or Formula 13 (SEQ ID NO: 21) wherein either Xaa₁₄ or Xaa₁₅ is Aib and either Xaa₂₀ or Xaa₂₁ is Aib, Xaa₂₈ is Gln and Xaa₂₉ is Lys or absent, and a C-terminal extension selected from: GGPSSGAPPPS (SEQ ID NO: 10), GGPSSGAPPPS-NH₂ (SEQ ID NO: 11), GGPSSGAPPPC (SEQ ID NO: 22), and GGPSSGAPPPC-NH₂ (SEQ ID NO: 23), GRPSSGAPPPS (SEQ ID NO: 16) and GRPSSGAPPPS-NH₂ (SEQ ID NO: 17), and wherein the PEGylated VPAC2 receptor peptide agonist further comprises a N-terminal modification which modification is the addition of hexanoyl or acetyl.

In yet another preferred embodiment, there is provided a PEGylated VPAC2 receptor peptide agonist comprising an amino acid sequence of Formula 10 (SEQ ID NO: 18), Formula 12 (SEQ ID NO: 20) or Formula 13 (SEQ ID NO: 21) wherein either Xaa₁₄ or Xaa₁₅ is Aib and either Xaa₂₀ or Xaa₂₁ is Aib, Xaa₁₂ is hR or Orn, Xaa₂₇ is hR or Orn and Xaa₂₉ is hR or Orn, and a C-terminal extension selected from: GGPSSGAPPPS (SEQ ID NO: 10), GGPSSGAPPPS-NH₂ (SEQ ID NO: 11), GGPSSGAPPPC (SEQ ID NO: 22), and GGPSSGAPPPC-NH₂ (SEQ ID NO: 23), GRPSSGAPPPS (SEQ ID NO: 16) and GRPSSGAPPPS-NH₂ (SEQ ID NO: 17) and wherein the VPAC2 receptor peptide agonist further comprises a N-terminal modification which modification is the addition of hexanoyl or acetyl.

In yet further preferred embodiment, there is provided a PEGylated VPAC2 receptor peptide agonist comprising an amino acid sequence of Formula 10 (SEQ ID NO: 18), Formula 12 (SEQ ID NO: 20) or Formula 13 (SEQ ID NO: 21) wherein Xaa₁₅ is Aib, Xaa₂₀ is Aib, Xaa₁₂, Xaa₂₁, Xaa₂₇, and Xaa₂₈ are all Orn, Xaa₈ is Glu, Xaa₉ is Gln, and Xaa₁₀ is Tyr(OMe), and a C-terminal extension selected from: GGPSSGAPPPS (SEQ ID NO: 10), GGPSSGAPPPS-NH₂ (SEQ ID NO: 11), GGPSSGAPPPC (SEQ ID NO: 22), and GGPSSGAPPPC-NH₂ (SEQ ID NO: 23), GRPSSGAPPPS (SEQ ID NO: 16) and GRPSSGAPPPS-NH₂ (SEQ ID NO: 17) and wherein the VPAC2 receptor peptide agonist further comprises a N-terminal modification which modification is the addition of hexanoyl or acetyl.

In combination with any one of the preferred embodiments described above, it is preferred that there is at least one PEG molecule covalently attached to Xaa₂₅ or any subsequent residue in Formula 10, 12, 13 or 16 and/or there is at least one PEG molecule covalently attached to a residue in the C-terminal extension of the peptide agonist. It is also preferred that one or two of the Cys residues in the peptide agonist are covalently attached to a PEG molecule, or one or two of the Lys residues in the peptide agonist are covalently attached to a PEG molecule.

A preferred alternative sequence for selective PEGylated VPAC2 receptor peptide agonists of the present invention comprises an amino acid sequence of the Formula 1 (SEQ ID NO: 4), provided that if Xaa₂₉ or Xaa₃₀ of Formula 1 is absent each amino acid downstream is absent and wherein the C-terminal amino acid may be amidated.

Throughout this specification, with respect to when an Xaa is absent, the next amino acid present downstream is the next amino acid in the sequence or is also absent. For example, if Xaa₂₉ is Lys and Xaa₃₀ is absent, the next amino acid bonded to Lys at position 29 is an amino acid listed for position 31 or absent, and so forth.

Another alternative preferred sequence for selective PEGylated VPAC2 receptor peptide agonists of the present invention comprises an amino acid sequence of the Formula 1 (SEQ ID NO: 4), wherein: Xaa₂ is: Ser, -Val, or dA; Xaa₁₂ is: Arg or Lys; Xaa₁₄ is: Arg, Leu, or Lys; Xaa₁₅ is: Lys, Ala, or Arg; Xaa₁₆ is: Gln, Lys, or Ala; Xaa₁₇ is: Met, Val, Ala, or Leu; Xaa₁₉ is: Val, Ala or Leu; Xaa₂₀ is: Lys, Gln, or Arg; Xaa₂₁ is: Lys or Arg; Xaa₂₄ is: Asn or Gln; Xaa₂₅ is: Ser, Phe, Ile, Leu, Thr, Val, Trp, Gln, Asn, or Tyr; Xaa₂₆ is: Ile, Leu, Thr, Val, Trp, or Tyr; Xaa₂₇ is: Leu, hR, Arg, or Lys; Xaa₂₉ is: Lys, Ser, Arg, or absent; Xaa₃₀ is: Arg, Lys, or absent; and Xaa₃₁ is: Tyr, Phe, or absent.

Another alternative preferred sequence for selective PEGylated VPAC2 receptor peptide agonists of the present invention comprises an amino acid sequence of the Formula 1 (SEQ ID NO: 4), wherein: Xaa₂ is: Val or dA; Xaa₁₄ is: Leu; Xaa₁₅ is: Ala; Xaa₁₆ is: Lys; Xaa₁₇ is: Ala; Xaa₂₀ is: Gln; Xaa₂₅ is: Phe, Ile, Leu, Val, Trp, or Tyr; Xaa₂₆ is: Thr, Trp, or Tyr; Xaa₂₇ is: hR; and Xaa₃₁ is: Phe.

Another alternative preferred sequence for selective PEGylated VPAC2 receptor peptide agonists of the present invention comprises an amino acid sequence of the Formula 1 (SEQ ID NO: 4), wherein: Xaa₂ is: Ser, Val, or dA; Xaa₁₂ is: Arg, Lys, hR, Orn, or Lys (isopropyl); Xaa₁₄ is: Arg, Leu, or Lys; Xaa₁₅ is: Lys, Ala, or Arg; Xaa₁₆ is:

Gln, Lys, or Ala; Xaa₁₇ is: Met, Val, Ala, or Leu; Xaa₁₈ is: Val, Ala, or Leu; Xaa₂₀ is: Lys, Gln, or Arg; Xaa₂₁ is: Lys or Arg; Xaa₂₄ is: Asn or Gln, Xaa₂₅ is: Ser, Phe, Ile, Leu, Val, Trp, Tyr, Thr, Gln, or Asn; Xaa₂₆ is: Ile, Thr, Trp, Tyr, Leu, or Val; Xaa₂₇ is: Leu, Lys, hR, or Arg; Xaa₂₉ is: Lys, Ser, Arg, hR, or absent; Xaa₃₀ is: Arg, Lys, or absent; and Xaa₃₁ is: Tyr, Phe, or absent.

Yet another alternative preferred sequence for selective PEGylated VPAC2 receptor peptide agonists of the present invention comprises an amino acid sequence of the Formula 1 (SEQ ID NO: 4), wherein: Xaa₁₄ is Leu when Xaa₁₅ is Ala and Xaa₁₆ is Lys. Even more preferably, Xaa₁₄ is Leu when Xaa₁₅ is Ala, Xaa₁₆ is Lys, Xaa₁₇ is Leu, and Xaa₂₀ is Gln.

Another alternative preferred peptide sequence for selective PEGylated VPAC2 receptor peptide agonists of the present invention comprises an amino acid sequence of the Formula 2 (SEQ ID NO: 5), provided that if Xaa₂₉ or Xaa₃₀ of Formula 2 is absent each amino acid downstream is absent and wherein the C-terminal amino acid may be amidated.

Another preferred alternative peptide sequence for selective PEGylated VPAC2 receptor peptide agonists of the present invention comprises an amino acid sequence of the Formula 3, (SEQ ID NO: 6), provided that if Xaa₂₉, Xaa₃₀, Xaa₃₁, Xaa₃₂; Xaa₃₃, Xaa₃₄, Xaa₃₅, Xaa₃₆, Xaa₃₇, Xaa₃₈, or Xaa₃₉ of Formula 3 is absent, the next amino acid present downstream is the next amino acid in the sequence and wherein the C-terminal amino acid may be amidated.

Preferred alternative peptide sequences for selective PEGylated VPAC2 receptor peptide agonists include:

!SEQ ID NO? Sequence

| | |
|---|---|
| 381. | HSDAVFTDNYTRLRKQMAVKKYLNSIKK-NH₂ |
| 382. | HSDAVFTDNYTRLRKQMAVKKYLNSIKKGGT |
| 383. | HSDAVFTENYTKLRKQLAAKKYLNDLLNGGT |
| 384. | HSDAVFTDNYTKLRKQLAAKKYLNDILNGGT |
| 385. | HSDAVFTENYTKLRKQLAAKKYLNDLKKGGTSWCEPGWCR |
| 386. | HSDAVFTDNYTRLRKQLAAKKYLNSIKKGGT |
| 387. | HSDAVFTDNYTRLRKQLAAKKYLNDIKNGGT |
| 388. | HSDAVFTDNYTRLRKQLAVKKYLNSIKKGGT |
| 389. | HSDAVFTDNYTRLRKQMAAKKYLNSIKKGGT |
| 390. | HSDAVFTDNYTRLRKQLAVKKYLNDIKNGGT |
| 391. | HSDAVFTDNYTRLRKQLAAKKYLNSIKNGGT |
| 392. | HSDAVFTDNYTRLRKQLAAKKYLNDIKKKRY |
| 393. | HSDAVFTDNYTRLRKQMAVKKYLNSIKK |
| 394. | HSDAVFTDNYTRLRKQMAVKKYLNSIKN |
| 395. | HSDAVFTDNYTRLRKQMAVKKYLNSILK |
| 396. | HSDAVFTDNYTELRKQMAVKKYLNSILN |
| 397. | HSDAVFTDNYTRLRKQMAVKKYLNDILN |
| 398. | HSDAVFTDNYTRLRKQMAAKKYLNSIKN |
| 399. | HSDAVFTDNYTRLRKQMAAKKYLNSILK |
| 400. | HSDAVFTDNYTRLRKQMAAKKYLNSIKK |
| 401. | HSDAVFTDNYTRLRKQMAAKKYLNSIKKKRY |
| 402. | HSDAVFTDNYTRLRKQMAAKKYLNSIKKKR |
| 403. | HSDAVFTDNYTRLRKQMAAKKYLNSIKKK |
| 404. | HSDAVFTDNYTRLRKQMAAKKYLNSIKNKRY |
| 405. | HSDAVFTDNYTRLRKQMAVKKYLNSIKKKRY |
| 406. | HSDAVFTDNYTRLRKQMAVKKYLNSIKKRK |
| 407. | HSDAVFTDNYTRLRKQMAVKKYLNSIKKK |
| 408. | HSDAVFTDNYTRLRKQMAVKKYLNSIKNKRY |
| 409. | HSDAVFTDNYTRLRKQVAAKKYLQSIKK |
| 410. | HSDAVFTDNYTRLRKQIAAKKYLQTIKK |
| 411. | HSDAVFTENYTRLRKQMAVKKYLNSLKK-NH₂ |
| 412. | HSDAVFTDNYTRLRKQLAAKKYLNDILKGGT |
| 413. | HSDAVFTDNYTRLRKQLAAKKYLNDILNGGT |
| 414. | HSDAVFTDNYTRLRKQLAVKKYLNDILKGGT |
| 415. | HSDAVFTDNYTRLRKQVAAKKYLNSIKK |
| 416. | HSDAVFTDNYTRLRKQMAAKKYLNSIKNKR |
| 417. | HSDAVFTDNYTRLRKQVAAKKYLQSIKNKRY |
| 418. | HSDAVFTDNYTRLRKQLAAKKYLNTIKNKRY |
| 419. | HSDAVFTDNYTRLRKQVAAKKYLNSIKNKRY |
| 420. | HSDAVFTDNYTRLRKQMAAKKYLQSIKNKRY |
| 421. | HSDAVFTDNYTRLRKQMAAKKYLNTIKNKRY |
| 422. | HSDAVFTDQYTRLRKQMAAKKYLNSIKNKRY |
| 423. | HSDAVFTDQYTRLRKQLAAKKYLNTIKNKRY |
| 424. | HSDAVFTDNYTRLRKQMAAHKYLNSIKNKRY |
| 425. | HSDAVFTDNYTRLRKQMAAKHYLNSIKNKRY |
| 426. | HSDAVFTDQYTRLRKQLAAHKYLNTIKNKRY |
| 427. | HSDAVFTDQYTRLRKQLAAKHYLNTIKNKRY |
| 428. | HSDAVFTDNYTRLRKQVAAKKYLQSIKKKR |
| 429. | HSDAVFTDNYTRLRKQVAAKKYLNSIKKKR |
| 430. | HSDAVFTDNYTRLRKQVAAKKYLNSIKNKRY |
| 431. | HSDAVFTDNYTRLRKQVAVKKYLQSIKKKR |
| 432. | HSDAVFTDNYTRLRKQVAVKKYLQSIKKK |
| 433. | HSDAVFTDNYTRLRKQVAVKKYLQSIKNKRY |
| 434. | HSDAVFTDNYTRLRKQVAAKKYLQSILKKRY |
| 435. | HSDAVFTDNYTRLRKQVAAKKYLQSILKKR |
| 436. | HSDAVFTDNYTRLRKQVAAKKYLQSILKK |
| 437. | HSDAVFTDNYTRLRKQVAAKKYLQSIKNK |
| 438. | HSDAVFTDNYTRLRKQVAVKKYLQSILKKRY |
| 439. | HSDAVFTDNYTRLRKQVAVKKYLQSILKKR |
| 440. | HSDAVFTDNYTRLRKQVAVKKYLQSILKK |
| 441. | HSDAVFTDNYTRLRKQVAVKKYLQSIKNK |
| 442. | HSDAVFTDNYTRLRKQVAAKKYLQSILNKRY |
| 443. | HSDAVFTDNYTRLRKQVAAKKYLQSILNKR |
| 444. | HSDAVFTDNYTRLRKQVAAKKYLQSILNK |
| 445. | HSDAVFTDNYTRLRKQMAEKKYLNSIKNKR |
| 446. | HSDAVFTDNYTRLRKQMAFKKYLNSIKNKR |
| 447. | HSDAVFTDNYTRLRKQMAGKKYLNSIKNKR |
| 448. | HSDAVFTDNYTRLRKQMAHKKYLNSIKNKR |
| 449. | HSDAVFTDNYTRLRKQMAIKKYLNSIKNKR |
| 450. | HSDAVFTDNYTRLRKQMAKKKYLNSIKNKR |
| 451. | HSDAVFTDNYTRLRKQMALKKYLNSIKNKR |
| 452. | HSDAVFTDNYTRLRKQMAMKKYLNSIKNKR |
| 453. | HSDAVFTDNYTRLRKQMANKKYLNSIKNKR |
| 454. | HSDAVFTDNYTRLRKQMAPKKYLNSIKNKR |
| 455. | HSDAVFTDNYTRLRKQMAQKKYLNSIKNKR |
| 456. | HSDAVFTDNYTRLRKQMARKKYLNSIKNKR |
| 457. | HSDAVFTDNYTRLRKQMASKKYLNSIKNKR |
| 458. | HSDAVFTDNYTRLRKQMATKKYLNSIKNKR |
| 459. | HSDAVFTDNYTRLRKQMAVKKYLNSIKNKR |
| 460. | HSDAVFTDNYTRLRKQMAWKKYLNSIKNKR |
| 461. | HSDAVFTDNYTRLRKQMAYKKYLNSIKNKR |
| 462. | HSDAVFTDNYTRLRKQMAAKKYLNSIANKR |
| 463. | HSDAVFTDNYTRLRKQMAAKKYLNSIDNKR |
| 464. | HSDAVFTDNYTRLRKQMAAKKYLNSIENKR |
| 465. | HSDAVFTDNYTRLRKQMAAKKYLNSIFNKR |
| 466. | HSDAVFTDNYTRLRKQMAAKKYLNSIGNKR |
| 467. | HSDAVFTDNYTRLRKQMAAKKYLNSIHNKR |
| 468. | HSDAVFTDNYTRLRKQMAAKKYLNSIINKR |
| 469. | HSDAVFTDNYTRLRKQMAAKKYLNSIMNKR |
| 470. | HSDAVFTDNYTRLRKQMAAKKYLNSINNKR |
| 471. | HSDAVFTDNYTRLRKQMAAKKYLNSIPNKR |
| 472. | HSDAVFTDNYTRLRKQMAAKKYLNSIQNKR |
| 473. | HSDAVFTDNYTRLRKQMAAKKYLNSIRNKR |
| 474. | HSDAVFTDNYTRLRKQMAAKKYLNSISNKR |
| 475. | HSDAVFTDNYTRLRKQMAAKKYLNSITNKR |
| 476. | HSDAVFTDNYTRLRKQMAAKKYLNSIVNKR |
| 477. | HSDAVFTDNYTRLRKQMAAKKYLNSTWNKR |
| 478. | HSDAVFTDNYTRLRKQMAAKKYLNSIYNKR |
| 479. | HSDAVFTDNYTRLRKQMAAKKYLNSIKNAR |
| 480. | HSDAVFTDNYTRLRKQMAAKKYLNSIKNDR |
| 481. | HSDAVFTDNYTRLRKQMAAKKYLNSIKKER |
| 482. | HSDAVFTDNYTRLRKQMAAKKYLNSIKNFR |
| 483. | HSDAVFTDNYTRLRKQMAAKKYLNSIKNGR |
| 484. | HSDAVFTDNYTRLRKQMAAKKYLNSIKNHR |
| 485. | HSDAVFTDNYTRLRKQMAAKKYLNSIKNIR |
| 486. | HSDAVFTDNYTRLRKQMAAKKYLNSIKNLR |
| 487. | HSDAVFTDNYTRLRKQMAAKKYLNSIKNMR |
| 488. | HSDAVFTDNYTRLRKQMAAKKYLNSIKNNR |
| 489. | HSDAVFTDNYTRLRKQMAAKKYLNSIKNPR |
| 490. | HSDAVFTDNYTRLRKQMAAKKYLNSIKNQR |
| 491. | HSDAVFTDNYTRLRKQMAAKKYLNSIKNRR |
| 492. | HSDAVFTDNYTRLRKQMAAKKYLNSIKNSR |
| 493. | HSDAVFTDNYTRLRKQMAAKKYLNSIKNTR |
| 494. | HSDAVFTDNYTRLRKQMAAKKYLNSIKNVR |
| 495. | HSDAVFTDNYTRLRKQMAAKKYLNSIKNWR |
| 496. | HSDAVFTDNYTRLRKQMAAKKYLNSIKNYR |
| 497. | HSDAVFTDNYTRLRKQMAAKKYLNSIKNKA |
| 498. | HSDAVFTDNYTRLRKQMAAKKYLNSIKNKD |
| 499. | HSDAVFTDNYTRLRKQMAAKKYLNSIKNKE |
| 500. | HSDAVFTDNYTRLRKQMAAKKYLNSIKNKF |
| 501. | HSDAVFTDNYTRLRKQMAAKKYLNSIKNKG |
| 502. | HSDAVFTDNYTRLRKQMAAKKYLNSIKNKH |
| 503. | HSDAVFTDNYTRLRKQMAAKKYLNSIKNKI |
| 504. | HSDAVFTDNYTRLRKQMAAKKYLNSIKNKK |
| 505. | HSDAVFTDNYTRLRKQMAAKKYLNSIKNKL |
| 506. | HSDAVFTDNYTRLRKQMAAKKYLNSIKNKM |
| 507. | HSDAVFTDNYTRLRKQMAAKKYLNSIKNKN |
| 508. | HSDAVFTDNYTRLRKQMAAKKYLNSIKNKP |
| 509. | HSDAVFTDNYTRLRKQMAAKKYLNSIKNKQ |
| 510. | HSDAVFTDNYTRLRKQMAAKKYLNSIKNKS |
| 511. | HSDAVFTDNYTRLRKQMAAKKYLNSIKNKT |
| 512. | HSDAVFTDNYTRLRKQMAAKKYLNSIKNKV |
| 513. | HSDAVFTDNYTRLRKQMAAKKYLNSIKNKW |
| 514. | HSDAVFTDNYTRLRKQMAAKKYLNSIKNKY |
| 515. | HSDAVFTDNYTRLRKQVAAKKYLQSIKNKRYSWCEPGWCR |
| 516. | HSDAVFTDDYTRLRKEVAAKKYLESIKDKRY |
| 517. | HSDAVFTDNYTRLRKQMAAKKYLNSIKNRI |
| 518. | HSDAVFTDNYTRLRKQMAGKKYLNSIKNRI |
| 519. | HSDAVFTDNYTRLRKQMAKKKYLNSIKNRI |
| 520. | HSDAVFTDNYTRLRKQMARKKYLNSIKNRI |
| 521. | HSDAVFTDNYTRLRKQMASKKYLNSIKNRI |
| 522. | HSDAVFTDNYTRLRKQMAAKKYLNSIPNRI |
| 523. | HSDAVFTDNYTRLRKQMAGKKYLNSIPNRI |
| 524. | HSDAVFTDNYTRLRKQMAKKKYLNSIPNRI |
| 525. | HSDAVFTDNYTRLRKQMARKKYLNSIPNRI |
| 526. | HSDAVFTDNYTRLRKQMASKKYLNSIPNRI |
| 527. | HSDAVFTDNYTRLRKQMAAKKYLNSIQNRI |
| 528. | HSDAVFTDNYTRLRKQMAGKKYLNSIQNRI |
| 529. | HSDAVFTDNYTRLRKQMAKKKYLNSIQNRI |
| 530. | HSDAVFTDNYTRLRKQMARKKYLNSIQNRI |
| 531. | HSDAVFTDNYTRLRKQMASKKYLNSIQNRI |
| 532. | HSDAVFTDNYTRLRKQMAAKKYLNSIRNRI |
| 533. | HSDAVFTDNYTRLRKQMAGKKYLNSIRNRI |
| 534. | HSDAVFTDNYTRLRKQMAKKKYLNSIRNRI |
| 535. | HSDAVFTDNYTRLRKQMARKKYLNSIRNRI |
| 536. | HSDAVFTDNYTRLRKQMASKKYLNSIRNRI |
| 537. | HSDAVFTENYTKLRKQLAAKKYLNDLKKGGT-NH₂ |
| 538. | HSDAVFTENYTKLRKQLAAKKYLNDLKKGGT |
| 539. | HSDAVFTENYTKLRKQLAAKKYLNDLKKGGT |
| 540. | HSDAVFTENYTKLRKQLAAKKYLNDLKK |
| 541. | HSDAVFTDNYTRLRKQLAAKKYLNDIKKGGT |
| 542. | HSDAVFTDNYTRLRKQLAAKKYLNDIKK-NH₂ |
| 543. | HSDAVFTDNYTRLRKQMAVKKYLNDLKKGGT |
| 544. | HSDAVFTDNYTRLRKQMAAKKYLNDIKKGGT |
| 545. | HSDAVFTDNYTRLRKQLAVKKYLNDIKKGGT |
| 546. | HSDAVFTDNYTRLRKQLAAKKYLNDIKKGG |
| 547. | HSDAVFTDNYTRLRKQLAAKKYLNDIKKG |
| 548. | HSDAVFTDNYTRLRKQLAAKKYLNDIKK |
| 549. | HSDAVFTDNYTRLRKQLAAKKYLNDIKKQ |
| 550. | HSDAVFTDNYTRLRKQLAAKKYLNDIKKNQ |
| 551. | HSDAVFTDNYTRLREQMAVKKYLNSILN |
| 552. | HSDAVFTDNYTRLRKQLAVKKYLNSILN |
| 553. | HSDAVFTDNYTRLRKQMAAKKYLNSILN |
| 554. | HSDAVFTENYTKLRKQLAAKKYLNDLKKGGT |
| 555. | HSDAVFTDNYTRLRKQMACKKYLNSIKNKR |
| 556. | HSDAVFTDNYTRLRKQMADKKYLNSIKNKR |
| 557. | HSDAVFTDNYTRLRKQMAAKKYLNSICNKR |
| 558. | HSDAVFTDNYTRLRKQMAAKKYLNSIKNCR |
| 559. | HSDAVFTDQYTRLRKQVAAKKYLQSIKQKRY |
| 560. | HSDAVFTDQYTRLRKQVAAKKYLQSIKQKRY |
| 561. | HSDAVFTDQYTRLRKQMAAKKYLQSIKQKRY |
| 562. | HSDAVFTDQYTRLRKQVAAKKYLQSIKQK |
| 563. | HTEAVFTDQYTRLRKQVAAKKYLQSIKQKRY |
| 564. | HSDAVFTDQYTRLRKQLAVKKYLQDIKQGGT |
| 565. | HSDAVFTDQYTRLRKQMAAKKYLQSIKQKR |
| 566. | HSDAVFTDQYTRLRKQLAAKKYLQTIKQKRY |
| 567. | HSDAVFTDQYTRLRKQMAAKKYLQTIKQKRY |
| 568. | HSDAVFTDQYTRLRKQMAAHKYLQSIKQKRY |
| 569. | HSDAVFTDQYTRLRKQMAAKHYLQSIKQKRY |
| 570. | HSDAVFTDQYTRLRKQMAGKKYLQSIKQKR |
| 571. | HSDAVFTDQYTRLRKQMAKKKYLQSIKQKR |
| 572. | HSDAVFTDQYTRLRKQMARKKYLQSIKQKR |
| 573. | HSDAVFTDQYTRLRKQMASKKYLQSIKQKR |
| 574. | HSDAVFTDQYTRLRKQMAAKKYLQSIPQKR |
| 575. | HSDAVFTDQYTRLRKQMAAKKYLQSIQQKR |
| 576. | HSDAVFTDQYTRLRKQMAAKKYLQSIRQKR |
| 577. | HSDAVFTDQYTRLRKQMAAKKYLQSIKQRR |
| 578. | HSDAVFTDQYTRLRKQMAAKKYLQSIKQKA |
| 579. | HSDAVFTDQYTRLRKQMAAKKYLQSIKQKF |
| 580. | HSDAVFTDQYTRLRKQMAAKKYLQSIKQKH |
| 581. | HSDAVFTDQYTRLRKQMAAKKYLQSIKQKI |
| 582. | HSDAVFTDQYTRLRKQMAAKKYLQSIKQKK |
| 583. | HSDAVFTDQYTRLRKQMAAKKYLQSIKQKL |
| 584. | HSDAVFTDQYTRLRKQMAAKKYLQSIKQKM |
| 585. | HSDAVFTDQYTRLRKQMAAKKYLQSIKQKP |
| 586. | HSDAVFTDQYTRLRKQMAAKKYLQSIKQKQ |
| 587. | HSDAVFTDQYTRLRKQMAAKKYLQSIKQKS |
| 588. | HSDAVFTDQYTRLRKQMAAKKYLQSIKQKT |
| 589. | HSDAVFTDQYTRLRKQMAAKKYLQSIKQKV |
| 590. | HSDAVFTDQYTRLRKQMAAKKYLQSIKQKW |
| 591. | HSDAVFTDQYTRLRKQMAAKKYLQSIKQKY |
| 592. | HSDAVFTDQYTRLRKQMAGKKYLQSIKQRI |
| 593. | HSDAVFTDQYTRLRKQMAKKKYLQSIKQRI |
| 594. | HSDAVFTDQYTRLRKQMASKKYLQSIKQRI |
| 595. | HSDAVFTDQYTRLRKQMAAKKYLQSIPQRI |
| 596. | HSDAVFTDQYTRLRKQMASKKYLQSIRQRI |
| 597. | HSDAVFTDNYTRLRKQVAAKKYLQSIKQKRY |
| 598. | HSDAVFTDNYTRLRKQVAAKKYLQSIKQKRY |
| 599. | HSDAVFTDNYTRLRKQMAAKKYLQSIKQKRY |
| 600. | HSDAVFTDNYTRLRKQVAAKKYLQSIKQK |
| 601. | HTEAVFTDNYTRLRKQVAAKKYLQSIKQKRY |
| 602. | HSDAVFTDNYTRLRKQLAVKKYLQDIKQGGT |
| 603. | HSDAVFTDNYTRLRKQMAAKKYLQSIKQKR |
| 604. | HSDAVFTDNYTRLRKQLAAKKYLQTIKQKRY |
| 605. | HSDAVFTDNYTRLRKQMAAKKYLQTIKQKRY |
| 606. | HSDAVFTDNYTRLRKQMAAHKYLQSIKQKRY |
| 607. | HSDAVFTDNYTRLRKQMAAKHYLQSIKQKRY |
| 608. | HSDAVFTDNYTRLRKQMAGKKYLQSIKQKR |
| 609. | HSDAVFTDNYTRLRKQMAKKKYLQSIKQKR |
| 610. | HSDAVFTDNYTRLRKQMARKKYLQSIKQKR |
| 611. | HSDAVFTDNYTRLRKQMASKKYLQSIKQKR |
| 612. | HSDAVFTDNYTRLRKQMAAKKYLQSIPQKR |
| 613. | HSDAVFTDNYTRLRKQMAAKKYLQSIQQKR |
| 614. | HSDAVFTDNYTRLRKQMAAKKYLQSIRQKR |
| 615. | HSDAVFTDNYTRLRKQMAAKKYLQSIKQRR |
| 616. | HSDAVFTDNYTRLRKQMAAKKYLQSIKQKA |
| 617. | HSDAVFTDNYTRLRKQMAAKKYLQSIKQKF |
| 618. | HSDAVFTDNYTRLRKQMAAKKYLQSIKQKH |
| 619. | HSDAVFTDNYTRLRKQMAAKKYLQSIKQKI |
| 620. | HSDAVFTDNYTRLRKQMAAKKYLQSIKQKK |
| 621. | HSDAVFTDNYTRLRKQMAAKKYLQSIKQKL |
| 622. | HSDAVFTDNYTRLRKQMAAKKYLQSIKQKM |
| 623. | HSDAVFTDNYTRLRKQMAAKKYLQSIKQKP |
| 624. | HSDAVFTDNYTRLRKQMAAKKYLQSIKQKQ |
| 625. | HSDAVFTDNYTRLRKQMAAKKYLQSIKQKS |
| 626. | HSDAVFTDNYTRLRKQMAAKKYLQSIKQKT |
| 627. | HSDAVFTDNYTRLRKQMAAKKYLQSIKQKV |
| 628. | HSDAVFTDNYTRLRKQMAAKKYLQSIKQKW |
| 629. | HSDAVFTDNYTRLRKQMAAKKYLQSIKQKY |
| 630. | HSDAVFTDNYTRLRKQMAGKKYLQSIKQRI |
| 631. | HSDAVFTDNYTRLRKQMAKKKYLQSIKQRI |
| 632. | HSDAVFTDNYTRLRKQMASKKYLQSIKQRI |
| 633. | HSDAVFTDNYTRLRKQMAAKKYLQSIPQRI |
| 634. | HSDAVFTDNYTRLRKQMASKKYLQSIRQRI |
| 635. | HSDAVFTDQYTRLRKQVAAKKYLQSIKNKRY |
| 636. | HTDAVFTDQYTRLRKQVAAKKYLQSIKNKRY |
| 637. | HSDAVFTDQYTRLRKQMAAKKYLQSIKNKRY |
| 638. | HSDAVFTDQYTRLRKQVAAKKYLQSIKNK |
| 639. | HTEAVFTDQYTRLRKQVAAKKYLQSIKNKRY |
| 640. | HSDAVFTDQYTRLRKQLAVKKYLQDIKNGGT |
| 641. | HSDAVFTDQYTRLRKQMAAKKYLQSIKNKR |
| 642. | HSDAVFTDQYTRLRKQLAAKKYLQTIKNKRY |
| 643. | HSDAVFTDQYTRLRKQMAAKKYLQTIKNKRY |
| 644. | HSDAVFTDQYTRLRKQMAAHKYLQSIKNKRY |
| 645. | HSDAVFTDQYTRLRKQMAAKHYLQSIKNKRY |
| 646. | HSDAVFTDQYTRLRKQMAGKKYLQSIKNKR |
| 647. | HSDAVFTDQYTRLRKQMAKKKYLQSIKNKR |
| 648. | HSDAVFTDQYTRLRKQMARKKYLQSIKNKR |
| 649. | HSDAVFTDQYTRLRKQMASKKYLQSIKNKR |
| 650. | HSDAVFTDQYTRLRKQMAAKKYLQSIPNKR |
| 651. | HSDAVFTDQYTRLRKQMAAKKYLQSIQNKR |
| 652. | HSDAVFTDQYTRLRKQMAAKKYLQSIRNKR |
| 653. | HSDAVFTDQYTRLRKQMAAKKYLQSIKNRR |
| 654. | HSDAVFTDQYTRLRKQMAAKKYLQSIKNKA |
| 655. | HSDAVFTDQYTRLRKQMAAKKYLQSIKNKF |
| 656. | HSDAVFTDQYTRLRKQMAAKKYLQSIKNKH |
| 657. | HSDAVFTDQYTRLRKQMAAKKYLQSIKNKI |
| 658. | HSDAVFTDQYTRLRKQMAAKKYLQSIKNKK |
| 659. | HSDAVFTDQYTRLRKQMAAKKYLQSIKNKL |
| 660. | HSDAVFTDQYTRLRKQMAAKKYLQSIKNKM |
| 661. | HSDAVFTDQYTRLRKQMAAKKYLQSIKNKP |
| 662. | HSDAVFTDQYTRLRKQMAAKKYLQSIKNKQ |
| 663. | HSDAVFTDQYTRLRKQMAAKKYLQSIKNKS |
| 664. | HSDAVFTDQYTRLRKQMAAKKYLQSIKNKT |
| 665. | HSDAVFTDQYTRLRKQMAAKKYLQSIKNKV |
| 666. | HSDAVFTDQYTRLRKQMAAKKYLQSIKNKW |
| 667. | HSDAVFTDQYTRLRKQMAAKKYLQSIKNKY |
| 668. | HSDAVFTDQYTRLRKQMAGKKYLQSIKNRI |
| 669. | HSDAVFTDQYTRLRKQMAKKKYLQSIKNRI |
| 670. | HSDAVFTDQYTRLRKQMASKKYLQSIKNRI |
| 671. | HSDAVFTDQYTRLRKQMAAKKYLQSIPNRI |
| 672. | HSDAVFTDQYTRLRKQMASKKYLQSIRNRI |
| 673. | HSDAVFYDQYTRLRKQVAAKKYLQSIKQKRYC |
| 674. | HTDAVFTDQYTRLRKQVAAKKYLQSIKQKRYC |
| 675. | HSDAVFTDQYTRLRKQMAAKKYLQSIKQKRYC |
| 676. | HSDAVFTDQYTRLRKQVAAKKYLQSIKQKC |
| 677. | HTEAVFTDQYTRLRKQVAAKKYLQSIKQKRYC |
| 678. | HSDAVFTDQYTRLRKQLAVKKYLQDIKQGGTC |
| 679. | HSDAVFTDQYTRLRKQMAAKKYLQSIKQKRC |
| 680. | HSDAVFTDQYTRLRKQLAAKKYLQTIKQKRYC |
| 681. | HSDAVFTDQYTRLRKQMAAKKYLQTIKQKRYC |
| 682. | HSDAVFTDQYTRLRKQMAAHKYLQSIKQKRYC |
| 683. | HSDAVFTDQYTRLRKQMAAKHYLQSIKQKRYC |
| 684. | HSDAVFTDQYTRLRKQMAGKKYLQSIKQKRC |
| 685. | HSDAVFTDQYTRLRKQMAKKKYLQSIKQKRC |
| 686. | HSDAVFTDQYTRLRKQMARKKYLQSIKQKRC |
| 687. | HSDAVFTDQYTRLRKQMASKKYLQSIKQKRC |
| 688. | HSDAVFTDQYTRLRKQMAAKKYLQSIPQKRC |
| 689. | HSDAVFTDQYTRLRKQMAAKKYLQSIQQKRC |
| 690. | HSDAVFTDQYTRLRKQMAAKKYLQSIRQKRC |
| 691. | HSDAVFTDQYTRLRKQMAAKKYLQSIKQRRC |
| 692. | HSDAVFTDQYTRLRKQMAAKKYLQSIKQKAC |
| 693. | HSDAVFTDQYTRLRKQMAAKKYLQSIKQKFC |
| 694. | HSDAVFTDQYTRLRKQMAAKKYLQSIKQKHC |
| 695. | HSDAVFTDQYTRLRKQMAAKKYLQSIKQKIC |
| 696. | HSDAVFTDQYTRLRKQMAAKKYLQSIKQKKC |
| 697. | HSDAVFTDQYTRLRKQMAAKKYLQSIKQKLC |
| 698. | HSDAVFTDQYTRLRKQMAAKKYLQSIKQKMC |
| 700. | HSDAVFTDQYTRLRKQMAAKKYLQSIKQKQC |
| 701. | HSDAVFTDQYTRLRKQMAAKKYLQSIKQKSC |
| 702. | HSDAVFTDQYTRLRKQMAAKKYLQSIKQKTC |
| 703. | HSDAVFTDQYTRLRKQMAAKKYLQSIKQKVC |
| 704. | HSDAVFTDQYTRLRKQMAAKKYLQSIKQKWC |
| 705. | HSDAVFTDQYTRLRKQMAAKKYLQSIKQKYC |
| 706. | HSDAVFTDQYTRLRKQMAGKKYLQSIKQRIC |
| 707. | HSDAVFTDQYTRLRKQMAKKKYLQSIKQRIC |
| 708. | HSDAVFTDQYTRLRKQMASKKYLQSIKQRIC |
| 709. | HSDAVFTDQYTRLRKQMAAKKYLQSIPQRIC |
| 710. | HSDAVFTDQYTRLRKQMASKKYLQSIRQRIC |

Another preferred alternative sequence for selective VPAC2 receptor peptide agonists of the present invention comprises an amino acid sequence of the Formula 4 (SEQ ID NO: 7), provided that if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈, or Xaa₃₉ of Formula 4 is absent, the next amino acid present downstream is the next amino acid in the sequence and wherein the C-terminal amino acid may be amidated.

Preferably, the C-terminal extension for an alternative embodiment of the present invention comprises an amino acid sequence of the Formula 5 (SEQ ID NO: 8), provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ of Formula 5 is absent, the next amino acid present downstream is the next amino acid in the C-terminal extension and wherein the C-terminal amino acid may be amidated. For example, if Xaa₁ is Gly and Xaa₂ is absent, the next amino acid bonded to Gly at position 1 is an amino acid listed for position 3 or, if position 3 is also absent, an amino acid listed for position 4 is bonded to Gly at position 1, and so forth. Additionally, for example, if Xaa₁ is Gly and Xaa₂ through Xaa₁₃ are absent, Gly may be the C-terminal amino acid and may be amidated.

Also, the C-terminal extension for an alternative embodiment of the present invention preferably comprises an amino acid sequence of the Formula 6 (SEQ ID NO: 9), provided that if Xaa₁, Xaa₂, Xaa₃, Xaa₄, Xaa₅, Xaa₆, Xaa₇, Xaa₈, Xaa₉, or Xaa₁₀ of Formula 6 is absent, the next amino acid present downstream is the next amino acid in the sequence and wherein the C-terminal amino acid may be amidated. For example, if Xaa₁ is Gly and Xaa₂ is absent, the next amino acid bonded to Gly at position 1 is an amino acid listed for position 3 or, if position 3 is also absent, an amino acid listed for position 4 is bonded to Gly at position 1, and so forth. Additionally, for example, if Xaa₁ is Gly and Xaa₂ through Xaa₁₁ are absent, Gly may be the C-terminal amino acid and may be amidated.

More preferably, the C-terminal extension of an alternative embodiment of the present invention includes the following sequences:

| | |
|---|---|
| SEQ ID # | Sequence |

| | |
|---|---|
| SEQ ID NO: 10 | GGPSSGAPPPS |
| SEQ ID NO: 11 | GGPSSGAPPPS-NH₂ |

Preferably, the C-terminal extension differs from SEQ ID NO: 10 or SEQ ID NO: 11 by no more than eight amino acids, still preferably by no more than seven amino acids, yet still preferably by no more than six amino acids, more preferably by no more than five amino acids, even more preferably by no more than four amino acids, still more preferably by no more than three amino acids, yet more preferably by no more than two amino acids, and most preferably by no more than one amino acid.

Another alternative more preferable C-terminal extension of the present invention can also include variants of these sequences, including:

| | |
|---|---|
| SEQ ID # | Sequence |

| | |
|---|---|
| SEQ ID NO: 12 | GGPSSGAPPS-NH2 |
| SEQ ID NO: 13 | GGPSSGAPPPS-OH |

These sequences contain the standard single letter codes for the twenty naturally occurring amino acids. SEQ ID NO: 11 and SEQ ID NO: 12 contain sequences that are amidated at the C-terminus of the sequence.

Preferably, the C-terminal extension differs from SEQ ID NO: 12, or SEQ ID NO: 13 by no more than eight amino acids, still preferably by no more than seven amino acids, yet still preferably by no more than six amino acids, more preferably by no more than five amino acids, even more preferably by no more than four amino acids, still more preferably by no more than three amino acids, yet more preferably by no more than two amino acids, and most preferably by no more than one amino acid.

Another alternative preferred C-terminal extension of the present invention comprises as amino acid sequence of the Formula 7 (SEQ ID NO: 15), provided that if Xaa₁, Xaa₂, Xaa₃, Xaa₄, Xaa₅, Xaa₆, Xaa₇, Xaa₈, Xaa₉, or Xaa₁₀ of Formula 7 is absent, the next amino acid present downstream is the next amino acid in the C-terminal extension and wherein the C-terminal amino acid may be amidated.

Another alternative preferred C-terminal extension of the present invention includes (Lys), or (Glu), wherein n is the number of lysine or glutamic acid residues added to the C-terminus and wherein n can be anywhere from one to eight residues.

The following alternative selective PEGylated VPAC2 receptor peptide agonists are preferred:

| | SEQ | |
|---|---|---|
| Agonist | ID | |
| # | NO: | Sequence |
| PEG-P164 | 711 | C6-HSDAVFTDNYTRLLAKLALQKYLQSIKNKRYGGPSSGAPPPC(PEG) |
| PEG-P120 | 712 | C6-HSDAVFTDNYTRLRKQVAAKKYLQSIKNKRYGGPSSGAPPPC(PEG) |
| PEG-P119a | 713 | C6-HSDAVFTDNYTRLLAK(PEG)LALQRYLQSIRNKRYGGPSSGAPPPS |
| PEG-P119b | 714 | C6-HSDAVFTDNYTRLLAKLALQK(PEG)YLQSIRNKRYGGPSSGAPPPS |
| PEG-P31a | 715 | C6-HSDAVFTDNYTRLRK(PEG)QVAAKKYLQSIKNKRYGGPSSGAPPPS |
| PEG-P31b | 716 | C6-HSDAVFTDNYTRLRRQVAAK(PEG)RYLQSIKNKRYGGPSSGAPPPS |
| PEG-P31c | 717 | C6-HSDAVFTDNYTRLRRQVAARK(PEG)YLQSIRNKRYGGPSSGAPPPS |
| PEG-P31d | 718 | C6-HSDAVFTDNYTRLRK(PEG)QVAAK(PEG)RYLQSIRNKRYGGPSSGAPPPS |
| PEG-P213 | 719 | C₆-HSDAVFTDNYTRLRK(PEG)QVAAK(PEG)RYLQSIRNGGPSSGAPPPS |
| PEG-P214 | 720 | C6-HSDAVFTDNYTRLRK(PEG)QVAARRYLQSLRNGGPSSGAPPPS |
| PEG-P215 | 721 | C6-HSDAVFTDNYTRLRRQVAAK(PEG)RYLQSIRNGGPSSGAPPPS |
| PEG-P216 | 24 | C6-HSDAVFTDNYTRLRRQVAAK(PEG)YLQSIRNGGPSSGAPPPS |
| PEG-P201 | 25 | 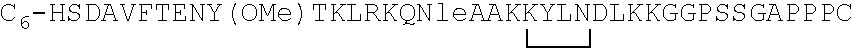 |

PEGylation of proteins may overcome many of the pharmacological and toxicological/immunological problems associated with using peptides or proteins as therapeutics. However, for any individual peptide it is uncertain whether the PEGylated form of the peptide will have significant loss in bioactivity as compared to the unPEGylated form of the peptide.

The bioactivity of PEGylated proteins can be affected by factors such as: i) the size of the PEG molecule; ii) the particular sites of attachment; iii) the degree of modification; iv) adverse coupling conditions; v) whether a linker is used for attachment or whether the polymer is directly attached; vi) generation of harmful co-products; vii) damage inflicted by the activated polymer; or viii) retention of charge. Work performed on the PEGylation of cytokines, for example, shows the effect PEGylation may have. Depending on the coupling reaction used, polymer modification of cytokines has resulted in dramatic reductions in bioactivity. [Francis, G. E., et al., (1998) PEGylation of cytokines and other therapeutic proteins and peptides: the importance of biological optimization of coupling techniques, Intl. J. Hem. 68:1-18]. Maintaining the bioactivity of PEGylated peptides is even more problematic than for proteins. As peptides are smaller than proteins, modification by PEGylation may potentially have a greater effect on bioactivity.

The VPAC2 receptor peptide agonists of the present invention are modified by the covalent attachment of one or more molecules of a polyethylene glycol (PEG) and generally have improved pharmacokinetic profiles due to slower proteolytic degradation and renal clearance. Attachment of PEG molecule(s) (PEGylation) will increase the apparent size of the VPAC2 receptor peptide agonists, thus reducing renal filtration and altering biodistribution. PEGylation can shield antigenic epitopes of the VPAC2 receptor peptide agonists, thus reducing reticuloendothelial clearance and recognition by the immune system and also reducing degradation by proteolytic enzymes, such as DPP-IV.

Covalent attachment of one or more molecules of polyethylene glycol to a small, biologically active VPAC2 receptor peptide agonist poses the risk of adversely affecting the agonist, for example, by destabilising the inherent secondary structure and bioactive conformation and reducing bioactivity, so as to make the agonist unsuitable for use as a therapeutic. The present invention, however, is based on the finding that covalent attachment of one or more molecules of PEG to particular residues of a VPAC2 receptor peptide agonist surprisingly results in a biologically active, PEGylated VPAC2 receptor peptide agonist with an extended half-life and reduced clearance when compared to that of non PEGylated VPAC2 receptor peptide agonists. The compounds of the present invention include selective PEGylated VPAC2 receptor peptide agonists.

In order to determine the potential PEGylation sites in a VPAC2 receptor peptide agonist, serine scanning may be conducted. A Ser residue is substituted at a particular position in the peptide and the Ser-modified peptide is tested for potency and selectivity. If the Ser substitution has minimal impact on potency and the Ser-modified peptide is selective for the VPAC2 receptor, the Ser residue is then substituted for a Cys or Lys residue, which serves as a direct or indirect PEGylation site. Indirect PEGylation of a residue is the PEGylation of a chemical group or residue which is bonded to the PEGylation site residue. Indirect PEGylation of Lys includes PEGylation of K(W) and K(CO(CH₂)₂SH).

The invention described herein provides VPAC2 receptor peptide agonists covalently attached to one or more molecules of polyethylene glycol (PEG), or a derivative thereof wherein each PEG is attached to a Cys or Lys amino acid, to a K(W) or a K(CO(CH₂)₂SH), or to the carboxy terminal amino acid of the peptide agonist. PEGylation can enhance the half-life of the selective VPAC2 receptor peptide agonists, resulting in PEGylated VPAC2 receptor peptide agonists with an elimination half-life of at least one hour, preferably at least 3, 5, 7, 10, 15, 20, or 24 hours and most preferably at least 48 hours. The PEGylated VPAC2 receptor peptide agonists of the present invention preferably have a clearance value of 200 ml/h/kg or less, more preferably 180, 150, 130, 100, 80, 60 ml/h/kg or less and most preferably less than 50, 40 or 20 ml/h/kg.

The present invention encompasses the discovery that specific amino acids added to the C-terminus of a peptide sequence for a PEGylated VPAC2 receptor peptide agonist provide features that may protect the peptide as well as may enhance activity, selectivity, and/or potency. For example, these C-terminal extensions may stabilize the helical structure of the peptide and sites within the peptide prone to enzymatic cleavage that are located near the C-terminus. Furthermore, many of the C-terminally extended peptides disclosed herein may be more selective for the VPAC2 receptor and can be more potent than VIP, PACAP, and other known VPAC2 receptor peptide agonists. An example of a preferred C-terminal extension is the extension peptide of exendin-4 as the C-capping sequence. Exendin-4 is found in the salivary excretions from the Gila Monster, Heloderma Suspectum, (Eng et al., J. Biol. Chem., 267(11):7402-7405 (1992)).

It has furthermore been discovered that modification of the N-terminus of the VPAC2 receptor peptide agonist may enhance potency and/or provide stability against DPP-IV cleavage.

VIP and some known VPAC2 receptor peptide agonists are susceptible to cleavage by various enzymes and, thus, have a short in vivo half-life. Various enzymatic cleavage sites in the VPAC2 receptor peptide agonists are discussed below. The cleavage sites are discussed relative to the amino acid positions in VIP (SEQ ID NO: 1), and are applicable to the sequences noted herein.

Cleavage of the peptide agonist by the enzyme dipeptidyl-peptidase-IV (DPP-IV) occurs between position 2 (serine in VIP) and position 3 (aspaitic acid in VIP). The addition of a N-terminal modification and/or various substitutions at position 2 may improve stability against DPP-IV cleavage. Examples of amino acids at position 2 that may improve stability against DPP-IV inactivation preferably include valine, D-alanine, or D-serine. More preferably, position 2 is valine or D-alanine. Examples of N-terminal modifications that may improve stability against DPP-IV inactivation include the addition of acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid and —C(═NH)—NH₂. Preferably, the N-terminal modification is the addition of acetyl or hexanoyl. There are chymosrypsin cleavage sites in wild-type VIP between the amino acids 10 and 11 (tyrosine and threonine) and those at 22 and 23 (tyrosine and leucine). Making substitutions at position 10 and/or 11 and position 22 and/or 23 may increase the stability of the peptide at these sites.

There is also a trypsin cleavage site between arginine at position 12 and leucine at position 13 of wild-type VIP. Examples of substitutions which render the peptide resistant to cleavage by trypsin at this site include substitution of the arginine at position 12 with ornithine and substitution of leucine at position 13 with amino isobutyric acid.

In wild-type VIP, and in numerous VPAC2 receptor peptide agonists known in the art, there are cleavage sites between the basic amino acids at positions 14 and 15 and between those at positions 20 and 21. The selective VPAC2 receptor agonists of the present invention generally have improved proteolytic stability in vivo due to substitutions in these sites. These substitutions can render the peptide resistant to cleavage by trypsin-like enzymes, including trypsin. Examples of amino acids at position 14 that confer some resistance to cleavage by trypsin-like enzymes alone or in combination with the amino acids specified for position 15 below include glutamine, amino isobutyric acid, homoarginine, ornithine, citrulline, lysine, alanine and leucine. Also, position 14 may be arginine when position 15 is an amino acid other than lysine. Also, position 14 can be arginine when position 15 is lysine, but this specific combination does not address enzymatic cleavage. Examples of amino acids at position 15 that confer some resistance to cleavage by trypsin-like enzymes alone or in combination with amino acids specified above for position 14 include amino isobutyric acid, ornithine and arginine. Also, position 15 may be lysine when position 14 is an amino acid other than arginine. Also, position 15 can be lysine when position 14 is arginine, but this specific combination does not address enzymatic cleavage. Examples of amino acids at position 20 that confer some resistance to cleavage by trypsin-like enzymes alone or in combination with amino acids specified for position 21 include valine, leucine, amino isobutyric acid, alanine, glutamine, and arginine. Also, position 20 may be lysine when position 21 is an amino acid other than lysine. Also, position 20 can be lysine when position 21 is lysine, but this specific combination does not address enzymatic cleavage. An example of an amino acid at position 21 that confers some resistance to cleavage by trypsin-like peptides alone or in combination with amino acids specified for position 20 include amino isobutyric acid, ornithine, alanine, glutamino, or arginine. Also position 21 may be lysine when position 20 is an amino acid other than lysine. Also, position 21 can be lysine when position 20 is lysine, but this specific combination does not address enzymatic cleavage. The improved stability of a representative number of selective PEGylated VPAC2 receptor peptide agonists with resistance to peptidase cleavage and encompassed by the present invention is demonstrated in Example 6.

The bond between the amino acids at positions 25 and 26 of wild-type VIP is susceptible to enzymatic cleavage. This cleavage site can be completely or partially eliminated through substitution of the amino acid at position 25 and/or the amino acid at position 26. Examples of amino acids at position 25 that confer at least some resistance to enzymatic cleavage include phenylalanine, isoleucine, leucine, threonine, valine, tryptophan, glutamine, asparagine, tyrosine, or amino isobutyric acid. Also, position 25 may be serine when position 26 is an amino acid other than isoleucine. Also, position 25 can be serine when position 26 is isoleucine, but this specific combination does not address enzymatic cleavage. Examples of amino acids at position 26 that confer at least some resistance to enzymatic cleavage alone or in combination with the amino acids specified above for position 25 include leucine, threonine, valine, tryptophan, tyrosine, phenylalanine, or amino isobutyric acid. Also, position 26 may be isoleucine when position 25 is an amino acid other than serine. Also, position 26 can be isoleucine when position 25 is serine, but this specific combination does not address enzymatic cleavage.

The region of the VPAC2 receptor peptide agonist encompassing the amino acids at positions 27, 28, 29, 30 and 31 is also susceptible to enzyme cleavage. The addition of a C-terminal extension peptide may render the peptide agonist more stable against neutroendopeptidase (NEP). The addition of the extension peptide may also increase selectivity for the VPAC2 receptor. Trypsin-like enzymes may also attack these positions. If that occurs, the peptide agonist may lose its C-terminal extension with the additional carboxypeptidase activity leading to an inactive form of the peptide.

In addition to selective VPAC2 receptor peptide agonists with resistance to cleavage by various peptidases, the selective PEGylated VPAC2 peptide receptor agonists of the present invention may also encompass peptides with enhanced selectivity for the VPAC2 receptor, increased potency, and/or increased stability compared with some peptides known in the art. Examples of amino acid positions that may affect such properties include positions: 3, 8, 12, 14, 15, 16, 17, 20, 21, 27, 28, and 29 of Formula 10, 12, or 13. For example, the amino acid at position 3 is preferably aspartic acid or glutamic acid; the amino acid at position 8 is preferably aspartic acid or glutamic acid; the amino acid at position 12 is preferably arginine, homoarginine, ornithine, or lysine; the amino acid at position 14 is preferably arginine, glutamine, amino isobutyric acid, homoarginine, ornithine, citrulline, lysine, alanine, or leucine; the amino acid at position 15 is preferably lysine, amino isobutyric acid, ornithine, or arginine; the amino acid at position 16 is preferably glutamine or lysine; the amino acid at position 17 is preferably valine, alanine, leucine, isoleucine, lysine, or norleucine; the amino acid at position 20 is preferably lysine, valine, leucine, amino isobutyric acid, alanine, glutamine, or arginine; the amino acid at position 21 is preferably lysine, amino isobutyric acid, ornithine, alanine, glutamine, or arginine; the amino acid at position 27 is preferably lysine, ornithine, homoarginine, or arginine; the amino acid at position 28 is preferably asparagine, glutamine, lysine, homoarginine, amino isobutyric acid, proline, or ornithine; and, if present, the amino acid at position 29 is preferably lysine, ornithine, or homoarginine. Preferably, these amino acid substitutions may be combined with substitutions at positions that affect the five aforementioned regions susceptible to cleavage by various enzymes.

The increased potency and selectivity for various VPAC2 receptor peptide agonists of the present invention is demonstrated in Examples 3 and 4. For example, Table 1 in Example 3 provides a list of selective PEGylated VPAC2 receptor peptide agonists and their corresponding in vitro potency results. Preferably, the selective PEGylated VPAC2 receptor peptide agonists of the present invention have an EC₅₀ value less than 200 nM. More preferably, the EC₅₀ value is less than 50 nM. Even more preferably, the EC₅₀ value is less than 30 nM. Still more preferably, the EC₅₀ value is less than 10 nM.

Table 2 in Example 4 provides a list of VPAC2 receptor peptide agonists and their corresponding selectivity results for human VPAC2, VPAC1, and PAC1. See Example 4 for further details of these assays. These results are provided as a ratio of VPAC2 binding affinity to VPAC1 binding affinity and as a ratio of VPAC2 binding affinity to PAC1 binding affinity. Preferably, the agonists of the present invention have a selectivity ratio where the affinity for VPAC2 is at least 50 times greater than for VPAC1 and/or for PAC1. More preferably, the affinity is at least 100 times greater than for VPAC1 and/or for PAC1. Even more preferably, the affinity is at least 200 times greater than for VPAC1 and/or for PAC1. Still more preferably, the affinity is at least 500 times greater than for VPAC1 and/or for PAC1. Yet more preferably, the affinity is at least 1000 times greater than for VPAC1 and/or for PAC1.

As used herein, “selective VPAC2 receptor peptide agonists” also include pharmaceutically acceptable salts of the compounds described herein. A selective VPAC2 receptor peptide agonist of this invention can possess a sufficiently acidic, a sufficiently basic, or both functional groups, and accordingly react with any of a number of inorganic bases, and inorganic and organic acids, to form a salt. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, trifluoroacetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

The selective PEGylated VPAC2 receptor peptide agonists of the present invention can be administered parenterally. Parenteral administration can include, for example, systemic administration, such as by intramuscular, intravenous, subcutaneous, intradermal, or intraperitoneal injection. These agonists can be administered to the subject in conjunction with an acceptable pharmaceutical carrier, diluent, or excipient as part of a pharmaceutical composition for treating NIDDM, or the disorders discussed below. The pharmaceutical composition can be a solution or, if administered parenterally, a suspension of the VPAC2 receptor peptide agonist or a suspension of the VPAC2 receptor peptide agonist complexed with a divalent metal cation such as zinc. Suitable pharmaceutical carriers may contain inert ingredients which do not interact with the peptide or peptide derivative. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Some examples of suitable excipients include lactose, dextrose, sucrose, trehalose, sorbitol, and mannitol.

Standard pharmaceutical formulation techniques may be employed such as those described in Remington's Pharmaceutical Sciences, Mack Publishing Company, Easton, Pa. The selective PEGylated VPAC2 receptor peptide agonists of the present invention may be formulated for administration through the buccal, topical, oral, transdermal, nasal, or pulmonary route.

The PEGylated VPAC2 receptor peptide agonists of the invention may be formulated for administration such that blood plasma levels are maintained in the efficacious range for extended time periods. The main barrier to effective oral peptide drug delivery is poor bioavailability due to degradation of peptides by acids and enzymes, poor absorption through epithelial membranes, and transition of peptides to an insoluble form after exposure to the acidic pH environment in the digestive tract. Oral delivery systems for peptides such as those encompassed by the present invention are known in the art. For example, PEGylated VPAC2 receptor peptide agonists can be encapsulated using microspheres and then delivered orally. For example, PEGylated VPAC2 receptor peptide agonists can be encapsulated into microspheres composed of a commercially available, biocompatible, biodegradable polymer, poly(lactide-co-glycolide)-COOH and olive oil as a filler (see Joseph, et al. Diabetologia 43:1319-1328 (2000)). Other types of microsphere technology is also available commercially such as Medisorb® and Prolease® biodegradable polymers from Alkermas. Medisorb® polymers can be produced with any of the lactide isomers. Lactide:glycolide ratios can be varied between 0:100 and 100:0 allowing for a broad range of polymer properties. This allows for the design of delivery systems and implantable devices with resorption times ranging from weeks to months. Emisphere has also published numerous articles discussing oral delivery technology for peptides and proteins. For example, see WO 95/28838 by Leone-bay et al. which discloses specific carriers comprised of modified amino acids to facilitate absorption.

The selective PEGylated VPAC2 receptor peptide agonists described herein can be used to treat subjects with a wide variety of diseases and conditions. Agonists encompassed by the present invention exert their biological effects by acting at a receptor referred to as the VPAC2 receptor. Subjects with diseases and/or conditions that respond favourably to VPAC2 receptor stimulation or to the administration of VPAC2 receptor peptide agonists can therefore be treated with the VPAC2 agonists of the present invention. These subjects are said to “be in need of treatment with VPAC2 agonists” or “in need of VPAC2 receptor stimulation”.

The selective PEGylated VPAC2 receptor peptide agonists of the present invention may be employed to treat diabetes, including both type 1 and type 2 diabetes (non-insulin dependent diabetes mellitus or NIDDM). Also included are subjects requiring prophylactic treatment with a VPAC2 receptor agonist, e.g., subjects at risk for developing NIDDM. Such treatment may also delay the onset of diabetes and diabetic complications. Additional subjects include those with impaired glucose tolerance or impaired fasting glucose, subjects whose body weight is about 25% above normal body weight for the subject's height and body build, subjects having one or more parents with NIDDM, subjects who have had gestational diabetes, and subjects with metabolic disorders such as those resulting from decreased endogenous insulin secretion. The selective VPAC2 receptor peptide agonists may be used to prevent subjects with impaired glucose tolerance from proceeding to develop type 2 diabetes, prevent pancreatic β-cell deterioration, induce β-cell proliferation, improve β-cell function, activate dormant β-cells, differentiate cells into β-cells, stimulate P-cell replication, and inhibit β-cell apoptosis. Other diseases and conditions that may be treated or prevented using compounds of the invention in methods of the invention include: Maturity-Onset Diabetes of the Young (MODY) (Herman, et al., Diabetes 43:40, 1994); Latent Autoimmune Diabetes Adult (LADA)(Zimmet, et al., Diabetes Med. 11:299, 1994); impaired glucose tolerance (IGT) (Expert Committee on Classification of Diabetes Mellitus, Diabetes Care 22 (Supp. 1):S5, 1999); impaired fasting glucose (IFG) (Charles, et al., Diabetes 40:796, 1991); gestational diabetes (Metzger, Diabetes, 40:197, 1991); metabolic syndrome X, dyslipidemia, hyperglycemia, hyperinsulinemia, hypertriglyceridemia, and insulin resistance.

The selective PEGylated VPAC2 receptor peptide agonists of the invention may also be used in methods of the invention to treat secondary causes of diabetes (Expert Committee on Classification of Diabetes Mellitus, Diabetes Care 22 (Supp. 1):S5, 1999). Such secondary causes include glucocorticoid excess, growth hormone excess, pheochromocytoma, and drug-induced diabetes. Drugs that may induce diabetes include, but are not limited to, pyriminil, nicotinic acid, glucocorticoids, phenyloin, thyroid hormone, β-adrenergic agents, α-interferon and drugs used to treat HIV infection.

The selective PEGylated VPAC2 receptor peptide agonists of the present invention may be effective in the suppression of food intake and the treatment of obesity.

The selective PEGylated VPAC2 receptor peptide agonists of the present invention may also be effective in the prevention or treatment of such disorders as atherosclerotic disease, hyperlipidemia, hypercholesteremia, low HDL levels, hypertension, primary pulmonary hypertension, cardiovascular disease (including atherosclerosis, coronary heart disease, coronary artery disease, and hypertension), cerebrovascular disease and peripheral vessel disease; and for the treatment of lupus, polycystic ovary syndrome, carcinogenesis, and hyperplasia, asthma, male and female reproduction problems, sexual disorders, ulcers, sleep disorders, disorders of lipid and carbohydrate metabolism, circadian dysfunction, growth disorders, disorders of energy homeostasis, immune diseases including autoimmune diseases (e.g., systemic lupus erythematosus), as well as acute and chronic inflammatory diseases, rheumatoid arthritis, and septic shock.

The selective PEGylated VPAC2 receptor peptide agonists of the present invention may also be useful for treating physiological disorders related to, for example, cell differentiation to produce lipid accumulating cells, regulation of insulin sensitivity and blood glucose levels, which are involved in, for example, abnormal pancreatic β-cell function, insulin secreting tumors and/or autoimmune hypoglycemia due to autoantibodies to insulin, autoantibodies to the insulin receptor, or autoantibodies that are stimulatory to pancreatic β-cells, macrophage differentiation which leads to the formation of atherosclerotic plaques, inflammatory response, carcinogenesis, hyperplasia, adipocyte gene expression, adipocyte differentiation, reduction in the pancreatic β-cell mass, insulin secretion, tissue sensitivity to insulin, liposarcoma cell growth, polycystic ovarian disease, chronic anovulation, hyperandrogenism, progesterone production, steroidogenesis, redox potential and oxidative stress in cells, nitric oxide synthase (NOS) production, increased gamma glutamyl transpeptidase, catalase, plasma triglycerides, HDL, and LDL cholesterol levels, and the like.

In addition, the selective VPAC2 receptor peptide agonists of the invention may be used for treatment of asthma (Bolin, et al., Biopolymer 37:57-66 (1995); U.S. Pat. No. 5,677,419; showing that polypeptide R3PO is active in relaxing guinea pig tracheal smooth muscle); hypotension induction (VIP induces hypotension, tachycardia, and facial flushing in asthmatic patients (Morice, et al., Peptides 7:279-280 (1986); Morice, et al., Lancet 2:1225-1227 (1983)); male reproduction problems (Siow, et al., Arch. Androl. 43(1):67-71 (1999)); as an anti-apoptosis/neuroprotective agent (Brenneman, et al., Ann. N.Y. Acad. Sci. 865:207-12 (1998)); cardioprotection during ischemic events (Kalfin, et al., J. Pharmacol. Exp. Ther. 1268(2):952-8 (1994); Das, et al., Ann. N.Y. Acad. Sci. 865:297-308 (1998)), manipulation of the circadian clock and its associated disorders (Hamar, et al., Cell 109:497-508 (2002); Shen, et al., Proc. Natl. Acad. Sci. 97:11575-80, (2000)), and as an anti-ulcer agent (Tuncel, et al., Ann. N.Y. Acad. Sci. 865:309-22, (1998)).

An “effective amount” of a selective PEGylated VPAC2 receptor peptide agonist is the quantity that results in a desired therapeutic and/or prophylactic effect without causing unacceptable side effects when administered to a subject in need of VPAC2 receptor stimulation. A “desired therapeutic effect” includes one or more of the following: 1) an amelioration of the symptom(s) associated with the disease or condition; 2) a delay in the onset of symptoms associated with the disease or condition; 3) increased longevity compared with the absence of the treatment; and 4) greater quality of life compared with the absence of the treatment. For example, an “effective amount” of a VPAC2 agonist for the treatment of NIDDM is the quantity that would result in greater control of blood glucose concentration than in the absence of treatment, thereby resulting in a delay in the onset of diabetic complications such as retinopathy, neuropathy, or kidney disease. An “effective amount” of a selective VPAC2 receptor peptide agonist for the prevention of NIDDM is the quantity that would delay, compared with the absence of treatment, the onset of elevated blood glucose levels that require treatment with anti-hypoglycemic drugs such as sulfonylureas, thiazolidinediones, insulin, and/or bisguanidines.

An “effective amount” of the selective PEGylated VPAC2 receptor peptide agonist administered to a subject will also depend on the type and severity of the disease and on the characteristics of the subject, such as general health, age, sex, body weight and tolerance to drugs. The dose of selective VPAC2 peptide receptor agonist effective to normalize a patient's blood glucose will depend on a number of factors, among which are included, without limitation, the subject's sex, weight and age, the severity of inability to regulate blood glucose, the route of administration and bioavailability, the pharmacokinetic profile of the peptide, the potency, and the formulation.

A typical dose range for the selective PEGylated VPAC2 receptor peptide agonists of the present invention will range from about 1 μg per day to about 5000 μg per day. Preferably, the dose ranges from about 1 μg per day to about 2500 μg per day, more preferably from about 1 μg per day to about 1000 μg per day. Even more preferably, the dose ranges from about 5 μg per day to about 100 μg per day. A further preferred dose range is from about 10 μg per day to about 50 μg per day. Most preferably, the dose is about 20 μg per day.

A “subject” is a mammal, preferably a human, but can also be an animal, e.g., companion animals (e.g., dogs, cats, and the like), farm animals (e.g., cows, sheep, pigs, horses, and the like) and laboratory animals (e.g., rats, mice, guinea pigs, and the like).

The selective VPAC2 receptor peptide agonists of the present invention can be prepared by using standard methods of solid-phase peptide synthesis techniques. Peptide synthesizers are commercially available from, for example, Rainin-PTI Symphony Peptide Synthesizer (Tucson, Ariz.). Reagents for solid phase synthesis are commercially available, for example, from Glycopep (Chicago, Ill.). Solid phase peptide synthesizers can be used according to manufacturers instructions for blocking interfering groups, protecting the amino acid to be reacted, coupling, decoupling, and capping of unreacted amino acids.

Typically, an ic-N-protected amino acid and the N-terminal amino acid on the growing peptide chain on a resin is coupled at room temperature in an inert solvent such as dimethylformamide, N-methylpyrrolidone or methylene chloride in the presence of coupling agents such as dicyclohexylcarbodiimide and 1-hydroxybenzotriazole and a base such as diisopropylethylamine. The α-N-protecting group is removed from the resulting peptide resin using a reagent such as trifluoroacetic acid or piperidine, and the coupling reaction repeated with the next desired N-protected amino acid to be added to the peptide chain. Suitable amine protecting groups are well known in the art and are described, for example, in Green and Wuts, “Protecting Groups in Organic Synthesis”, John Wiley and Sons, 1991. Examples include t-butyloxycarbonyl (tBoc) and fluorenylmethoxycarbonyl (Fmoc).

The selective VPAC2 receptor peptide agonists are also synthesized using standard automated solid-phase synthesis protocols using t-butoxycarbonyl- or fluorenylmethoxycarbonyl-alpha-amino acids with appropriate side-chain protection. After completion of synthesis, peptides are cleaved from the solid-phase support with simultaneous side-chain deprotection using standard hydrogen fluoride methods or trifluoroacetic acid (TFA). Crude peptides are then further purified using Reversed-Phase Chromatography on Vydac C18 columns using acetonitrile gradients in 0.1% trifluoroacetic acid (TFA). To remove acetonitrile, peptides are lyophilized from a solution containing 0.1% TFA, acetonitrile and water. Purity can be verified by analytical reversed phase chromatography. Identity of peptides can be verified by mass spectrometry. Peptides can be solubilized in aqueous buffers at neutral pH.

The peptide agonists of the present invention may also be made by recombinant methods known in the art using both eukaryotic and prokaryotic cellular hosts.

Once a peptide for use in the present invention is prepared and purified, it is modified by covalently linking at least one PEG molecule to Cys or Lys residues, to K(W) or K(CO(CH₂)₂SH), or to the carboxy-terminal amino acid. A wide variety of methods have been described in the art to produce peptides covalently conjugated to PEG and the specific method used for the present invention is not intended to be limiting (for review article see, Roberts, M. et al. Advanced Drug Delivery Reviews, 54:459-476, 2002).

An example of a PEG molecule which may be used is methoxy-PEG2-MAL-40K, a bifurcated PEG maleimide (Nektar, Huntsville, Ala.). Other examples include, but are not limited to bulk mPEG-SBA-20K (Nelctar) and mPEG2-ALD-40K (Nektar).

Carboxy-terminal attachment of PEG may be attached via enzymatic coupling using recombinant VPAC2 receptor peptide agonist as a precursor or alternative methods known in the art and described, for example, in U.S. Pat. No. 4,343,898 or Intl. J. Pept. & Prot. Res. 43:127-38 (1994).

One method for preparing the PEGylated VPAC2 receptor peptide agonists of the present invention involves the use of PEG-maleimide to directly attach PEG to a thiol group of the peptide. The introduction of a thiol functionality can be achieved by adding or inserting a Cys or hC residue onto or into the peptide at positions described above. A thiol functionality can also be introduced onto the side-chain of the peptide (e.g. acylation of lysine ε-amino group by a thiol-containing acid, such as mercaptopropionic acid). A PEGylation process of the present invention utilizes Michael addition to form a stable thioether linker. The reaction is highly specific and takes place under mild conditions in the presence of other functional groups. PEG maleimide has been used as a reactive polymer for preparing well-defined, bioactive PEG-protein conjugates. It is preferable that the procedure uses a molar excess, preferably from 1 to 10 molar excess, of a thiol-containing VPAC2 receptor peptide agonist relative to PEG maleimide to drive the reaction to completion. The reactions are preferably performed between pH 4.0 and 9.0 at room temperature for 10 minutes to 40 hours. The excess of unPEGylated thiol-containing peptide is readily separated from the PEGylated product by conventional separation methods. The PEGylated VPAC2 receptor peptide agonist is preferably isolated using reverse-phase HPLC or size exclusion chromatography. Specific conditions required for PEGylation of VPAC2 receptor peptide agonists are set forth in Example 7. Cysteine PEGylation may be performed using PEG maleimide or bifurcated PEG maleimide.

An alternative method for preparing the PEGylated VPAC2 receptor peptide agonists of the invention, involves PEGylating a lysine residue using a PEG-succinimidyl derivative. In order to achieve site specific PEGylation, the Lys residues which are not used for PEGylation are substituted for Arg residues.

Another approach for PEGylation is via Pictet Spengler reaction. A Trp residue with its free amine is needed to incorporate the PEG molecule onto a VPAC2 receptor selective peptide. One approach to achieve this is to site specifically introduce a Trp residue onto the amine of a Lys sidechain via an amide bond during the solid phase synthesis (see Example 9).

Various preferred features and embodiments of the present invention will now be described with reference to the following non-limiting examples.

### EXAMPLE 1

### Preparation of the Selective VPAC2 Receptor Peptide Agonists by Solid Phase t-Boc Chemistry

Selective VPAC2 receptor peptide agonists may be prepared using the following method and then PEGylating using one of the methods described in Examples 7, 8 and 9.

Approximately 0.5-0.6 grams (0.38-0.45 mmole) Boc Ser(Bzl)-PAM resin is placed in a standard 60 mL reaction vessel. Double couplings are run on an Applied Biosystems ABI430A peptide synthesizer. The following side-chain protected amino acids (2 mmole cartridges of Boc amino acids) are obtained from Midwest Biotech (Fishers, Ind.) and are used in the synthesis:

Arg-Tosyl (TOS), Asp-δ-cyclohexyl ester (OcHx), Glu-δ-cyclohexyl ester (OcHx), His-benzyloxymethyl(BOM), Lys-2-chlorobenzyloxycarbonyl (2Cl-Z), Ser-O-benzyl ether (OBzl), Thr-O-benzyl ether (OBzl), Trp-formyl (CHO) and Tyr-2-bromobenzyloxycarbonyl (2Br-Z) and Boc Gly PAM resin. Trifluoroacetic acid (TFA), di-isopropylethylamine (DIEA), 0.5 M hydroxybenzotriazole (HOBt) in DMF and 0.5 M dicyclohexylcarbodiimide (DCC) in dichloromethane are purchased from PE-Applied Biosystems (Foster City, Calif.). Dimethylformamide (DMF-Burdick and Jackson) and dichloromethane (DCM-Mallinkrodt) is purchased from Mays Chemical Co. (Indianapolis, Ind.).

Standard double couplings are run using either symmetric anhydride or HOBt esters, both formed using DCC. At the completion of the syntheses, the N-terminal Boc group is removed and the peptidyl resins are treated with 20% piperidine in DMF to deformylate the Trp side chain if Trp is present in the sequence. For the N-terminal acylation, four-fold excess of symmetric anhydride of the corresponding acid is added onto the peptide resin. The symmetric anhydride is prepared by diisopropyicarbodiimde (DIC) activation in DCM. The reaction is allowed to proceed for 4 hours and monitored by ninhydrin test. After washing with DCM, the resins are transferred to a TEFLON reaction vessel and are dried in vacuo.

Cleavages are done by attaching the reaction vessels to a HF (hydrofluoric acid) apparatus (Penninsula Laboratories). 1 mL m-cresol per gram/resin is added and 10 mL HF (purchased from AGA, Indianapolis, Ind.) is condensed into the pre-cooled vessel. 1 mL DMS per gram resin is added when methionine is present. The reactions are stirred one hour in an ice bath. The HF is removed in vacuo. The residues are suspended in ethyl ether. The solids are filtered and are washed with ether. Each peptide is extracted into aqueous acetic acid and either is freeze dried or is loaded directly onto a reverse-phase column.

Purifications are run on a 2.2×25 cm VYDAC C18 column in buffer A (0.1% Trifluoroacteic acid in water, B: 0.1% TFA in acetonitrile). A gradient of 20% to 90% B is run on an HPLC (Waters) over 120 minutes at 10 mL/minute while monitoring the UV at 280 nm (4.0 A) and collecting one minute fractions. Appropriate fractions are combined, frozen and lyophilized. Dried products are analyzed by HPLC (0.46×15 cm METASIL AQ C18) and MALDI mass spectrometry.

### EXAMPLE 2

### Preparation of the Selective VPAC2 Receptor Peptide Agonists by Solid Phase FMoc Chemistry

Selective VPAC2 receptor peptide agonists may be prepared using the following method and then PEGylating using one of the methods described in Examples 7, 8 and 9.

Approximately 114 mg (50 mMole) FMOC Ser(tBu) WANG resin (purchased from GlycoPep, Chicago, Ill.) is placed in each reaction vessel. The synthesis is conducted on a Rainin Symphony Peptide Synthesizer. Analogs with a C-terminal amide are prepared using 75 mg (50 μmole) Rink Amide AM resin (Rapp Polymere. Tuebingen, Germany).

The following FMOC amino acids are purchased from GlycoPep (Chicago, Ill.), and NovaBiochem (La Jolla, Calif.): Arg-2,2,4,6,7-pentamethyldihydrobenzofuran-5-sulfonyl (Pbf). Asn-trityl (Trt), Asp-B-t-Butyl ester (tBu), Glu-ε-t-butyl ester (tBu), Glu trityl (Trt), His-trityl (Trt), Lys-t-butyloxycarbonyl (Boc), Ser-t-butyl ether (OtBu), Thr-t-butyl ether (OtBu), Trp-t-butyloxycarbonyl (Boc), Tyr-t-butyl ether (OtBu).

Solvents dimethylformamide (DMF-Burdick and Jackson), N-methylpyrrolidone (NMP-Burdick and Jackson), dichloromethane (DCM-Mallinkrodt) are purchased from Mays Chemical Co. (Indianapolis, Ind.).

Hydroxybenzotrizole (HOBt), di-isopropylcarbodiimde (DIC), di-isopropylethylamine (DIEA), and piperidine (Pip) are purchased from Aldrich Chemical Co (Milwaukee, Wis.).

All amino acids are dissolved in 0.3 M in DMF. Three hour DIC/HOBt activated couplings are run after 20 minutes deprotection using 20% Piperidine/DMF. Each resin is washed with DMF after deprotections and couplings. After the last coupling and deprotection, the peptidyl resins are washed with DCM and are dried in vacuo in the reaction vessel. For the N-terminal acylation, four-fold excess of symmetric anhydride of the corresponding acid is added onto the peptide resin. The symmetric anhydride is prepared by diisopropylcarbodiimde (DIC) activation in DCM. The reaction is allowed to proceed for 4 hours and monitored by ninhydrin test. The peptide resin is then washed with DCM and dried in vacuo.

The cleavage reaction is mixed for 2 hours with a cleavage cocktail consisting of 0.2 mL thioanisole, 0.2 mL methanol, 0.4 mL triisopropylsilane, per 10 mL trifluoroacetic acid (TFA), all purchased from Aldrich Chemical Co., Milwaukee, Wis. If Cys is present in the sequence, 2% of ethanedithiol is added. The TFA filtrates are added to 40 mL ethyl ether. The precipitants are centrifuged 2 minutes at 2000 rpm. The supernatants are decanted. The pellets are resuspended in 40 mL ether, re-centrifuged, re-decanted, dried under nitrogen and then in vacuo.

0.3-0.6 mg of each product is dissolved in 1 mL 0.1% TFA/acetonitrile(ACN), with 20 μL being analyzed on HPLC [0.46×15 cm METASIL AQ C18, 1 mL/min, 45 C.°, 214 nM (0.2 A), A=0.1% TFA, B=0.1% TFA/50% ACN. Gradient=50% B to 90% B over 30 minutes].

Purifications are run on a 2.2×25 cm VYDAC C18 column in buffer A (0.1% trifluoroacteic acid in water, B: 0.1% TFA in acetonitrile). A gradient of 20% to 90% B is on an HPLC (Waters) over 120 minutes at 10 mL/minute while monitoring the UV at 280 nm (4.0 A) and collecting 1 minute fractions. Appropriate fractions are combined, frozen and lyophilized. Dried products are analyzed by HPLC (0.46×15 cm METASIL AQ C18) and MALDI mass spectrometry.

### EXAMPLE 3 In Vitro Potency

Alpha screen: Cells are washed in the culture flask once with PBS. The cells are then rinsed with enzyme free dissociation buffer. The dissociated cells are removed. The cells are then spun down and washed in stimulation buffer. For each data point, 50,000 cells suspended in stimulation buffer are used. To this buffer, Alpha screen acceptor beads are added along with the stimuli. This mixture is incubated for 60 minutes. Lysis buffer and Alpha screen donor beads are added and are incubated for 60 to 120 minutes. The Alpha screen signal (indicative of intracellular cAMP levels) is read in a suitable instrument (e.g. AlphaQuest from Perkin-Elmer). Steps including Alpha screen donor and acceptor beads are performed in reduced light. The EC₅₀ for cAMP generation is calculated from the raw signal or is based on absolute cAMP levels as determined by a standard curve performed on each plate.

Results for each agonist are, at minimum, from two analyses performed in a single run. For some agonists, the results are the mean of more than one run. The tested peptide concentrations are: 10000, 1000, 100, 10, 3, 1, 0.1, 0.01, 0.003, 0.001, 0.0001 and 0.00001 nM.

The activity (EC₅₀ (nM)) for the human VPAC2 receptor is reported in Table 1.

TABLE 1
Human VPAC2-R mediated cAMP generation (EC50; nM)

| | |
|---|---|
| VIP | 1.00 |
| PACAP-27 | 2.33 |
| P137 | 8.31 |
| P190 | 2.14 |
| P217 | >3000 |
| P219 | 25.46 |
| P245 | 450.14 |
| P246 | 398.31 |
| P247 | 132.87 |
| P283 | 31.71 |
| P286 | 6.65 |
| P300 | 6.62 |
| P328 | 6.61 |
| P331 | 9.74 |
| P337 | 7.57 |
| P340 | 7.74 |
| P373 | 33.86 |
| P378 | 37.35 |
| P380 | 12.07 |
| P399 | 9.76 |
| P404 | 17.01 |
| P411 | 25.23 |
| P413 | 28.37 |
| P415 | 13.38 |
| P420 | 19.21 |
| P426 | 47.66 |
| P428 | 61.06 |
| P430 | 50.50 |
| P432 | 20.10 |
| P434 | 29.04 |
| P436 | >10000 |
| P438 | 37.54 |
| P443 | 26.08 |
| P447 | 84.14 |
| P449 | 94.12 |
| P452 | 24.51 |
| P456 | 25.22 |
| P461 | 6.73 |
| P462 | 94.31 |
| P466 | >10000 |

EC₅₀ values given are single results or the mean of two or more independent runs.

### EXAMPLE 4

### Selectivity

Binding assays: Membrane prepared from a stable VPAC2 cell line (see Example 3) or from cells transiently transfected with human VPAC1 or PAC1 are used. A filter binding assay is performed using 125I-labeled VIP for VPAC1 and VPAC2 and 125I-labeled PACAP-27 for PAC1 as the tracers.

For this assay, the solutions and equipment include:

Presoak solution: 0.5% Polyethyleneamine in Aqua dest.

Buffer for flushing filter plates: 25 mM HEPES pH 7.4

Blocking buffer: 25 mM HEPES pH 7.4; 0.2% protease free BSA

Assay buffer: 25 mM HEPES pH 7.4; 0.5% protease free BSA

Dilution and assay plate: PS-Microplate, U form

Filtration Plate Multiscreen FB Opaque Plate; 1.0 μM Type B Glasfiber filter

In order to prepare the filter plates, the presoak solution is aspirated by vacuum filtration. The plates are flushed twice with 200 μL flush buffer. 200 μL blocking buffer is added to the filter plate. The filter plate is then incubated with 200 μL presoak solution for 1 hour at room temperature.

The assay plate is filled with 25 mL assay buffer, 25 mL membranes (2.5 μg) suspended in assay buffer, 25 μL compound (agonist) in assay buffer, and 25 μL tracer (about 40000 cpm) in assay buffer. The filled plate is incubated for 1 hour with shaking.

The transfer from assay plate to filter plate is conducted. The blocking buffer is aspirated by vacuum filtration and washed two times with flush buffer. 90 μL is transferred from the assay plate to the filter plate. The 90 μL transferred from assay plate is aspirated and washed three times with 200 μL flush buffer. The plastic support is removed. It is dried for 1 hour at 60° C. 30 μL Microscint is added. The count is performed.

The selectivity (IC₅₀) for human VPAC2, VPAC1, and PAC1 is reported in Table 2. Values reported are single results or the mean of two or more independent runs.

TABLE 2
Human receptor binding (IC50; nM)

| Agonist # | VPAC2 | VPAC1 | PAC1 |
|---|---|---|---|
| PACAP-27 | 2.52 | 4.0 | 9.5 |
| VIP | 5.06 | 3.3 | >1000 |
| P137 | 0.64 | 852.7 | >25000 |
| P190 | 4.44 | >3000 | >25000 |
| P219 | 67.92 | >25000 | n.d. |
| P245 | 920.53 | >25000 | n.d. |
| P246 | >1000 | >25000 | n.d. |
| P247 | 352.78 | >25000 | n.d. |
| P283 | 17.69 | >3000 | >25000 |
| P286 | 0.46 | >3000 | >25000 |
| P300 | 1.76 | >3000 | >25000 |
| P328 | 4.54 | >3000 | >25000 |
| P331 | 15.09 | >3000 | >25000 |
| P337 | 5.07 | >3000 | >25000 |
| P340 | 4.76 | >3000 | >25000 |
| P373 | 111.29 | >25000 | >25000 |
| P378 | 95.59 | n.d. | >25000 |
| P380 | 15.86 | >3000 | >25000 |
| P399 | 10.06 | >3000 | >25000 |
| P404 | 13.1 | >3000 | >25000 |
| P411 | 49.1 | n.d. | >25000 |
| P413 | 31.22 | >3000 | >25000 |
| P415 | 14.28 | >3000 | >25000 |
| P420 | 48.26 | >3000 | >25000 |
| P426 | 38.07 | >3000 | >25000 |
| P428 | 76.53 | >3000 | >25000 |
| P430 | 49.70 | >3000 | >25000 |
| P432 | 130.00 | >3000 | >25000 |
| P434 | 14.24 | >3000 | >25000 |
| P436 | >100 | >3000 | >25000 |
| P438 | 56.78 | >3000 | >25000 |
| P443 | 13.93 | >3000 | >25000 |
| P447 | 13.95 | >3000 | >25000 |
| P449 | >100 | >3000 | >25000 |
| P452 | 93.87 | >3000 | >25000 |
| P456 | 217.34 | >3000 | >25000 |
| P461 | 7.42 | >3000 | >25000 |
| P462 | 42.33 | >3000 | >25000 |
| P466 | >100 | >3000 | >25000 |

n.d. = Not determined

Rat receptor selectivity is estimated by comparing functional potency (cAMP generation) in CHO-PO cells transiently expressing rat VPAC1 or rat VPAC2 receptors. CHO-PO cells transiently expressing rat VPAC1 or VPAC2, are seeded with 10,000 cells/well three days before the assay. The cells are kept in 200 μL culture medium. On the day of the experiment, the medium is removed and the cells are washed twice. The cells are incubated in assay buffer plus IBMX for 15 minutes at room temperature. Afterwards, the stimuli are added and are dissolved in assay buffer. The stimuli are present for 30 minutes. The assay buffer is then gently removed. The cell lysis reagent of the DiscoveRx cAMP kit is added. Thereafter, the standard protocol for developing the cAMP signal as described by the manufacturer is used (DiscoveRx Inc., USA). EC₅₀ values for cAMP generation are calculated from the raw signal or are based on absolute cAMP levels as determined by a standard curve performed on each plate.

Results for each agonist are the mean of two independent runs. The typically tested concentrations of peptide are: 1000, 300, 100, 10, 1, 0.3, 0.1, 0.01, 0.001, 0.0001 and 0 nM.

TABLE 3
Rat VPAC1 and VPAC2 In vitro potency (cAMP generation).
CHO-PO cells are transiently transfected with rat VPAC1
or VPAC2 receptor DNA. The activity (EC₅₀ in nM) for
these receptors is reported in the table below.

| Agonist # | rVPAC1 | rVPAC2 |
|---|---|---|
| VIP | 0.015 | 0.67 |
| PACAP-27 | 0.07 | n.d. |
| P137 | 29.7 | 8.0 |
| P300 | n.d. | 5.8 |
| P328 | 212.5 | 7.5 |
| P337 | 114.2 | 5.6 |
| P340 | 116.5 | 4.0 |
| P373 | 349.6 | 72.2 |
| P378 | 529.8 | 119.4 |
| P380 | >1000 | 66.4 |
| P399 | 170.6 | 18.4 |
| P404 | 23.4 | 24.4 |
| P411 | 266.4 | 55.3 |
| P413 | 219.5 | 71.1 |
| P415 | 47.6 | 9.8 |
| P420 | >1000 | 35.6 |
| P426 | >200 | 56.8 |
| P428 | >1000 | 77.2 |
| P430 | 315.0 | 49.9 |
| P432 | >100 | 16.7 |
| P434 | >500 | 39.8 |
| P436 | >1000 | >1000 |
| P438 | >200 | 50.8 |
| P443 | 304.9 | 28.4 |
| P447 | >1000 | 144.7 |
| P449 | 421.4 | 123.7 |
| P452 | 189.2 | 38.6 |
| P456 | >1000 | 80.2 |
| P461 | 90.4 | 5.6 |
| P462 | >1000 | 119.5 |
| P466 | >1000 | 12.4 |

EC₅₀ values given are from one single determination or the mean of two or more independent EC₅₀ determinations.

### EXAMPLE 5

### In Vivo Assays

Intravenous glucose tolerance test (IVGTT): Normal Wistar rats are fasted overnight and are anesthetized prior to the experiment. A blood sampling catheter is inserted into the rats. The compound is given in the jugular vein. Blood samples are taken from the carotid artery. A blood sample is drawn immediately prior to the injection of glucose along with the compound. After the initial blood sample, glucose mixed with compound is injected intravenously (i.v.). Compound may also be injected intravenously or subcutaneously prior to the glucose challenge. A glucose challenge of 0.5 g/kg body weight is given, injecting a total of 1.5 mL vehicle with glucose and agonist per kg body weight. The peptide concentrations are varied to produce the desired dose in μg/kg. Blood samples are drawn at 2, 4, 6 and 10 minutes after giving glucose. The control group of animals receives the same vehicle along with glucose, but with no compound added. In some instances, a 30 minute post-glucose blood sample is drawn. Aprotinin is added to the blood sample (250-500 kIU/ml blood). The serum is then analyzed for glucose and insulin using standard methodologies.

The assay uses a formulated and calibrated peptide stock in PBS. Normally, this stock is a prediluted 100 μM stock. However, a more concentrated stock with approximately 1 mg agonist per 1 mL is used. The specific concentration is always known. Variability in the maximal response is mostly due to variability in the vehicle dose. Protocol details are as follows:

| | |
|---|---|
| SPECIES/STRAIN/WEIGHT | Rat/Wistar Unilever/approximately |
| | 275-300 g |
| TREATMENT DURATION | Single dose |
| DOSE VOLUME/ROUTE | 1.5 mL/kg/iv |
| VEHICLE | 8% PEG300, 0.1% BSA in water |
| FOOD/WATER REGIMEN | Rats are fasted overnight prior to |
| | surgery. |
| LIVE-PHASE PARAMETERS | Animals are sacrificed at the end of |
| | the test. |
| IVGTT: Performed on rats | Glucose IV bolus: 500 mg/kg as 10% |
| (with two catheters, | solution (5 mL/kg) at time = 0. |
| jugular vein and carotid | Compound iv: 0-240 min prior to |
| artery) of each group, | glucose Blood samplings (300 μL from |
| under pentobarbital | carotid artery; EDTA as anticoagulant; |
| anesthesia. | aprotinin and PMSF as antiproteolytics; |
| | kept on ice): 0, 2, 4, 6, and 10 minutes. |
| | Parameter determined: Insulin. |
| TOXICOKINETICS | Plasma samples remaining after insulin |
| | measurements are kept at −20° C. and |
| | compound levels are determined. |

TABLE 4a

| | Time | % increase | % increase | % increase | |
|---|---|---|---|---|---|
| | between | AUC: | AUC: | AUC: | |
| | glucose & | Dose = | Dose = | Dose = | IVGTT |
| Peptide | compound | 10 μg/kg | 30 μg/kg | 100 μg/kg | (ED₅₀; μg/kg) |
| P137 | 0 h | +366 | n.d | n.d. | n.d. |
| P137 | 4 h | +193 | +321 | +487 | n.d. |
| P190 | 0 h | +73 | n.d. | n.d. | n.d. |
| P461 | 24 h* | n.d. | n.d. | +71 | n.d. |

*Compound given subcutaneously
AUC = Area under curve (insulin, 0-10 min after glucose)

Glucose lowering in diabetic ZDF rats. ZDF rats, 8-9 weeks old with fed glucose levels of approximately 300 mg/dl are used for this experiment. The animals are randomised into control (vehicle) and treatment group(s) on the day of the experiment and are conscious throughout the experiment. The compound is injected intravenously at the start of the experiment and blood samples are drawn from the tail vein immediately prior to compound injection and then 0.5, 1, 2, 3, 4 and 24 h after compound injection. The animals are deprived of food during the first 2 or 4 h of the experiment. The blood samples are collected in EDTA tubes, aprotinin added and immediately put on ice pending insulin and glucose analysis using standard methods.

TABLE 4b
Glucose lowering in conscious food-deprived ZDF rats

| | | Time after | | | | | | |
|---|---|---|---|---|---|---|---|---|
| P137 | | injection | | | | | | |
| (μg/kg) | Analyte | 0 h | 0.5 h | 1 h | 2 h | 3 h | 4 h | 24 h |
| vehicle | glucose | 343 | 284 | 289 | 305 | n.d. | 317 | 339 |
| | (mg/dl) | | | | | | | |
| vehicle | insulin | 12.6 | 12.8 | 13.5 | 13.4 | n.d. | 17.9 | 15.3 |
| | (ng/ml) | | | | | | | |
| 10 | glucose | 344 | 277 | 244 | 199 | n.d. | 361 | 336 |
| | (mg/dl) | | | | | | | |
| 10 | insulin | 12.5 | 21.2 | 18.5 | 20.0 | n.d. | 18.5 | 15.0 |
| | (ng/ml) | | | | | | | |
| 30 | glucose | 329 | 254 | 218 | 156 | n.d. | 347 | 347 |
| | (mg/dl) | | | | | | | |
| 30 | insulin | 10.9 | 27.0 | 23.7 | 23.1 | n.d. | 20.8 | 16.0 |
| | (ng/ml) | | | | | | | |
| Vehicle | glucose | 298 | n.d. | 332 | 253 | 219 | 215 | 366 |
| | (mg/dl) | | | | | | | |
| Vehicle | insulin | 17.1 | n.d. | 13.0 | 10.5 | 9.3 | [symbol] .6 | 10.3 |
| | (ng/ml) | | | | | | | |
| 100 | glucose | 307 | n.d. | 362 | 360 | 323 | 304 | 400 |
| | (mg/dl) | | | | | | | |
| 100 | insulin | 17.8 | n.d. | 19.9 | 15.9 | 15.4 | 15.1 | 11.1 |
| | (ng/ml) | | | | | | | |

Animals are given access to food after the 2 h timepoint (10 & 30 μg/kg) or after the 4 h time point (100 μg/kg). [symbol]
indicates data missing or illegible when filed

Pharmacokinetic profiles of PEGylated peptides. Healthy Fisher 344 rats (3 animals per group) are injected with 100 μg compound/kg (compound amount based on peptide content and dissolved in PBS buffer). Blood samples are drawn 3, 12, 24, 48, 72, 96 and 168 hour post dosing and the peptide content in plasma is analysed by a radio-immunoassay (RIA) directed against the N-terminus of the peptide. PK parameters are then calculated using a model-independent method (WinNonlin Pro, Pharsight Corp., Mountain View, Calif., USA).

TABLE 4c
PK parameters of PEGylated compounds.
Mean and (SD) values for N = 3.

| | Cmax | Tmax | AUC₀₋ₗₐₛₜ | T½ | Cl/F | Vd/F |
|---|---|---|---|---|---|---|
| Compound | (ng/mL) | (h) | (ng*h/mL) | (h) | (mL/h/kg) | (mL/kg) |
| P217 | 247 | 20 | 13086 | 49 | 7 | 421 |
| | (140) | (7) | (6079) | (17) | (5) | (110) |
| P300 | 46 | 10 | 1343 | 15 | 72 | 1557 |
| | (16) | (3) | (245) | (2) | (12) | (359) |
| P328 | 94 | 12 | 3244 | 19 | 31 | 803 |
| | (21) | (0) | (500) | (2) | (5) | (32) |
| P337 | 122 | 13 | 3531 | 13 | 31 | 574 |
| | (52) | (11) | (1385) | (1) | (12) | (270) |
| P340 | 44 | 6 | 515 | NC* | NC | NC |
| | (27) | (5) | (189) | NC | NC | NC |
| P373 | 240 | 13 | 10803 | 31 | 10 | 424 |
| | (159) | (11) | (2919) | (2) | (2) | (99) |
| P399 | 200 | 16 | 7066 | 15 | 14 | 308 |
| | (45) | (7) | (1136) | (2) | (2) | (53) |
| P404 | 133 | 20 | 6070 | 23 | 17 | 556 |
| | (68) | (7) | (2814) | (4) | (6) | (207) |
| P430 | 159 | 20 | 10122 | 35 | 10 | 500 |
| | (31) | (7) | (1848) | (6) | (2) | (166) |
| P432 | 392 | 16 | 21471 | 35 | 5 | 229 |
| | (160) | (7) | (5061) | (9) | (1) | (23) |
| P447 | 239 | 16 | 11727 | 28 | 8 | 328 |
| | (71) | (7) | (2293) | (3) | (1) | (27) |

*NC = not calculated due to insufficient data

### EXAMPLE 6

### Rat Serum Stability Studies

In order to determine the stability of VPAC2 receptor peptide agonists in rat serum, obtain CHO-VPAC2 cells clone #6 (96 well plates/50,000 cells/well and 1 day culture), PBS 1× (Gibco), the peptides for the analysis in a 100 μM stock solution, rat serum from a sacrificed normal Wistar rat, aprotinin, and a DiscoveRx assay kit. The rat serum is stored at 4° C. until use and is used within two weeks.

On Day 0, two 100 μL aliquots of 10 μM peptide in rat serum are prepared by adding 10 μL peptide stock to 90 μL rat serum for each aliquot. 250 kIU aprotinin/mL is added to one of these aliquots. The aliquot is stored with aprotinin at 4° C. The aliquot is stored without aprotinin at 37° C. The aliquots are incubated for 18 hours.

On Day 1, after incubation of the aliquots prepared on day 0 for 24 hours, an incubation buffer containing PBS+1.3 mM CaCl₂, 1.2 mM MgCl₂, 2 mM glucose, and 0.25 mM IBMX is prepared. A plate with 11 serial 5× dilutions of peptide for the 4° C. and 37° C. aliquot is prepared for each peptide studied. 2000 nM is used as the maximal concentration if the peptide has an EC₅₀ above 1 nM and 1000 nM as maximal concentration if the peptide has an EC₅₀ below 1 nM from the primary screen (see Example 3). The plate(s) are washed with cells twice in incubation buffer. The plates are allowed to hold 50 μL incubation media per well for 15 minutes. 50 μL solution per well is transferred to the cells from the plate prepared with 11 serial 5× dilutions of peptide for the 4° C. and 37° C. aliquot for each peptide studied, using the maximal concentrations that are indicated by the primary screen, in duplicate. This step dilutes the peptide concentration by a factor of two. The cells are incubated at room temperature for 30 minutes. The supernatant is removed. 40 μL/well of the DiscoveRx antibody/extraction buffer is added. The cells are incubated on the shaker (300 rpm) for 1 hour. Normal procedure with the DiscoveRx kit is followed. cAMP standards are included in column 12. EC₅₀ values are determined from the cAMP assay data. The remaining amount of active peptide is estimated by the formula EC₅₀, 4C/EC_{50, 37C} for each condition.

TABLE 5
Estimated peptide stability after 24 h in rat serum at 37 C.

| Agonist # | Estimated serum stability (%)¹ |
|---|---|
| P137 | 114.8 |
| P219 | 56.5 |
| P300 | 161.9 |
| P306 | 142.4 |
| P328 | 374.2 |
| P373 | 73.6 |
| P380 | 299.7 |
| P399 | 204.1 |
| P404 | 320.7 |
| P415 | 220.9 |
| P420 | 104.6 |
| P426 | 46.9 |
| P428 | 68.3 |
| P430 | 54.9 |
| P432 | 102.1 |
| P434 | 61.9 |
| P436 | 71.6 |
| P438 | 31.2 |
| P443 | 74.9 |
| P449 | 59.7 |
| P452 | 62.8 |
| P456 | 55.5 |
| P461 | 67.3 |
| P466 | 777.0 |

¹Values >100% may represent release of intact peptide from the PEG conjugate

TABLE 6
Estimated peptide stability after 72 h in rat serum at 37 C.

| Compound | |
|---|---|
| # | Estimated 72 h stability (%)¹ |
| P137 | 52.8 |
| P219 | 45.6 |
| P247 | 16.7 |
| P286 | 60.5 |
| P328 | 348.0 |
| P337 | 158.1 |
| P373 | 47.1 |

¹Values >100% may represent release of intact peptide from the PEG conjugate

### EXAMPLE 7

### PEGylation of Selective VPAC2 Receptor Peptide Agonists Using Thiol-Based Chemistry

PEGylation reactions are run under conditions that permit the formation of a thioether bond. Specifically, the pH of the solution ranges from about 4 to 9 and the thiol-containing peptide concentrations range from 1 to 10 molar excess of methoxy-PEG2-MAL concentration. The PEGylation reactions are normally run at room temperature. The PEGylated VPAC2 receptor peptide agonist is then isolated using reverse-phase HPLC or size exclusion chromatography (SEC). PEGylated peptide analogues are characterized using analytical RP-HPLC, HPLC-SEC, SDS-PAGE, and/or MALDI Mass Spectrometry.

Usually a thiol function is introduced into or onto a selective VPAC2 receptor peptide agonist by adding a cysteine or a homocysteine or a thiol-containing moiety at either or both termini or by inserting a cysteine or a homocysteine or a thiol-containing moiety into the sequence. Thiol-containing VPAC2 receptor peptide agonists are reacted with 40 kDa polyethylene glycol-maleimide (PEG-maleimide) to produce derivatives with PEG covalently attached via a thioether bond. For example, P164, [CH₃—(CH₂)₄—CO-HSDAVFTDNYTRLLAKLALQKYLQSIKNKRYGGPSSGAPPPC, 42 AA, 23 mg, 4.8 umol], is dissolved in 4 mL of 200 mM phosphate buffer containing 20 mM EDTA, pH 7.5. The solution is then purged with argon. To this solution is added 230 mg of methoxy-PEG2-MAL-40K, a bifurcated PEG maleimide (Lot# PT-06D-01, Nektar, Huntsville, Ala.) (1:1 ratio of PEG to peptide). The reaction is performed for 2 hours. Then 86 mg of the PEGylated peptide (P190) is obtained after preparative RP-HPLC. The peptide conjugate is characterized by size-exclusion HPLC, and tested for in vitro activity.

Starting from P120, [CH₃—(CH₂)₄—CO-HSDAVFTDNYTRLRKQVAAKKYLQSIKNKRYGGPSSGAPPPC, 42 AA, 22 mg, 4.6 umol] and 200 mg of methoxy-PEG2-MAL-40K, 131.4 mg of PEGylated material (P137) is obtained according to the procedure described above.

Using the same method, 65 mg of PEGylated conjugate (P201) is obtained by reacting 11.3 mg of P200, [CH₃—(CH₂)₄—CO—HSDAVFTENY(OMe)TKLRKQNleAAKKYLNDLKKGGPSSGAPPPC,

[symbol]

2.6 umol] with 98 mg of methoxy-PEG2-MAL-40K).

20 mg of P327, [CH₃—(CH₂)₄—CO—HSDAVFTDNYTOrnLRAibQVAAAibKYLQSIOmNOrnGGPSSGAPPPC-NH₂] is dissolved in 3 ml of 100 mM NH₄Ac buffer containing 10 mM EDTA, pH 6.8. The solution is purged with argon. 175 mg of methoxy-PEG2-MAL-40K is added to the solution. The reaction is performed for 1 hour. 106 mg of the PEGylated peptide (P138) is isolated by preparative RP-HPLC, characterised by SE-HPLC, and tested for in-vitro activity.

### EXAMPLE 8

### PEGylation Via Acylation on the Sidechain of Lysine

In order to achieve site-specific PEGylation of selective VPAC2 receptor peptide agonists, all the Lys residues are changed into Arg residues except for these Lys residues where PEGylation is intended. For example, the following peptides are used for single or dual site PEGylation: CH₃—(CH₂)₄—CO-HSDAVFTDNYTRLRKQVAAKRYLQSIRNGGPSSGAPPPS (P213), CH₃—(CH₂)₄—CO-HSDAVFTDNYTRLRKQVAARRYLQSIRNGGPSSGAPPPS (P214), CH₃—(CH₂)₄—CO-HSDAVFTDNYTRLRRQVAAKRYLQSIRNGGPSSGAPPPS (P215), CH₃—(CH₂)₄—CO—HSDAVFTDNYTRLRRQVAARKYLQSIRNGGPSSGAPPPS (P216).

For the PEGylation of P213, the peptide is dissolved in 200 mM sodium borate buffer at pH 8.5 and a 1.5-fold molar excess of bulk mPEG-SBA-20K (Nektar, Lot#: PT-04E-11) is added (see scheme below). The reaction is allowed to stir at room temperature for 2-3 hours and then purified by preparative HPLC.

### EXAMPLE 9

### PEGylation Via Pictet-Spengler Reaction

For PEGylation via Pictet-Spengler reaction to occur, a Trp residue with its free amine is needed to incorporate the PEG molecule onto the selective VPAC2 receptor peptide agonist. One approach to achieve this is to add a Lys residue onto the C-terminus of the peptide and then to couple a Trp residue onto the sidechain of Lys. The extensive SAR indicates that this modification does not change the properties of the parent peptide in terms of its in vitro potency and selectivity.

PEG with a functional aldehyde, for example mPEG2-ALD-40K (Nektar, Lot #: PT-6C-05), is used for the reaction. The site specific PEGylation involves the formation a tetracarboline ring between PEG and the peptide. PEGylation is conducted in glacial acetic acid at room temperature for 1 to 48 hours. A 1 to 10 molar excess of the PEG aldehyde is used in the reaction. After the removal of acetic acid, the PEGylation VPAC2 receptor peptide agonist is isolated by preparative RP-HPLC.

Other modifications of the present invention will be apparent to those skilled in the art without departing from the scope of the invention.

1-48. (canceled)

49. A PEGylated VPAC2 receptor peptide agonist, comprising the amino acid sequence shown in SEQ ID NO: 28:
His-Ser-Xaa₃-Ala-Val-Phe-Thr-Xaa₈-Xaa₉-Xaa₁₀-Thr-
Xaa₁₂-Xaa₁₃-Xaa₁₄-Xaa₁₅-Xaa₁₆-Xaa₁₇-Ala-Xaa₁₉-
Xaa₂₀-Xaa₂₁-Xaa₂₂-Leu-Xaa₂₄-Xaa₂₅-Xaa₂₆-Xaa₂₇-
Xaa₂₈-Xaa₂₉-Xaa₃₀-Xaa₃₁-Xaa₃₂
wherein:
Xaa₃ is: Asp, or Glu;
Xaa₈ is: Asp, or Glu;
Xaa₉ is: Asn, Gln, or Cys;
Xaa₁₀ is: Tyr, or Tyr(OMe);
Xaa₁₂ is: Arg, Orn, or hR;
Xaa₁₃ is: Leu, Cys, or K(CO(CH₂)₂SH);
Xaa₁₄ is: Arg, Leu, or Aib;
Xaa₁₅ is: Lys, Ala, Arg, Aib, or K(W);
Xaa₁₆ is: Gln, Lys, K(CO(CH₂)₂SH), or Cys;
Xaa₁₇ is: Val, Leu, Cys, or K(CO(CH₂)₂SH);
Xaa₁₈ is: Ala, Leu, Cys, or K(CO(CH₂)₂SH);
Xaa₂₀ is: Lys, Gln, Arg, Aib, or Cys;
Xaa₂₁ is: Lys, Arg, Aib, or Orn;
Xaa₂₂ is: Tyr, or Tyr(OMe);
Xaa₂₄ is: Gln, Cys, or K(CO(CH₂)₂SH);
Xaa₂₅ is: Ser, Cys, or K(CO(CH₂)₂SH);
Xaa₂₆ is: Ile, Cys, or K(CO(CH₂)₂SH);
Xaa₂₇ is: Lys, Arg, Orn, or hR;
Xaa₂₈ is: Asn, hR, Orn, Cys, or K(CO(CH₂)₂SH);
Xaa₂₉ is: Orn, Lys, hR, or is absent;
Xaa₃₀ is: Arg, hR, or is absent;
Xaa₃₁ is: Tyr, or is absent; and
Xaa₃₂ is: Cys, or is absent,
provided that if Xaa₂₉, Xaa₃₀, or Xaa₃₁ is absent, the next amino acid present downstream is the next amino acid in SEQ ID NO: 28, and
a C-terminal extension, wherein the N-terminus of said C-terminal extension is linked to the C-terminus of said peptide of SEQ ID NO: 28, wherein said C-terminal extension is selected from the group consisting of GGPSSGAPPPS (SEQ ID NO: 10), GGPSSGAPPPS-NH₂ (SEQ ID NO: 11), GGPSSGAPPPC (SEQ ID NO: 22), GGPSSGAPPPC-NH₂ (SEQ ID NO: 23), GRPSSGAPPPS (SEQ ID NO: 16), and GRPSSGAPPPS-NH₂ (SEQ ID NO: 17), and wherein:
at least one of the Cys residues in said peptide of SEQ ID NO: 28 is covalently attached to a PEG molecule, or
at least one of the Lys residues in said peptide of SEQ ID NO: 28 is covalently attached to a PEG molecule, or
at least one of the K(CO(CH₂)₂SH) in said peptide of SEQ ID NO: 28 is covalently attached to a PEG molecule, or
the K(W) in said peptide of SEQ ID NO: 28 is covalently attached to a PEG molecule, or
the carboxy-terminal amino acid of said VPAC2 receptor peptide agonist is covalently attached to a PEG molecule, or
a combination thereof, or
a pharmaceutically acceptable salt thereof.

50. The PEGylated VPAC2 receptor peptide agonist according to claim 49 wherein said PEG molecule is branched.

51. The PEGylated VPAC2 receptor peptide agonist according to claim 49, wherein said PEG molecule is linear.

52. The PEGylated VPAC2 receptor peptide agonist according to claim 49, wherein said PEG molecule is 20,000, 40,000 or 60,000 daltons in molecular weight.

53. The PEGylated VPAC2 receptor peptide agonist according to claim 49, wherein two PEG molecules are present, and each of said PEG molecules is 20,000 daltons in molecular weight.

54. The PEGylated VPAC2 receptor peptide agonist according to claim 49, further comprising an N-terminal modification, wherein said N-terminal modification is the addition of a group selected from the group consisting of acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid, and —C(═NH)—NH₂.

55. The PEGylated VPAC2 receptor peptide agonist according to claim 54, wherein said N-terminal modification is the addition of acetyl or hexanoyl.

56. The PEGylated VPAC2 receptor peptide agonist according to claim 49, comprising the amino acid sequence shown in SEQ ID NO: 362:
hexanoyl-HSDAVFTEQY(OMe)TOrnLRAibQVAAAibOrn-
YLQSIOrnOrnGGPSSGAPPPC(PEG40K)-NH_{2.}

57. A pharmaceutical composition, comprising a PEGylated VPAC2 receptor peptide agonist according to claim 49, or a pharmaceutically acceptable salt thereof, and a pharmaceutically acceptable carrier, diluent or excipient.

58. A method of treating non-insulin-dependent diabetes or insulin-dependent diabetes in a mammal in need thereof, comprising administering to said mammal an effective amount of a PEGylated VPAC2 receptor peptide agonist according to claim 49.

59. The method of claim 58, wherein said mammal is a human.
